(19) United States
(12) Patent Application Publication
(10) Pub. No.: US 2024/0041929 A1
(43) Pub. Date: Feb. 8, 2024

(54) CHIMERIC ANTIGEN RECEPTORS SPECIFIC FOR GPRC5D AND BCMA

(71) Applicant: Juno Therapeutics, Inc., Seattle, WA (US)

(72) Inventors: Brian Joshua BELMONT, Seattle, WA (US); Kimberly HARRINGTON, Seattle, WA (US); Cyr Clovis Chua DE IMUS, Kenmore, WA (US); Jon Christopher JONES, Burien, WA (US); Eric William JEFFERY, Seattle, WA (US); Yeonjoo OH, Seattle, WA (US); Heather STRIEGEL, Everett, WA (US)

(73) Assignee: Juno Therapeutics, Inc., Seattle, WA (US)

(21) Appl. No.: 18/365,950

(22) Filed: Aug. 4, 2023

Related U.S. Application Data
(60) Provisional application No. 63/395,702, filed on Aug. 5, 2022.

(51) Int. Cl. A61K 35/17; C07K 16/28; C12N 15/86; A61K 39/00; A61P 35/00; A61K 31/7076; A61K 31/675; A61K 31/4184

(52) U.S. Cl. CPC A61K 35/17; C07K 16/2878; C12N 15/86; A61K 39/4611; A61K 39/4631; A61K 39/464417; A61P 35/00; A61K 31/7076; A61K 31/675; A61K 31/4184; A61K 2239/38; A61K 2239/22; A61K 2239/21; A61K 2239/17; A61K 2239/13; A61K 2239/29; A61K 2239/48

(57) ABSTRACT

Provided are chimeric antigen receptors (CARs), which contain extracellular antigen-binding domains that bind to G Protein-Coupled Receptor Class C Group 5 Member D (GPRC5D) and B-cell maturation antigen (BCMA). The disclosure further relates to genetically engineered cells expressing such CARs, and uses thereof in adoptive cell therapy.

### CROSS-REFERENCE TO RELATED APPLICATIONS

This application claims priority from U.S. provisional application No. 63/395,702, filed Aug. 5, 2022, entitled “CHIMERIC ANTIGEN RECEPTORS SPECIFIC FOR GPRC5D AND BCMA,” the contents of which are incorporated by reference in their entirety.

### FIELD

The present disclosure relates in some aspects to chimeric antigen receptors (CARs), which contain extracellular antigen-binding domains that bind to G Protein-Coupled Receptor Class C Group 5 Member D (GPRC5D) and B-cell maturation antigen (BCMA). The disclosure further relates to genetically engineered cells expressing such CARs, and uses thereof in adoptive cell therapy.

### INCORPORATION BY REFERENCE OF SEQUENCE LISTING

The present application is being filed along with a Sequence Listing in electronic format. The Sequence Listing is provided as a file entitled 735042026300SeqList.xml, created Aug. 4, 2023, which is 224,174 bytes in size. The information in the electronic format of the Sequence Listing is incorporated by reference in its entirety.

### BACKGROUND

G-protein coupled receptor class C group 5 member D (GPRC5D) is a G-protein coupled receptor, which is highly expressed in bone marrow samples of patients with multiple myeloma (MM) compared to the minimal expression of GPRC5D in bone marrow samples of patients with other hematological malignancies. B-cell maturation antigen (BCMA) is a transmembrane type III protein expressed on mature B lymphocytes. Various GPRC5D-binding chimeric antigen receptors (CARs), BCMA-binding CARs, and cells expressing such CARs, are available. However, there remains a need for improved CARs binding both GPRC5D and BCMA, and engineered cells expressing the same, such as for use in adoptive cell therapy. Provided herein are embodiments that meet such needs.

### SUMMARY

Provided herein are bispecific chimeric antigen receptors (CARs) comprising an extracellular domain comprising a GPRC5D-binding domain that binds to GPRC5D and a BCMA-binding domain that binds to BCMA.

Also provided herein is a bispecific chimeric antigen receptor (CAR) comprising: (a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus: (i) one of the VH region and the VL region of the GPRC5D-binding domain, one of the VH region and the VL region of the BCMA-binding domain, the other of the VH region and the VL region of the BCMA-binding domain, and the other of the VH region and the VL region of the GPRC5D-binding domain; or (ii) one of the VH region and the VL region of the BCMA-binding domain, one of the VH region and the VL region of the GPRC5D-binding domain, the other of the VH region and the VL region of the GPRC5D-binding domain, and the other of the VH region and the VL region of the BCMA-binding domain; (b) a spacer; (c) a transmembrane domain; and (d) an intracellular signaling domain.

In some embodiments, the extracellular domain comprises, in order from amino to carboxy terminus, (i). In some embodiments, the extracellular domain comprises, in order from amino to carboxy terminus: the VH region of the GPRC5D-binding domain, the VH region of the BCMA-binding domain, the VL region of the BCMA-binding domain, and the VL region of the GPRC5D-binding domain.

Also provided herein is a bispecific chimeric antigen receptor (CAR) comprising: (a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus: the VH region of the GPRC5D-binding domain, the VH region of the BCMA-binding domain, the VL region of the BCMA-binding domain, and the VL region of the GPRC5D-binding domain; (b) a spacer; (c) a transmembrane domain; and (d) an intracellular signaling domain.

In some embodiments, the extracellular domain comprises, in order from amino to carboxy terminus: the VH region of the GPRC5D-binding domain, the VL region of the BCMA-binding domain, the VH region of the BCMA-binding domain, and the VL region of the GPRC5D-binding domain.

Also provided herein is a bispecific chimeric antigen receptor (CAR) comprising: (a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus: the VH region of the GPRC5D-binding domain, the VL region of the BCMA-binding domain, the VH region of the BCMA-binding domain, and the VL region of the GPRC5D-binding domain; (b) a spacer; (c) a transmembrane domain; and (d) an intracellular signaling domain.

In some embodiments, the extracellular domain comprises, in order from amino to carboxy terminus: the VL region of the GPRC5D-binding domain, the VH region of the BCMA-binding domain, the VL region of the BCMA-binding domain, and the VH region of the GPRC5D-binding domain.

Also provided herein is a bispecific chimeric antigen receptor (CAR) comprising: (a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VL region of the GPRC5D-binding domain, the VH region of the BCMA-binding domain, the VL region of the BCMA-binding domain, and the VH region of the GPRC5D-binding domain; (b) a spacer; (c) a transmembrane domain; and (d) an intracellular signaling domain.

In some embodiments, the extracellular domain comprises in order from amino to carboxy terminus: the VL region of the GPRC5D-binding domain, the VL region of the BCMA-binding domain, the VH region of the BCMA-binding domain, and the VH region of the GPRC5D-binding domain.

Also provided herein is a bispecific chimeric antigen receptor (CAR) comprising: (a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VL region of the GPRC5D-binding domain, the VL region of the BCMA-binding domain, the VH region of the BCMA-binding domain, and the VH region of the GPRC5D-binding domain; (b) a spacer; (c) a transmembrane domain; and (d) an intracellular signaling domain.

In some embodiments, the extracellular domain comprises, in order from amino to carboxy terminus, (ii). In some embodiments, the extracellular domain comprises in order from amino to carboxy terminus: the VH region of the BCMA-binding domain, the VH region of the GPRC5D-binding domain, the VL region of the GPRC5D-binding domain, and the VL region of the BCMA-binding domain.

Also provided herein is a bispecific chimeric antigen receptor (CAR), comprising: (a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VH region of the BCMA-binding domain, the VH region of the GPRC5D-binding domain, the VL region of the GPRC5D-binding domain, and the VL region of the BCMA-binding domain; (b) a spacer; (c) a transmembrane domain; and (d) an intracellular signaling domain.

In some embodiments, the extracellular domain comprises in order from amino to carboxy terminus: the VH region of the BCMA-binding domain, the VL region of the GPRC5D-binding domain, the VH region of the GPRC5D-binding domain, and the VL region of the BCMA-binding domain.

Also provided herein is a bispecific chimeric antigen receptor (CAR), comprising: (a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VH region of the BCMA-binding domain, the VL region of the GPRC5D-binding domain, the VH region of the GPRC5D-binding domain, and the VL region of the BCMA-binding domain; (b) a spacer; (c) a transmembrane domain; and (d) an intracellular signaling domain.

In some embodiments, the extracellular domain comprises in order from amino to carboxy terminus: the VL region of the BCMA-binding domain, the VH region of the GPRC5D-binding domain, the VL region of the GPRC5D-binding domain, and the VH region of the BCMA-binding domain.

Also provided herein is a bispecific chimeric antigen receptor comprising: (a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VL region of the BCMA-binding domain, the VH region of the GPRC5D-binding domain, the VL region of the GPRC5D-binding domain, and the VH region of the BCMA-binding domain; (b) a spacer; (c) a transmembrane domain; and (d) an intracellular signaling domain.

In some embodiments, the extracellular domain comprises in order from amino to carboxy terminus: the VL region of the BCMA-binding domain, the VL region of the GPRC5D-binding domain, the VH region of the GPRC5D-binding domain, and the VH region of the BCMA-binding domain.

Also provided herein is a bispecific chimeric antigen receptor (CAR) comprising: (a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VL region of the BCMA-binding domain, the VL region of the GPRC5D-binding domain, the VH region of the GPRC5D-binding domain, and the VH region of the BCMA-binding domain; (b) a spacer; (c) a transmembrane domain; and (d) an intracellular signaling domain.

In some embodiments, (a) the VH region or the VL region of the GPRC5D-binding domain; and (b) the VH region or the VL region of the BCMA-binding domain are joined by a linker.

In some embodiments, the linker is a flexible peptide linker. In some embodiments, the linker is 4 to 12 amino acids in length. In some embodiments, the linker is or comprises the amino acid sequence set forth in SEQ ID NO:19, SEQ ID NO:21, or SEQ ID NO:22. In some embodiments, the linker is or comprises the amino acid sequence set forth in SEQ ID NO:19. In some embodiments, the linker is or comprises the amino acid sequence set forth in SEQ ID NO:21. In some embodiments, the linker is or comprises the amino acid sequence set forth in SEQ ID NO:22.

In some embodiments, (a) the VH region and the VL region of the GPRC5D-binding domain are joined by a linker; or (b) the VH region and the VL region of the BCMA-binding domain are joined by a linker. In some embodiments, the VH region and the VL region of the GPRC5D-binding domain are joined by a linker. In some embodiments, the VH region and the VL region of the BCMA-binding domain are joined by a linker.

In some embodiments, the linker comprises the amino acid sequence set forth in SEQ ID NO:17 or SEQ ID NO:18. In some embodiments, the linker comprises the amino acid sequence set forth in SEQ ID NO:17. In some embodiments, the linker comprises the amino acid sequence set forth in SEQ ID NO:18.

Also provided herein is a bispecific chimeric antigen receptor (CAR) comprising: (a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus: (i) the VH region of the GPRC5D-binding domain; (ii) the linker set forth in SEQ ID NO:21; (iii) the VL region of the BCMA-binding domain; (iv) the linker set forth in SEQ ID NO:17; (v) the VH region of the BCMA-binding domain; (vi) the linker set forth in SEQ ID NO:21; and (vii) the VL region of the GPRC5D-binding domain; (b) a spacer; (c) a transmembrane domain; and (d) an intracellular signaling domain.

Also provided herein is a bispecific chimeric antigen receptor (CAR) comprising: (a) an extracellular domain comprising (i) a GPRC5D-binding domain that binds to GPRC5D comprising a heavy chain variable (VH) region and a light chain variable (VL) region; and (ii) a BCMA-binding domain that binds to BCMA comprising a VH region and a VL region, wherein the extracellular domain comprises, in order from amino to carboxy terminus: one of the VH region and the VL region of the BCMA-binding domain; the other of the VH region and the VL region of the BCMA-binding domain; one of the VH region and the VL region of the GPRC5D-binding domain; and the other of the VH region and the VL region of the GPRC5D-binding domain; (b) a spacer; (c) a transmembrane domain; and (d) an intracellular signaling domain.

Also provided herein is a bispecific chimeric antigen receptor (CAR) comprising: (a) an extracellular domain comprising (i) a GPRC5D-binding domain that binds to GPRC5D comprising a heavy chain variable (VH) region and a light chain variable (VL) region; and (ii) a BCMA-binding domain that binds to BCMA comprising a VH region and a VL region, wherein the extracellular domain comprises, in order from amino to carboxy terminus: the VL region of the GPRC5D-binding domain; the VH region of the GPRC5D-binding domain; one of the VH region and the VL region of the BCMA-binding domain; and the other of the VH and the VL region of the BCMA-binding domain; (b) a spacer; (c) a transmembrane domain; and (d) an intracellular signaling domain.

In some embodiments, the GPRC5D-binding region and the BCMA-binding region are joined by a linker. In some embodiments, the linker is a flexible peptide linker. In some embodiments, the linker is 4 to 12 amino acids in length. In some embodiments, the linker comprises the amino acid sequence set forth in SEQ ID NO:19, SEQ ID NO:21, or SEQ ID NO:24. In some embodiments, the linker comprises the amino acid sequence set forth in SEQ ID NO:19. In some embodiments, the linker comprises the amino acid sequence set forth in SEQ ID NO:21. In some embodiments, the linker comprises the amino acid sequence set forth in SEQ ID NO:24. In some embodiments, the VH region and the VL region of the BCMA-binding domain are joined by a linker comprising the amino acid sequence set forth in SEQ ID NO:17.

Also provided herein is a bispecific chimeric antigen receptor (CAR) comprising: (a) an extracellular domain comprising (i) a GPRC5D-binding domain that binds to GPRC5D comprising a heavy chain variable (VH) region and a light chain variable (VL) region; and (ii) a BCMA-binding domain that binds to BCMA comprising a VH region and a VL region, wherein the extracellular domain comprises in order from amino to carboxy terminus: the VH region of the GPRC5D-binding domain; the VL region of the GPRC5D-binding domain; one of the VH region and the VL region of the BCMA-binding domain; and the other of the VH and the VL region of the BCMA-binding domain; (b) a spacer; (c) a transmembrane domain; and (d) an intracellular signaling domain, wherein the GPRC5D-binding domain and the BCMA-binding domain are joined by a linker comprising the sequence set forth in SEQ ID NO:19 or SEQ ID NO:21.

In some embodiments, the VH region of the GPRC5D-binding domain comprises a CDR-1, a CDR-2, and a CDR-3 comprising the amino acid sequences set forth in SEQ ID NO:1, SEQ ID NO:2, and SEQ ID NO:3, respectively. In some embodiments, the VL region of the GPRC5D-binding domain comprises a CDR-1, a CDR-2, and a CDR-3 comprising the amino acid sequences set forth in SEQ ID NO:4, SEQ ID NO:5, and SEQ ID NO:6, respectively. In some embodiments, the VH region of the GPRC5D-binding domain comprises a CDR-1, a CDR-2, and a CDR-3 comprising the amino acid sequences set forth in SEQ ID NO:1, SEQ ID NO:2, and SEQ ID NO:3, respectively; and the VL region of the GPRC5D-binding domain comprises a CDR-1, a CDR-2, and a CDR-3 comprising the amino acid sequences set forth in SEQ ID NO:4, SEQ ID NO:5, and SEQ ID NO:6, respectively. In some embodiments, the VH region of the GPRC5D-binding domain an amino acid sequence having at least about 90% sequence identity to the amino acid sequence set forth in SEQ ID NO:7. In some embodiments, the VL region of the GPRC5D-binding domain comprises an amino acid sequence having at least about 90% sequence identity to the amino acid sequence set forth in SEQ ID NO:8. In some embodiments, the VH region of the GPRC5D-binding domain comprises an amino acid sequence having at least about 90% sequence identity to the amino acid sequence set forth in SEQ ID NO:7; and the VL region of the GPRC5D-binding domain comprises an amino acid sequence having at least about 90% sequence identity to the amino acid sequence set forth in SEQ ID NO:8. In some embodiments, the VH region of the GPRC5D-binding domain comprises the amino acid sequence set forth in SEQ ID NO:7. In some embodiments, the VL region of the GPRC5D-binding domain comprises the amino acid sequence set forth in SEQ ID NO:8. In some embodiments, the VH region of the GPRC5D-binding domain comprises the amino acid sequence set forth in SEQ ID NO:7; and the VL region of the GPRC5D-binding domain comprises the amino acid sequence set forth in SEQ ID NO:8.

In some embodiments, the VH region of the BCMA-binding domain comprises a CDR-1, a CDR-2, and a CDR-3 comprising the amino acid sequences set forth in SEQ ID NO:9, SEQ ID NO:10, and SEQ ID NO:11, respectively. In some embodiments, the VL region of the BCMA-binding domain comprises a CDR-1, a CDR-2, and a CDR-3 comprising the amino acid sequences set forth in SEQ ID NO:12, SEQ ID NO:13, and SEQ ID NO: 14, respectively. In some embodiments, the VH region of the BCMA-binding domain comprises an amino acid sequence having at least about 90% sequence identity to the amino acid sequence set forth in SEQ ID NO:15. In some embodiments, the VL region of the BCMA-binding domain comprises an amino acid sequence having at least about 90% sequence identity to the amino acid sequence set forth in SEQ ID NO:16. In some embodiments, the VH region of the BCMA-binding domain comprises an amino acid sequence having at least about 90% sequence identity to the amino acid sequence set forth in SEQ ID NO:15; and the VL region of the BCMA-binding domain comprises an amino acid sequence having at least about 90% sequence identity to the amino acid sequence set forth in SEQ ID NO:16. In some embodiments, the VH region of the BCMA-binding domain comprises the amino acid sequence set forth in SEQ ID NO:15. In some embodiments, the VL region of the BCMA-binding domain comprises the amino acid sequence set forth in SEQ ID NO:16. In some embodiments, the VH region of the BCMA-binding domain comprises the amino acid sequence set forth in SEQ ID NO:15; and the VL region of the BCMA-binding domain comprises the amino acid sequence set forth in SEQ ID NO:16.

In some embodiments, the extracellular binding domain of the bispecific CAR comprises the amino acid sequence set forth in any one of SEQ ID NO:77, 78, 79, and 80. In some embodiments, the extracellular binding domain of the bispecific CAR comprises the amino acid sequence set forth in SEQ ID NO:81, 82, 83, 84, 85, 86, 87, 88, 89, and 90. In some embodiments, the extracellular binding domain of the bispecific CAR comprises the amino acid sequence set forth in SEQ ID NO: 83. In some embodiments, the extracellular binding domain of the bispecific CAR comprises the amino acid sequence set forth in SEQ ID NO: 84. In some embodiments, the extracellular binding domain of the bispecific CAR comprises the amino acid sequence set forth in SEQ ID NO: 87. In some embodiments, the extracellular binding domain of the bispecific CAR comprises the amino acid sequence set forth in SEQ ID NO: 81. In some embodiments, the extracellular binding domain of the bispecific CAR comprises the amino acid sequence set forth in SEQ ID NO: 85. In some embodiments, the extracellular binding domain of the bispecific CAR comprises the amino acid sequence set forth in SEQ ID NO: 86. In some embodiments, the extracellular binding domain of the bispecific CAR comprises the amino acid sequence set forth in SEQ ID NO: 90.

In some embodiments, the spacer comprises at least a portion of an immunoglobulin or a variant thereof. In some embodiments, the spacer comprises a hinge region of an immunoglobulin or a variant thereof. In some embodiments, the hinge region of an immunoglobulin is an IgG4 hinge region. In some embodiments, the hinge region comprises a human IgG4 hinge region, or a variant thereof.

In some embodiments, the spacer is less than at or about 15 amino acids in length. In some embodiments, the spacer is between 12 and 15 amino acids in length. In some embodiments, the spacer is about 12 amino acids in length. In some embodiments, the spacer is about 13 amino acids in length. In some embodiments, the spacer is about 14 amino acids in length. In some embodiments, the spacer is about 15 amino acids in length. In some embodiments, the spacer comprises the amino acid sequence set forth in SEQ ID NO:25, or an amino acid sequence having at least about 90% sequence identity to the amino acid sequence set forth in SEQ ID NO:25. In some embodiments, the spacer comprises the amino acid sequence set forth in SEQ ID NO:25. In some embodiments, the spacer comprises a CH3 region of an immunoglobulin. In some embodiments, the spacer is between about 100 and 125 amino acids in length. In some embodiments, the spacer is about 119 amino acids in length. In some embodiments, the spacer comprises the amino acid sequence set forth in SEQ ID NO:26, or an amino acid sequence having at least about 90% sequence identity to the amino acid sequence set forth in SEQ ID NO:26. In some embodiments, the spacer comprises the amino acid sequence set forth in SEQ ID NO:26. In some embodiments, the spacer is between 200 and 250 amino acids in length. In some embodiments, the spacer is between 220 and 240 amino acids in length. In some embodiments, the spacer comprises a hinge region of an immunoglobulin, a CH2 region of an immunoglobulin or a chimeric CH2 region of two different immunoglobulins, and a CH3 region of an immunoglobulin. In some embodiments, the spacer comprises IgG4 hinge region or a variant thereof, a chimeric CH2 region comprising a portion of an IgG4 CH2 and a portion of an IgG2 CH2 (IgG2/4 CH2 region), and an IgG4 CH3 region. In some embodiments, the spacer comprises the amino acid sequence set forth in SEQ ID NO:27, or an amino acid sequence having at least about 90% sequence identity to the amino acid sequence set forth in SEQ ID NO:27. In some embodiments, the spacer comprises the amino acid sequence set forth in SEQ ID NO:27.

In some embodiments, the transmembrane domain is or comprises a transmembrane domain from CD4, CD28, or CD8. In some embodiments, the transmembrane domain is or comprises a transmembrane domain from human CD4, human CD28 or human CD8. In some embodiments, the transmembrane domain is or comprises a transmembrane domain from human CD4. In some embodiments, the transmembrane domain is or comprises a transmembrane domain from human CD28. In some embodiments, the transmembrane domain is or comprises a transmembrane domain from human CD8. In some embodiments, the transmembrane domain comprises the amino acid sequence set forth in SEQ ID NO:28, or an amino acid sequence having at least about 90% sequence identity to the amino acid sequence set forth in SEQ ID NO:28. In some embodiments, the transmembrane domain comprises the amino acid sequence set forth in SEQ ID NO:28.

In some embodiments, the intracellular signaling domain is a domain from a T cell receptor (TCR) component or comprises an immunoreceptor tyrosine-based activation motif (ITAM). In some embodiments, the intracellular signaling domain comprises a cytoplasmic signaling domain of a CD3-zeta chain. In some embodiments, the intracellular signaling domain comprises a cytoplasmic signaling domain of a human CD3-zeta chain. In some embodiments, the intracellular signaling domain comprises the amino acid sequence set forth in SEQ ID NO:30, or an amino acid sequence having at least about 90% sequence identity to the amino acid sequence set forth in SEQ ID NO:30. In some embodiments, the intracellular signaling domain comprises the amino acid sequence set forth in SEQ ID NO:30. In some embodiments, the intracellular signaling domain further comprises a costimulatory signaling region. In some embodiments, the costimulatory signaling region is located between the transmembrane region and the intracellular signaling domain. In some embodiments, the costimulatory signaling region comprises an intracellular signaling domain of a T cell costimulatory molecule or a signaling portion thereof. In some embodiments, the costimulatory signaling region comprises an intracellular signaling domain of CD28, 4-1BB, or ICOS, or a signaling portion thereof. In some embodiments, the costimulatory signaling region comprises an intracellular signaling domain of human CD28, human 4-1BB, or human ICOS. In some embodiments, the costimulatory signaling region comprises an intracellular signaling domain of 4-1BB or a signaling portion thereof. In some embodiments, the costimulatory signaling region comprises an intracellular signaling domain of human 4-1BB. In some embodiments, the costimulatory signaling region comprises the amino acid sequence set forth in SEQ ID NO:29, or an amino acid sequence having at least about 90% sequence identity to the amino acid sequence set forth in SEQ ID NO:29. In some embodiments, the costimulatory signaling region comprises the amino acid sequence set forth in SEQ ID NO:29.

In some embodiments, the CAR comprises the amino acid sequence that has at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 98% sequence identity to any one of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, or SEQ ID NO:44.

In some embodiments, the CAR comprises the amino acid sequence set forth in SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, or SEQ ID NO:44. In some embodiments, the CAR comprises the amino acid sequence set forth in SEQ ID NO:31. In some embodiments, the CAR comprises the amino acid sequence set forth in SEQ ID NO:32. In some embodiments, the CAR comprises the amino acid sequence set forth in SEQ ID NO:33. In some embodiments, the CAR comprises the amino acid sequence set forth in SEQ ID NO:34. In some embodiments, the CAR comprises the amino acid sequence set forth in SEQ ID NO:35. In some embodiments, the CAR comprises the amino acid sequence set forth in SEQ ID NO:36. In some embodiments, the CAR comprises the amino acid sequence set forth in SEQ ID NO:37. In some embodiments, the CAR comprises the amino acid sequence set forth in SEQ ID NO:38. In some embodiments, the CAR comprises the amino acid sequence set forth in SEQ ID NO:39. In some embodiments, the CAR comprises the amino acid sequence set forth in SEQ ID NO:40. In some embodiments, the CAR comprises the amino acid sequence set forth in SEQ ID NO:41. In some embodiments, the CAR comprises the amino acid sequence set forth in SEQ ID NO:42. In some embodiments, the CAR comprises the amino acid sequence set forth in SEQ ID NO:43. In some embodiments, the CAR comprises the amino acid sequence set forth in SEQ ID NO:44.

Also provided herein is a bispecific chimeric antigen receptor (CAR) comprising: (a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus: (i) the VH region of the GPRC5D-binding domain comprising a CDR-1, a CDR-2, and a CDR-3 comprising the amino acid sequences set forth in SEQ ID NO:1, SEQ ID NO:2, and SEQ ID NO:3, respectively; (ii) the linker set forth in SEQ ID NO:21; (iii) the VL region of the BCMA-binding domain comprising a CDR-1, a CDR-2, and a CDR-3 comprising the amino acid sequences set forth in SEQ ID NO:12, SEQ ID NO:13, and SEQ ID NO:14, respectively; (iv) the linker set forth in SEQ ID NO:17; (v) the VH region of the BCMA-binding domain comprising a CDR-1, a CDR-2, and a CDR-3 comprising the amino acid sequences set forth in SEQ ID NO:9, SEQ ID NO:10, and SEQ ID NO:11, respectively; (vi) the linker set forth in SEQ ID NO:21; and (vii) the VL region of the GPRC5D-binding domain comprising a CDR-1, a CDR-2, and a CDR-3 comprising the amino acid sequences set forth in SEQ ID NO:4, SEQ ID NO:5, and SEQ ID NO:6, respectively; (b) a spacer comprising the amino acid sequence set forth in SEQ ID NO:27; (c) a transmembrane domain comprising the amino acid sequence set forth in SEQ ID NO:28; and (d) an intracellular signaling domain comprising the amino acid sequences set forth in SEQ ID NOS:29 and 30.

In some embodiments, the extracellular binding domain of the bispecific CAR comprises the amino acid sequence set forth in SEQ ID NO: 83. In some embodiments, the CAR comprises the amino acid sequence set forth in SEQ ID NO:37. In some embodiments, the bispecific CAR is encoded by the nucleotide sequence set forth in SEQ ID NO:119.

Also provided herein is a bispecific chimeric antigen receptor (CAR) comprising: (a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus: (i) the VL region of the BCMA-binding domain comprising a CDR-1, a CDR-2, and a CDR-3 comprising the amino acid sequences set forth in SEQ ID NO:12, SEQ ID NO:13, and SEQ ID NO:14, respectively; (ii) the linker set forth in SEQ ID NO:21; (iii) the VL region of the GPRC5D-binding domain comprising a CDR-1, a CDR-2, and a CDR-3 comprising the amino acid sequences set forth in SEQ ID NO:4, SEQ ID NO:5, and SEQ ID NO:6, respectively; (iv) the linker set forth in SEQ ID NO:17; (v) the VH region of the GPRC5D-binding domain comprising a CDR-1, a CDR-2, and a CDR-3 comprising the amino acid sequences set forth in SEQ ID NO:1, SEQ ID NO:2, and SEQ ID NO:3, respectively; (vi) the linker set forth in SEQ ID NO:21; and (vii) the VH region of the BCMA-binding domain comprising a CDR-1, a CDR-2, and a CDR-3 comprising the amino acid sequences set forth in SEQ ID NO:9, SEQ ID NO:10, and SEQ ID NO:11, respectively; (b) a spacer comprising the amino acid sequence set forth in SEQ ID NO:27; (c) a transmembrane domain comprising the amino acid sequence set forth in SEQ ID NO:28; and (d) an intracellular signaling domain comprising the amino acid sequences set forth in SEQ ID NOS:29 and 30.

In some embodiments, the extracellular binding domain of the bispecific CAR comprises the amino acid sequence set forth in SEQ ID NO: 86. In some embodiments, CAR comprises the amino acid sequence set forth in SEQ ID NO:40. In some embodiments, the bispecific CAR is encoded by the nucleotide sequence set forth in SEQ ID NO:120.

Also provided herein is a polynucleotide encoding any of the CARs provided herein. In some embodiments, the polynucleotide comprises the nucleotide sequence set forth in any one of SEQ ID NOS:105-120. Also provided herein is a polynucleotide comprising the nucleotide sequence set forth in any one of SEQ ID NOS:105-120. In some embodiments, the polynucleotide comprises the nucleotide sequence set forth in SEQ ID NO:5. In some embodiments, the polynucleotide comprises the nucleotide sequence set forth in SEQ ID NO:6. In some embodiments, the polynucleotide comprises the nucleotide sequence set forth in SEQ ID NO:7. In some embodiments, the polynucleotide comprises the nucleotide sequence set forth in SEQ ID NO:8. In some embodiments, the polynucleotide comprises the nucleotide sequence set forth in SEQ ID NO:9. In some embodiments, the polynucleotide comprises the nucleotide sequence set forth in SEQ ID NO:10. In some embodiments, the polynucleotide comprises the nucleotide sequence set forth in SEQ ID NO:11. In some embodiments, the polynucleotide comprises the nucleotide sequence set forth in SEQ ID NO:12. In some embodiments, the polynucleotide comprises the nucleotide sequence set forth in SEQ ID NO:13. In some embodiments, the polynucleotide comprises the nucleotide sequence set forth in SEQ ID NO:14. In some embodiments, the polynucleotide comprises the nucleotide sequence set forth in SEQ ID NO:15. In some embodiments, the polynucleotide comprises the nucleotide sequence set forth in SEQ ID NO:16. In some embodiments, the polynucleotide comprises the nucleotide sequence set forth in SEQ ID NO:17. In some embodiments, the polynucleotide comprises the nucleotide sequence set forth in SEQ ID NO:18. In some embodiments, the polynucleotide comprises the nucleotide sequence set forth in SEQ ID NO:19. In some embodiments, the polynucleotide comprises the nucleotide sequence set forth in SEQ ID NO:20. In some embodiments, the polynucleotide is optimized by splice site elimination. In some embodiments, the polynucleotide is codon-optimized for expression in a human cell. In some embodiments, the polynucleotide comprises the nucleotide sequence set forth in SEQ ID NO:119. In some embodiments, the polynucleotide comprises the nucleotide sequence set forth in SEQ ID NO:120.

Also provided herein is a vector comprising any of the polynucleotides provided herein. In some embodiments, the vector is a viral vector. In some embodiments, the vector is a retroviral vector. In some embodiments, the vector is a lentiviral vector or an adeno-associated viral (AAV) vector. In some embodiments, the vector is a lentiviral vector. In some embodiments, the vector is an adeno-associated viral (AAV) vector.

Also provided herein is a cell comprising any of the CARs provided herein.

Also provided herein is a cell comprising any of the polynucleotides provided herein.

Also provided herein is a cell comprising any of the vectors provided herein. In some embodiments, the cell is an immune cell. In some embodiments, the cell is a lymphocyte. In some embodiments, the cell is a NK cell or a T cell. In some embodiments, the cell is a T cell. In some embodiments, the T cell is a CD4+ T cell or a CD8+ T cell. In some embodiments, the T cell is a CD4+ T cell. In some embodiments, the T cell is a CD8+ T cell. In some embodiments, the T cell is a primary T cell. In some embodiments, the cell is a stem cell. In some embodiments, the stem cell is a multipotent and pluripotent stem cell. In some embodiments, the stem cell is an induced pluripotent stem cell (iPSC). In some embodiments, the cell has been differentiated from an induced pluripotent stem cell. In some embodiments, the cell is an allogeneic cell. In some embodiments, the cell is engineered to be hypoimmune.

In some embodiments, the cell exhibits cytotoxic activity against GPRC5D+ cells, BCMA+ cells, or GPRC5D+/BCMA+ cells. In some embodiments, the cell exhibits cytotoxic activity against GPRC5D+ cells. In some embodiments, the cell exhibits cytotoxic activity against BCMA+ cells. In some embodiments, the cell exhibits cytotoxic activity against GPRC5D+/BCMA+ cells. In some embodiments, the cell exhibits cytotoxic activity against GPRC5D+ cells, BCMA+ cells, and GPRC5D+/BCMA+ cells.

Also provided herein is a composition comprising a plurality of any of the cells provided herein. In some embodiments, the composition comprises a pharmaceutically acceptable excipient.

Also provided herein is a pharmaceutical composition comprising a plurality of any of the cells provided herein, and a pharmaceutically acceptable excipient.

In some embodiments, the composition comprises CD4+ T cells and CD8+ T cells. In some embodiments, the composition comprises a ratio of CD4+ T cells to CD8+ T cells that is between about 1:3 and about 3:1. In some embodiments, the composition comprises a ratio of CD4+ T cells to CD8+ T cells that is between about 1:2 and about 2:1. In some embodiments, the composition comprises a ratio of CD4+ T cells to CD8+ T cells that is about 1:1.

In some embodiments, greater than about 90%, greater than about 95% or greater than about 99% of cells in the composition are CD3+ T cells. In some embodiments, at least about 40%, at least about 50%, at least about 60%, at least about 70%, at least about 80%, or at least about 90% of cells in the composition express the CAR. In some embodiments, among a plurality of the cells in the composition expressing the CAR, less than about 10%, about 9%, about 8%, about 7%, about 5%, about 4%, about 3%, about 2%, or about 1% of the cells exhibit tonic signaling.

In some embodiments, the composition comprises between about 1.0×10⁷ CAR-expressing T cells and 1.2×10⁹ CAR-expressing T cells, between about 1.0×10⁷ CAR-expressing T cells and 6.5×108 CAR-expressing T cells, between about 1.5×10⁷ CAR-expressing T cells and 6.5×10⁸ CAR-expressing T cells, between about 1.5×10⁷ CAR-expressing T cells and 6.0×10⁸ CAR-expressing T cells, between about 2.5×10⁷ CAR-expressing T cells and 6.0×10⁸ CAR-expressing T cells, between about 5.0×10⁷ CAR-expressing T cells and 6.0×10⁸ CAR-expressing T cells, between about 1.25×10⁷ CAR-expressing T cells and 1.2×10⁹ CAR-expressing T cells, between about 1.5×10⁷ CAR-expressing T cells and 1.2×10⁹ CAR-expressing T cells, between about 5.0×10⁷ CAR-expressing T cells and 4.5×10⁸ CAR-expressing T cells, or between about 1.5×10⁸ CAR-expressing T cells and 3.0×10⁸ CAR-expressing T cells, each inclusive. In some embodiments, the composition comprises at or about 1.5×10⁷, at or about 2.5×10⁷, at or about 5.0×10⁷, at or about 7.5×10⁷, at or about 1.0×10⁸, at or about 1.25×10⁸, at or about 1.5×10⁸, at or about 1.75×10⁸, at or about 2×10⁸, at or about 2.25×10⁸, at or about 2.5×10⁸, at or about 3.0×10⁸, at or about 3.5×10⁸, at or about 4×10⁸, at or about 4.5×10⁸, at or about 6.0×10⁸, at or about 8.0×10⁸, or at or about 1.2×10⁹ CAR-expressing T cells.

Also provided herein is a method of treating a disease or condition comprising administering any of the cells provided herein to a subject. In some embodiments, the cell is administered to the subject at a dose of from at or about 1×10⁷ CAR-expressing T cells and 1×10⁹ CAR-expressing T cells. In some embodiments, the cell is administered to the subject at a dose of from or from about 2.5×10⁷ CAR-expressing T cells to about 4.5×10⁸ CAR-expressing T cells. In some embodiments, the cell is administered to the subject at a dose of or about 2.5×10⁷ CAR-expressing T cells. In some embodiments, the cell is administered to the subject at a dose of or about 7.5×10⁷ CAR-expressing T cells. In some embodiments, the cell is administered to the subject at a dose of or about 1.5×10⁸ CAR-expressing T cells. In some embodiments, the cell is administered to the subject at a dose of or about 3.0×10⁸ CAR-expressing T cells. In some embodiments, the cell is administered to the subject at a dose of or about 4.5×10⁸ CAR-expressing T cells.

In some embodiments, of claims 133-140, the method further comprises administering a lymphodepleting therapy to the subject prior to administration of the dose of the CAR-expressing T cells. In some embodiments, the lymphodepleting therapy is completed within about 7 days prior to initiation of the administration of the dose of the CAR-expressing T cells. In some embodiments, the administration of the lymphodepleting therapy is completed within about 2 to 7 days prior to initiation of the administration of the dose of engineered T cells. In some embodiments, the lymphodepleting therapy comprises the administration of fludarabine and/or cyclophosphamide. In some embodiments, the lymphodepleting therapy comprises the administration of fludarabine and cyclophosphamide. In some embodiments, the lymphodepleting therapy comprises administration of cyclophosphamide at or about 200-400 mg/m² inclusive daily. In some embodiments, the lyphodepleting therapy comprises administration of cyclophosphamide at or about 300 mg/m² daily. In some embodiments, the lyphodepleting therapy comprises administration of fludarabine at or about 20-40 mg/m² inclusive daily. In some embodiments, the lyphodepleting therapy comprises administration of fludarabine at or about 30 mg/m² daily. In some embodiments, the lyphodepleting therapy comprises administration of fludarabine and cyclophosphamide for 2-4 days. In some embodiments, the lyphodepleting therapy comprises administration of fludarabine and cyclophosphamide for 3 days.

In some embodiments, the lymphodepleting therapy comprises the administration of bendamustine. In some embodiments, the lymphodepleting therapy comprises administration of bendamustine at or about 50-130 mg/m² inclusive daily. In some embodiments, the lyphodepleting therapy comprises administration of bendamustine at or about 90 mg/m² daily. In some embodiments, the lyphodepleting therapy comprises administration of bendamustine for 1-3 days. In some embodiments, the lyphodepleting therapy comprises administration of bendamustine for 2 days.

Also provided herein is use of any of the cells provided herein for manufacture of a medicament for treating a disease or condition in a subject. Also provided herein is use of any of the cells provided herein for treatment of a disease or condition in a subject. Also provided herein is any of the cells provided herein for treatment of a disease or condition in a subject.

Also provided herein is a method of treating a disease or condition comprising administering any of the compositions provided herein to a subject. Also provided herein is use of any of the compositions provided herein for manufacture of a medicament for treating a disease or condition in a subject. Also provided herein is use of any of the compositions provided herein for treatment of a disease or condition in a subject. Also provided herein is any of the compositions provided herein for treatment of a disease or condition in a subject.

In some embodiments, the disease or condition is a cancer. In some embodiments, the disease or condition is a plasma cell malignancy. In some embodiments, the disease or condition is a BCMA-expressing cancer and/or a GPRC5D-expressing cancer. In some embodiments, the disease or condition is a BCMA-expressing cancer. In some embodiments, the disease or condition is a GPRC5D-expressing cancer. In some embodiments, the disease or condition is a BCMA-expressing cancer and a GPRC5D-expressing cancer. In some embodiments, the disease or condition is a multiple myeloma. In some embodiments, the disease or condition is a relapsed/refractory multiple myeloma.

In some embodiments, the subject has received one or more prior therapies. In some embodiments, the subject has received at least 1, but no more than 3, prior therapies. In some embodiments, the prior therapies are an proteasome inhibitor, an immumodulatory agent, an anti-CD38 antibody, a prior therapy that included autologous hematopoietic stem cell transplantation (HSCT), or a combination of any of the foregoing. In some embodiments, the cells or the composition may be used for the manufacture of a medicament for treating a disease or condition in a subject. In some embodiments, the cells or the composition may be used for treatment of a disease or condition in a subject. In some embodiments, the disease or condition is a cancer, optionally a plasma cell malignancy. In some embodiments, the disease or condition is a BCMA-expressing cancer and/or a GPRC5D-expressing cancer. In some embodiments, the disease or condition is a multiple myeloma. In some embodiments, the disease or condition is a relapsed/refractory multiple myeloma (RRMM).

In some embodiments, the subject has received one or more prior therapies. In some embodiments, the subject has received at least 1, but no more than 3, prior therapies. In some embodiments, the prior therapies are an proteasome inhibitor, an immumodulatory agent, an anti-CD38 antibody, a prior therapy that included autologous hematopoietic stem cell transplantation (HSCT), or a combination of any of the foregoing. In some embodiments, the cell or the composition may be for treatment of a disease or condition in a subject. In some embodiments, the disease or condition is a cancer, optionally a plasma cell malignancy. In some embodiments, the disease or condition is a BCMA-expressing cancer and/or a GPRC5D-expressing cancer. In some embodiments, the disease or condition is a multiple myeloma. In some embodiments, the disease or condition is a relapsed/refractory multiple myeloma (RRMM). In some embodiments, the subject has received one or more prior therapies. In some embodiments, the subject has received at least 1, but no more than 3, prior therapies. In some embodiments, the prior therapies are an proteasome inhibitor, an immumodulatory agent, an anti-CD38 antibody, a prior therapy that included autologous hematopoietic stem cell transplantation (HSCT), or a combination of any of the foregoing.

Also provided herein is a kit comprising any of the CARs, polynucleotides, vectors, cells, or compositions provided herein, and instructions for use. In some embodiments, the instructions are for administering the CAR, the cell, or the composition. In some embodiments, the instructions specify administering the CAR, the cell, or the composition to a subject having a disease or disorder.

Also provided herein is an article of manufacture comprising any of the CARs, polynucleotides, vectors, cells, compositions, or kits provided herein.

### BRIEF DESCRIPTION OF THE DRAWINGS

FIGS. 1A and 1B show the expression of human GPRC5D and human BCMA on various tumor cell lines as assessed by flow cytometry.

FIG. 2A shows structures of exemplary generated bispecific, linear tandem CARs targeting GPRC5D and BCMA having either the GPRC5D-binding domain (left panel) or the BCMA-binding domain (right panel) proximal to the cell membrane.

FIG. 2B shows structures of exemplary generated bispecific, loop tandem CARs targeting GPRC5D and BCMA having either the GPRC5D-binding domain (left panel) or the BCMA-binding domain (right panel) proximal to the cell membrane.

FIGS. 3A and 3B show antigen-independent (tonic) signaling (FIG. 3A) and antigen-dependent signaling (FIG. 3B), respectively, of Nurkat reporter cells expressing exemplary generated bispecific tandem CARs alone or following co-culture with target cells expressing GPRC5D and BCMA.

FIGS. 4A and 4B show antigen-dependent activation of Nurkat reporter cells expressing exemplary generated bispecific tandem CARs through a single antigen, following co-culture with MM.1S or OPM-2 cells, respectively, which were knocked out for GPRC5D or BCMA.

FIG. 5 shows the percentage of cells surface positive for expression of each CAR, as assessed by flow cytometry (designations of the top 14 tandem CAR constructs are indicated by numbers).

FIG. 6A shows the ability of T cells expressing the indicated tandem CAR constructs to lyse target cells (left to right: MM.1S, MM.1S BCMA KO, and MM.1S GPRC5D KO) following 21 days of co-culture. *CAR T cells failed to survive to day 21 against MM.1S BCMA KO cells. \hat{ }CAR T cells failed to survive to day 21 against MM.1S GPRC5D KO cells.

FIGS. 6B and 6C show the proliferation of CAR T cells expressing linear tandem CAR constructs (round dots), loop tandem CAR constructs (round dots), singly-targeting (GPRC5D or BCMA) CAR constructs (square and diamond, respectively), or the bicistronic CAR construct (triangle), following 7, 14, and 21 days of co-culture with MM.1 S cells knocked out for BCMA (FIG. 6B) or GPRC5D (FIG. 6C), respectively.

FIGS. 7A and 7B show individual plots of tumor burden through day 49 in a MM.1S mouse model of multiple myeloma, following treatment with a high (2×10⁶) (FIG. 7A) or low (0.5×10⁶) (FIG. 7B) dose of T cells expressing the indicated CARs, respectively.

FIGS. 8A and 8B show the tumor control index (TCI) through day 49 in a MM.1S mouse model of multiple myeloma, following treatment with a high (2×10⁶) or low (0.5×10⁶) dose of T cells expressing the indicated CARs, respectively.

FIGS. 9A and 9B show individual plots of tumor burden through day 49 in a RPMI-8226 mouse model of multiple myeloma, following treatment with a high (2×10⁶) (FIG. 9A) or low (0.5×10⁶) (FIG. 9B) dose of T cells expressing the indicated CARs, respectively.

FIGS. 10A and 10B show the tumor control index (TCI) through day 49 in a RPMI-8226 mouse model of multiple myeloma, following treatment with a high (2×10⁶) or low (0.5×10⁶) dose of T cells expressing the indicated CARs, respectively.

FIG. 11A shows individual plots of tumor burden through day 28 in a mouse model of multiple myeloma antigen heterogeneity, following treatment with a 4×10⁶ T cells expressing the indicated CARs (solid lines) or mock-processed T cells (dotted lines).

FIGS. 11B and 11C show tumor control index (TCI) and tumor burden by bioluminescent imaging (BLI), respectively, through day 28 in a mouse model of multiple myeloma antigen heterogeneity, following treatment with 4×10⁶ T cells expressing the indicated CARs.

FIGS. 12A and 12B shows expression of both the anti-GPRC5D scFv (y-axis) and anti-BCMA scFv (x-axis) in T cells from three human donors transduced with tandem CAR 5, an anti-BCMA CAR, or an anti-GPRC5D CAR, or mock transduced cells.

FIGS. 13A and 13B show proliferation and CD25 expression, respectively, of T cells transduced with bispecific tandem CAR 5, the anti-BCMA CAR, or the anti-GPRC5D CAR, or mock transduced T cells, following co-culture with various cell lines.

FIGS. 13C and 13D show secretion of IFNγ (FIG. 13C, top panel), IL-2 (FIG. 13C, bottom panel), and TNFα (FIG. 13D) by T cells transduced with bispecific tandem CAR 5, the anti-BCMA CAR, or the anti-GPRC5D CAR, or mock transduced T cells, following co-culture with various cell lines. Graphs show mean concentrations of pro-inflammatory cytokines and data points represent cytokine levels from individual donors.

FIG. 14A shows expression of CD25 by T cells transduced with bispecific tandem CAR 5, the anti-BCMA CAR, or the anti-GPRC5D CAR, or mock transduced T cells, following co-culture with various cell lines. Data points represent values from CAR T cells from individual donors.

FIGS. 14B and 14C show secretion of IFNγ, IL-2 and TNFα (left, middle, and right panel, respectively) by T cells transduced with bispecific tandem CAR 5 (FIG. 14B), the anti-BCMA CAR (FIG. 14B), or the anti-GPRC5D CAR (FIG. 14C), following co-culture with various cell lines. Graphs show mean concentrations of pro-inflammatory cytokines and data points represent values from individual donors.

FIG. 15 shows the cytotoxic activity of CAR 5, anti-BCMA CAR and anti-GPRC5D CAR T cells against tumor cell lines expressing variable levels of BCMA and GPRC5D. Data are plotted as mean and standard deviation across three donors.

FIG. 16A shows the number of CAR+ human CD3+ T cells per microliter peripheral blood in MM.1S xenograft mice treated with 5×10⁵ (low dose; left panel) or 2×10⁶ (high dose; right panel) CAR T cells.

FIG. 16B (top panels) shows the mean tumor volume for groups of MM.1S xenograft mice treated with 5×10⁵ (low dose; left panel) or 2×10⁶ (high dose; right panel) bispecific tandem CAR 5, anti-BCMA CAR, or anti-GPRC5D CAR T cells, or mock transduced T cells. FIG. 16B (bottom panels) shows the individual tumor volumes for MM.1S xenograft mice treated with 5×10⁵ (low dose; left panel) or 2×10⁶ (high dose; right panel) anti-GPRC5D CAR T cells or mock transduced T cells.

FIG. 16C (top panels) shows the individual tumor volumes for MM.1S xenograft mice treated with 5×10⁵ (low dose; left panel) or 2×10⁶ (high dose; right panel) anti-BCMA CAR T cells or mock transduced T cells. FIG. 16C (bottom panels) shows the individual tumor volumes for MM.1S xenograft mice treated with 5×10⁵ (low dose; left panel) or 2×10⁶ (high dose; right panel) bispecific tandem CAR 5 T cells or mock transduced T cells.

FIG. 16D shows the tumor control index for MM.1S xenograft mice treated with 5×10⁵ (low dose) or 2×10⁶ (high dose) tandem CAR 5, anti-BCMA CAR, or anti-GPRC5D CAR T cells, or mock transduced T cells.

FIG. 16E shows probability of survival for MM.1S xenograft mice treated with 5×10⁵ (low dose) or 2×10⁶ (high dose) tandem CAR 5, anti-BCMA CAR, or anti-GPRC5D CAR T cells, or mock transduced T cells.

FIG. 17A (top panels) shows the mean tumor burden for groups of OPM-2 xenograft mice treated with 5×10⁵ (low dose) or 2×10⁶ (high dose) tandem CAR 5, anti-BCMA CAR, or anti-GPRC5D CAR T cells, or mock transduced T cells. FIG. 17A (bottom panels) shows the individual tumor burden for OPM-2 xenograft mice treated with 5×10⁵ (low dose) or 2×10⁶ (high dose) anti-GPRC5D CAR T cells, or mock transduced T cells.

FIG. 17B (top panels) shows the individual tumor burden for OPM-2 xenograft mice treated with 5×10⁵ (low dose) or 2×10⁶ (high dose) anti-BCMA CAR T cells, or mock transduced T cells. FIG. 17B (bottom panels) shows the individual tumor burden for OPM-2 xenograft mice treated with 5×10⁵ (low dose) or 2×10⁶ (high dose) tandem CAR 5 T cells, or mock transduced T cells.

FIG. 17C shows the tumor control index for OPM-2 xenograft mice treated with 5×10⁵ (low dose) or 2×10⁶ (high dose) tandem CAR 5, anti-BCMA CAR, or anti-GPRC5D CAR T cells, or mock transduced T cells.

FIG. 17D shows the survival probability for OPM-2 xenograft mice treated with 5×10⁵ (low dose) or 2×10⁶ (high dose) tandem CAR 5, anti-BCMA CAR, or anti-GPRC5D CAR T cells, or mock transduced T cells.

### DETAILED DESCRIPTION

Provided herein are bispecific chimeric antigen receptors (CARs) (also referred to as “dual-targeting” CARs) targeting or directed to G Protein-Coupled Receptor Class C Group 5 Member D (GPRC5D) and B cell maturation antigen (BCMA). In some embodiments, the provided bispecific CARs target or are directed to GPRC5D- and/or BCMA-expressing cells and diseases. Also provided are cells, such as T cells, engineered to express a provided bispecific CAR and compositions containing such cells. It is observed that GPRC5D is expressed, e.g., heterogeneously expressed, in certain diseases and conditions such as malignancies, or on tissues or cells thereof, e.g., on malignant plasma cells such as from relapsed or newly diagnosed myeloma patients, for example, with little expression on normal tissues. Among the provided embodiments are approaches useful in the treatment of diseases and conditions and/or for targeting such cell types, including nucleic acid molecules that encode GPRC5D- and BCMA-binding domains, including chimeric antigen receptors (CARs), and the encoded receptors such as the encoded CARs, and compositions and articles of manufacture comprising the same. The receptors generally can contain antibodies (including antigen-binding antibody fragments, such as heavy chain variable (VH) regions, single domain antibody fragments and single chain fragments, including scFvs) specific for GPRC5D and BCMA. Also provided are cells, such as engineered or recombinant cells expressing such GPRC5D- and BCMA-binding receptors, e.g., bispecific CARs and/or containing nucleic acids encoding such receptors, and compositions and articles of manufacture and therapeutic doses containing such cells.

The provided embodiments relate to CAR T cells targeting both GPRC5D and BCMA for treatment of multiple myeloma. GPRC5D (Uniprot Acc. No. Q9NZD1, e.g. set forth in SEQ ID NO:59) is a G protein coupled receptor class C, group 5 member D that belongs to the RAIG (retinoic acid-inducible gene-1) family. It is a seven transmembrane helix 39 kDa G-protein coupled receptor with two reported isoforms, with the isoform differences occurring in the intracellular C terminus of the protein. Results herein show that GPRC5D is expressed at high levels in multiple myeloma and, overall, it is expressed at low levels in most normal tissues. BCMA (Uniprot Acc. No. Q02223, e.g. set forth in SEQ ID NO:60) is a transmembrane type III protein expressed on mature B lymphocytes. Following binding of BCMA to its ligands, B cell activator of the TNF family (BAFF) or a proliferation inducing ligand (APRIL), a pro-survival cell signal is delivered to the B cell which has been found to be required for plasma cell survival.

Multiple myeloma (MM) is a hematological malignancy characterized by uncontrolled proliferation of monoclonal plasma cells in the bone marrow resulting in the over-production of monoclonal immunoglobulin and immunosuppression (Al-Hujaily 2016; Dimopoulos, 2015). Adoptive T cell therapies, such as CAR-T cell therapies, have shown promise for treating multiple myeloma, with clinical efforts primarily focused on targeting the B cell maturation antigen (BCMA). Indeed, there have recently several advances in treatment options for MM including FDA approval of two chimeric antigen receptor (CAR) T cell therapy targeting B-cell maturation antigen (BCMA). However, although BCMA is expressed on many malignant plasma cells, expression levels, in some cases, can be heterogeneous. In some aspects, heterogeneity in target antigen expression can lead to variable or inconsistent response. In some aspects, it also has been observed that expression of BCMA on the cell surface varies over time due to gamma secretase-mediated shedding of the extracellular domain. While several clinical trials have demonstrated high overall response rates, most patients eventually relapse and diminished BCMA expression following CAR T cell therapy has been observed (Brudno et al. (2018) J. Clin. Oncol., JCO2018778084, Cohen et al. (2017) Blood 130:505). Targeting a second antigen in MM could overcome antigen downregulation or loss thus decreasing the opportunity for immune escape. For instance, both BCMA and GPRC5D are highly expressed in MM, but their expression is independent of one another making them a promising combination for dual targeting (Smith et al., Sci Transl Med (2019) 11(485):aau7746). Notably, the CARs provided herein do not demonstrate appreciable recombination (e.g., homologous recombination). By contrast, dual-targeting CARs formatted in a bicistronic arrangement to allow expression of two independent CARs from a single vector can exhibit unexpected or unwanted recombination due to high sequence homology among different portions of the vector (e.g., portions encoding the same or similar components of each independent CAR). Lam et al., Blood (2021) 138 (Suppl. 1):4808.

Also, in some contexts, recombinant receptors can exhibit antigen-independent activity or signaling (also known as “tonic signaling”), which could lead to undesirable effects, such as due to increased differentiation and/or exhaustion of T cells that express the recombinant receptor. In some aspects, such activities may limit the T cell's activity, effect or potency. In some cases, during engineering and ex vivo expansion of the cells for recombinant receptor expression, the cells may exhibit phenotypes indicative of exhaustion, due to tonic signaling through the recombinant receptor. In some cases, alternative or additional MM-targeted T cell therapy approaches are needed.

Among provided engineered cells are those that include chimeric antigen receptors that display high expression of both BCMA- and GPRC5D-binding domains, as well as low tonic signaling, thereby minimizing possibility of antigen-independent (tonic) signaling. In particular, the bispecific CARs provided herein include CARs with high antigen-dependent activation and minimal tonic signaling.

Provided are monotherapy approaches utilizing bispecific CARs targeting both GPRC5D and BCMA expressed on autologous primary T cells for use as a therapeutic agent against multiple myeloma plasma cells. In some embodiments, a monotherapy approach may be desirable in subjects known or suspected or selected as having low or heterogeneous BCMA-expressing MM plasma cells. It is observed that GPRC5D and BCMA are expressed, e.g., heterogeneously expressed, in certain diseases and conditions such as malignancies, or on tissues or cells thereof, e.g., on malignant plasma cells such as from relapsed or newly diagnosed myeloma patients, for example, with little or low expression on normal tissues. Due to the roles of GPRC5D and BCMA in various diseases and conditions, including cancer, both GPRC5D and BCMA are therapeutic targets.

In some cases, simultaneously targeting both antigens as provided herein may improve the depth and durability of responses across patients, in addition to minimizing relapse due to antigen escape. A mechanism of resistance to CAR T-cell therapies, as evidenced by data from CAR T-cell trials in B-cell malignancies, may be the loss or downregulation (“escape”) of the target antigen. (Robbie G. Majzner and Crystal L. Mackall, Cancer Discov Aug. 22, 2018; DOI 10.1158/2159-8290.CD-18-0442). Such a dual targeting strategy may achieve synergistic or improved tumor responses based on targeting two antigens compared to approaches involving only single antigen targeting. A dual targeting approach may be advantageous to overcome problems due to potential for antigen loss and/or to maximize antigen targeting in MM.

Further, the CARs provided herein displayed strong in vitro function against three different multiple myeloma cell lines, and robust in vivo efficacy across three different multiple myeloma models, evidencing their suitability in the presence of varied antigen levels, up to and including complete antigen loss. To this end, observations herein indicate that the provided CARs are highly functional when signaling through a single binding domain, consistent with an observation that the CARs would exhibit antitumor efficacy in the presence of only a single antigen (i.e. GPRC5D or BCMA), such as in the event of antigen loss.

Among the provided embodiments are approaches useful in the treatment of diseases and conditions and/or for targeting such cell types, including nucleic acid molecules that encode bispecific chimeric antigen receptors (CARs) that bind to both GPRC5D and BCMA, and the encoded receptors such as the encoded CARs, and compositions and articles of manufacture comprising the same. The receptors generally can contain antibodies (including antigen-binding antibody fragments, such as heavy chain variable (V_{H}) regions, single domain antibody fragments and single chain fragments, including single chain variable fragments (scFvs)) specific for GPRC5D and BCMA. Also provided are cells, such as engineered or recombinant cells expressing such CARs, and/or containing nucleic acids encoding such receptors, and compositions and articles of manufacture and therapeutic doses containing such cells.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Recombinant Receptors (e.g., Chimeric Antigen Receptors)

Provided in some aspects are GPRC5D- and BCMA-binding agents, such as recombinant receptors or chimeric antigen receptors (CARs) comprising extracellular binding domains that bind to both GPRC5D and BCMA. The extracellular binding domains comprise a GPRC5D-binding domain that binds to GPRC5D and a BCMA-binding domain that binds to BCMA. The GPRC5D-binding domain includes cell surface proteins containing antibodies (e.g., antigen-binding antibody fragments) and/or other binding peptides that specifically bind to GPRC5D (e.g., human GPRC5D protein). The BCMA-binding domain includes cell surface proteins containing antibodies (e.g., antigen-binding antibody fragments) and/or other binding peptides that specifically bind to BCMA (e.g., human BCMA). In some aspects, the binding domain binds to an extracellular portion of GPRC5D. In some aspects, the GPRC5D-binding domain binds to an extracellular portion of GPRC5D. In some aspects, the binding domain binds to an extracellular portion of BCMA. In some aspects, the BCMA-binding domain binds to an extracellular portion of BCMA.

Among the provided polynucleotides are those that encode recombinant receptors, such as antigen receptors, that specifically bind GPRC5D and BCMA. In some aspects, the encoded receptors, such as those containing GPRC5D- and BCMA-binding polypeptides, and compositions and articles of manufacture and uses of the same, also are provided.

Among the GPRC5D- and BCMA-binding domains are antibodies, such as single-chain antibodies (e.g., antigen binding antibody fragments), or portions thereof. In some examples, the recombinant receptors are chimeric antigen receptors, such as those containing anti-GPRC5D antibodies or antigen-binding fragments thereof and anti-BCMA antibodies or antigen-binding fragments thereof, such as in tandem. The provided polynucleotides can be incorporated into constructs, such as deoxyribonucleic acid (DNA) or RNA constructs, such as those that can be introduced into cells for expression of the encoded recombinant GPRC5D- and BCMA-binding domains.

The provided recombinant receptors generally contain an extracellular binding domain and an intracellular signaling domain. Among the provided receptors are polypeptides containing antibodies, such as an anti-GPRC5D antibody and an anti-BCMA antibody. Such receptors include chimeric antigen receptors that contain such antibodies.

Among the provided recombinant receptors are extracellular binding domains that include a GPRC5D-binding domain and BCMA-binding domain. The recombinant receptors include GPRC5D-binding domains that specifically bind to GPRC5D, such as anti-GPRC5D antibodies, e.g., GPRC5D antigen-binding fragments. The recombinant receptors also include BCMA-binding domains that specifically bind to BCMA, such as anti-BCMA antibodies, e.g., BCMA antigen-binding fragments. Among the antigen receptors are functional non-TCR antigen receptors, such as chimeric antigen receptors (CARs). Also provided are cells expressing the recombinant receptors and uses thereof in adoptive cell therapy, such as treatment of diseases and disorders associated with GPRC5D expression, BCMA expression, or both, e.g., multiple myeloma

Among the chimeric receptors are chimeric antigen receptors (CARs). The CARs generally include an extracellular binding domain that include a GPRC5D-binding domain and BCMA-binding domain, a transmembrane domain and an intracellular signaling domain. The CARs generally also include a spacer sequence (e.g. containing a hinge sequence) between the extracellular binding domain and transmembrane domain. Exemplary features of provided CARs are described in the following subsections.

1. Extracellular Antigen-Binding Domains

The chimeric receptors, such as CARs, generally include an extracellular binding domain that includes, is, or comprises an anti-GPRC5D antibody and an anti-BCMA antibody. Thus, the chimeric receptors, e.g., CARs, typically include in their extracellular portions a GPRC5D-binding domain and a BCMA-binding domain, such as antigen-binding fragments, domains, or portions, or one or more antibody variable regions, and/or antibody molecules, such as those described herein.

In some embodiments, the extracellular antigen binding domain comprises a GPRC5D-binding domain and a BCMA-binding domain. In some embodiments, the GPRC5D-binding domain comprises an anti-GPRC5D antibody or antigen-binding fragment thereof. In some embodiments, the BCMA-binding domain comprises an anti-BCMA antibody of antigen-binding fragment thereof.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., J Mol Biol, 1997; 273(4):927-48 (“Chothia” numbering scheme); MacCallum et al., J. Mol. Biol, 1996; 262:732-745.” (“Contact” numbering scheme); Lefranc M P et al., Dev Comp Immunol, 2003; 27(1):55-77 (“IMGT” numbering scheme); Honegger A and Plückthun A, J Mol Biol, 2001; 309(3):657-70, (“Aho” numbering scheme); Martin et al., PNAS, 1989; 86(23):9268-9272, (“AbM” numbering scheme); and Ye et al., Nucleic Acids Res. 2013; 41 (Web Server issue):W34-40, (“IgBLAST numbering scheme). Details regarding various numbering schemes are also described in, for example, Jarasch et al., Proteins, 2017; 85(1):65-71; Martin et al., Bioinformatics tools for antibody engineering. In: Dübel, S. (editor) Handbook of Therapeutic Antibodies, Vol. 1. Wiley-VCH, Weinheim, Germany; Martin, A. C. R. (2010). Protein Sequence and Structure Analysis of Antibody Variable Domains. In: Kontermann, R., Dübel, S. (eds) Antibody Engineering. Springer Protocols Handbooks. Springer, Berlin, Heidelberg; and Martin, A C R, Antibody Information: How to identify the CDRs by looking at a sequence [online]bioinf.org.uk/abs/info.html, all of which are incorporated by reference in their entireties. Various prediction algorithm tools are available and known for numbering antibody residues and CDRs (e.g., AbYsis, Abnum, AbYmod, AbRSA, IgBLAST, IMGT, or ANARCI).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, in some cases with insertions. Insertions in the sequence relative to the standard numbering scheme are indicated using insertion letter codes. For example, residues that are inserted between residues L30 and L31 are indicated as L31A, L31B, etc. Deletions in the sequence relative to the standard scheme are accommodated by skipping numbers. The two schemes place certain insertions and deletions (“indels”) at different positions, resulting in differential numbering. For instance, the Chothia numbering scheme is nearly identical to the Kabat numbering scheme, except that insertions are placed at structural positions and topologically equivalents residues do get assigned the same numbers. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software. The IgBLAST scheme is based on matching to germline V, D and J genes, and can be determined using National Center for Biotechnology Information (NCBI)'s IgBLAST tool.

In some embodiments, Kabat numbering can be determined by known sequence rules as described in, for example, Kabat et al. (1991), “Sequences of Proteins of Immunological Interest,” 5th Ed. Public Health Service, National Institutes of Health, Bethesda, MD. In some embodiments, the Kabat numbering scheme in some aspects can include any of the following rules to designate CDRs: CDR-L1 starts at approximately residue 24 of the light chain, always has a preceding C residue, and always has a following W residue; the end of CDR-L1 is defined by a stretch of 3 residues, where the W residue can be followed by Y, L, or F, followed by Q or L; CDR-1 has a length of 10 to 17 residues; CDR-L2 always starts 16 residues after the end of CDR-L1; the two residues before CDR-L2 are I and Y but can also be V and Y, I and K, or I and F; CDR-L2 is always 7 residues long; CDR-L3 always starts 33 residues after the end of CDR-L2, always has a preceding C residue, and is strictly followed by a F-G-X-G sequence motif, where X is any amino acid; CDR-L3 has a length of 7 to 11 residues; CDR-H1 starts at approximately position 26 of the heavy chain; the first amino acid in CDR-H1 is always 9 residues after a conserved C residue; CDR-H1 is followed by an invariant W residue followed by typically V, but also can be I or A; CDR-H1 has a length of 5 to 7 residues; CDR-H2 always starts at 15 residues after the end of CDR-H1; the first residue in CDR-H2 is usually preceded by the sequence motif L-E-W-I-G but a number of variations exist; the end of CDR-H2 is defined by a motif of 3 residues—the first residue of the motif of 3 residues can be either K or R, the second residue of the motif of 3 residues can be L, I, V, F, T, or A, the third residue of the motif of 3 residues can be T, S, I, or A; CDR-H2 has a length of 16 to 19 residues; CDR-H3 always starts 33 residues after the end of CDR-H2 and is always 3 residues after a C residue—the first residue of CDR-H3 is preceded by the conserved C residue followed by two residues, which are usually A-R; the residues following CDR-H3 is strictly followed by a W-G-X-G sequence motif, where the X is any amino acid; CDR-H3 typically has a length of 3 to 25 residues; CDR-H3 can be much longer than 25 residues.

In some cases, according to the Chothia numbering scheme, exact boundary positions of certain CDRs can differ based on different definitions for the CDRs (See e.g., Martin, A C R, Antibody Information: How to identify the CDRs by looking at a sequence [online] bioinf.org.uk/abs/info.html). For example, in some instances, the boundary positions for CDR-L1 according to Chothia numbering can be L26-L32 (Chothia et al., Science, 1986; 233(4765):755-8 and Chothia C. and Lesk A. M. J Mol Biol, 1987; 196(4):901-17). In some instances, the boundary positions for CDR-L1 can be L25-L32 (Al-Lazikani et al., J Mol Biol, 1997; 273(4):927-48). In some instances, the boundary positions for CDR-L2 can be L50-L52 and for CDR-L3 can be L91-L96 (Chothia et al., Science, 1986; 233(4765):755-8; Chothia C. and Lesk A. M. J Mol Biol, 1987; 196(4):901-17; and Al-Lazikani et al., J Mol Biol, 1997; 273(4):927-48). In some instances, the boundary positions for CDR-H1 according to Chothia numbering can be H26-H32 (Chothia et al., Science, 1986; 233(4765):755-8; Chothia C. and Lesk A. M. J Mol Biol, 1987; 196(4):901-17; and Al-Lazikani et al., J Mol Biol, 1997; 273(4):927-48). In some instances, the boundary positions for CDR-H2 can be H53-H55 (Chothia et al., Science, 1986; 233(4765):755-8 and Chothia C. and Lesk A. M. J Mol Biol, 1987, 196(4):901-17); H52a-H55 (Tramontano et al., J Mol Biol, 1990, 215(1): 175-82), or H52-H56 (Al-Lazikani et al., J Mol Biol., 1997; 273(4):927-48). In some instances, the boundary positions for CDR-H3 can be H96-H101 (Chothia et al., Science, 1986; 233(4765):755-8 and Chothia C. and Lesk A. M. J Mol Biol., 1987; 196(4):901-17). In some instances, the boundary positions for CDR-H3 can be H92-H104 (Morea et al., Biophys Chem, 1997; 68(1-3): 9-16 and Morea et al., J Mol Biol., 1998; 275(2): 269-94).

Table 1, below, exemplifies exemplary numbering and lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-L1 | L24-L34 | L24-L34 | L24-L34 | L30-L36 |
| CDR-L2 | L50-L56 | L50-L56 | L50-L56 | L46-L55 |
| CDR-L3 | L89-L97 | L89-L97 | L89-L97 | L89-L96 |
| CDR-H1 | H31-H35B | H26-H32 . . . 34 | H26-H35B | H30-H35B |
| (Kabat Numbering¹) | | | | |
| CDR-H1 | H31-H35 | H26-H32 | H26-H35 | H30-H35 |
| (Chothia Numbering²) | | | | |
| CDR-H2 | H50-H65 | H52-H56 | H50-H58 | H47-H58 |
| CDR-H3 | H95-H102 | H95-H102 | H95-H102 | H93-H101 |

¹Kabat et al. (1991), “Sequences of Proteins of Immunological Interest,” 5th Ed. Public Health Service, National Institutes of Health, Bethesda, MD
²Al-Lazikani et al., J Mol Biol., 1997; 273(4):927-48).

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, where it is stated that an antibody or antigen-binding fragment thereof comprises a CDR-H1, a CDR-H2, and a CDR-H3 as contained within a given V_{H} region amino acid sequence and a CDR-L1, a CDR-L2, and a CDR-L3 as contained within a given V_{L} region amino acid sequence, the CDRs can be defined by any of the aforementioned schemes, such as Kabat, Chothia, AbM, IgBLAST, IMGT, or Contact method, or other known scheme. In some embodiments, specific CDR sequences are specified. Exemplary CDR sequences of provided antibodies are described using various numbering schemes, although it is understood that a provided antibody can include CDRs as described according to any of the other aforementioned numbering schemes or other known numbering schemes.

Likewise, unless otherwise specified, a FR or individual specified FR(s) (e.g., FR-H1, FR-H2, FR-H3, FR-H4, FR-L1, FR-L2, FR-L3, and/or FR-L4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IgBLAST, IMGT, or Contact method, or other known schemes. In other cases, the particular amino acid sequence of a CDR or FR is given. In some embodiments, where it is stated that an antibody or antigen-binding fragment thereof comprises a FR-H1, a FR-H2, a FR-H3, and a FR-H4 as contained within a given V_{H} region amino acid sequence and a FR-L1, a FR-L2, a FR-L3, and a FR-L4 as contained within a given V_{L} region amino acid sequence, the FRs can be defined by any of the aforementioned schemes, such as Kabat, Chothia, AbM, IgBLAST, IMGT, or Contact method, or other known scheme.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs (See, e.g., Kindt et al. Kuby Immunology, 6th ed., W.H. Freeman and Co., page 91 (2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the provided antibodies are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region; and multispecific antibodies formed from antibody fragments. In some embodiments, the antibody is or comprises an antibody fragment comprising a variable heavy chain (V_{H}) and a variable light chain (V_{L}) region. In particular embodiments, the antibodies are single-chain antibody fragments comprising a heavy chain variable (V_{H}) region and/or a light chain variable (V_{L}) region, such as scFvs.

Single-domain antibodies (sdAbs) are antibody fragments comprising all or a portion of the heavy chain variable region or all or a portion of the light chain variable region of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

In some embodiments, the GPRC5D-binding domain comprises a heavy chain variable (V_{H}) region and a light chain variable (V_{L}) region. In some embodiments, the BCMA-binding domain comprises a heavy chain variable (V_{H}) region and a light chain variable (V_{L}) region.

In some embodiments, the extracellular binding domain comprises a loop format. In some embodiments, from N-terminus to C-terminus, the extracellular binding domain comprises: one of the V_{H} region and the V_{L} region of the BCMA-binding domain; one of the V_{H} region and the V_{L} region of the GPRC5D-binding domain; the other of the V_{H} region and the V_{L} region of the GPRC5D-binding domain; and the other of the V_{H} region and the V_{L} region of the BCMA-binding domain.

In some embodiments, the extracellular binding domain comprises a loop format. In some embodiments, from N-terminus to C-terminus, the extracellular binding domain comprises: one of the V_{H} region and the V_{L} region of the GPRC5D-binding domain; one of the V_{H} region and the V_{L} region of the BCMA-binding domain; the other of the V_{H} region and the V_{L} region of the BCMA-binding domain; and the other of the V_{H} region and the V_{L} region of the GPRC5D-binding domain.

In some embodiments, the extracellular binding domain comprises a linear format. In some embodiments, from N-terminus to C-terminus, the extracellular binding domain comprises: one of the V_{H} region and the V_{L} region of the GPRC5D-binding domain; the other of the V_{H} region and the V_{L} region of the GPRC5D-binding domain; one of the V_{H} region and the V_{L} region of the BCMA-binding domain; and the other of the V_{H} region and the V_{L} region of the BCMA-binding domain.

In some embodiments, the extracellular binding domain comprises a linear format. In some embodiments, from N-terminus to C-terminus, the extracellular binding domain comprises: one of the V_{H} region and the V_{L} region of the BCMA-binding domain; the other of the V_{H} region and the V_{L} region of the BCMA-binding domain; one of the V_{H} region and the V_{L} region of the GPRC5D-binding domain; and the other of the V_{H} region and the V_{L} region of the GPRC5D-binding domain.

a. GPRC5D-Binding Domain

In some embodiments, the provided GPRC5D-binding domain of the provided CARs contain an antibody, such as an anti-GPRC5D antibody, or an antigen-binding fragment thereof that confers the GPRC5D-binding properties of the provided CAR. In some embodiments, the CAR includes a GPRC5D-binding domain comprising an antibody, such as a heavy chain variable (V_{H}) region and/or light chain variable (V_{L}) region of the antibody. In some embodiments, the (V_{H}) region and (V_{L}) region of the GPRC5D-binding domain are part of the dual targeting CAR in a tandem format with the BCMA-binding domain. In some embodiments, the (V_{H}) region and the (V_{L}) region of the GPRC5D-binding domain are joined by a linker. In some embodiments, the (V_{H}) region and the (V_{L}) region of the GPRC5D-binding domain comprise an scFv antibody fragment. In some embodiments, the antibody or antigen-binding domain can be any anti-GPRC5D antibody described or derived from any anti-GPRC5D antibody described (see, e.g., WO 2016/090312, WO 2016/090329, WO 2018/017786, WO2020148677, WO2019154890, WO2021018859, WO2021018925, and WO2018147245). Any of such anti-GPRC5D antibodies or antigen-binding fragments can be used in the provided CARs. In some embodiments, the CAR contains a variable heavy (V_{H}) and/or a variable light (V_{L}) region derived from an antibody described in WO 2016/090312, WO 2016/090329, WO 2018/017786, WO2020148677, WO2019154890, WO2021018859, WO2021018925, or WO2018147245.

In some embodiments, the antibody, e.g., the anti-GPRC5D antibody, or antigen-binding fragment, contains a heavy and/or light chain variable (V_{H} or V_{L}) region sequence as described, or a sufficient antigen-binding portion thereof. In some embodiments, the anti-GPRC5D antibody, e.g., antigen-binding fragment, contains a V_{H} region sequence or sufficient antigen-binding portion thereof that contains a CDR-H1, CDR-H2 and/or CDR-H3 as described. In some embodiments, the anti-GPRC5D antibody, e.g., antigen-binding fragment, contains a V_{L} region sequence or sufficient antigen-binding portion that contains a CDR-L1, CDR-L2 and/or CDR-L3 as described. In some embodiments, the anti-GPRC5D antibody, e.g., antigen-binding fragment, contains a V_{H} region sequence that contains a CDR-H1, CDR-H2 and/or CDR-H3 as described and contains a V_{L} region sequence that contains a CDR-L1, CDR-L2 and/or CDR-L3 as described. Also among the antibodies are those having sequences at least at or about 90%, at or about 91%, at or about 92%, at or about 93%, at or about 94%, at or about 95%, at or about 96%, at or about 97%, at or about 98%, or at or about 99% identical to such a sequence.

In some embodiments, the antibody or antibody fragment, in the provided CAR, has a V_{H} region of any of the antibodies or antibody binding fragments described in any of WO 2016/090312, WO 2016/090329, WO 2018/017786, WO2020148677, WO2019154890, WO2021018859, WO2021018925, and WO2018147245.

In some embodiments, the CAR contains an antibody or antigen-binding fragment thereof, that has a heavy chain variable (V_{H}) region having the amino acid sequence set forth in SEQ ID NO:7, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 7, or contains a CDR-H1, CDR-H2, and/or CDR-H3 present in such a V_{H} sequence.

In some embodiments, the V_{H} region of an antibody or antigen-binding fragment thereof comprises a CDR-H1, CDR-H2, and/or CDR-H3 according to Kabat numbering. In some embodiments, the V_{H} region of an antibody or antigen-binding fragment thereof comprises a CDR-H1, CDR-H2, and/or CDR-H3 according to Chothia numbering. In some embodiments, the V_{H} region of an antibody or antigen-binding fragment thereof comprises a CDR-H1, CDR-H2, and/or CDR-H3 according to AbM numbering.

In some embodiments, the CAR contains an antibody or antigen-binding fragment thereof, that has a variable heavy chain (V_{H}) region comprising a CDR-H1 comprising the amino acid sequence set forth in SEQ ID NO: 1, a CDR-H2 comprising the amino acid sequence set forth in SEQ ID NO: 2, and a CDR-H3 comprising the amino acid sequence set forth in SEQ ID NO:3.

In some embodiments, the antibody or antigen-binding fragment thereof comprises a V_{H} region comprising a CDR-H1, a CDR-H2, and a CDR-H3 comprising the amino acid sequence set forth in SEQ ID NO:1, 2, and 3, respectively.

In some embodiments, the antibody or antigen-binding fragment thereof comprises a V_{H} region comprising the amino acid sequence set forth in SEQ ID NO:1, 2, and 3.

In some embodiments, the antibody or antigen-binding fragment thereof comprises a CDR-H1, CDR-H2 and CDR-H3, respectively, comprising the amino acid sequence of a CDR-H1, a CDR-H2, and a CDR-H3 contained within the V_{H} region amino acid sequence set forth in SEQ ID NO: 7.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO:7.

In some embodiments, the antibody or antigen-binding fragment thereof comprises a V_{H} region comprising the amino acid sequence set forth in SEQ ID NO: 7.

In some embodiments, the antibody or antibody fragment, in the provided CAR comprising a V_{H} region further comprises a light chain or a sufficient antigen binding portion thereof. For example, in some embodiments, the antibody or antigen-binding fragment thereof contains a V_{H} region and a V_{L} region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, a CAR provided herein, contains an antibody such as an anti-GPRC5D antibody, or antigen-binding fragment thereof that contains any of the above V_{H} region and contains a variable light chain region or a sufficient antigen binding portion thereof. For example, in some embodiments, the CAR contains an antibody or antigen-binding fragment thereof that contains a V_{H} region and a variable light chain (V_{L}) region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, the antibody or antigen-binding fragment has a V_{L} region described in any of WO 2016/090312, WO 2016/090329, WO 2018/017786, WO2020148677, WO2019154890, WO2021018859, WO2021018925, and WO2018147245.

In some embodiments, the CAR contains an antibody or antigen-binding fragment thereof, that has a light chain variable (V_{L}) region having the amino acid sequence set forth in SEQ ID NO:8, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 8, or contains a CDR-L1, CDR-L2, and/or CDR-L3 present in such a V_{L} sequence.

In some embodiments, the V_{L} region of an antibody or antigen-binding fragment thereof comprises a CDR-L1, CDR-L2, and/or CDR-L3 according to Kabat numbering. In some embodiments, the V_{L} region of an antibody or antigen-binding fragment thereof comprises a CDR-L1, CDR-L2, and/or CDR-L3 according to Chothia numbering. In some embodiments, the V_{L} region of an antibody or antigen-binding fragment thereof comprises a CDR-L1, CDR-L2, and/or CDR-L3 according to AbM numbering.

In some embodiments, the CAR contains an antibody or antigen-binding fragment thereof, that has a variable light chain (V_{L}) region comprising a CDR-L1 comprising the amino acid sequence set forth in SEQ ID NO: 4, a CDR-L2 comprising the amino acid sequence set forth in SEQ ID NO:5, and a CDR-L3 comprising the amino acid sequence set forth in SEQ ID NO: 6.

In some embodiments, the antibody or antigen-binding fragment thereof comprises a V_{L} region comprising a CDR-L1, a CDR-L2, and a CDR-L3 comprising the amino acid sequence set forth in SEQ ID NO:4, 5, and 6, respectively.

In some embodiments, the antibody or antigen-binding fragment thereof contains a CDR-L1, CDR-L2, and CDR-L3, respectively, contained within the V_{L} region amino acid sequence set forth in SEQ ID NO: 8.

Among the CARs provided herein is a CAR in which the antibody, such as an anti-GPRC5D antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 7, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 8.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 7; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 8.

In some embodiments, the V_{H} region of the antibody or antigen-binding fragment thereof comprise the amino acid sequence set forth in SEQ ID NO: 7, and the V_{L} region of the antibody or antigen-binding fragment comprises the amino acid sequence set forth in SEQ ID NO:8. In some embodiments, the V_{H} and V_{L} regions of the antibody or antigen-binding fragment thereof comprise the amino acid sequences set forth in SEQ ID NO: 7 and 8, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 7 and 8, respectively.

Among the provided CARs is a CAR in which the GPRC5D-binding domain contains a V_{H} region comprising the sequence set forth in SEQ ID NO:7 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:7; and contains a V_{L} region comprising the sequence set forth in SEQ ID NO:8 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:8. In some embodiments, the GPRC5D-binding domain of the provided CAR contains a V_{H} region that has a CDRH1, a CDRH2 and a CDRH3 comprising the amino acid sequence of SEQ ID NOS: 1, 2, and 3, respectively and a V_{L} region that has a CDRL1, a CDRL2 and a CDRL3 comprising the amino acid sequence of SEQ ID NOS: 4, 5, and 6, respectively. In some embodiments, the V_{H} region comprises the sequence set forth in SEQ ID NO:7 and the V_{L} region comprises the sequence set forth in SEQ ID NO:8.

In some embodiments, the GPRC5D-binding domain in the provided CAR is an antibody or antigen-binding fragment thereof that is a single-chain antibody fragment, such as a single chain variable fragment (scFv) or a diabody or a single domain antibody (sdAb). In some embodiments, the antibody or antigen-binding fragment is a single domain antibody comprising only the V_{H} region. In some embodiments, the antibody or antigen binding fragment comprises the heavy chain variable (V_{H}) region and light chain variable (V_{L}) region. In some embodiments, the antibody or antigen binding fragment is an scFv comprising a heavy chain variable (V_{H}) region and a light chain variable (V_{L}) region. In some embodiments, the single-chain antibody fragment (e.g., scFv) includes one or more linkers joining two antibody domains or regions, such as a heavy chain variable (V_{H}) region and a light chain variable (V_{L}) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline.

Accordingly, in some embodiments, the provided CARs contain anti-GPRC5D antibodies that include single-chain antibody fragments, such as scFvs and diabodies, particularly human single-chain antibody fragments, typically comprising linker(s) joining two antibody domains or regions, such V_{H} and V_{L} regions. In some embodiments, the provided CARs contain anti-BCMA antibodies that include single-chain antibody fragments, such as scFvs and diabodies, particularly human single-chain antibody fragments, typically comprising linker(s) joining two antibody domains or regions, such V_{H} and V_{L} regions. The linker typically is a peptide linker, e.g., a flexible and/or soluble peptide linker, such as one rich in glycine and serine.

In some embodiments the V_{H} and V_{L} region sequences of the GPRC5D-binding domain are connected in sequence by at least one intervening V_{H} and V_{L} region sequences of the BCMA-binding domain. In some embodiments, the extracellular antigen binding domain of the CAR has a loop format in which with the V_{H} and V_{L} region of the GPRC5D-binding domain is separated by one of the V_{H} and V_{L} region of the other BCMA-binding domain as a loop CAR. In some embodiments, at least one of the V_{H} or V_{L} region sequence of the GPRC5D-binding domain is linked directly to the V_{H} and V_{L} region of the BCMA-binding domain via a linker.

In some embodiments, the CAR comprises a loop format. In some embodiments, the V_{H} or V_{L} region of the BCMA-binding domain is joined to the V_{H} or the V_{L} region of the GPRC5D-binding domain by a linker. In some embodiments, one of the V_{H} and the V_{L} region of the BCMA-binding domain is joined to the other of the V_{H} and the V_{L} region of the BCMA-binding domain by a linker. In some embodiments, one of the V_{H} and the V_{L} region of the GPRC5D-binding domain is joined to the other of the V_{H} and the V_{L} region of the GPRC5D-binding domain by a linker. In some embodiments, the linker is set forth in SEQ ID NO:17. In some embodiments, the linker is set forth in SEQ ID NO:18. In some embodiments, the linker is set forth in SEQ ID NO:19. In some embodiments, the linker is set forth in SEQ ID NO:21. In some embodiments, the linker is set forth in SEQ ID NO:22.

In some embodiments, the V_{H} region of the BCMA-binding domain is joined to the V_{L} region of the GPRC5D-binding domain by the linker set forth in SEQ ID NO:19. In some embodiments, the V_{H} region of the BCMA-binding domain is joined to the V_{L} region of the GPRC5D-binding domain by the linker set forth in SEQ ID NO:21. In some embodiments, the V_{H} region of the BCMA-binding domain is joined to the V_{L} region of the GPRC5D-binding domain by the linker set forth in SEQ ID NO:22. In some embodiments, the V_{H} region of the BCMA-binding domain is joined to the V_{H} region of the GPRC5D-binding domain by the linker set forth in SEQ ID NO: 19. In some embodiments, the V_{H} region of the BCMA-binding domain is joined to the V_{H} region of the GPRC5D-binding domain by the linker set forth in SEQ ID NO:21. In some embodiments, the V_{H} region of the BCMA-binding domain is joined to the V_{H} region of the GPRC5D-binding domain by the linker set forth in SEQ ID NO:22.

In some embodiments, the V_{L} region of the BCMA-binding domain is joined to the V_{L} region of the GPRC5D-binding domain by the linker set forth in SEQ ID NO:19. In some embodiments, the V_{L} region of the BCMA-binding domain is joined to the V_{L} region of the GPRC5D-binding domain by the linker set forth in SEQ ID NO:21. In some embodiments, the V_{L} region of the BCMA-binding domain is joined to the V_{L} region of the GPRC5D-binding domain by the linker set forth in SEQ ID NO:22. In some embodiments, the V_{L} region of the BCMA-binding domain is joined to the V_{H} region of the GPRC5D-binding domain by the linker set forth in SEQ ID NO: 19. In some embodiments, the V_{L} region of the BCMA-binding domain is joined to the V_{H} region of the GPRC5D-binding domain by the linker set forth in SEQ ID NO:21. In some embodiments, the V_{L} region of the BCMA-binding domain is joined to the V_{H} region of the GPRC5D-binding domain by the linker set forth in SEQ ID NO:22.

In some embodiments, the extracellular antigen binding domain of the CAR has a linear format in which with the V_{H} and V_{L} region of the GPRC5D-binding domain are directly linked in sequence by a linker (e.g. as an scFv) and the V_{H} and V_{L} region of the BCMA-binding domain are directly linked in sequence by a linker (e.g., as an scFv). In some embodiments, the GPRC5D-binding domain comprises a linker between the V_{H} and V_{L} regions. In some embodiments, in order from N- to C-terminus, the GPRC5D-binding domain comprises one of the V_{H} and V_{L} regions, a linker, and the other of the V_{H} and V_{L} regions. In some embodiments, the linker is set forth in SEQ ID NO:17. Thus, in some embodiments, in order from N- to C-terminus, the GPRC5D-binding domain comprises one of the V_{H} and V_{L} regions, the linker set forth in SEQ ID NO:17, and the other of the V_{H} and V_{L} regions.

In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 21) or GGGS (3GS; SEQ ID NO: 20), such as between 2, 3, 4, and 5 repeats of such a sequence. Exemplary linkers include those having or consisting of a sequence set forth in SEQ ID NO:22 (GGGGSGGGGS), SEQ ID NO: 23 (GGGGSGGGGSGGGGS), and SEQ ID NO: 24 (GGGGSGGGGSGGGGSGGGGS). Exemplary linkers further include those having or consisting of the sequence set forth in SEQ ID NO: 18 (GSTSGSGKPGSGEGSTKG), SEQ ID NO: 17 (GSRGGGGSGGGGSGGGGSLEMA), and SEQ ID NO:19 (EAAAK).

Accordingly, in some embodiments, the provided embodiments include single-chain antibody fragments, e.g., scFvs, comprising one or more of the aforementioned linkers, such as glycine/serine rich linkers, including linkers having repeats of GGGS (SEQ ID NO: 20), or GGGGS (SEQ ID NO: 21), such as the linker set forth in SEQ ID NO: 17, 18, 19, 22, 23, or 24. In some embodiments, the linker comprises the sequence set forth in SEQ ID NO: 17. In some embodiments, the linker comprises the sequence set forth in SEQ ID NO: 18. In some embodiments, the linker comprises the sequence set forth in SEQ ID NO: 19. In some embodiments, the linker comprises the sequence set forth in SEQ ID NO: 20. In some embodiments, the linker comprises the sequence set forth in SEQ ID NO: 21. In some embodiments, the linker comprises the sequence set forth in SEQ ID NO: 22. In some embodiments, the linker comprises the sequence set forth in SEQ ID NO: 23. In some embodiments, the linker comprises the sequence set forth in SEQ ID NO: 24.

In some embodiments, the V_{H} region may be amino terminal to the V_{L} region. In some embodiments, the V_{H} region may be carboxy terminal to the V_{L} region. In particular embodiments, the fragment, e.g., scFv, may include a V_{H} region or portion thereof, followed by the linker, followed by a V_{L} region or portion thereof. In other embodiments, the fragment, e.g., the scFv, may include the V_{L} region or portion thereof, followed by the linker, followed by the V_{H} region or portion thereof.

In some embodiments, the CAR comprises a linear format. Thus, in some embodiments, the CAR comprises an anti-GPRC5D scFv and an anti-BCMA scFv. In some embodiments, the anti-GPRC5D scFv and the anti-BCMA scFv are joined by a linker. In some embodiments, the linker is set forth in SEQ ID NO:19. In some embodiments, the linker is set forth in SEQ ID NO:21. In some embodiments, the linker is set forth in SEQ ID NO:24. In some embodiments, the V_{H} and V_{L} regions of the anti-GPRC5D scFv are joined by the linker set forth in SEQ ID NO:17. In some embodiments, the V_{H} and V_{L} regions of the anti-BCMA scFv are joined by the linker set forth in SEQ ID NO:17.

In some aspects, an scFv provided herein comprises the amino acid sequence set forth in SEQ ID NO:45 or SEQ ID NO:46, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:45 or SEQ ID NO:46. In some aspects, an scFv provided herein comprises the amino acid sequence set forth in SEQ ID NO:45, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:45. In some aspects, an scFv provided herein comprises the amino acid sequence set forth in SEQ ID NO:45. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:45. In some aspects, an scFv provided herein comprises the amino acid sequence set forth in SEQ ID NO:46, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:46. In some aspects, an scFv provided herein comprises the amino acid sequence set forth in SEQ ID NO:46. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:46.

Among the antibodies, e.g., antigen-binding fragments, in the provided CARs, are human antibodies. In some embodiments of a provided human anti-GPRC5D antibody, e.g., antigen-binding fragments, the human antibody contains a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and/or contains a V_{L} region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment. In some embodiments, the portion of the V_{H} region corresponds to the CDR-H1, CDR-H2 and/or CDR-H3. In some embodiments, the portion of the V_{H} region corresponds to the framework region 1 (FR1), FR2, FR2 and/or FR4. In some embodiments, the portion of the V_{L} region corresponds to the CDR-L1, CDR-L2 and/or CDR-L3. In some embodiments, the portion of the V_{L} region corresponds to the FR1, FR2, FR2 and/or FR4.

In some embodiments, the human antibody, e.g., antigen-binding fragment, contains a CDR-H1 having at least 95%, 96%, 97%, 98%, 99%, or 100% sequence identity to an amino acid sequence of the corresponding CDR-H1 region within a sequence encoded by a germline nucleotide human heavy chain V segment. For example, the human antibody in some embodiments contains a CDR-H1 having a sequence that is 100% identical or with no more than one, two or three amino acid differences as compared to the corresponding CDR-H1 region within a sequence encoded by a germline nucleotide human heavy chain V segment.

In some embodiments, the human antibody, e.g., antigen-binding fragment, contains a CDR-H2 having at least 95%, 96%, 97%, 98%, 99%, or 100% sequence identity to an amino acid sequence of the corresponding CDR-H2 region within a sequence encoded by a germline nucleotide human heavy chain V segment. For example, the human antibody in some embodiments contains a CDR-H2 having a sequence that is 100% identical or with no more than one, two or three amino acid difference as compared to the corresponding CDR-H2 region within a sequence encoded by a germline nucleotide human heavy chain V segment.

In some embodiments, the human antibody, e.g., antigen-binding fragment, contains a CDR-H3 having at least 95%, 96%, 97%, 98%, 99%, or 100% sequence identity to an amino acid sequence of the corresponding CDR-H3 region within a sequence encoded by a germline nucleotide human heavy chain V segment, D segment and J segment. For example, the human antibody in some embodiments contains a CDR-H3 having a sequence that is 100% identical or with no more than one, two or three amino acid differences as compared to the corresponding CDR-H3 region within a sequence encoded by a germline nucleotide human heavy chain V segment, D segment and J segment.

In some embodiments, the human antibody, e.g., antigen-binding fragment, contains a CDR-L1 having at least 95%, 96%, 97%, 98%, 99%, or 100% sequence identity to an amino acid sequence of the corresponding CDR-L1 region within a sequence encoded by a germline nucleotide human light chain V segment. For example, the human antibody in some embodiments contains a CDR-L1 having a sequence that is 100% identical or with no more than one, two or three amino acid differences as compared to the corresponding CDR-L1 region within a sequence encoded by a germline nucleotide human light chain V segment.

In some embodiments, the human antibody, e.g., antigen-binding fragment, contains a CDR-L2 having at least 95%, 96%, 97%, 98%, 99%, or 100% sequence identity to an amino acid sequence of the corresponding CDR-L2 region within a sequence encoded by a germline nucleotide human light chain V segment. For example, the human antibody in some embodiments contains a CDR-L2 having a sequence that is 100% identical or with no more than one, two or three amino acid difference as compared to the corresponding CDR-L2 region within a sequence encoded by a germline nucleotide human light chain V segment.

In some embodiments, the human antibody, e.g., antigen-binding fragment, contains a CDR-L3 having at least 95%, 96%, 97%, 98%, 99%, or 100% sequence identity to an amino acid sequence of the corresponding CDR-L3 region within a sequence encoded by a germline nucleotide human light chain V segment and J segment. For example, the human antibody in some embodiments contains a CDR-L3 having a sequence that is 100% identical or with no more than one, two or three amino acid differences as compared to the corresponding CDR-L3 region within a sequence encoded by a germline nucleotide human light chain V segment and J segment.

In some embodiments, the human antibody, e.g., antigen-binding fragment, contains a framework region that contains human germline gene segment sequences. For example, in some embodiments, the human antibody contains a V_{H} region in which the framework region, e.g. FR1, FR2, FR3 and FR4, has at least 95%, 96%, 97%, 98%, 99%, or 100% sequence identity to a framework region encoded by a human germline antibody segment, such as a V segment and/or J segment. In some embodiments, the human antibody contains a V_{L} region in which the framework region e.g. FR1, FR2, FR3 and FR4, has at least 95%, 96%, 97%, 98%, 99%, or 100% sequence identity to a framework region encoded by a human germline antibody segment, such as a V segment and/or J segment. For example, in some such embodiments, the framework region sequence contained within the V_{H} region and/or V_{L} region differs by no more than 10 amino acids, such as no more than 9, 8, 7, 6, 5, 4, 3, 2 or 1 amino acid, compared to the framework region sequence encoded by a human germline antibody segment

b. BCMA-Binding Domain

In some embodiments, the provided BCMA-binding domain of the provided CARs contain an antibody, such as an anti-BCMA antibody, or an antigen-binding fragment thereof that confers the BCMA-binding properties of the provided CAR. In some embodiments, the CAR includes a BCMA-binding domain comprising an antibody, such as a heavy chain variable (V_{H}) region and/or light chain variable (V_{L}) region of the antibody. In some embodiments, the (V_{H}) region and (V_{L}) region of the BCMA-binding domain are part of the dual targeting CAR in a tandem format with the GPRC5D-binding domain. In some embodiments, the (V_{H}) region and the (V_{L}) region of the BCMA-binding domain are joined by an intradomain linker. In some embodiments, the (V_{H}) region and the (V_{L}) region of the BCMA-binding domain comprise an scFv antibody fragment. In some embodiments, the antibody or antigen-binding domain can be any anti-BCMA antibody described or derived from any anti-BCMA antibody described (see, e.g., WO 2016/090320 or WO 2016/090327). Any of such anti-BCMA antibodies or antigen-binding fragments can be used in the provided CARs. In some embodiments, the CAR contains a variable heavy (V_{H}) and/or a variable light (V_{L}) region derived from an antibody described in WO 2016/090320 or WO 2016/090327.

In some embodiments, the antibody, e.g., the anti-BCMA antibody, or antigen-binding fragment, contains a heavy and/or light chain variable (V_{H} or V_{L}) region sequence as described, or a sufficient antigen-binding portion thereof. In some embodiments, the anti-BCMA antibody, e.g., antigen-binding fragment, contains a V_{H} region sequence or sufficient antigen-binding portion thereof that contains a CDR-H1, CDR-H2 and/or CDR-H3 as described. In some embodiments, the anti-BCMA antibody, e.g., antigen-binding fragment, contains a V_{L} region sequence or sufficient antigen-binding portion that contains a CDR-L1, CDR-L2 and/or CDR-L3 as described. In some embodiments, the anti-BCMA antibody, e.g., antigen-binding fragment, contains a V_{H} region sequence that contains a CDR-H1, CDR-H2 and/or CDR-H3 as described and contains a V_{L} region sequence that contains a CDR-L1, CDR-L2 and/or CDR-L3 as described. Also among the antibodies are those having sequences at least at or about 90%, at or about 91%, at or about 92%, at or about 93%, at or about 94%, at or about 95%, at or about 96%, at or about 97%, at or about 98%, or at or about 99% identical to such a sequence.

In some embodiments, the antibody or antibody fragment, in the provided CAR, has a V_{H} region of any of the antibodies or antibody binding fragments described in any of WO 2016/090320 or WO 2016/090327.

In some embodiments, the CAR contains an antibody or antigen-binding fragment thereof, that has a heavy chain variable (V_{H}) region having the amino acid sequence set forth in SEQ ID NO:15, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 15, or contains a CDR-H1, CDR-H2, and/or CDR-H3 present in such a V_{H} sequence.

In some embodiments, the V_{H} region of an antibody or antigen-binding fragment thereof comprises a CDR-H1, CDR-H2, and/or CDR-H3 according to Kabat numbering. In some embodiments, the V_{H} region of an antibody or antigen-binding fragment thereof comprises a CDR-H1, CDR-H2, and/or CDR-H3 according to Chothia numbering. In some embodiments, the V_{H} region of an antibody or antigen-binding fragment thereof comprises a CDR-H1, CDR-H2, and/or CDR-H3 according to AbM numbering.

In some embodiments, the CAR contains an antibody or antigen-binding fragment thereof, that has a variable heavy chain (V_{H}) region comprising a CDR-H1 comprising the amino acid sequence set forth in SEQ ID NO: 9, a CDR-H2 comprising the amino acid sequence set forth in SEQ ID NO:10, and a CDR-H3 comprising the amino acid sequence set forth in SEQ ID NO:11.

In some embodiments, the antibody or antigen-binding fragment thereof comprises a V_{H} region comprising a CDR-H1, a CDR-H2, and a CDR-H3 comprising the amino acid sequence set forth in SEQ ID NO:9, 10, and 11, respectively.

In some embodiments, the antibody or antigen-binding fragment thereof comprises a V_{H} region comprising the amino acid sequence set forth in SEQ ID NO:9, 10, and 11.

In some embodiments, the antibody or antigen-binding fragment thereof comprises a CDR-H1, CDR-H2 and CDR-H3, respectively, comprising the amino acid sequence of a CDR-H1, a CDR-H2, and a CDR-H3 contained within the V_{H} region amino acid sequence set forth in SEQ ID NO:15.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO:15.

In some embodiments, the antibody or antigen-binding fragment thereof comprises a V_{H} region comprising the amino acid sequence set forth in SEQ ID NO: 15.

In some embodiments, the antibody or antibody fragment, in the provided CAR comprising a V_{H} region further comprises a light chain or a sufficient antigen binding portion thereof. For example, in some embodiments, the antibody or antigen-binding fragment thereof contains a V_{H} region and a V_{L} region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, a CAR provided herein, contains an antibody such as an anti-BCMA antibody, or antigen-binding fragment thereof that contains any of the above V_{H} region and contains a variable light chain region or a sufficient antigen binding portion thereof. For example, in some embodiments, the CAR contains an antibody or antigen-binding fragment thereof that contains a V_{H} region and a variable light chain (V_{L}) region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, the antibody or antigen-binding fragment has a V_{L} region described in any of WO 2016/090320 or WO 2016/090327.

In some embodiments, the CAR contains an antibody or antigen-binding fragment thereof, that has a light chain variable (V_{L}) region having the amino acid sequence set forth in SEQ ID NO:16, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 16, or contains a CDR-L1, CDR-L2, and/or CDR-L3 present in such a V_{L} sequence.

In some embodiments, the V_{L} region of an antibody or antigen-binding fragment thereof comprises a CDR-L1, CDR-L2, and/or CDR-L3 according to Kabat numbering. In some embodiments, the V_{L} region of an antibody or antigen-binding fragment thereof comprises a CDR-L1, CDR-L2, and/or CDR-L3 according to Chothia numbering. In some embodiments, the V_{L} region of an antibody or antigen-binding fragment thereof comprises a CDR-L1, CDR-L2, and/or CDR-L3 according to AbM numbering.

In some embodiments, the CAR contains an antibody or antigen-binding fragment thereof, that has a variable light chain (V_{L}) region comprising a CDR-L1 comprising the amino acid sequence set forth in SEQ ID NO: 12, a CDR-L2 comprising the amino acid sequence set forth in SEQ ID NO: 13, and a CDR-L3 comprising the amino acid sequence set forth in SEQ ID NO: 14.

In some embodiments, the antibody or antigen-binding fragment thereof comprises a V_{L} region comprising a CDR-L1, a CDR-L2, and a CDR-L3 comprising the amino acid sequence set forth in SEQ ID NO:12, 13, and 14, respectively.

In some embodiments, the antibody or antigen-binding fragment thereof contains a CDR-L1, CDR-L2, and CDR-L3, respectively, contained within the V_{L} region amino acid sequence set forth in SEQ ID NO: 16.

Among the CARs provided herein is a CAR in which the antibody, such as an anti-BCMA antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 15, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 16.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 15; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 16.

In some embodiments, the V_{H} region of the antibody or antigen-binding fragment thereof comprise the amino acid sequence set forth in SEQ ID NO: 15, and the V_{L} region of the antibody or antigen-binding fragment comprises the amino acid sequence set forth in SEQ ID NO:16. In some embodiments, the V_{H} and V_{L} regions of the antibody or antigen-binding fragment thereof comprise the amino acid sequences set forth in SEQ ID NO: 15 and 16, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 15 and 16, respectively.

Among the provided CARs is a CAR in which the BCMA-binding domain contains a V_{H} region comprising the sequence set forth in SEQ ID NO:15 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 15; and contains a V_{L} region comprising the sequence set forth in SEQ ID NO:16 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:16. In some embodiments, the BCMA-binding domain of the provided CAR contains a V_{H} region that has a CDRH1, a CDRH2 and a CDRH3 comprising the amino acid sequence of SEQ ID NOS: 9, 10, and 11, respectively and a V_{L} region that has a CDRL1, a CDRL2 and a CDRL3 comprising the amino acid sequence of SEQ ID NOS: 12, 13, and 14, respectively. In some embodiments, the V_{H} region comprises the sequence set forth in SEQ ID NO:15 and the V_{L} region comprises the sequence set forth in SEQ ID NO:16.

In some embodiments, the BCMA-binding domain in the provided CAR is an antibody or antigen-binding fragment thereof that is a single-chain antibody fragment, such as a single chain variable fragment (scFv) or a diabody or a single domain antibody (sdAb). In some embodiments, the antibody or antigen-binding fragment is a single domain antibody comprising only the V_{H} region. In some embodiments, the antibody or antigen binding fragment comprises the heavy chain variable (V_{H}) region and light chain variable (V_{L}) region. In some embodiments, the antibody or antigen binding fragment is an scFv comprising a heavy chain variable (V_{H}) region and a light chain variable (V_{L}) region. In some embodiments, the single-chain antibody fragment (e.g., scFv) includes one or more linkers joining two antibody domains or regions, such as a heavy chain variable (V_{H}) region and a light chain variable (V_{L}) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline.

Accordingly, in some embodiments, the provided CARs contain anti-BCMA antibodies that include single-chain antibody fragments, such as scFvs and diabodies, particularly human single-chain antibody fragments, typically comprising linker(s) joining two antibody domains or regions, such V_{H} and V_{L} regions. The linker typically is a peptide linker, e.g., a flexible and/or soluble peptide linker, such as one rich in glycine and serine.

In some embodiments the V_{H} and V_{L} region sequences of the BCMA-binding domain are connected in sequence by at least one intervening V_{H} and V_{L} region sequences of the GPRC5D-binding domain. In some embodiments, the extracellular antigen binding domain of the CAR has a loop format in which with the V_{H} and V_{L} region of the BCMA-binding domain is separated by one of the V_{H} and V_{L} region of the other GPRC5D-binding domain as a loop CAR. In some embodiments, at least one of the V_{H} or V_{L} region sequence of the BCMA-binding domain is linked directly to the V_{H} and V_{L} region of the GPRC5D-binding domain via a linker.

In some embodiments, the CAR comprises a loop format. In some embodiments, the V_{H} or V_{L} region of the BCMA-binding domain is joined to the V_{H} or the V_{L} region of the GPRC5D-binding domain by a linker. In some embodiments, one of the V_{H} and the V_{L} region of the BCMA-binding domain is joined to the other of the V_{H} and the V_{L} region of the BCMA-binding domain by a linker. In some embodiments, one of the V_{H} and the V_{L} region of the GPRC5D-binding domain is joined to the other of the V_{H} and the V_{L} region of the GPRC5D-binding domain by a linker. In some embodiments, the linker is set forth in SEQ ID NO:17. In some embodiments, the linker is set forth in SEQ ID NO:18. In some embodiments, the linker is set forth in SEQ ID NO:19. In some embodiments, the linker is set forth in SEQ ID NO:21. In some embodiments, the linker is set forth in SEQ ID NO:22.

In some embodiments, the V_{H} region of the BCMA-binding domain is joined to the V_{L} region of the GPRC5D-binding domain by the linker set forth in SEQ ID NO:19. In some embodiments, the V_{H} region of the BCMA-binding domain is joined to the V_{L} region of the GPRC5D-binding domain by the linker set forth in SEQ ID NO:21. In some embodiments, the V_{H} region of the BCMA-binding domain is joined to the V_{L} region of the GPRC5D-binding domain by the linker set forth in SEQ ID NO:22. In some embodiments, the V_{H} region of the BCMA-binding domain is joined to the V_{H} region of the GPRC5D-binding domain by the linker set forth in SEQ ID NO: 19. In some embodiments, the V_{H} region of the BCMA-binding domain is joined to the V_{H} region of the GPRC5D-binding domain by the linker set forth in SEQ ID NO:21. In some embodiments, the V_{H} region of the BCMA-binding domain is joined to the V_{H} region of the GPRC5D-binding domain by the linker set forth in SEQ ID NO:22.

In some embodiments, the V_{L} region of the BCMA-binding domain is joined to the V_{L} region of the GPRC5D-binding domain by the linker set forth in SEQ ID NO:19. In some embodiments, the V_{L} region of the BCMA-binding domain is joined to the V_{L} region of the GPRC5D-binding domain by the linker set forth in SEQ ID NO:21. In some embodiments, the V_{L} region of the BCMA-binding domain is joined to the V_{L} region of the GPRC5D-binding domain by the linker set forth in SEQ ID NO:22. In some embodiments, the V_{L} region of the BCMA-binding domain is joined to the V_{H} region of the GPRC5D-binding domain by the linker set forth in SEQ ID NO: 19. In some embodiments, the V_{L} region of the BCMA-binding domain is joined to the V_{H} region of the GPRC5D-binding domain by the linker set forth in SEQ ID NO:21. In some embodiments, the V_{L} region of the BCMA-binding domain is joined to the V_{H} region of the GPRC5D-binding domain by the linker set forth in SEQ ID NO:22.

In some embodiments, the extracellular antigen binding domain of the CAR has a linear format in which with the V_{H} and V_{L} region of the BCMA-binding domain are directly linked in sequence by a linker (e.g. as an scFv) and the V_{H} and V_{L} region of the GPRC5D-binding domain are directly linked in sequence by a linker (e.g., as an scFv). In some embodiments, the BCMA-binding domain comprises a linker between the V_{H} and V_{L} regions. In some embodiments, in order from N- to C-terminus, the BCMA-binding domain comprises one of the V_{H} and V_{L} regions, a linker, and the other of the V_{H} and V_{L} regions. In some embodiments, the linker is set forth in SEQ ID NO:17. Thus, in some embodiments, in order from N- to C-terminus, the BCMA-binding domain comprises one of the V_{H} and V_{L} regions, the linker set forth in SEQ ID NO:17, and the other of the V_{H} and V_{L} regions.

In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 21) or GGGS (3GS; SEQ ID NO: 20), such as between 2, 3, 4, and 5 repeats of such a sequence. Exemplary linkers include those having or consisting of a sequence set forth in SEQ ID NO:22 (GGGGSGGGGS), SEQ ID NO: 23 (GGGGSGGGGSGGGGS), and SEQ ID NO: 24 (GGGGSGGGGSGGGGSGGGGS). Exemplary linkers further include those having or consisting of the sequence set forth in SEQ ID NO: 18 (GSTSGSGKPGSGEGSTKG), SEQ ID NO: 17 (GSRGGGGSGGGGSGGGGSLEMA), and SEQ ID NO:19 (EAAAK).

Accordingly, in some embodiments, the provided embodiments include single-chain antibody fragments, e.g., scFvs, comprising one or more of the aforementioned linkers, such as glycine/serine rich linkers, including linkers having repeats of GGGS (SEQ ID NO: 20), or GGGGS (SEQ ID NO: 21), such as the linker set forth in SEQ ID NO: 17, 18, 19, 22, 23, or 24. In some embodiments, the linker comprises the sequence set forth in SEQ ID NO: 17. In some embodiments, the linker comprises the sequence set forth in SEQ ID NO: 18. In some embodiments, the linker comprises the sequence set forth in SEQ ID NO: 19. In some embodiments, the linker comprises the sequence set forth in SEQ ID NO: 20. In some embodiments, the linker comprises the sequence set forth in SEQ ID NO: 21. In some embodiments, the linker comprises the sequence set forth in SEQ ID NO: 22. In some embodiments, the linker comprises the sequence set forth in SEQ ID NO: 23. In some embodiments, the linker comprises the sequence set forth in SEQ ID NO: 24.

In some embodiments, the V_{H} region may be amino terminal to the V_{L} region. In some embodiments, the V_{H} region may be carboxy terminal to the V_{L} region. In particular embodiments, the fragment, e.g., scFv, may include a V_{H} region or portion thereof, followed by the linker, followed by a V_{L} region or portion thereof. In other embodiments, the fragment, e.g., the scFv, may include the V_{L} region or portion thereof, followed by the linker, followed by the V_{H} region or portion thereof.

In some embodiments, the CAR comprises a linear format. Thus, in some embodiments, the CAR comprises an anti-GPRC5D scFv and an anti-BCMA scFv. In some embodiments, the anti-GPRC5D scFv and the anti-BCMA scFv are joined by a linker. In some embodiments, the linker is set forth in SEQ ID NO:19. In some embodiments, the linker is set forth in SEQ ID NO:21. In some embodiments, the linker is set forth in SEQ ID NO:24. In some embodiments, the V_{H} and V_{L} regions of the anti-GPRC5D scFv are joined by the linker set forth in SEQ ID NO:17. In some embodiments, the V_{H} and V_{L} regions of the anti-BCMA scFv are joined by the linker set forth in SEQ ID NO:17.

In some aspects, an scFv provided herein comprises the amino acid sequence set forth in SEQ ID NO:47 or SEQ ID NO:48, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:47 or SEQ ID NO:48. In some aspects, an scFv provided herein comprises the amino acid sequence set forth in SEQ ID NO:47, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:47. In some aspects, an scFv provided herein comprises the amino acid sequence set forth in SEQ ID NO:47. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:47. In some aspects, an scFv provided herein comprises the amino acid sequence set forth in SEQ ID NO:48, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:48. In some aspects, an scFv provided herein comprises the amino acid sequence set forth in SEQ ID NO:48. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:48.

Among the antibodies, e.g., antigen-binding fragments, in the provided CARs, are human antibodies. In some embodiments of a provided human anti-BCMA antibody, e.g., antigen-binding fragments, the human antibody contains a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and/or contains a V_{L} region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment. In some embodiments, the portion of the V_{H} region corresponds to the CDR-H1, CDR-H2 and/or CDR-H3. In some embodiments, the portion of the V_{H} region corresponds to the framework region 1 (FR1), FR2, FR2 and/or FR4. In some embodiments, the portion of the V_{L} region corresponds to the CDR-L1, CDR-L2 and/or CDR-L3. In some embodiments, the portion of the V_{L} region corresponds to the FR1, FR2, FR2 and/or FR4.

In some embodiments, the human antibody, e.g., antigen-binding fragment, contains a CDR-H1 having at least 95%, 96%, 97%, 98%, 99%, or 100% sequence identity to an amino acid sequence of the corresponding CDR-H1 region within a sequence encoded by a germline nucleotide human heavy chain V segment. For example, the human antibody in some embodiments contains a CDR-H1 having a sequence that is 100% identical or with no more than one, two or three amino acid differences as compared to the corresponding CDR-H1 region within a sequence encoded by a germline nucleotide human heavy chain V segment.

In some embodiments, the human antibody, e.g., antigen-binding fragment, contains a CDR-H2 having at least 95%, 96%, 97%, 98%, 99%, or 100% sequence identity to an amino acid sequence of the corresponding CDR-H2 region within a sequence encoded by a germline nucleotide human heavy chain V segment. For example, the human antibody in some embodiments contains a CDR-H2 having a sequence that is 100% identical or with no more than one, two or three amino acid difference as compared to the corresponding CDR-H2 region within a sequence encoded by a germline nucleotide human heavy chain V segment.

In some embodiments, the human antibody, e.g., antigen-binding fragment, contains a CDR-H3 having at least 95%, 96%, 97%, 98%, 99%, or 100% sequence identity to an amino acid sequence of the corresponding CDR-H3 region within a sequence encoded by a germline nucleotide human heavy chain V segment, D segment and J segment. For example, the human antibody in some embodiments contains a CDR-H3 having a sequence that is 100% identical or with no more than one, two or three amino acid differences as compared to the corresponding CDR-H3 region within a sequence encoded by a germline nucleotide human heavy chain V segment, D segment and J segment.

In some embodiments, the human antibody, e.g., antigen-binding fragment, contains a CDR-L1 having at least 95%, 96%, 97%, 98%, 99%, or 100% sequence identity to an amino acid sequence of the corresponding CDR-L1 region within a sequence encoded by a germline nucleotide human light chain V segment. For example, the human antibody in some embodiments contains a CDR-L1 having a sequence that is 100% identical or with no more than one, two or three amino acid differences as compared to the corresponding CDR-L1 region within a sequence encoded by a germline nucleotide human light chain V segment.

In some embodiments, the human antibody, e.g., antigen-binding fragment, contains a CDR-L2 having at least 95%, 96%, 97%, 98%, 99%, or 100% sequence identity to an amino acid sequence of the corresponding CDR-L2 region within a sequence encoded by a germline nucleotide human light chain V segment. For example, the human antibody in some embodiments contains a CDR-L2 having a sequence that is 100% identical or with no more than one, two or three amino acid difference as compared to the corresponding CDR-L2 region within a sequence encoded by a germline nucleotide human light chain V segment.

In some embodiments, the human antibody, e.g., antigen-binding fragment, contains a CDR-L3 having at least 95%, 96%, 97%, 98%, 99%, or 100% sequence identity to an amino acid sequence of the corresponding CDR-L3 region within a sequence encoded by a germline nucleotide human light chain V segment and J segment. For example, the human antibody in some embodiments contains a CDR-L3 having a sequence that is 100% identical or with no more than one, two or three amino acid differences as compared to the corresponding CDR-L3 region within a sequence encoded by a germline nucleotide human light chain V segment and J segment.

In some embodiments, the human antibody, e.g., antigen-binding fragment, contains a framework region that contains human germline gene segment sequences. For example, in some embodiments, the human antibody contains a V_{H} region in which the framework region, e.g. FR1, FR2, FR3 and FR4, has at least 95%, 96%, 97%, 98%, 99%, or 100% sequence identity to a framework region encoded by a human germline antibody segment, such as a V segment and/or J segment. In some embodiments, the human antibody contains a V_{L} region in which the framework region e.g. FR1, FR2, FR3 and FR4, has at least 95%, 96%, 97%, 98%, 99%, or 100% sequence identity to a framework region encoded by a human germline antibody segment, such as a V segment and/or J segment. For example, in some such embodiments, the framework region sequence contained within the V_{H} region and/or V_{L} region differs by no more than 10 amino acids, such as no more than 9, 8, 7, 6, 5, 4, 3, 2 or 1 amino acid, compared to the framework region sequence encoded by a human germline antibody segment.

c. Exemplary Dual-Targeting Extracellular Antigen-Binding Domain

In some embodiments, the extracellular binding domain comprises a loop format. In some embodiments, from N-terminus to C-terminus, the extracellular binding domain comprises: one of the V_{H} region and the V_{L} region of the GPRC5D-binding domain; one of the V_{H} region and the V_{L} region of the BCMA-binding domain; the other of the V_{H} region and the V_{L} region of the BCMA-binding domain; and the other of the V_{H} region and the V_{L} region of the GPRC5D-binding domain. In some embodiments, the extracellular binding domain contains an inter-domain linker (e.g. a first and second inter-domain linker) separating the V_{H} region or the V_{L} region of the GPRC5D-binding domain from the V_{H} region or the V_{L} region of the BCMA-binding domain. In some embodiments, there is a first and second inter-domain linker and the linkers are the same. In some embodiments, the linker is any as described herein. In some embodiments, the inter-domain linker is set forth in any one of SEQ ID NOS: 19, 21, 22 or 24. In some embodiments, the extracellular binding domain contains an intradomain linker separating the V_{H} region and the V_{L} region of the BCMA-binding domain. In some embodiments, the intradomain linker is any as described herein. In some embodiments, the intradomain linker is set forth in SEQ ID NO:17 or SEQ ID NO:18. In some embodiments, the extracellular binding domain has the sequence of amino acids set forth in SEQ ID NO:83 or a sequence of amino acids that exhibits at least 85%, 86%, 87%, 88%, 89%, 90%, 91%, 92%, 93%, 94%, 95%, 96%, 97%, 98%, or 99% sequence identity SEQ ID NO:83. In some embodiments, the extracellular binding domain has the sequence of amino acids set forth in SEQ ID NO:84 or a sequence of amino acids that exhibits at least 85%, 86%, 87%, 88%, 89%, 90%, 91%, 92%, 93%, 94%, 95%, 96%, 97%, 98%, or 99% sequence identity SEQ ID NO:84. In some embodiments, the extracellular binding domain has the sequence of amino acids set forth in SEQ ID NO:87 or a sequence of amino acids that exhibits at least 85%, 86%, 87%, 88%, 89%, 90%, 91%, 92%, 93%, 94%, 95%, 96%, 97%, 98%, or 99% sequence identity SEQ ID NO:87. In some of any such embodiments, the extracellular antigen binding domain targets binding of the CAR for dual targeting of GPRC5D and BCMA.

In some embodiments, the extracellular binding domain comprises a loop format. In some embodiments, from N-terminus to C-terminus, the extracellular binding domain comprises: one of the V_{H} region and the V_{L} region of the BCMA-binding domain; one of the V_{H} region and the V_{L} region of the GPRC5D-binding domain; the other of the V_{H} region and the V_{L} region of the GPRC5D-binding domain; and the other of the V_{H} region and the V_{L} region of the BCMA-binding domain. In some embodiments, the extracellular binding domain contains an inter-domain linker (e.g. a first and second inter-domain linker) separating the V_{H} region or the V_{L} region of the BCMA-binding domain from the V_{H} region or the V_{L} region of the GPRC5D-binding domain. In some embodiments, the linker is any as described herein. In some embodiments, there is a first and second inter-domain linker and the linkers are the same. In some embodiments, the inter-domain linker is set forth in any one of SEQ ID NOS: 19, 21, 22 or 24. In some embodiments, the extracellular binding domain contains an intradomain linker separating the V_{H} region and the V_{L} region of the GPRC5D-binding domain. In some embodiments, the intradomain linker is any as described herein. In some embodiments, the intradomain linker is set forth in SEQ ID NO:17 or SEQ ID NO:18. In some embodiments, the extracellular binding domain has the sequence of amino acids set forth in SEQ ID NO:81 or a sequence of amino acids that exhibits at least 85%, 86%, 87%, 88%, 89%, 90%, 91%, 92%, 93%, 94%, 95%, 96%, 97%, 98%, or 99% sequence identity SEQ ID NO:81. In some embodiments, the extracellular binding domain has the sequence of amino acids set forth in SEQ ID NO:82 or a sequence of amino acids that exhibits at least 85%, 86%, 87%, 88%, 89%, 90%, 91%, 92%, 93%, 94%, 95%, 96%, 97%, 98%, or 99% sequence identity SEQ ID NO:82. In some embodiments, the extracellular binding domain has the sequence of amino acids set forth in SEQ ID NO:85 or a sequence of amino acids that exhibits at least 85%, 86%, 87%, 88%, 89%, 90%, 91%, 92%, 93%, 94%, 95%, 96%, 97%, 98%, or 99% sequence identity SEQ ID NO:85. In some embodiments, the extracellular binding domain has the sequence of amino acids set forth in SEQ ID NO:86 or a sequence of amino acids that exhibits at least 85%, 86%, 87%, 88%, 89%, 90%, 91%, 92%, 93%, 94%, 95%, 96%, 97%, 98%, or 99% sequence identity SEQ ID NO:86. In some embodiments, the extracellular binding domain has the sequence of amino acids set forth in SEQ ID NO:88 or a sequence of amino acids that exhibits at least 85%, 86%, 87%, 88%, 89%, 90%, 91%, 92%, 93%, 94%, 95%, 96%, 97%, 98%, or 99% sequence identity SEQ ID NO:88. In some embodiments, the extracellular binding domain has the sequence of amino acids set forth in SEQ ID NO:89 or a sequence of amino acids that exhibits at least 85%, 86%, 87%, 88%, 89%, 90%, 91%, 92%, 93%, 94%, 95%, 96%, 97%, 98%, or 99% sequence identity SEQ ID NO:89. In some embodiments, the extracellular binding domain has the sequence of amino acids set forth in SEQ ID NO:90 or a sequence of amino acids that exhibits at least 85%, 86%, 87%, 88%, 89%, 90%, 91%, 92%, 93%, 94%, 95%, 96%, 97%, 98%, or 99% sequence identity SEQ ID NO:90. In some of any such embodiments, the extracellular antigen binding domain targets binding of the CAR for dual targeting of GPRC5D and BCMA.

In some embodiments, the extracellular binding domain comprises a linear format. In some embodiments, from N-terminus to C-terminus, the extracellular binding domain comprises: one of the V_{H} region and the V_{L} region of the GPRC5D-binding domain; the other of the V_{H} region and the V_{L} region of the GPRC5D-binding domain; one of the V_{H} region and the V_{L} region of the BCMA-binding domain; and the other of the V_{H} region and the V_{L} region of the BCMA-binding domain. In some embodiments, the extracellular binding domain contains an intradomain linker separating the V_{H} region and the V_{L} region of the GPRC5D-binding domain. In some embodiments, the extracellular binding domain contains an intradomain linker separating the V_{H} region and the V_{L} region of the BCMA-binding domain. In some embodiments, the intradomain linker is any as described herein. In some embodiments, the intradomain linker is set forth in SEQ ID NO:17 or SEQ ID NO:18. In some embodiments, the extracellular binding domain contains an inter-domain linker separating the V_{H} region or the V_{L} region of the GPRC5D-binding domain from the V_{H} region or the V_{L} region of the BCMA-binding domain. In some embodiments, the linker is any as described herein. In some embodiments, there is a first and second inter-domain linker and the linkers are the same. In some embodiments, the inter-domain linker is set forth in any one of SEQ ID NOS: 19, 21, 22 or 24. In some embodiments, the extracellular binding domain has the sequence of amino acids set forth in SEQ ID NO:77 or a sequence of amino acids that exhibits at least 85%, 86%, 87%, 88%, 89%, 90%, 91%, 92%, 93%, 94%, 95%, 96%, 97%, 98%, or 99% sequence identity SEQ ID NO:77. In some embodiments, the extracellular binding domain has the sequence of amino acids set forth in SEQ ID NO:78 or a sequence of amino acids that exhibits at least 85%, 86%, 87%, 88%, 89%, 90%, 91%, 92%, 93%, 94%, 95%, 96%, 97%, 98%, or 99% sequence identity SEQ ID NO:78. In some embodiments, the extracellular binding domain has the sequence of amino acids set forth in SEQ ID NO:79 or a sequence of amino acids that exhibits at least 85%, 86%, 87%, 88%, 89%, 90%, 91%, 92%, 93%, 94%, 95%, 96%, 97%, 98%, or 99% sequence identity SEQ ID NO:79. In some embodiments, the extracellular binding domain has the sequence of amino acids set forth in SEQ ID NO:80 or a sequence of amino acids that exhibits at least 85%, 86%, 87%, 88%, 89%, 90%, 91%, 92%, 93%, 94%, 95%, 96%, 97%, 98%, or 99% sequence identity SEQ ID NO:80. In some of any such embodiments, the extracellular antigen binding domain targets binding of the CAR for dual targeting of GPRC5D and BCMA.

In some embodiments, the extracellular binding domain comprises a linear format. In some embodiments, from N-terminus to C-terminus, the extracellular binding domain comprises: one of the V_{H} region and the V_{L} region of the BCMA-binding domain; the other of the V_{H} region and the V_{L} region of the BCMA-binding domain; one of the V_{H} region and the V_{L} region of the GPRC5D-binding domain; and the other of the V_{H} region and the V_{L} region of the GPRC5D-binding domain. In some of any such embodiments, the extracellular antigen binding domain targets binding of the CAR for dual targeting of GPRC5D and BCMA.

In some embodiments, the extracellular binding domain comprises the amino acid sequence set forth in any one of SEQ ID NOS:77-90. In some embodiments, the extracellular binding domain comprises the amino acid sequence set forth in SEQ ID NO:77. In some embodiments, the extracellular binding domain comprises the amino acid sequence set forth in SEQ ID NO:78. In some embodiments, the extracellular binding domain comprises the amino acid sequence set forth in SEQ ID NO:79. In some embodiments, the extracellular binding domain comprises the amino acid sequence set forth in SEQ ID NO:80. In some embodiments, the extracellular binding domain comprises the amino acid sequence set forth in SEQ ID NO:81. In some embodiments, the extracellular binding domain comprises the amino acid sequence set forth in SEQ ID NO:82. In some embodiments, the extracellular binding domain comprises the amino acid sequence set forth in SEQ ID NO:83. In some embodiments, the extracellular binding domain comprises the amino acid sequence set forth in SEQ ID NO:84. In some embodiments, the extracellular binding domain comprises the amino acid sequence set forth in SEQ ID NO:85. In some embodiments, the extracellular binding domain comprises the amino acid sequence set forth in SEQ ID NO:86. In some embodiments, the extracellular binding domain comprises the amino acid sequence set forth in SEQ ID NO:87. In some embodiments, the extracellular binding domain comprises the amino acid sequence set forth in SEQ ID NO:88. In some embodiments, the extracellular binding domain comprises the amino acid sequence set forth in SEQ ID NO:89. In some embodiments, the extracellular binding domain comprises the amino acid sequence set forth in SEQ ID NO:90.

In some embodiments, the extracellular binding domain comprises the amino acid sequence set forth in any one of SEQ ID NOS:77-80, 83, 84, and 87. In some embodiments, the extracellular binding domain comprises the amino acid sequence set forth in SEQ ID NO:77. In some embodiments, the extracellular binding domain comprises the amino acid sequence set forth in SEQ ID NO:78. In some embodiments, the extracellular binding domain comprises the amino acid sequence set forth in SEQ ID NO:79. In some embodiments, the extracellular binding domain comprises the amino acid sequence set forth in SEQ ID NO:80. In some embodiments, the extracellular binding domain comprises the amino acid sequence set forth in SEQ ID NO:83. In some embodiments, the extracellular binding domain comprises the amino acid sequence set forth in SEQ ID NO:84. In some embodiments, the extracellular binding domain comprises the amino acid sequence set forth in SEQ ID NO:87.

In some embodiments, the extracellular binding domain is configured so the GPRC5D-targeted binding domain is proximal to the transmembrane domain.

In some embodiments, the extracellular binding domain comprises the amino acid sequence set forth in any one of SEQ ID NO: 83, 84, and 87. In some embodiments, the extracellular binding domain comprises the amino acid sequence set forth in SEQ ID NO:83. In some embodiments, the extracellular binding domain comprises the amino acid sequence set forth in SEQ ID NO:84. In some embodiments, the extracellular binding domain comprises the amino acid sequence set forth in SEQ ID NO:87.

In some embodiments, the extracellular binding domain is configured so the BCMA-targeted binding domain is proximal to the transmembrane domain.

In some embodiments, the extracellular binding domain comprises the amino acid sequence set forth in any one of SEQ ID NOS:77-80. In some embodiments, the extracellular binding domain comprises the amino acid sequence set forth in SEQ ID NO:77. In some embodiments, the extracellular binding domain comprises the amino acid sequence set forth in SEQ ID NO:78. In some embodiments, the extracellular binding domain comprises the amino acid sequence set forth in SEQ ID NO:79. In some embodiments, the extracellular binding domain comprises the amino acid sequence set forth in SEQ ID NO:80.

In some embodiments, the extracellular binding domain comprises the amino acid sequence set forth in any one of SEQ ID NOS:81, 82, 85, 86, 88, 89, and 90. In some embodiments, the extracellular binding domain comprises the amino acid sequence set forth in SEQ ID NO:81. In some embodiments, the extracellular binding domain comprises the amino acid sequence set forth in SEQ ID NO:82. In some embodiments, the extracellular binding domain comprises the amino acid sequence set forth in SEQ ID NO:85. In some embodiments, the extracellular binding domain comprises the amino acid sequence set forth in SEQ ID NO:86. In some embodiments, the extracellular binding domain comprises the amino acid sequence set forth in SEQ ID NO:88. In some embodiments, the extracellular binding domain comprises the amino acid sequence set forth in SEQ ID NO:89. In some embodiments, the extracellular binding domain comprises the amino acid sequence set forth in SEQ ID NO:90.

2. Spacer

In some embodiments, the recombinant receptor such as a CAR comprising extracellular antigen-binding domain provided herein, further includes a spacer. In some embodiments, the spacer is or includes at least a portion of an immunoglobulin constant region or variant or modified version thereof. In some embodiments, the portion of the immunoglobulin constant region includes a hinge region, e.g., an IgG4 hinge region, and/or a C_{H}1, C_{H}2 or C_{H}3 and/or Fc region. In some embodiments, the constant region or portion is of a human IgG, such as IgG4 or IgG1. In some aspects, the portion of the constant region serves as a spacer region between the antigen-binding domain or a portion thereof (e.g., a V_{H} or VL of the GPRC5D-binding domain or the BCMA-binding domain) and transmembrane domain. In some embodiments, the length of the spacer is adjusted to optimize the biophysical synapse distance between the CAR-expressing cell, such as a CAR-expressing T-cell, and the target of the CAR, such as a GPRC5D-expressing or BCMA-expressing cell. In some embodiments, the CAR is expressed by a T-cell, and the length of the spacer is adjusted to a length that is compatible for T-cell activation or to optimize CAR T-cell performance.

In some embodiments, the spacer can be of a length that provides for increased responsiveness of the cell following antigen binding, as compared to in the absence of the spacer or as compared to an alternative spacer of a different length (e.g. shorter in length). In some examples, the spacer is at or about 12 amino acids in length or is no more than 12 amino acids in length. In some embodiments, the spacer is at least 100 amino acids in length, such as at least 110, 125, 130, 135, 140, 145, 150, 160, 170, 180, 190, 200, 210, 220, 230, 240, or 250 amino acids in length. Exemplary spacers include those having at least about 10 to 300 amino acids, about 10 to 200 amino acids, about 50 to 175 amino acids, about 50 to 150 amino acids, about 10 to 125 amino acids, about 50 to 100 amino acids, about 100 to 300 amino acids, about 100 to 250 amino acids, about 125 to 250 amino acids, or about 200 to 250 amino acids, and including any integer between the endpoints of any of the listed ranges. In some embodiments, a spacer region is at least about 12 amino acids, at least about 119 amino acids, at least about 125 amino acids, at least about 200 amino acids, or at least about 220 amino acids, or at least about 225 amino acids in length.

In some embodiments, the spacer has a length of 125 to 300 amino acids in length, 125 to 250 amino acids in length, 125 to 230 amino acids in length, 125 to 200 amino acids in length, 125 to 180 amino acids in length, 125 to 150 amino acids in length, 150 to 300 amino acids in length, 150 to 250 amino acids in length, 150 to 230 amino acids in length, 150 to 200 amino acids in length, 150 to 180 amino acids in length, 180 to 300 amino acids in length, 180 to 250 amino acids in length, 180 to 230 amino acids in length, 180 to 200 amino acids in length, 200 to 300 amino acids in length, 200 to 250 amino acids in length, 200 to 230 amino acids in length, 230 to 300 amino acids in length, 230 to 250 amino acids in length or 250 to 300 amino acids in length. In some embodiments, the spacer is at least or at least about or is or is about 130, 140, 150, 160, 170, 180, 190, 200, 210, 220, 221, 222, 223, 224, 225, 226, 227, 228 or 229 amino acids in length, or a length between any of the foregoing.

Exemplary spacers include an IgG hinge alone, an IgG hinge linked to one or more of a C_{H}2 and C_{H}3 domain, or IgG hinge linked to the C_{H}3 domain. In some embodiments, the IgG hinge, C_{H}2 and/or C_{H}3 can be derived all or in part from IgG4 or IgG2, such as all or in part from human IgG4 or human IgG2. In some embodiments, the spacer can be a chimeric polypeptide containing one or more of a hinge, C_{H}2 and/or C_{H}3 sequence(s) derived from IgG4, IgG2, and/or IgG2 and IgG4. In some embodiments, the hinge region comprises all or a portion of an IgG4 hinge region and/or of an IgG2 hinge region, wherein the IgG4 hinge region is optionally a human IgG4 hinge region and the IgG2 hinge region is optionally a human IgG2 hinge region; the C_{H}2 region comprises all or a portion of an IgG4 C_{H}2 region and/or of an IgG2 C_{H}2 region, wherein the IgG4 C_{H}2 region is optionally a human IgG4 C_{H}2 region and the IgG2 C_{H}2 region is optionally a human IgG2 C_{H}2 region; and/or the C_{H}3 region comprises all or a portion of an IgG4 C_{H}3 region and/or of an IgG2 C_{H}3 region, wherein the IgG4 C_{H}3 region is optionally a human IgG4 C_{H}3 region and the IgG2 C_{H}3 region is optionally a human IgG2 C_{H}3 region. In some embodiments, the hinge, C_{H}2 and C_{H}3 comprises all or a portion of each of a hinge region, C_{H}2 and C_{H}3 from IgG4. In some embodiments, the hinge region is chimeric and comprises a hinge region from human IgG4 and human IgG2; the C_{H}2 region is chimeric and comprises a C_{H}2 region from human IgG4 and human IgG2; and/or the C_{H}3 region is chimeric and comprises a C_{H}3 region from human IgG4 and human IgG2. In some embodiments, the spacer comprises an IgG4/2 chimeric hinge or a modified IgG4 hinge comprising at least one amino acid replacement compared to human IgG4 hinge region; an human IgG2/4 chimeric C_{H}2 region; and a human IgG4 C_{H}3 region.

In some embodiments, the spacer can be derived all or in part from IgG4 and/or IgG2 and can contain mutations, such as one or more single amino acid mutations in one or more domains. In some examples, the amino acid modification is a substitution of a proline (P) for a serine (S) in the hinge region of an IgG4. In some embodiments, the amino acid modification is a substitution of a glutamine (Q) for an asparagine (N) to reduce glycosylation heterogeneity, such as an N177Q mutation at position 177, in the C_{H}2 region, of the full-length IgG4 Fe sequence set forth in SEQ ID NO: 75, or an N176Q at position 176, in the C_{H}2 region, of the full-length IgG2 Fe sequence set forth in SEQ ID NO: 76. In some embodiments, the spacer is or comprises an IgG4/2 chimeric hinge or a modified IgG4 hinge; an IgG2/4 chimeric C_{H}2 region; and an IgG4 C_{H}3 region. In some embodiments, the spacer is about 228 amino acids in length. In some embodiments, the spacer is set forth in SEQ ID NO: 27. In some embodiments, the spacer comprises the amino acid sequence

(SEQ ID NO: 27)
ESKYGPPCPPCPAPPVAGPSVFLFPPKPKDTLMISRTPEVTCVVVDVSQE
DPEVQFNWYVDGVEVHNAKTKPREEQFQSTYRVVSVLTVLHQDWLNGKEY
KCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQE
GNVFSCSVMHEALHNHYTQKSLSLSLGK

In some embodiments, the spacer is encoded by a polynucleotide that has been optimized for codon expression and/or to eliminate splice sites such as cryptic splice sites. In some embodiments, the coding sequence for the spacer comprises the nucleic acid sequence set forth in SEQ ID NO: 49. In some embodiments, the coding sequence for the spacer comprises the nucleic acid sequence set forth in SEQ ID NO: 50. In some embodiments, the coding sequence for the spacer comprises the nucleic acid sequence set forth in SEQ ID NO: 73. In some embodiments, the coding sequence for the spacer comprises the nucleic acid sequence set forth in SEQ ID NO: 74.

Additional exemplary spacers include, but are not limited to, those described in Hudecek et al. (2013) Clin. Cancer Res., 19:3153, Hudecek et al. (2015) Cancer Immunol. Res., 3(2):125-135, or international patent application publication number WO2014031687. In some embodiments, the nucleotide sequence of the spacer is optimized to reduce RNA heterogeneity upon expression. In some embodiments, the nucleotide sequence of the spacer is optimized to reduce cryptic splice sites or reduce the likelihood of a splice event at a splice site.

In some embodiments, the spacer has the amino acid sequence set forth in SEQ ID NO:25, and is encoded by the polynucleotide sequence set forth in SEQ ID NO:51. In some embodiments, the spacer has the amino acid sequence set forth in SEQ ID NO:26. In some embodiments, the spacer has the amino acid sequence set forth in SEQ ID NO:52. In some embodiments, the spacer has the amino acid sequence set forth in SEQ ID NO: 54, and is encoded by the polynucleotide sequence set forth in SEQ ID NO: 53.

In some embodiments, the spacer has an amino acid sequence that exhibits at least 85%, 86%, 87%, 88%, 89%, 90%, 91%, 92%, 93%, 94%, 95%, 96%, 97%, 98%, 99% or more sequence identity to SEQ ID NO: 27. In some embodiments, the spacer has the amino acid sequence set forth in SEQ ID NO: 27. In some embodiments, the spacer is encoded by the polynucleotide sequence set forth in SEQ ID NO: 49, 50, 73, or 74 or a polynucleotide that exhibits at least 85%, 86%, 87%, 88%, 89%, 90%, 91%, 92%, 93%, 94%, 95%, 96%, 97%, 98%, 99% or more sequence identity to SEQ ID NO: 49, 50, 73, or 74.

In some embodiments, the spacer is encoded by a polynucleotide that has been optionally optimized for codon usage and/or to reduce RNA heterogeneity. Methods to reduce RNA heterogeneity, such as by removing cryptic splice donor and/or acceptor sites, are described below. Observations have shown that cryptic splice donor and/or acceptor sites are present in the spacer region of certain immunoglobulin spacers when present in a CAR. In some embodiments, the spacer in a provided CAR is encoded by a polynucleotide in which one or more cryptic splice donor and/or acceptor sites are eliminated and/or are modified to reduce heterogeneity of the RNA transcribed from the construct, such as mRNA, following expression in a cell. In some embodiments, the spacer is encoded by the nucleotide sequence set forth in SEQ ID NO:49. In some embodiments, the spacer is encoded by the nucleotide sequence set forth in SEQ ID NO:50. In some embodiments, the spacer is encoded by the nucleotide sequence set forth in SEQ ID NO:73. In some embodiments, the spacer is encoded by the nucleotide sequence set forth in SEQ ID NO:74.

3. Transmembrane Domain and Intracellular Signaling Components

The extracellular antigen-binding domain (i.e., the GPRC5D- and BCMA-binding domains) generally is linked to one or more intracellular signaling components, such as signaling components that mimic activation through an antigen receptor complex, such as a TCR complex, in the case of a CAR, and/or signal via another cell surface receptor. Thus, in some embodiments, a GPRC5D-binding domain or a component thereof, or a BCMA-binding domain or a component thereof, (e.g., antibody or antigen binding fragment thereof) is linked to one or more transmembrane domains such as those described herein and intracellular signaling domains comprising one or more intracellular components such as those described herein. In some embodiments, the V_{H} or the VL of the binding domain most proximal to the cellular membrane is linked to the transmembrane domain. Typically, the binding domain or component thereof (e.g. V_{H} region or V_{L} region sequence) is linked to the transmembrane domain indirectly via the spacer sequence (e.g., Section 1.2). In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane domains include those derived from (i.e. comprise at least the transmembrane domain(s) of) the alpha, beta or zeta chain of the T-cell receptor, CD3 epsilon, CD4, CD5, CD8, CD9, CD16, CD22, CD28, CD33, CD37, CD45, CD64, CD80, CD86, CD134, CD137, and/or CD154. For example, in some embodiments, the transmembrane domain can be a CD28 transmembrane domain that comprises the sequence of amino acids set forth in SEQ ID NO: 18, such as may be encoded by the nucleic acid sequence set forth in SEQ ID NO: 55 or SEQ ID NO: 56. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine may be found at each end of a synthetic transmembrane domain. In some embodiments, the linkage is by linkers, spacers, and/or transmembrane domain(s).

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the intracellular signaling domain of the CAR.

The receptor, e.g., the CAR, generally includes an intracellular signaling region comprising at least one intracellular signaling component or components. In some embodiments, the receptor includes an intracellular component or signaling domain of a TCR complex, such as a TCR CD3 chain that mediates T-cell activation and cytotoxicity, e.g., CD3 zeta (CD3-ζ) chain. Thus, in some aspects, the GPRC5D- or BCMA-binding antibody is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR includes a chimeric molecule between CD3-zeta (CD3-ζ) or Fc receptor γ and CD8, CD4, CD25 or CD16.

In some embodiments, upon ligation of the CAR, the cytoplasmic domain or intracellular signaling domain of the CAR stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such classes of cytoplasmic signaling sequences.

In some aspects, the CAR includes a primary cytoplasmic signaling sequence that regulates primary stimulation and/or activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, CD3 gamma, CD3 delta and CD3 epsilon. In some embodiments, the intracellular signaling region in the CAR contain(s) a cytoplasmic signaling domain, portion thereof, or sequence derived from CD3 zeta. In some embodiments the CD3 zeta comprises the sequence of amino acids set forth in SEQ ID NO: 30. In some embodiments, the CD3 zeta is encoded by the nucleic acid sequence set forth in SEQ ID NO: 55 or SEQ ID NO: 56.

In some embodiments, the CAR includes a signaling domain (e.g., an intracellular or cytoplasmic signaling domain) and/or transmembrane portion of a costimulatory molecule, such as a T cell costimulatory molecule. Exemplary costimulatory molecules include CD28, 4-1BB, OX40, DAP10, and ICOS. For example, a costimulatory molecule can be derived from 4-1BB and can comprise the amino acid sequence set forth in SEQ ID NO: 29. In some embodiments, the 4-1BB is encoded by the nucleotide sequence set forth in SEQ ID NO: 57 or SEQ ID NO: 58. In some cases, a costimulatory molecule can be derived from CD28 and can comprise the amino acid sequence set forth in SEQ ID NO: 100. In some aspects, the same CAR includes both the stimulatory or activating components (e.g., cytoplasmic signaling sequence) and costimulatory components.

In some embodiments, the stimulatory or activating components are included within one CAR, whereas the costimulatory component is provided by another CAR recognizing another antigen. In some embodiments, the CARs include activating or stimulatory CARs, and costimulatory CARs, both expressed on the same cell (see WO 2014/055668). In some aspects, the GPRC5D-targeting CAR is the stimulatory or activating CAR; in other aspects, it is the costimulatory CAR. In some embodiments, the cells further include inhibitory CARs (iCARs, see Fedorov et al., Sci. Transl. Medicine, 5(215) (December, 2013), such as a CAR recognizing an antigen other than GPRC5D, whereby a stimulatory or an activating signal delivered through the GPRC5D-targeting CAR is diminished or inhibited by binding of the inhibitory CAR to its ligand, e.g., to reduce off-target effects.

In certain embodiments, the intracellular signaling region comprises a CD28 transmembrane and signaling domain linked to a CD3 (e.g., CD3-zeta) intracellular domain. In some embodiments, the intracellular signaling domain comprises a chimeric CD28 and 4-1BB (CD137; TNFRSF9) co-stimulatory domains, linked to a CD3 zeta intracellular domain.

In some embodiments, the CAR encompasses one or more, e.g., two or more, costimulatory domains and a stimulatory or an activation domain, e.g., primary activation domain, in the cytoplasmic portion. Exemplary CARs include intracellular components of CD3-zeta, CD28, and 4-1BB.

In some embodiments, provided embodiments of anti-GPRC5D CAR contains an extracellular antigen-binding domain containing any of the anti-GPRC5D antibody or antigen-binding fragments described herein, such as in Section I.1a; a spacer comprising an IgG4/2 chimeric hinge or a modified IgG4 hinge, an IgG2/4 chimeric C_{H}2 region, and an IgG4 C_{H}3 region, such as one that is about 228 amino acids in length, or a spacer set forth in SEQ ID NO:27, such as encoded by the nucleotide sequence set forth in any of SEQ ID NOS: 49, 50, 73 and 74; a transmembrane domain, such as a transmembrane domain from a human CD28; and an intracellular signaling region comprising a cytoplasmic signaling domain of a CD3-zeta (CD3ζ) chain and an intracellular signaling domain of a T cell costimulatory molecule. Also provided are polynucleotides encoding such a chimeric antigen receptor. In some embodiments, the transmembrane domain is or comprises the sequence set forth in SEQ ID NO:28. In some embodiments, the intracellular signaling domain of a T cell costimulatory molecule is an intracellular signaling domain of human CD28, human 4-1BB or human ICOS or a signaling portion thereof. In some embodiments, the intracellular signaling domain is an intracellular signaling domain of human 4-1BB. In some embodiments, the intracellular signaling domain is or comprises the sequence set forth in SEQ ID NO:29. In some embodiments, the cytoplasmic signaling domain is a human CD3-zeta cytoplasmic signaling domain, such as set forth in SEQ ID NO:30. In some embodiments, the intracellular signaling region comprises the sequences set forth in SEQ ID NO:30 and SEQ ID NO:29.

4. Exemplary CARs

In some embodiments, the CAR includes an extracellular antigen-binding domain that comprises, in order from N- to C-terminus: the V_{L} region of the GPRC5D-binding domain, the linker set forth in SEQ ID NO:17, the V_{H} region of the GPRC5D-binding domain, the linker set forth in SEQ ID NO:19, the V_{H} region of the BCMA-binding domain, the linker set forth in SEQ ID NO: 17, and the V_{L} region of the BCMA-binding domain.

In some embodiments, the CAR includes an extracellular antigen-binding domain that comprises, in order from N- to C-terminus: the V_{L} region of the GPRC5D-binding domain, the linker set forth in SEQ ID NO:17, the V_{H} region of the GPRC5D-binding domain, the linker set forth in SEQ ID NO:24, the V_{H} region of the BCMA-binding domain, the linker set forth in SEQ ID NO: 17, and the V_{L} region of the BCMA-binding domain.

In some embodiments, the CAR includes an extracellular antigen-binding domain that comprises, in order from N- to C-terminus: the V_{H} region of the GPRC5D-binding domain, the linker set forth in SEQ ID NO:17, the V_{L} region of the GPRC5D-binding domain, the linker set forth in SEQ ID NO:21, the V_{H} region of the BCMA-binding domain, the linker set forth in SEQ ID NO: 17, and the V_{L} region of the BCMA-binding domain.

In some embodiments, the CAR includes an extracellular antigen-binding domain that comprises, in order from N- to C-terminus: the V_{H} region of the GPRC5D-binding domain, the linker set forth in SEQ ID NO:17, the V_{L} region of the GPRC5D-binding domain, the linker set forth in SEQ ID NO:24, the V_{L} region of the BCMA-binding domain, the linker set forth in SEQ ID NO:17, and the V_{H} region of the BCMA-binding domain.

In some embodiments, the CAR includes an extracellular antigen-binding domain that comprises, in order from N- to C-terminus: the V_{H} region of the BCMA-binding domain, the linker set forth in SEQ ID NO:19, the V_{L} region of the GPRC5D-binding domain, the linker set forth in SEQ ID NO:17, the V_{H} region of the GPRC5D-binding domain, the linker set forth in SEQ ID NO:19, and the V_{L} region of the BCMA-binding domain.

In some embodiments, the CAR includes an extracellular antigen-binding domain that comprises, in order from N- to C-terminus: the V_{L} region of the BCMA-binding domain, the linker set forth in SEQ ID NO:19, the V_{H} region of the GPRC5D-binding domain, the linker set forth in SEQ ID NO:24, the V_{L} region of the GPRC5D-binding domain, the linker set forth in SEQ ID NO:19, and the V_{H} region of the BCMA-binding domain.

In some embodiments, the includes an extracellular antigen-binding domain that CAR comprises, in order from N- to C-terminus: the V_{H} region of the GPRC5D-binding domain, the linker set forth in SEQ ID NO:21, the V_{L} region of the BCMA-binding domain, the linker set forth in SEQ ID NO:17, the V_{H} region of the BCMA-binding domain, the linker set forth in SEQ ID NO:21, and the V_{L} region of the GPRC5D-binding domain.

In some embodiments, the CAR includes an extracellular antigen-binding domain that comprises, in order from N- to C-terminus: the V_{L} region of the GPRC5D-binding domain, the linker set forth in SEQ ID NO:21, the V_{H} region of the BCMA-binding domain, the linker set forth in SEQ ID NO:17, the V_{L} region of the BCMA-binding domain, the linker set forth in SEQ ID NO:21, and the V_{H} region of the GPRC5D-binding domain.

In some embodiments, the CAR includes an extracellular antigen-binding domain that comprises, in order from N- to C-terminus: the V_{L} region of the BCMA-binding domain, the linker set forth in SEQ ID NO:21, the V_{H} region of the GPRC5D-binding domain, the linker set forth in SEQ ID NO:17, the V_{L} region of the GPRC5D-binding domain, the linker set forth in SEQ ID NO:21, and the V_{H} region of the BCMA-binding domain.

In some embodiments, the CAR includes an extracellular antigen-binding domain that comprises, in order from N- to C-terminus: the V_{L} region of the BCMA-binding domain, the linker set forth in SEQ ID NO:21, the V_{L} region of the GPRC5D-binding domain, the linker set forth in SEQ ID NO:17, the V_{H} region of the GPRC5D-binding domain, the linker set forth in SEQ ID NO:21, and the V_{H} region of the BCMA-binding domain.

In some embodiments, the CAR includes an extracellular antigen-binding domain that comprises, in order from N- to C-terminus: the V_{H} region of the GPRC5D-binding domain, the linker set forth in SEQ ID NO:22, the V_{L} region of the BCMA-binding domain, the linker set forth in SEQ ID NO:17, the V_{H} region of the BCMA-binding domain, the linker set forth in SEQ ID NO:22, and the V_{L} region of the GPRC5D-binding domain.

In some embodiments, the CAR includes an extracellular antigen-binding domain that comprises, in order from N- to C-terminus: the V_{H} region of the BCMA-binding domain, the linker set forth in SEQ ID NO:22, the V_{H} region of the GPRC5D-binding domain, the linker set forth in SEQ ID NO:17, the V_{L} region of the GPRC5D-binding domain, the linker set forth in SEQ ID NO:22, and the V_{L} region of the BCMA-binding domain.

In some embodiments, the CAR includes an extracellular antigen-binding domain that comprises, in order from N- to C-terminus: the V_{L} region of the BCMA-binding domain, the linker set forth in SEQ ID NO:22, the V_{H} region of the GPRC5D-binding domain, the linker set forth in SEQ ID NO:17, the V_{L} region of the GPRC5D-binding domain, the linker set forth in SEQ ID NO:22, and the V_{H} region of the BCMA-binding domain.

In some embodiments, the CAR includes an extracellular antigen-binding domain that comprises, in order from N- to C-terminus: the V_{L} region of the BCMA-binding domain, the linker set forth in SEQ ID NO:22, the V_{H} region of the GPRC5D-binding domain, the linker set forth in SEQ ID NO:24, the V_{L} region of the GPRC5D-binding domain, the linker set forth in SEQ ID NO:22, and the V_{H} region of the BCMA-binding domain.

In some embodiments, the CAR includes an extracellular antigen-binding domain that comprises the spacer set forth in SEQ ID NO:27. In some embodiments, the CAR comprises the transmembrane domain set forth in SEQ ID NO:28. In some embodiments, the CAR comprises an intracellular signaling domain comprising the amino acid sequences set forth in SEQ ID NOS: 29 and 30.

In some embodiments, the CAR comprises the amino acid sequence set forth in any one of SEQ ID NOS:31-44, or is encoded by the nucleotide sequence set forth in any one of SEQ ID NOS:105-120. In some embodiments, the CAR comprises the amino acid sequence set forth in any one of SEQ ID NOS:31-44. In some embodiments, the CAR is encoded by the nucleotide sequence set forth in any one of SEQ ID NOS:105-120.

In some embodiments, the CAR comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 31. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 105. In some embodiments, the CAR comprises the amino acid sequence set forth in SEQ ID NO:31, or is encoded by the nucleotide sequence set forth in SEQ ID NO:105. In some embodiments, the CAR is encoded by the nucleotide sequence set forth in SEQ ID NO:105. In some embodiments, the CAR comprises the amino acid sequence set forth in SEQ ID NO:31. In some of any such embodiments, the CAR is a dual-targeting CAR that directs binding to GPRC5D and BCMA, such as expressed on the surface of cells (e.g., cancer cell, such as plasma cells from subjects with multiple myeloma).

In some embodiments, the CAR comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 32. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 10⁶. In some embodiments, the CAR comprises the amino acid sequence set forth in SEQ ID NO:32, or is encoded by the nucleotide sequence set forth in SEQ ID NO:10⁶. In some embodiments, the CAR comprises the amino acid sequence set forth in SEQ ID NO:32. In some embodiments, the CAR is encoded by the nucleotide sequence set forth in SEQ ID NO:10⁶. In some of any such embodiments, the CAR is a dual-targeting CAR that directs binding to GPRC5D and BCMA, such as expressed on the surface of cells (e.g., cancer cell, such as plasma cells from subjects with multiple myeloma).

In some embodiments, the CAR comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 33. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 10⁷. In some embodiments, the CAR comprises the amino acid sequence set forth in SEQ ID NO:33, or is encoded by the nucleotide sequence set forth in SEQ ID NO:10⁷. In some embodiments, the CAR comprises the amino acid sequence set forth in SEQ ID NO:33. In some embodiments, the CAR is encoded by the nucleotide sequence set forth in SEQ ID NO:10⁷. In some of any such embodiments, the CAR is a dual-targeting CAR that directs binding to GPRC5D and BCMA, such as expressed on the surface of cells (e.g., cancer cell, such as plasma cells from subjects with multiple myeloma).

In some embodiments, the CAR comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 34. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 10⁸. In some embodiments, the CAR comprises the amino acid sequence set forth in SEQ ID NO:34, or is encoded by the nucleotide sequence set forth in SEQ ID NO:10⁸. In some embodiments, the CAR comprises the amino acid sequence set forth in SEQ ID NO:34. In some embodiments, the CAR is encoded by the nucleotide sequence set forth in SEQ ID NO:10⁸. In some of any such embodiments, the CAR is a dual-targeting CAR that directs binding to GPRC5D and BCMA, such as expressed on the surface of cells (e.g., cancer cell, such as plasma cells from subjects with multiple myeloma).

In some embodiments, the CAR comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 35. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 10⁹. In some embodiments, the CAR comprises the amino acid sequence set forth in SEQ ID NO:35, or is encoded by the nucleotide sequence set forth in SEQ ID NO:10⁹. In some embodiments, the CAR comprises the amino acid sequence set forth in SEQ ID NO:35. In some embodiments, the CAR is encoded by the nucleotide sequence set forth in SEQ ID NO:10⁹. In some of any such embodiments, the CAR is a dual-targeting CAR that directs binding to GPRC5D and BCMA, such as expressed on the surface of cells (e.g., cancer cell, such as plasma cells from subjects with multiple myeloma).

In some embodiments, the CAR comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 36. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 110. In some embodiments, the CAR comprises the amino acid sequence set forth in SEQ ID NO:36, or is encoded by the nucleotide sequence set forth in SEQ ID NO:110. In some embodiments, the CAR comprises the amino acid sequence set forth in SEQ ID NO:36. In some embodiments, the CAR is encoded by the nucleotide sequence set forth in SEQ ID NO:110. In some of any such embodiments, the CAR is a dual-targeting CAR that directs binding to GPRC5D and BCMA, such as expressed on the surface of cells (e.g., cancer cell, such as plasma cells from subjects with multiple myeloma).

In some embodiments, the CAR comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 37. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 111. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 119. In some embodiments, the CAR comprises the amino acid sequence set forth in SEQ ID NO:37, or is encoded by the nucleotide sequence set forth in SEQ ID NO:111. In some embodiments, the CAR comprises the amino acid sequence set forth in SEQ ID NO:37, or is encoded by the nucleotide sequence set forth in SEQ ID NO:119. In some embodiments, the CAR comprises the amino acid sequence set forth in SEQ ID NO:37. In some embodiments, the CAR is encoded by the nucleotide sequence set forth in SEQ ID NO:111. In some embodiments, the CAR is encoded by the nucleotide sequence set forth in SEQ ID NO:119. In some of any such embodiments, the CAR is a dual-targeting CAR that directs binding to GPRC5D and BCMA, such as expressed on the surface of cells (e.g., cancer cell, such as plasma cells from subjects with multiple myeloma).

In some embodiments, the CAR comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 38. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 112. In some embodiments, the CAR comprises the amino acid sequence set forth in SEQ ID NO:38, or is encoded by the nucleotide sequence set forth in SEQ ID NO:112. In some embodiments, the CAR comprises the amino acid sequence set forth in SEQ ID NO:38. In some embodiments, the CAR is encoded by the nucleotide sequence set forth in SEQ ID NO:112. In some of any such embodiments, the CAR is a dual-targeting CAR that directs binding to GPRC5D and BCMA, such as expressed on the surface of cells (e.g., cancer cell, such as plasma cells from subjects with multiple myeloma).

In some embodiments, the CAR comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 39. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 113. In some embodiments, the CAR comprises the amino acid sequence set forth in SEQ ID NO:39, or is encoded by the nucleotide sequence set forth in SEQ ID NO:113. In some embodiments, the CAR comprises the amino acid sequence set forth in SEQ ID NO:39. In some embodiments, the CAR is encoded by the nucleotide sequence set forth in SEQ ID NO:113. In some embodiments, the CAR is encoded by the nucleotide sequence set forth in SEQ ID NO:112. In some of any such embodiments, the CAR is a dual-targeting CAR that directs binding to GPRC5D and BCMA, such as expressed on the surface of cells (e.g., cancer cell, such as plasma cells from subjects with multiple myeloma).

In some embodiments, the CAR comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 40. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 114. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 120. In some embodiments, the CAR comprises the amino acid sequence set forth in SEQ ID NO:40, or is encoded by the nucleotide sequence set forth in SEQ ID NO:114. In some embodiments, the CAR comprises the amino acid sequence set forth in SEQ ID NO:40, or is encoded by the nucleotide sequence set forth in SEQ ID NO:120. In some embodiments, the CAR comprises the amino acid sequence set forth in SEQ ID NO:40. In some embodiments, the CAR is encoded by the nucleotide sequence set forth in SEQ ID NO:114. In some embodiments, the CAR is encoded by the nucleotide sequence set forth in SEQ ID NO:120. In some of any such embodiments, the CAR is a dual-targeting CAR that directs binding to GPRC5D and BCMA, such as expressed on the surface of cells (e.g., cancer cell, such as plasma cells from subjects with multiple myeloma).

In some embodiments, the CAR comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 41. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 115. In some embodiments, the CAR comprises the amino acid sequence set forth in SEQ ID NO:41, or is encoded by the nucleotide sequence set forth in SEQ ID NO:115. In some embodiments, the CAR comprises the amino acid sequence set forth in SEQ ID NO:41. In some embodiments, the CAR is encoded by the nucleotide sequence set forth in SEQ ID NO:115. In some embodiments, the CAR is encoded by the nucleotide sequence set forth in SEQ ID NO:120. In some of any such embodiments, the CAR is a dual-targeting CAR that directs binding to GPRC5D and BCMA, such as expressed on the surface of cells (e.g., cancer cell, such as plasma cells from subjects with multiple myeloma).

In some embodiments, the CAR comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 42. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 116. In some embodiments, the CAR comprises the amino acid sequence set forth in SEQ ID NO:42, or is encoded by the nucleotide sequence set forth in SEQ ID NO:116. In some embodiments, the CAR comprises the amino acid sequence set forth in SEQ ID NO:42. In some embodiments, the CAR is encoded by the nucleotide sequence set forth in SEQ ID NO:116. In some of any such embodiments, the CAR is a dual-targeting CAR that directs binding to GPRC5D and BCMA, such as expressed on the surface of cells (e.g., cancer cell, such as plasma cells from subjects with multiple myeloma).

In some embodiments, the CAR comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 43. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 117. In some embodiments, the CAR comprises the amino acid sequence set forth in SEQ ID NO:43, or is encoded by the nucleotide sequence set forth in SEQ ID NO:117. In some embodiments, the CAR comprises the amino acid sequence set forth in SEQ ID NO:43. In some embodiments, the CAR is encoded by the nucleotide sequence set forth in SEQ ID NO:117. In some of any such embodiments, the CAR is a dual-targeting CAR that directs binding to GPRC5D and BCMA, such as expressed on the surface of cells (e.g., cancer cell, such as plasma cells from subjects with multiple myeloma).

In some embodiments, the CAR comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 44. In some embodiments, the CAR is encoded by a nucleotide sequence that exhibits at least 85%, 86%, 87%, 88%, 89%, 90%, 91%, 92%, 93%, 94%, 95%, 96%, 97%, 98% or 99% sequence identity to SEQ ID NO: 118. In some embodiments, the CAR comprises the amino acid sequence set forth in SEQ ID NO:44, or is encoded by the nucleotide sequence set forth in SEQ ID NO:118. In some embodiments, the CAR comprises the amino acid sequence set forth in SEQ ID NO:44. In some embodiments, the CAR is encoded by the nucleotide sequence set forth in SEQ ID NO:118. n some of any such embodiments, the CAR is a dual-targeting CAR that directs binding to GPRC5D and BCMA, such as expressed on the surface of cells (e.g., cancer cell, such as plasma cells from subjects with multiple myeloma).

5. Exemplary Features

In some of or any of the provided embodiments, the bispecific CAR and/or the GPRC5D-binding domain, antibody or antigen binding fragment, specifically binds to GPRC5D, such as GPRC5D on the surface of a multiple myeloma plasma cell. In some embodiments binding can be to a human GPRC5D, a mouse GPRC5D protein, or a non-human primate (e.g., cynomolgus monkey) GPRC5D protein. In some embodiments, among provided bispecific CARs and/or GPRC5D-binding domain are those that bind human GPRC5D protein. The observation that an antibody or other binding molecule binds to GPRC5D protein or specifically binds to GPRC5D protein does not necessarily mean that it binds to a GPRC5D protein of every species. For example, in some embodiments, features of binding to GPRC5D protein, such as the ability to specifically bind thereto and/or to compete for binding thereto with a reference antibody, and/or to bind with a particular affinity or compete to a particular degree, in some embodiments, refers to the ability with respect to a human GPRC5D protein and the antibody may not have this feature with respect to a GPRC5D protein of another species, such as mouse.

In some embodiments, the antibodies specifically bind to human GPRC5D protein, such as to an epitope or region of human GPRC5D protein, such as the human GPRC5D protein comprising the amino acid sequence of SEQ ID NO:59 (Uniprot Q9NZD1), or an allelic variant or splice variant thereof.

In one embodiment, the extent of binding of an anti-GPRC5D antibody or antigen-binding domain or CAR to an unrelated, non-GPRC5D protein, such as a non-human GPRC5D protein or other non-GPRC5D protein, is less than at or about 10% of the binding of the antibody or antigen-binding domain or CAR to human GPRC5D protein or human membrane-bound GPRC5D as measured, e.g., by a radioimmunoassay (RIA). In some embodiments, among the antibodies or antigen-binding domains in the provided CARs, are antibodies or antigen-binding domains or CARs in which binding to mouse GPRC5D protein is less than or at or about 10% of the binding of the antibody to human GPRC5D protein. In some embodiments, among the antibodies or antigen-binding domains in the provided CARs, are antibodies in which binding to cynomolgus monkey GPRC5D protein is less than or at or about 10% of the binding of the antibody to human GPRC5D protein. In some embodiments, among the antibodies or antigen-binding domains in the provided CARs, are antibodies in which binding to cynomolgus monkey GPRC5D protein and/or a mouse GPRC5D protein is similar to or about the same as the binding of the antibody to human GPRC5D protein.

In some embodiments, the antibodies, in the provided CARs, are capable of binding GPRC5D protein, such as human GPRC5D protein, with at least a certain affinity, as measured by any of a number of known methods. In some embodiments, the affinity is represented by an equilibrium dissociation constant (K_{D}); in some embodiments, the affinity is represented by EC₅₀.

A variety of assays are known for assessing binding affinity and/or determining whether a binding molecule (e.g., an antibody or fragment thereof) specifically binds to a particular ligand (e.g., an antigen, such as a GPRC5D protein). It is within the level of a skilled artisan to determine the binding affinity of a binding molecule, e.g., an antibody, for an antigen, e.g., GPRC5D, such as human GPRC5D or cynomolgus GPRC5D or mouse GPRC5D, such as by using any of a number of binding assays that are well known in the art. For example, in some embodiments, a BIAcore® instrument can be used to determine the binding kinetics and constants of a complex between two proteins (e.g., an antibody or fragment thereof, and an antigen, such as a GPRC5D protein), using surface plasmon resonance (SPR) analysis (see, e.g., Scatchard et al., Ann. N. Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

In some of or any of the provided embodiments, the bispecific CAR and/or the BCMA-binding domain, antibody or antigen binding fragment, specifically binds to BCMA, such as BCMA on the surface of a multiple myeloma plasma cell. In some embodiments binding can be to a human BCMA, a mouse BCMA protein, or a non-human primate (e.g., cynomolgus monkey) BCMA protein. In some embodiments, among provided bispecificCARs and/or BCMA-binding domain are those that bind human BCMA protein. The observation that an antibody or other binding molecule binds to BCMA protein or specifically binds to BCMA protein does not necessarily mean that it binds to a BCMA protein of every species. For example, in some embodiments, features of binding to BCMA protein, such as the ability to specifically bind thereto and/or to compete for binding thereto with a reference antibody, and/or to bind with a particular affinity or compete to a particular degree, in some embodiments, refers to the ability with respect to a human BCMA protein and the antibody may not have this feature with respect to a BCMA protein of another species, such as mouse.

In some embodiments, the antibodies specifically bind to human BCMA protein, such as to an epitope or region of human BCMA protein, such as the human BCMA protein comprising the amino acid sequence of SEQ ID NO:60 (Uniprot Q02223), or an allelic variant or splice variant thereof.

In one embodiment, the extent of binding of an anti-BCMA antibody or antigen-binding domain or CAR to an unrelated, non-BCMA protein, such as a non-human BCMA protein or other non-BCMA protein, is less than at or about 10% of the binding of the antibody or antigen-binding domain or CAR to human BCMA protein or human membrane-bound BCMA as measured, e.g., by a radioimmunoassay (RIA). In some embodiments, among the antibodies or antigen-binding domains in the provided CARs, are antibodies or antigen-binding domains or CARs in which binding to mouse BCMA protein is less than or at or about 10% of the binding of the antibody to human BCMA protein. In some embodiments, among the antibodies or antigen-binding domains in the provided CARs, are antibodies in which binding to cynomolgus monkey BCMA protein is less than or at or about 10% of the binding of the antibody to human BCMA protein. In some embodiments, among the antibodies or antigen-binding domains in the provided CARs, are antibodies in which binding to cynomolgus monkey BCMA protein and/or a mouse BCMA protein is similar to or about the same as the binding of the antibody to human BCMA protein.

In some embodiments, the antibodies, in the provided CARs, are capable of binding BCMA protein, such as human BCMA protein, with at least a certain affinity, as measured by any of a number of known methods. In some embodiments, the affinity is represented by an equilibrium dissociation constant (K_{D}); in some embodiments, the affinity is represented by EC₅₀.

A variety of assays are known for assessing binding affinity and/or determining whether a binding molecule (e.g., an antibody or fragment thereof) specifically binds to a particular ligand (e.g., an antigen, such as a BCMA protein). It is within the level of a skilled artisan to determine the binding affinity of a binding molecule, e.g., an antibody, for an antigen, e.g., BCMA, such as human BCMA or cynomolgus BCMA or mouse BCMA, such as by using any of a number of binding assays that are well known in the art. For example, in some embodiments, a BIAcore® instrument can be used to determine the binding kinetics and constants of a complex between two proteins (e.g., an antibody or fragment thereof, and an antigen, such as a BCMA protein), using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed polynucleotides or binding of proteins.

In some embodiments, the binding molecule, e.g., antibody or fragment thereof or antigen-binding domain of a CAR, binds, such as specifically binds, to an antigen, e.g., a GPRC5D protein or an epitope therein, with an affinity or K_{A} (i.e., an equilibrium association constant of a particular binding interaction with units of 1/M; equal to the ratio of the on-rate [kₒₙ or kₐ] to the off-rate [k_{off} or k_{d}] for this association reaction, assuming bimolecular interaction) equal to or greater than 10⁵ M. In some embodiments, the antibody or fragment thereof or antigen-binding domain of a CAR exhibits a binding affinity for the peptide epitope with a K_{D} (i.e., an equilibrium dissociation constant of a particular binding interaction with units of M; equal to the ratio of the off-rate [k_{off} or k_{d}] to the on-rate [kₒₙ or k_{d}] for this association reaction, assuming bimolecular interaction) of equal to or less than 10⁻⁵ M. For example, the equilibrium dissociation constant K_{D} ranges from 10⁻⁵ M to 10⁻¹³ M, such as 10⁻⁷ M to 10⁻¹¹ M, 10⁻⁸ M to 10⁻¹⁰ M, or 10⁻⁹ M to 10⁻¹⁰ M. The on-rate (association rate constant; kₒₙ or kₐ; units of 1/Ms) and the off-rate (dissociation rate constant; k_{off} or k_{d}; units of 1/s) can be determined using any of the assay methods known in the art, for example, surface plasmon resonance (SPR).

In some embodiments, the binding affinity (EC₅₀) and/or the dissociation constant of the antibody (e.g. antigen-binding fragment) or antigen-binding domain of a CAR to GPRC5D protein, such as human GPRC5D protein, is from or from about 0.01 nM to about 500 nM, from or from about 0.01 nM to about 400 nM, from or from about 0.01 nM to about 100 nM, from or from about 0.01 nM to about 50 nM, from or from about 0.01 nM to about 10 nM, from or from about 0.01 nM to about 1 nM, from or from about 0.01 nM to about 0.1 nM, from or from about 0.1 nM to about 500 nM, from or from about 0.1 nM to about 400 nM, from or from about 0.1 nM to about 100 nM, from or from about 0.1 nM to about 50 nM, from or from about 0.1 nM to about 10 nM, from or from about 0.1 nM to about 1 nM, from or from about 0.5 nM to about 200 nM, from or from about 1 nM to about 500 nM, from or from about 1 nM to about 100 nM, from or from about 1 nM to about 50 nM, from or from about 1 nM to about 10 nM, from or from about 2 nM to about 50 nM, from or from about 10 nM to about 500 nM, from or from about 10 nM to about 100 nM, from or from about 10 nM to about 50 nM, from or from about 50 nM to about 500 nM, from or from about 50 nM to about 100 nM or from or from about 100 nM to about 500 nM. In certain embodiments, the binding affinity (EC₅₀) and/or the equilibrium dissociation constant, K_{D}, of the antibody to a GPRC5D protein, such as human GPRC5D protein, is at or less than or about 400 nM, 300 nM, 200 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. In some embodiments, the antibodies bind to a GPRC5D protein, such as human GPRC5D protein, with a sub-nanomolar binding affinity, for example, with a binding affinity less than about 1 nM, such as less than about 0.9 nM, about 0.8 nM, about 0.7 nM, about 0.6 nM, about 0.5 nM, about 0.4 nM, about 0.3 nM, about 0.2 nM or about 0.1 nM or less.

In some embodiments, the binding affinity may be classified as high affinity or as low affinity. In some cases, the binding molecule (e.g. antibody or fragment thereof) or antigen-binding domain of a CAR that exhibits low to moderate affinity binding exhibits a K_{A} of up to 10⁷ M⁻¹, up to 10⁶ M⁻¹, up to 105 M⁻¹. In some cases, a binding molecule (e.g. antibody or fragment thereof) that exhibits high affinity binding to a particular epitope interacts with such epitope with a K_{A} of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In some embodiments, the binding affinity (EC₅₀) and/or the equilibrium dissociation constant, K_{D}, of the binding molecule, e.g., anti-GPRC5D antibody or fragment thereof or antigen-binding domain of a CAR, to a GPRC5D protein, is from or from about 0.01 nM to about 1 μM, 0.1 nM to 1 μM, 1 nM to 1 M, 1 nM to 500 nM, 1 nM to 100 nM, 1 nM to 50 nM, 1 nM to 10 nM, 10 nM to 500 nM, 10 nM to 100 nM, 10 nM to 50 nM, 50 nM to 500 nM, 50 nM to 100 nM or 100 nM to 500 nM. In certain embodiments, the binding affinity (EC₅₀) and/or the dissociation constant of the equilibrium dissociation constant, K_{D}, of the binding molecule, e.g., anti-GPRC5D antibody or fragment thereof or antigen-binding domain of a CAR, to a GPRC5D protein, is at or about or less than at or about 1 μM, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. The degree of affinity of a particular antibody can be compared with the affinity of a known antibody, such as a reference antibody.

In some embodiments, the binding affinity of a binding molecule, such as an anti-GPRC5D antibody or antigen-binding domain of a CAR, for different antigens, e.g., GPRC5D proteins from different species can be compared to determine the species cross-reactivity. For example, species cross-reactivity can be classified as high cross reactivity or low cross reactivity. In some embodiments, the equilibrium dissociation constant, K_{D}, for different antigens, e.g., GPRC5D proteins from different species such as human, cynomolgus monkey or mouse, can be compared to determine species cross-reactivity. In some embodiments, the species cross-reactivity of an anti-GPRC5D antibody or antigen-binding domain of a CAR can be high, e.g., the anti-GPRC5D antibody binds to human GPRC5D and a species variant GPRC5D to a similar degree, e.g., the ratio of K_{D} for human GPRC5D and K_{D} for the species variant GPRC5D is or is about 1. In some embodiments, the species cross-reactivity of an anti-GPRC5D antibody or antigen-binding domain of a CAR can be low, e.g., the anti-GPRC5D antibody has a high affinity for human GPRC5D but a low affinity for a species variant GPRC5D, or vice versa. For example, the ratio of K_{D} for the species variant GPRC5D and K_{D} for the human GPRC5D is more than 10, 15, 20, 25, 30, 40, 50, 60, 70, 80, 90, 100, 200, 500, 1000, 2000 or more, and the anti-GPRC5D antibody has low species cross-reactivity. The degree of species cross-reactivity can be compared with the species cross-reactivity of a known antibody, such as a reference antibody.

Among the provided bispecific CARs are CARs that exhibit antigen-dependent activity or signaling, i.e. signaling activity that is measurably absent or at background levels in the absence of antigen, e.g. GPRC5D. Thus, in some aspects, provided CARs do not exhibit, or exhibit no more than background or a tolerable or low level of, tonic signaling or antigen-independent activity or signaling in the absence of antigen, e.g. GPRC5D, being present. In some embodiments, the provided bispecific CAR-expressing cells exhibit biological activity or function, including cytotoxic activity, cytokine production, and ability to proliferate.

In some embodiments, the binding molecule, e.g., antibody or fragment thereof or antigen-binding domain of a CAR, binds, such as specifically binds, to an antigen, e.g., a BCMA protein (e.g., SEQ ID NO:60) or an epitope therein, with an affinity or KA (i.e., an equilibrium association constant of a particular binding interaction with units of 1/M; equal to the ratio of the on-rate [kₒₙ or kₐ] to the off-rate [k_{off} or k_{d}] for this association reaction, assuming bimolecular interaction) equal to or greater than 10⁵ M⁻¹. In some embodiments, the antibody or fragment thereof or antigen-binding domain of a CAR exhibits a binding affinity for the peptide epitope with a K_{D} (i.e., an equilibrium dissociation constant of a particular binding interaction with units of M; equal to the ratio of the off-rate [kₒₙ or kₐ] to the on-rate [kₒₙ or kₐ] for this association reaction, assuming bimolecular interaction) of equal to or less than 10⁻⁵ M. For example, the equilibrium dissociation constant K_{D} ranges from 10⁻⁵ M to 10⁻¹³ M, such as 10⁻⁷ M to 10⁻¹¹ M, 10⁻⁸ M to 10⁻¹⁰ M, or 10⁻⁹ M to 10⁻¹⁰ M. The on-rate (association rate constant; kₒₙ or kₐ; units of 1/Ms) and the off-rate (dissociation rate constant; k_{off} or k_{d}; units of 1/s) can be determined using any of the assay methods known in the art, for example, surface plasmon resonance (SPR).

In some embodiments, the binding affinity (EC₅₀) and/or the dissociation constant of the antibody (e.g. antigen-binding fragment) or antigen-binding domain of a CAR to BCMA protein, such as human BCMA protein, is from or from about 0.01 nM to about 500 nM, from or from about 0.01 nM to about 400 nM, from or from about 0.01 nM to about 100 nM, from or from about 0.01 nM to about 50 nM, from or from about 0.01 nM to about 10 nM, from or from about 0.01 nM to about 1 nM, from or from about 0.01 nM to about 0.1 nM, is from or from about 0.1 nM to about 500 nM, from or from about 0.1 nM to about 400 nM, from or from about 0.1 nM to about 100 nM, from or from about 0.1 nM to about 50 nM, from or from about 0.1 nM to about 10 nM, from or from about 0.1 nM to about 1 nM, from or from about 0.5 nM to about 200 nM, from or from about 1 nM to about 500 nM, from or from about 1 nM to about 100 nM, from or from about 1 nM to about 50 nM, from or from about 1 nM to about 10 nM, from or from about 2 nM to about 50 nM, from or from about 10 nM to about 500 nM, from or from about 10 nM to about 100 nM, from or from about 10 nM to about 50 nM, from or from about 50 nM to about 500 nM, from or from about 50 nM to about 100 nM or from or from about 100 nM to about 500 nM. In certain embodiments, the binding affinity (EC₅₀) and/or the equilibrium dissociation constant, K_{D}, of the antibody to a BCMA protein, such as human BCMA protein, is at or less than or about 400 nM, 300 nM, 200 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. In some embodiments, the antibodies bind to a BCMA protein, such as human BCMA protein, with a sub-nanomolar binding affinity, for example, with a binding affinity less than about 1 nM, such as less than about 0.9 nM, about 0.8 nM, about 0.7 nM, about 0.6 nM, about 0.5 nM, about 0.4 nM, about 0.3 nM, about 0.2 nM or about 0.1 nM or less.

In some embodiments, the binding affinity may be classified as high affinity or as low affinity. In some cases, the binding molecule (e.g. antibody or fragment thereof) or antigen-binding domain of a CAR that exhibits low to moderate affinity binding exhibits a K_{A} of up to 10⁷ M⁻¹, up to 10⁶ M⁻¹, up to 10⁵ M⁻¹. In some cases, a binding molecule (e.g. antibody or fragment thereof) that exhibits high affinity binding to a particular epitope interacts with such epitope with a K_{A} of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M¹. In some embodiments, the binding affinity (EC₅₀) and/or the equilibrium dissociation constant, K_{D}, of the binding molecule, e.g., anti-BCMA antibody or fragment thereof or antigen-binding domain of a CAR, to a BCMA protein, is from or from about 0.01 nM to about 1 μM, 0.1 nM to 1 μM, 1 nM to 1 μM, 1 nM to 500 nM, 1 nM to 100 nM, 1 nM to 50 nM, 1 nM to 10 nM, 10 nM to 500 nM, 10 nM to 100 nM, 10 nM to 50 nM, 50 nM to 500 nM, 50 nM to 100 nM or 100 nM to 500 nM. In certain embodiments, the binding affinity (EC₅₀) and/or the dissociation constant of the equilibrium dissociation constant, K_{D}, of the binding molecule, e.g., anti-BCMA antibody or fragment thereof or antigen-binding domain of a CAR, to a BCMA protein, is at or about or less than at or about 1 μM, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. The degree of affinity of a particular antibody can be compared with the affinity of a known antibody, such as a reference antibody.

In some embodiments, the binding affinity of a binding molecule, such as an anti-BCMA antibody or antigen-binding domain of a CAR, for different antigens, e.g., BCMA proteins from different species can be compared to determine the species cross-reactivity. For example, species cross-reactivity can be classified as high cross reactivity or low cross reactivity. In some embodiments, the equilibrium dissociation constant, K_{D}, for different antigens, e.g., BCMA proteins from different species such as human, cynomolgus monkey or mouse, can be compared to determine species cross-reactivity. In some embodiments, the species cross-reactivity of an anti-BCMA antibody or antigen-binding domain of a CAR can be high, e.g., the anti-BCMA antibody binds to human BCMA and a species variant BCMA to a similar degree, e.g., the ratio of K_{D} for human BCMA and K_{D} for the species variant BCMA is or is about 1. In some embodiments, the species cross-reactivity of an anti-BCMA antibody or antigen-binding domain of a CAR can be low, e.g., the anti-BCMA antibody has a high affinity for human BCMA but a low affinity for a species variant BCMA, or vice versa. For example, the ratio of K_{D} for the species variant BCMA and K_{D} for the human BCMA is more than 10, 15, 20, 25, 30, 40, 50, 60, 70, 80, 90, 100, 200, 500, 1000, 2000 or more, and the anti-BCMA antibody has low species cross-reactivity. The degree of species cross-reactivity can be compared with the species cross-reactivity of a known antibody, such as a reference antibody.

Among the provided bispecific CARs are CARs that exhibit antigen-dependent activity or signaling, i.e. signaling activity that is measurably absent or at background levels in the absence of antigen, e.g. BCMA. Thus, in some aspects, provided CARs do not exhibit, or exhibit no more than background or a tolerable or low level of, tonic signaling or antigen-independent activity or signaling in the absence of antigen, e.g. BCMA, being present. In some embodiments, the provided bispecific CAR-expressing cells exhibit biological activity or function, including cytotoxic activity, cytokine production, and ability to proliferate.

In some embodiments, biological activity or functional activity of a chimeric receptor, such as cytotoxic activity, can be measured using any of a number of known methods. The activity can be assessed or determined either in vitro or in vivo. In some embodiments, activity can be assessed once the cells are administered to the subject (e.g., human). Parameters to assess include specific binding of an engineered or natural T cell or other immune cell to antigen, e.g., in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as interlekukin-2 (IL-2), interferon-gamma (IFNγ), interleukin-4 (IL-4), TNF-alpha (TNFα), interleukin-6 (IL-6), interleukin-10 (IL-10), interleukin-12 (IL-12), granulocyte-macrophage colony-stimulating factor (GM-CSF), CD107a, and/or TGF-beta (TGFβ). Assays to measure cytokines are well known in the art, and include but are not limited to, ELISA, intracellular cytokine staining, cytometric bead array, RT-PCR, ELISPOT, flow cytometry and bio-assays in which cells responsive to the relevant cytokine are tested for responsiveness (e.g. proliferation) in the presence of a test sample. In some aspects, the biological activity is measured by assessing clinical outcome, such as reduction in tumor burden or load.

In some aspects, a reporter cell line can be employed to monitor antigen-independent activity and/or tonic signaling through bispecific CAR-expressing cells. In some embodiments, a T cell line, such as a Jurkat cell line (which is BCMA-negative/GPRC5D-negative), contains a reporter molecule, such as a fluorescent protein or other detectable molecule, such as a red fluorescent protein, expressed under the control of the endogenous Nur77 transcriptional regulatory elements. In some embodiments, the Nur77 reporter expression is cell intrinsic and dependent upon signaling through a recombinant reporter containing a primary activation signal in a T cell, a signaling domain of a T cell receptor (TCR) component, and/or a signaling domain comprising an immunoreceptor tyrosine-based activation motif (ITAM), such as a CD3ζ chain. Nur77 expression is generally not affected by other signaling pathways such as cytokine signaling or toll-like receptor (TLR) signaling, which may act in a cell extrinsic manner and may not depend on signaling through the recombinant receptor. Thus, only cells that express the exogenous recombinant receptor, e.g. bispecific CAR, containing the appropriate signaling regions is capable of expressing Nur77 upon stimulation (e.g., binding of the specific antigen). In some cases, Nur77 expression also can show a dose-dependent response to the amount of stimulation (e.g., antigen).

In some embodiments, the provided bispecific CARs exhibit improved expression on the surface of cells, such as compared to an alternative CAR that has an identical amino acid sequence but that is encoded by non-splice site eliminated and/or a non-codon-optimized nucleotide sequence. In some embodiments, the expression of the recombinant receptor on the surface of the cell can be assessed. Approaches for determining expression of the recombinant receptor on the surface of the cell may include use of chimeric antigen receptor (CAR)-specific antibodies (e.g., Brentjens et al., Sci. Transl. Med. 2013 March; 5(177): 177ra38), Protein L (Zheng et al., J. Transl. Med. 2012 February; 10:29), epitope tags, and monoclonal antibodies that specifically bind to a CAR polypeptide (see international patent application Pub. No. WO2014190273). In some embodiments, the expression of the recombinant receptor on the surface of the cell, e.g., primary T cell, can be assessed, for example, by flow cytometry, using binding molecules that can bind to the recombinant receptor or a portion thereof that can be detected. In some embodiments, the binding molecules used for detecting expression of the recombinant receptor is or comprises an anti-idiotypic antibody, e.g., an anti-idiotypic agonist antibody specific for a binding domain, e.g., scFv, or a portion thereof. In some embodiments, the binding molecule is or comprises an isolated or purified antigen, e.g., recombinantly expressed antigen.

### II. Polynucleotides Encoding Recombinant Receptor(s)

Also provided are polynucleotides encoding the chimeric antigen receptors and/or portions, e.g., chains, thereof. Among the provided polynucleotides are those encoding the bispecific chimeric antigen receptors (e.g., antigen-binding fragment) binding GPRC5D and BCMA described herein. The polynucleotides may include those encompassing natural and/or non-naturally occurring nucleotides and bases, e.g., including those with backbone modifications. The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

In some embodiments, the extracellular binding domains comprises, from amino- to carboxy-terminus: one of the V_{H} region and the V_{L} region of the GPRC5D-binding domain; the other of the V_{H} region and the V_{L} region of the GPRC5D-binding domain; one of the V_{H} region and the V_{L} region of the BCMA-binding domain; and the other of the V_{H} region and the V_{L} region of the BCMA-binding domain. In some embodiments, the extracellular binding domains comprises, from amino- to carboxy-terminus: one of the V_{H} region and the V_{L} region of the GPRC5D-binding domain; one of the V_{H} region and the V_{L} region of the BCMA-binding domain; the other of the V_{H} region and the V_{L} region of the BCMA-binding domain; and the other of the V_{H} region and the V_{L} region of the GPRC5D-binding domain. In some cases, the polynucleotide encoding the GPRC5D-binding domain contains a signal sequence that encodes a signal peptide, in some cases encoded upstream of the nucleic acid sequences encoding the GPRC5D-binding domain, or joined at the 5′ terminus of the nucleic acid sequences encoding the GPRC5D-binding domain. In some cases, the polynucleotide containing nucleic acid sequences encoding the GPRC5D-binding domain contains a signal sequence that encodes a signal peptide. In some aspects, the signal sequence may encode a signal peptide derived from a native polypeptide. In other aspects, the signal sequence may encode a heterologous or non-native signal peptide. In some aspects, non-limiting exemplary signal peptide include a signal peptide of the IgG kappa chain set forth in SEQ ID NO: 92, or encoded by the nucleotide sequence set forth in SEQ ID NO: 91 or 93-96. In some aspects, a non-limiting exemplary signal peptide includes a signal peptide of a GMCSFR alpha chain set forth in SEQ ID NO:98 and encoded by the nucleotide sequence set forth in SEQ ID NO:97. In some aspects, a non-limiting exemplary signal peptide includes a signal peptide of a CD8 alpha signal peptide set forth in SEQ ID NO:99. In some aspects, a non-limiting exemplary signal peptide includes a signal peptide of a CD33 signal peptide set forth in SEQ ID NO:72. In some cases, the polynucleotide encoding the GPRC5D-binding domain can contain nucleic acid sequence encoding additional molecules, such as a surrogate marker or other markers, or can contain additional components, such as promoters, regulatory elements and/or multicistronic elements. In some embodiments, the nucleic acid sequence encoding the GPRC5D-binding domain can be operably linked to any of the additional components.

In some embodiments, the extracellular binding domains comprises, from amino- to carboxy-terminus: one of the V_{H} region and the V_{L} region of the BCMA-binding domain; the other of the V_{H} region and the V_{L} region of the BCMA-binding domain; one of the V_{H} region and the V_{L} region of the GPRC5D-binding domain; and the other of the V_{H} region and the V_{L} region of the GPRC5D-binding domain. In some embodiments, the extracellular binding domains comprises, from amino- to carboxy-terminus: one of the V_{H} region and the V_{L} region of the BCMA-binding domain; one of the V_{H} region and the V_{L} region of the GPRC5D-binding domain; the other of the V_{H} region and the V_{L} region of the GPRC5D-binding domain; and the other of the V_{H} region and the V_{L} region of the BCMA-binding domain. In some cases, the polynucleotide encoding the BCMA-binding domain contains a signal sequence that encodes a signal peptide, in some cases encoded upstream of the nucleic acid sequences encoding the BCMA-binding domain, or joined at the 5′ terminus of the nucleic acid sequences encoding the BCMA-binding domain. In some cases, the polynucleotide containing nucleic acid sequences encoding the BCMA-binding domain contains a signal sequence that encodes a signal peptide. In some aspects, the signal sequence may encode a signal peptide derived from a native polypeptide. In other aspects, the signal sequence may encode a heterologous or non-native signal peptide. In some aspects, non-limiting exemplary signal peptide include a signal peptide of the IgG kappa chain set forth in SEQ ID NO: 92, or encoded by the nucleotide sequence set forth in SEQ ID NO: 271 or 93-96. In some aspects, a non-limiting exemplary signal peptide includes a signal peptide of a GMCSFR alpha chain set forth in SEQ ID NO:98 and encoded by the nucleotide sequence set forth in SEQ ID NO:97. In some aspects, a non-limiting exemplary signal peptide includes a signal peptide of a CD8 alpha signal peptide set forth in SEQ ID NO:99. In some aspects, a non-limiting exemplary signal peptide includes a signal peptide of a CD33 signal peptide set forth in SEQ ID NO:72. In some cases, the polynucleotide encoding the BCMA-binding domain can contain nucleic acid sequence encoding additional molecules, such as a surrogate marker or other markers, or can contain additional components, such as promoters, regulatory elements and/or multicistronic elements. In some embodiments, the nucleic acid sequence encoding the BCMA-binding domain can be operably linked to any of the additional components.

In some embodiments, a CAR provided herein is encoded by the nucleotide sequence set forth in any one of SEQ ID NOS:105-120. In some embodiments, the CAR is encoded by the nucleotide sequence set forth in SEQ ID NO:105. In some embodiments, the CAR is encoded by the nucleotide sequence set forth in SEQ ID NO:106. In some embodiments, the CAR is encoded by the nucleotide sequence set forth in SEQ ID NO:107. In some embodiments, the CAR is encoded by the nucleotide sequence set forth in SEQ ID NO:108. In some embodiments, the CAR is encoded by the nucleotide sequence set forth in SEQ ID NO:109. In some embodiments, the CAR is encoded by the nucleotide sequence set forth in SEQ ID NO:110. In some embodiments, the CAR is encoded by the nucleotide sequence set forth in SEQ ID NO:111. In some embodiments, the CAR is encoded by the nucleotide sequence set forth in SEQ ID NO:112. In some embodiments, the CAR is encoded by the nucleotide sequence set forth in SEQ ID NO:113. In some embodiments, the CAR is encoded by the nucleotide sequence set forth in SEQ ID NO:114. In some embodiments, the CAR is encoded by the nucleotide sequence set forth in SEQ ID NO:115. In some embodiments, the CAR is encoded by the nucleotide sequence set forth in SEQ ID NO:116. In some embodiments, the CAR is encoded by the nucleotide sequence set forth in SEQ ID NO:117. In some embodiments, the CAR is encoded by the nucleotide sequence set forth in SEQ ID NO: 118. In some embodiments, the CAR is encoded by the nucleotide sequence set forth in SEQ ID NO:119. In some embodiments, the CAR is encoded by the nucleotide sequence set forth in SEQ ID NO:120.

In some embodiments, among CARs provided herein are those encoded by polynucleotides that are optimized, or contain certain features designed for optimization, such as for codon usage, to reduce RNA heterogeneity and/or to modify, e.g., increase or render more consistent among cell product lots, expression, such as surface expression, of the encoded receptor. In some embodiments, polynucleotides, encoding GPRC5D-binding domains or BCMA-binding domains, are modified as compared to a reference polynucleotide, such as to remove cryptic or hidden splice sites, to reduce RNA heterogeneity. In some embodiments, polynucleotides, encoding GPRC5D-binding and BCMA-binding domains, are codon optimized, such as for expression in a mammalian, e.g., human, cell, such as in a human T cell. In some aspects, the modified polynucleotides result in in improved, e.g., increased or more uniform or more consistent level of, expression, e.g., surface expression, when expressed in a cell. Such polynucleotides can be utilized in constructs for generation of engineered cells that express the encoded GPRC5D-binding and BCMA-binding domains. Thus, also provided are cells expressing the recombinant receptors encoded by the polynucleotides provided herein and uses thereof in adoptive cell therapy, such as treatment of diseases and disorders associated with GPRC5D and/or BCMA expression, e.g., multiple myeloma.

Also provided are cells, such as T cells, engineered to express a polynucleotide encoding a provided polynucleotide, including polynucleotides encoding a GPRC5D-binding domain and a BCMA-binding domain, and compositions containing such cells. In some embodiments, the polynucleotide constructs are codon optimized for expression in a human cell. In some embodiments, one or more splice donor and/or acceptor sites in a polynucleotide construct is modified to reduce heterogeneity of the RNA transcribed from the construct, such as mRNA, following expression in a cell.

1. Codon Optimization

In some embodiments the polynucleotides are modified by optimization of the codons for expression in humans. In some aspects, codon optimization can be considered before and/or after the steps for splice site identification and/or splice site elimination, and/or at each of the iterative steps for reducing RNA heterogeneity. Codon optimization generally involves balancing the percentages of codons selected with the abundance, e.g., published abundance, of human transfer RNAs, for example, so that none is overloaded or limiting. In some cases, such balancing is necessary or useful because most amino acids are encoded by more than one codon, and codon usage generally varies from organism to organism. Differences in codon usage between transfected or transduced genes or nucleic acids and host cells can have effects on protein expression from the nucleic acid molecule. Table 2 below sets forth an exemplary human codon usage frequency table. In some embodiments, to generate codon-optimized nucleic acid sequences, codons are chosen to select for those codons that are in balance with human usage frequency. The redundancy of the codons for amino acids is such that different codons code for one amino acid, such as depicted in Table 2. In selecting a codon for replacement, it is desired that the resulting mutation is a silent mutation such that the codon change does not affect the amino acid sequence. Generally, the last nucleotide of the codon (e.g., at the third position) can remain unchanged without affecting the amino acid sequence.

TABLE 2
Human Codon Usage Frequency

| Human | amino | freq./ | | Human | amino | freq./ | |
|---|---|---|---|---|---|---|---|
| codon | acid | 1000 | number | codon | acid | 1000 | number |
| TTT | F | 17.6 | 714298 | TCT | S | 15.2 | 618711 |
| TTC | F | 20.3 | 824692 | TCC | S | 17.7 | 718892 |
| TTA | L | 7.7 | 311881 | TCA | S | 12.2 | 496448 |
| TTG | L | 12.9 | 525688 | TCG | S | 4.4 | 179419 |
| CTT | L | 13.2 | 536515 | CCT | P | 17.5 | 713233 |
| CTC | L | 19.6 | 796638 | CCC | P | 19.8 | 804620 |
| CTA | L | 7.2 | 290751 | CCA | P | 16.9 | 688038 |
| CTG | L | 39.6 | 1611801 | CCG | P | 6.9 | 281570 |
| ATT | I | 16 | 650473 | ACT | T | 13.1 | 533609 |
| ATC | I | 20.8 | 846466 | ACC | T | 18.9 | 768147 |
| ATA | I | 7.5 | 304565 | ACA | T | 15.1 | 614523 |
| ATG | M | 22 | 896005 | ACG | T | 6.1 | 246105 |
| GTT | V | 11 | 448607 | GCT | A | 18.4 | 750096 |
| GTC | V | 14.5 | 588138 | GCC | A | 27.7 | 1127679 |
| GTA | V | 7.1 | 287712 | GCA | A | 15.8 | 643471 |
| GTG | V | 28.1 | 1143534 | GCG | A | 7.4 | 299495 |
| TAT | Y | 12.2 | 495699 | TGT | C | 10.6 | 430311 |
| TAC | Y | 15.3 | 622407 | TGC | C | 12.6 | 513028 |
| TAA | * | 1 | 40285 | TGA | * | 1.6 | 63237 |
| TAG | * | 0.8 | 32109 | TGG | W | 13.2 | 535595 |
| CAT | H | 10.9 | 441711 | CGT | R | 4.5 | 184609 |
| CAC | H | 15.1 | 613713 | CGC | R | 10.4 | 423516 |
| CAA | Q | 12.3 | 501911 | CGA | R | 6.2 | 250760 |
| CAG | Q | 34.2 | 1391973 | CGG | R | 11.4 | 464485 |
| AAT | N | 17 | 689701 | AGT | S | 12.1 | 493429 |
| AAC | N | 19.1 | 776603 | AGC | S | 19.5 | 791383 |
| AAA | K | 24.4 | 993621 | AGA | R | 12.2 | 494682 |
| AAG | K | 31.9 | 1295568 | AGG | R | 12 | 486463 |
| GAT | D | 21.8 | 885429 | GGT | G | 10.8 | 437126 |
| GAC | D | 25.1 | 1020595 | GGC | G | 22.2 | 903565 |
| GAA | E | 29 | 1177632 | GGA | G | 16.5 | 669873 |
| GAG | E | 39.6 | 1609975 | GGG | G | 16.5 | 669768 |

For example, the codons TCT, TCC, TCA, TCG, AGT and AGC all code for Serine (note that T in the DNA equivalent to the U in RNA). From a human codon usage frequency, such as set forth in Table 2 above, the corresponding usage frequencies for these codons are 15.2, 17.7, 12.2, 4.4, 12.1, and 19.5, respectively. Since TCG corresponds to 4.4%, if this codon were commonly used in a gene synthesis, the tRNA for this codon would be limiting. In codon optimization, the goal is to balance the usage of each codon with the normal frequency of usage in the species of animal in which the transgene is intended to be expressed.

2. Splice Sites

Provided herein are polynucleotides in which one or more potential splice donor and/or splice acceptor sites have been identified and the nucleic acid sequence at or near the one or more of the identified splice donor sites has been modified. In some embodiments, the resulting modified nucleic acid sequence(s) is/are then synthesized and used to transduce cells to test for splicing as indicated by RNA heterogeneity.

Also provided here are polynucleotides, such as those encoding any of the antibodies, receptors (such as antigen receptors such as chimeric antigen receptors) and/or GPRC5D-specific and/or BCMA-specific binding domains provided herein, that are or have been modified to reduce heterogeneity or contain one or more nucleic acid sequences observed herein (such as by the optimization methods) to result in improved features of the polypeptides, such as the CARs, as compared to those containing distinct, reference, sequences or that have not been modified. Among such features include improvements in RNA heterogeneity, such as that resulting from the presence of one or more splice sites, such as one or more cryptic splice sites, and/or improved expression and/or surface expression of the encoded protein, such as increased levels, uniformity, or consistency of expression among cells or different therapeutic cell compositions engineered to express the polypeptides.

Splice sites may be identified in polynucleotide sequences by harvesting RNA from the expressing cells, amplifying by reverse transcriptase polymerase chain reaction (RT-PCR) and resolving by agarose gel electrophoresis to determine the heterogeneity of the RNA, compared to the starting sequence. In some cases, improved sequences can be resubmitted to the gene synthesis vendor for further codon optimization and splice site removal, followed by further cryptic splice site evaluation, modification, synthesis and testing, until the RNA on the agarose gel exhibits minimal RNA heterogeneity

Also provided are polynucleotides that have been modified to eliminate splice sites, such as cryptic splice sites. Genomic nucleic acid sequences generally, in nature, in a mammalian cell, undergo processing co-transcriptionally or immediately following transcription, wherein a nascent precursor messenger ribonucleic acid (pre-mRNA), transcribed from a genomic deoxyribonucleic acid (DNA) sequence, is in some cases edited by way of splicing, to remove introns, followed by ligation of the exons in eukaryotic cells. Consensus sequences for splice sites are known, but in some aspects, specific nucleotide information defining a splice site may be complex and may not be readily apparent based on available methods. Cryptic splice sites are splice sites that are not predicted based on the standard consensus sequences and are variably activated. Hence, variable splicing of pre-mRNA at cryptic splice sites leads to heterogeneity in the transcribed mRNA products upon expression in eukaryotic cells.

Polynucleotides generated for the expression of transgenes are typically constructed from nucleic acid sequences, such as complementary DNA (cDNA), or portions thereof, that do not contain introns. Thus, splicing of such sequences is not expected to occur. However, the presence of cryptic splice sites within the cDNA sequence can lead to unintended or undesired splicing reactions and heterogeneity in the transcribed mRNA. Such heterogeneity results in translation of unintended protein products, such as truncated protein products with variable amino acid sequences that exhibit modified expression and/or activity.

In some embodiments, eliminating splice sites, such as cryptic splice sites, can improve or optimize expression of a transgene product, such as a polypeptide translated from the transgene, such as a bispecific CAR polypeptide. Splicing at cryptic splice sites of an encoded transgene, such as an encoded CAR comprising a GPRC5D-binding domain and BCMA-binding domain, can lead to reduced protein expression, e.g., expression on cell surfaces, and/or reduced function, e.g., reduced intracellular signaling. Provided herein are polynucleotides, encoding bispecific CAR proteins that have been optimized to reduce or eliminate cryptic splice sites. Also provided herein are polynucleotides encoding bispecific CAR proteins that have been optimized for codon expression and/or in which one or more sequence, such as one identified by the methods or observations herein regarding splice sites, is present, and/or in which an identified splice site, such as any of the identified splice sites herein, is not present. Among the provided polynucleotides are those exhibiting below a certain degree of RNA heterogeneity or splice forms when expressed under certain conditions and/or introduced into a specified cell type, such as a human T cell, such as a primary human T cell, and cells and compositions and articles of manufacture containing such polypeptides and/or exhibiting such properties. In some embodiments, the RNA heterogeneity of transcribed RNA is reduced by greater than or greater than about 10%, 15%, 20%, 25%, 30%, 40%, 50% or more compared to a polynucleotide that has not been modified to remove cryptic splice sites and/or by codon optimization. In some embodiments, the provided polynucleotides encoding a bispecific CAR exhibit RNA homogeneity of transcribed RNA that is at least 70%, 75%, 80%, 85%, 90%, or 95% or greater.

RNA heterogeneity can be determined by any of a number of methods provided herein or described or known. In some embodiments, RNA heterogeneity of a transcribed nucleic acid is determined by amplifying the transcribed nucleic acid, such as by reverse transcriptase polymerase chain reaction (RT-PCR) followed by detecting one or more differences, such as differences in size, in the one or more amplified products. In some embodiments, the RNA heterogeneity is determined based on the number of differently sized amplified products, or the proportion of various differently sized amplified products. In some embodiments, RNA, such as total RNA or cytoplasmic polyadenylated RNA, is harvested from cells, expressing the transgene to be optimized, and amplified by reverse transcriptase polymerase chain reaction (RT-PCR) using a primer specific to the 5′ untranslated region (5′ UTR), in some cases corresponding to a portion of the promoter sequence in the expression vector, located upstream of the transgene in the transcribed RNA, and a primer specific to the 3′ untranslated region (3′ UTR), located downstream of the expressed transgene in the transcribed RNA sequence or a primer specific to a sequence within the transgene. In particular embodiments, at least one primer complementary to a sequence in the 5′ untranslated region (UTR) and at least one primer complementary to a sequence in the 3′ untranslated region (UTR) are employed to amplify the transgene. The skilled artisan can resolve RNA, such as messenger RNA, and analyze the heterogeneity thereof by several methods. Non-limiting, exemplary methods include agarose gel electrophoresis, chip-based capillary electrophoresis, analytical centrifugation, field flow fractionation, and chromatography, such as size exclusion chromatography or liquid chromatography.

In some aspects, the presence of potential cryptic splice sites (splice donor and/or acceptor sites that are present in a transcript, such as a transgene transcript, can result in RNA heterogeneity of the transcript following expression in a cell. In some embodiments, the one or more potential splice sites that can be present in the transgene transcript, that are not desired and/or that may be created in a transgene transcript from various underlying sequences are identified, following codon optimization of a transcript and/or by mutation or mistake or error in transcription. In some aspects of the provided embodiments, the splice donor sites and splice acceptor sites are identified independently. In some embodiments, the splice acceptor and/or donor site(s) is/are canonical, non-canonical, and/or cryptic splice acceptor and/or donor site(s).

In some embodiments, one or more potential splice site (e.g., canonical, non-canonical, and/or cryptic splice acceptor and/or donor site(s) or branch sites) in a polynucleotide, such as a polynucleotide encoding a transgene, such as a recombinant receptor, that may exhibit RNA heterogeneity, are identified and/or modified. Also provided are polypeptides having reduced numbers of such splice sites as compared to such reference polynucleotides.

In some aspects, identification of the one or more splice sites in a nucleic acid sequence is an iterative process. In some embodiments, splice sites can be identified using a splice site and/or codon optimization prediction tool, such as by submitting the starting or reference sequence encoding the transgene, such as a bispecific CAR, or a GPRC5D- or BCMA-binding domain comprised therein, to a database, a gene synthesis vendor or other source able to computationally or algorithmically compare the starting or reference sequence to identify or predict splice sites and/or for codon optimization and/or splice site removal. In some embodiments, after modifying the sequence for codon optimization and/or splice site removal, one or more further assessment of a sequence, such as a revised or modified nucleic acid sequence, is carried out to further evaluate for splice site removal, such as cryptic splice sites, using one or more other or additional splice site prediction tool(s).

In some aspects, RNA heterogeneity can be a result of the activity of the spliceosome present in a eukaryotic cell. In some aspects, splicing is typically carried out in a series of reactions catalyzed by the spliceosome. Consensus sequences for splice sites are known, but in some aspects, specific nucleotide information defining a splice site may be complex and may not be readily apparent based on available methods. Cryptic splice sites are splice sites that are not predicted based on the standard consensus sequences and are variably activated. Hence, variable splicing of pre-mRNA at cryptic splice sites leads to heterogeneity in the transcribed mRNA products following expression in eukaryotic cells. In some cases, within spliceosomal introns, a donor site (usually at the 5′ end of the intron), a branch site (near the 3′ end of the intron) and an acceptor site (3′ end of the intron) are required for a splicing event. The splice donor site can include a GU sequence at the 5′ end of the intron, with a large less highly conserved region. The splice acceptor site at the 3′ end of the intron can terminate with an AG sequence.

In some embodiments, splice sites, including potential cryptic splice sites can be identified by comparing sequences to known splice site sequences, such as those in a sequence database. In some embodiments, splice sites can be identified by computationally by submitting nucleotide sequences for analysis by splice site prediction tools, such as Human Splice Finder (Desmet et al., Nucl. Acids Res. 37(9):e67 (2009)), a neural network splice site prediction tool, NNSplice (Reese et al., J. Comput. Biol., 4(4):311 (1997)), GeneSplicer (Pertea et al., Nucleic Acids Res. 2001 29(5): 1185-1190) or NetUTR (Eden and Brunak, Nucleic Acids Res. 32(3):1131 (2004)), which identify potential splice sites and the probability of a splicing event at such sites. Additional splice prediction tools include RegRNA, ESEfinder, and MIT splice predictor. Splice site prediction tools such as GeneSplicer has been trained and/or tested successfully on databases for different species, such as human, Drosophila melanogaster, Plasmodium falciparum, Arabidopsis thaliana, and rice. In some embodiments, different prediction tools may be adapted for different extents on different database and/or for different species. In some embodiments, the one or more prediction tools are selected based upon their utility in certain database and/or for certain species. See, e.g., Saxonov et al., (2000) Nucleic Acids Res., 28, 185-190.

In some embodiments, one or more splice site prediction tools are used to determine potential splice donor and/or acceptor sites. In some embodiments, splice site prediction tools that can be run locally; that can be retrained with a set of data at the user site; that can use databases for particular species (such as human), that can be compiled for multiple platforms, that allow real-time predictions for sequence selections, and/or that is an OSI certified open source software such that particular tools or plugins can be modified, can be employed. Exemplary tools that can be employed include NNSplice, GeneSplicer or both.

In some aspects, the splice site prediction tools can be used to identify a list of potential splice donor and/or splice acceptor sites in a sequence such as a polynucleotide sequence containing transgene sequences. In some aspects, the prediction tools also can generate one or more prediction scores for one or more sequences in the polynucleotide, that can indicate the likelihoods of the one or more sequences being a splice donor or acceptor site sequence.

In some embodiments, the prediction score for a particular splice site is compared with a threshold score or reference score to determine or identify a particular splice sites that are candidate for elimination or removal. For example, in some embodiments, the predicted splice site is identified as a potential splice site when the prediction score is greater or no less than the threshold score or reference score. In some aspects, considerations for eliminating or removing a particular splice site include the prediction score as compared to a reference score or a threshold score; and whether a particular splice site is desired or intentional (for example, when the splicing event is more advantageous or is required for regulation of transcription and/or translation). In some aspects, the likelihood that the resulting splice variant loses the desired function or has compromised function can also be considered when determining particular donor and/or acceptor sites for elimination or removal. In some aspects, the one or more potential splice donor and/or splice acceptor sites exhibit a score about or at least about 0.7, 0.75, 0.8, 0.85, 0.9, 0.95, or 1.0 (e.g., on a scale with a maximum of 1.0) of a splice event or probability of a splice event, and the site can be a candidate for splice site elimination or removal. In some aspects, the score, e.g., used by GeneSplicer, at the one or more potential splice donor and/or splice site is based on the difference between the log-odds score returned for that sequence by the true Markov model and the score is computed by the false Markov model. In particular embodiments, the splice donor sites and splice acceptor sites are evaluated independently, or individually. In some embodiments, splice donor sites and splice acceptor sites are evaluated as a splice donor/acceptor pair.

In some embodiments, one or more splice donor and/or splice acceptor site(s), such as the potential splice donor and/or acceptor sites that may be involved in a cryptic splicing event that is not desired or that results in undesired RNA heterogeneity, is eliminated. In some embodiments, eliminating one or more splice sites comprises modifying one or more nucleotides (e.g., by substitution or replacement) in, at, containing or near the splice donor and/or acceptor sites that are candidates for removal. In some aspects, a particular nucleotide within a codon that is at, contains or is near the splice site is modified (e.g., substituted or replaced). In some aspects, the modification (such as substitution or replacement) retains or preserves the amino acid encoded by the particular codon at the site, at the same time removing the potential splice donor and/or acceptor sites.

In some embodiments, the codon at or near the splice site for modification comprises one or more codons that involve one or both of the two nucleotides at the potential splice site (in some cases referred to as “splice site codon”). When the potential splicing is predicted to occur between two nucleotides in a codon, the codon is the only splice site codon for this splice site. If the potential splicing is predicted to occur between two adjacent codons, for example, between the last nucleotide of the first codon and the first nucleotide of the next codon, the two codons are splice site codons. For example, for splice sites that are predicted to be at boundaries of two codons, the two adjacent codons can be candidates for nucleotide modification. In some embodiments, the one or more codons comprise one splice site codon. In some embodiments, the one or more codons comprise both splice site codons. In some embodiments, a potential splice donor site is eliminated by modifying one or both splice site codons. In some embodiments, a potential splice acceptor donor site is eliminated by modifying one or both splice site codons. In some embodiments, the one or both codons at the splice site is not modified, for example, when there are no synonymous codon for the splice site codon. In some embodiments, if there are no synonymous codons available for the particular splice site codon, one or more nucleotides in a nearby codon can be modified. In some embodiments, one or more codons that are modified include a splice site codon, wherein the modification comprises changing one or both nucleotides at the splice site to a different nucleotide or different nucleotides. In some embodiments, In some embodiments, the splice donor site is eliminated by modifying one or both splice site codons, wherein the modification does not change one or two of the nucleotides of the at the splice site to a different nucleotide, but a nearby nucleotide, e.g., a part of a codon adjacent to the splice site, is modified. In some embodiments, the nearby or adjacent nucleotides that can be modified include modification of a nucleotide that is a part of a nearby or adjacent codon, such as a codon that is within one, two, three, four, five, six, seven, eight, nine or ten codons upstream or downstream of the splice site codon.

In some cases, polynucleotides can be manually modified, while preserving the encoded amino acid sequence, to reduce the probability of a predicted splice site. In some embodiments, one or more of the predicted splice sites having at least 80%, 85%, 90%, or 95% probability of a splice site are manually modified to reduce the probability of the splicing event. In some embodiments, the one or more modification(s) is/are by nucleotide replacement or substitution of 1, 2, 3, 4, 5, 6 or 7 nucleotides. In some embodiments, the modification(s) is/are at the junction of the splice donor site or are at the junction of the splice acceptor site. In some embodiments, at least one of the one or more nucleotide modifications is within 1, 2, 3, 4, 5, 6, 7, 8, 9 or 10 residues of the splice site junction of the splice acceptor and/or splice donor site. In some embodiments, libraries of modified nucleic acid sequences can be generated with reduced probability of cryptic splice sites. In some embodiments, splice donor sites and splice acceptor sites are evaluated as a splice donor/acceptor pair. In particular embodiments, the splice donor sites and splice acceptor sites are evaluated independently, or individually, and not part as a splice donor/acceptor pair. In some embodiments, one or more predicted splice sites are not eliminated. In some embodiments, splice sites, such as known or predicted splice sites, within the promoter region of the transcript are not eliminated.

In some embodiments, one or more potential donor splice site is eliminated by modifying one or two splice site codons or one or more nearby or adjacent codons (for example, if a synonymous codon is not available for the splice site codon). In some embodiments, one or more potential acceptor splice site is eliminated by modifying one or two splice site codons or one or more nearby or adjacent codons (for example, if a synonymous codon is not available for the splice site codon). In some embodiments, the nearby or adjacent codon that is subject to modification include a codon that is within one, two, three, four, five, six, seven, eight, nine or ten codons upstream or downstream of the splice site codon, such as a codon that is within one, two or three codons from the splice site. In some embodiments, a potential branch site for splicing is removed or eliminated. In some aspects, a nucleotide within the codon at or near the branch site can be modified, e.g., substituted or replaced, to eliminate cryptic splicing and/or reduce RNA heterogeneity. In some embodiments, the modification of the one or more nucleotides can involve a substitution or replacement of one of the nucleotides that may be involved in splicing (such as at the splice donor site, splice acceptor site or splice branch site), such that the amino acid encoded by the codon is preserved, and the nucleotide substitution or replacement does not change the polypeptide sequence that is encoded by the polynucleotide. In some cases, the third position in the codon is more degenerate than the other two positions. Thus, various synonymous codons can encode a particular amino acid (see, e.g., Section II.1. above). In some embodiments, the modification includes replacing the codon with a synonymous codon used in the species of the cell into which the polynucleotide is introduced (e.g., human). In some embodiments, the species is human. In some embodiments, the one or more codon is replaced with a corresponding synonymous codons that the most frequently used in the species or synonymous codons that have a similar frequency of usage (e.g., most closest frequency of usage) as the corresponding codon (see, e.g., Section II.1. above).

In some embodiments, the transgene candidacy for the removal of splice sites is assessed, after initial proposed modification. In some aspects, the proposed modification can be evaluated again, to assess the proposed modification and identify any further potential splice sites after modification and/or codon optimization. In some aspects, after modifying the sequence for codon optimization and/or splice site removal, one or more further assessment of a sequence, such as a revised or modified nucleic acid sequence, is carried out to further evaluate for splice site removal, such as cryptic splice sites, using the same or one or more other or additional splice site prediction tool(s). In some aspects, proposed modifications are considered for subsequent steps, and iterative optimization can be used. In some aspects, the methods any of the identification and/or modification steps may be repeated, for example, until heterogeneity of the transcript is reduced compared to the heterogeneity of the transcript as initially determined. In some embodiments, a further or a different modification, such as with a different nucleotide replacement at the same codon or a modification at a different position or codon, can be done after an iterative evaluation and assessment. In some embodiments, corresponding different synonymous codon can be used, such as the second most frequently used in the particular species or a codon that has a similar frequency of usage (e.g., the next closest frequency of usage) as the corresponding codon (see, e.g., Section II.1 above).

In some aspects, a proposed modification can be further evaluated, for example, to assess whether the modification generates an undesired or additional restriction site in the polynucleotide. In some aspects, an additional restriction site may not be desired, and a further or a different modification (e.g., with a different nucleotide replacement at the same codon or a modification at a different position or codon) can be considered. In some aspects, particular restriction site, such as a designated restriction site, is avoided. In some aspects, if the modification does not substantially reduce the splice site prediction score, an additional or alternative modification can be proposed. In some embodiments, the splice site prediction score can be is reduced or lowered by at least about 5%, 10%, 15%, 20%, 25%, 30%, 35%, 40%, 45%, 50%, 55%, 60%, 65%, 70% or 75%, after one or more iteration of the methods.

In some embodiments, a computer system can be used to execute one or more steps, tools, functions, processes or scripts. In some embodiments, the splice site prediction, evaluation and modification for elimination or removal of a splice site can be performed by computer implemented methods and/or by methods which include steps that are computer implemented steps. In some embodiments, comparison of the sequences to a known database, calculating a splice site prediction score, determining potential nucleotide modifications, codon optimization and/or any one of the iterative steps can be implemented by a computer or using a computer-implemented steps, tools, functions, processes or scripts. In particular embodiments, a computer system comprising a processor and memory is provided, wherein the memory contains instructions operable to cause the processor to carry out any one or more of steps of the methods provided herein. In some embodiments, steps, functions, processes or scripts are performed computationally, e.g., performed using one or more computer programs and/or via the use of computational algorithms

Exemplary steps, functions, processes or scripts for identifying and/or removing possible splice sites include one or more steps of: selecting sequence, writing FASTA format sequences, loading codon table (e.g., from www.kazusa.or.jp/codon, running GeneSplicer, loading predictions, parsing codons, determining overlaps in prediction, identifying next highest usage synonymous codon, reviewing for restriction site, creating annotations or assessing other codons. Particular steps can assess both forward and reverse strands. In some aspects, previously annotated splice site modifications can also be considered, to allow for iterative optimization. In some embodiments, any one or more of the steps, functions, processes or scripts can be repeated.

In some embodiments, a provided polynucleotide encoding a CAR provided herein, or a construct provided herein, includes modifications to remove one or more splice donor and/or acceptor site that may contribute to splice events and/or reduced expression and/or increased RNA heterogeneity. In some embodiments, a CAR is encoded by the nucleotide sequence set forth in SEQ ID NO:119. In some embodiments, a CAR is encoded by the nucleotide sequence set forth in SEQ ID NO:120.

3. Other Features

Also provided are vectors containing the polynucleotides and host cells containing the vectors, e.g., for producing the chimeric antigen receptors. Also provided are methods for producing the chimeric antigen receptors. The nucleic acid may encode a chimeric antigen receptor comprising a VL region and/or a V_{H} region of an antibody (e.g., the light and/or heavy chains of the antibody). The nucleic acid may encode one or more amino binding domains (e.g., a BCMA-binding domain and a GPRC5D-binding domain) each comprising a VL region and/or a V_{H} region of an antibody (e.g., the light and/or heavy chains of the antibody). In a further embodiment, one or more vectors (e.g., expression vectors) comprising such polynucleotides are provided. In a further embodiment, a host cell comprising such polynucleotides is provided. In one such embodiment, a host cell comprises (e.g., has been transformed with) a vector comprising a nucleic acid that encodes chimeric antigen receptor comprising the V_{H} region of an antibody. In another such embodiment, a host cell comprises (e.g., has been transformed with) (1) a vector comprising a nucleic acid that encodes a chimeric antigen receptor comprising the VL region of the antibody and the V_{H} region of the antibody, or (2) a vector comprising a nucleic acid that encodes a chimeric antigen receptor comprising a first antibody and a second antibody. In some embodiments, a host cell comprises (e.g., has been transformed with) one or more vectors comprising one or more nucleic acid that encodes one or more chimeric antigen receptors. In some embodiments, one or more such host cells are provided. In some embodiments, a composition containing one or more such host cells are provided. In some embodiments, the one or more host cells can express different chimeric antigen receptors, or the same chimeric antigen receptor. In some embodiments, each of the host cells can express more than one chimeric antigen receptor.

Also provided are methods of making the bispecific chimeric antigen receptors that bind to BCMA and GPRC5D. For recombinant production of the chimeric receptors, a nucleic acid sequence encoding a chimeric receptor antibody, e.g., as described herein, may be isolated and inserted into one or more vectors for further cloning and/or expression in a host cell. Such nucleic acid sequences may be readily isolated and sequenced using conventional procedures (e.g., by using oligonucleotide probes that are capable of binding specifically to genes encoding the heavy and light chains of the antibody). In some embodiments, a method of making the bispecific chimeric antigen receptor is provided, wherein the method comprises culturing a host cell comprising a nucleic acid sequence encoding the antibodies (i.e., the BCMA-binding domain and the GPRC5D-binding domain), as provided above, under conditions suitable for expression of the receptor.

In some embodiments, a method of making a cellular composition comprising cells expressing the bispecific chimeric antigen receptor is provided.

In addition to prokaryotes, eukaryotic microbes such as filamentous fungi or yeast are suitable cloning or expression hosts for antibody-encoding vectors, including fungi and yeast strains whose glycosylation pathways have been modified to mimic or approximate those in human cells, resulting in the production of an antibody with a partially or fully human glycosylation pattern. See Gerngross, Nat. Biotech. 22:1409-1414 (2004), and Li et al., Nat. Biotech. 24:210-215 (2006).

Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells; and NSO cells. In some embodiments, the antibody heavy chains and/or light chains (e.g., V_{H} region and/or VL region) may be expressed in yeast (see, e.g., U.S. Publication No. US 2006/0270045 A1). In some embodiments, a particular eukaryotic host cell is selected based on its ability to make desired post-translational modifications to the heavy chains and/or light chains (e.g., V_{H} region and/or VL region). For example, in some embodiments, CHO cells produce polypeptides that have a higher level of sialylation than the same polypeptide produced in 293 cells. In particular examples immune cells, such as human immune cells are used to express the provided polypeptides encoding chimeric antigen receptors. In some examples, the immune cells are T cells, such as CD4+ and/or CD8+ immune cells

### III. Engineered Cells

Also provided are cells such as engineered cells that contain a recombinant receptor (e.g., a chimeric antigen receptor) such as one that contains an extracellular domain including both a GPRC5D-binding domain and a BCMA-binding domain as provided herein. Also provided are populations of such cells, compositions containing such cells and/or enriched for such cells, such as in which cells expressing the GPRC5D-binding domain and the BCMA binding domain make up at least 50, 60, 70, 80, 90, 91, 92, 93, 94, 95, 96, 97, 98, 99, or more percent of the total cells in the composition or cells of a certain type such as T cells, CD8+ cells or CD4+ cells.

Also provided are cells such as engineered cells that are engineered to contain a recombinant receptor (e.g., a CAR) comprising a GPRC5D-binding domain and a BCMA-binding domain. In some embodiments, the recombinant receptor is a tandem CAR comprising a GPRC5D-binding domain and BCMA-binding domain. The GPRC5D-binding domain can be any known GPRC5D-binding domain, such as included in an anti-GPRC5D CAR described herein or elsewhere (see, e.g., WO 2016/090312, WO 2016/090329, WO 2018/017786, WO2020148677, WO2019154890, WO2021018859, WO2021018925, and WO2018147245). Exemplary GPRC5D-binding domains are described in Section I. The BCMA-binding domain can be any known BCMA-binding domain, such as included in an anti-BCMA CAR described herein or elsewhere (see, e.g., WO 2013/154760, WO 2015/052538, WO 2015/090229, WO 2015/092024, WO 2015/158671, WO 2016/014565, WO 2016/014789, WO 2016/094304, WO 2016/166630, WO 2017/021450, WO 2017/083511, WO 2017/130223, WO 2017/211900, WO 2018/085690, WO 2018/028647). Exemplary BCMA-binding domains are described in Section I.

In some embodiments, the engineered cells provided herein can be combined with one or more engineered cell population(s) expressing one or more other recombinant receptor(s). Such engineered cell populations can be formulated in the same or separate compositions. Among the compositions are pharmaceutical compositions and formulations for administration, such as for adoptive cell therapy. Also provided are therapeutic methods for administering any of the cells or compositions provided herein to subjects, e.g., patients.

Thus, also provided are genetically engineered cells expressing the recombinant receptors containing the antibodies, e.g., cells containing the CARs. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include T cells. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells include CD4+ T cells. In some embodiments, the cells include CD8+ T cells. In some embodiments, the cells include CD4+ and CD8+ T cells. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more polynucleotides introduced via genetic engineering, and thereby express recombinant or genetically engineered products of such polynucleotides. In some embodiments, the polynucleotides are heterologous, i.e., normally not present in a cell or sample obtained from the cell, such as one obtained from another organism or cell, which for example, is not ordinarily found in the cell being engineered and/or an organism from which such cell is derived. In some embodiments, the polynucleotides are not naturally occurring, such as a polynucleotide not found in nature, including one comprising chimeric combinations of polynucleotides encoding various domains from multiple different cell types. In some embodiments, the cells (e.g., engineered cells) comprise a vector (e.g., a viral vector, expression vector, etc.) as described herein such as a vector comprising a nucleic acid encoding a recombinant receptor described herein.

A. Vectors and Methods for Genetic Engineering

Also provided are methods, polynucleotides, compositions, and kits, for expressing the bispecific recombinant receptors (e.g., CARs), and for producing the genetically engineered cells expressing such receptors. In some embodiments, one or more recombinant receptors (e.g., CARs) can be genetically engineered into cells or plurality of cells. The genetic engineering generally involves introduction of a nucleic acid encoding the recombinant or engineered component(s) into the cell, such as by lentiviral transduction, retroviral transduction, transfection, or transformation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example, in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphoribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some aspects, the cells further are engineered to promote expression of cytokines or other factors. Various methods for the introduction of genetically engineered components, e.g., antigen receptors, e.g., CARs, are well known and may be used with the provided methods and compositions. Exemplary methods include those for transfer of polynucleotides encoding the receptors, including via viral, e.g., retroviral or lentiviral, transduction, transposons, and electroporation.

In some embodiments, recombinant polynucleotides are transferred into cells using recombinant infectious virus particles, such as, e.g., vectors derived from simian virus 40 (SV40), adenoviruses, adeno-associated virus (AAV). In some embodiments, recombinant polynucleotides are transferred into T cells using recombinant lentiviral vectors, such as HIV-1 lentivirus-based vectors (lentivectors; see, e.g., Amado et al., Science. 1999 Jul. 30; 285(5428):674-676), or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557).

In some embodiments, the retroviral vector or lentiviral vector has a long terminal repeat sequence (LTR). In some embodiments the vector is derived from the Moloney murine leukemia virus (MoMLV), myeloproliferative sarcoma virus (MPSV), murine embryonic stem cell virus (MESV), murine stem cell virus (MSCV), spleen focus forming virus (SFFV), human immunodeficiency virus type 1 (HIV-1), human immunodeficiency virus type 2 (HIV-2/SIV) or adeno-associated virus (AAV). In some embodiments, the vectors are self-inactivating (SIN). In some embodiments, the vectors are conditionally replicating (mobilizable) vectors. Most lentiviral vectors are derived from human, feline or simian lentiviruses. Most retroviral vectors are derived from murine retroviruses. In some embodiments, the lentiviruses or retroviruses include those derived from any avian or mammalian cell source. The lentiviruses or retroviruses typically are amphotropic, meaning that they are capable of infecting host cells of several species, including humans. In one embodiment, the gene to be expressed replaces the retroviral gag, pol and/or env sequences. Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. A number of illustrative retroviral systems have also been described (e.g., Amado et al., (1999) Science 285(5428):674-676, U.S. Pat. Nos. 5,219,740; 6,207,453; 5,219,740; Miller and Rosman (1989) BioTechniques 7:980-990; Miller (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109).

In some embodiments, recombinant polynucleotides are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant polynucleotides are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston (1990) Nature 346: 776-777); and strontium phosphate DNA co-precipitation (Brash et al., (1987) Mol. Cell Biol. 7: 2031-2034). Other approaches and vectors for transfer of the polynucleotides encoding the recombinant products are those described, e.g., in international patent application, Publication No.: WO2014055668, and U.S. Pat. No. 7,446,190.

Among additional polynucleotides, e.g., genes for introduction are those to improve the outcome of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

In some embodiments, one or more recombinant receptors (e.g., CARs) can be genetically engineered to be expressed in cells or plurality of cells. In some embodiments, a recombinant receptor is a CAR. In some embodiments, the CAR comprises two antigen-binding domains. In some embodiments, the CAR comprises a GPRC5D-binding domain that binds to GPRC5D (e.g., human GPRC5D) and a BCMA-binding domain that binds to BCMA (e.g., human BCMA). In some embodiments, the GPRC5D-binding domain and the BCMA-binding domain of the CAR are separated by a linker, such as a polypeptide linker.

In some embodiments the vector or construct can contain a promoter and/or enhancer or regulatory elements to regulate expression of the encoded recombinant receptor. In some examples the promoter and/or enhancer or regulatory elements can be condition-dependent promoters, enhancers, and/or regulatory elements. In some examples these elements drive expression of the transgene. In some examples, the CAR transgene can be operatively linked to a promoter, such as an EF1alpha promoter with an HTLV1 enhancer (SEQ ID NO: 61). In some examples, the CAR transgene is operatively linked to a Woodchuck Hepatitis Virus (WHP) Posttranscriptional Regulatory Element (WPRE; SEQ ID NO: 62), located downstream of the transgene.

In some embodiments, the vector or construct can contain a single promoter that drives the expression of one or more nucleic acid molecules. In some embodiments, such nucleic acid molecules, e.g., transcripts, can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g., encoding a first and second chimeric receptor) by a message from a single promoter.

Alternatively, in some cases, a single promoter may direct expression of an RNA that contains, in a single open reading frame (ORF), two or three genes (e.g. encoding a first and second binding molecules, e.g., antibody recombinant receptor) separated from one another by sequences encoding a self-cleavage peptide (e.g., 2A cleavage sequences) or a protease recognition site (e.g., furin). The ORF thus encodes a single polypeptide, which, either during (in the case of T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and polynucleotides disclosed herein, without limitation, 2A sequences from the foot-and-mouth disease virus (F2A, e.g., SEQ ID NO: 63 or 64), equine rhinitis A virus (E2A, e.g., SEQ ID NO: 65 or 66), Thosea asigna virus (T2A, e.g., SEQ ID NO: 67, 68, or 69), and porcine teschovirus-1 (P2A, e.g., SEQ ID NO: 70 or 71) as described in U.S. Patent Publication No. 20070116690. In some embodiments, the one or more different or separate promoters drive the expression of one or more nucleic acid molecules encoding the one or more binding molecules, e.g., recombinant receptors.

Any of the recombinant receptors provided herein, e.g., bispecific CARs binding to GPRC5D and BCMA, can be encoded by polynucleotides containing one or more nucleic acid molecules encoding the receptors, in any combinations or arrangements. For example, one, two, three or more polynucleotides can encode one, two, three or more different receptors or domains. In some embodiments, one vector or construct contains nucleic acid molecules encoding one or more recombinant receptor(s), and a separate vector or construct contains nucleic acid molecules encoding an additional binding molecule, e.g., antibody and/or recombinant receptor. Each of the nucleic acid molecules can also encode one or more surrogate marker(s), such as fluorescent protein (e.g., green fluorescent protein (GFP)) or a cell surface marker (e.g., a truncated surface marker such as truncated EGFR (tEGFR), which may be used to confirm transduction or engineering of the cell to express the receptor. For example, in some aspects, extrinsic marker genes are utilized in connection with engineered cell therapies to permit detection or selection of cells and, in some cases, also to promote cell suicide by ADCC. Exemplary marker genes include truncated epidermal growth factor receptor (EGFRt), which can be co-expressed with a transgene of interest (e.g., a CAR or TCR) in transduced cells (see, e.g., U.S. Pat. No. 8,802,374). EGFRt contains an epitope recognized by the antibody cetuximab (Erbitux®). For this reason, Erbitux® can be used to identify or select cells that have been engineered with the EGFRt construct, including in cells also co-engineered with another recombinant receptor, such as a chimeric antigen receptor (CAR).

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

Also provided are compositions containing one or more of the nucleic acid molecules, vectors or constructs, such as any described above. In some embodiments, the nucleic acid molecules, vectors, constructs or compositions can be used to engineer cells, such as T cells, to express any of the binding molecules, e.g., antibody or recombinant receptor, and/or the additional binding molecules.

B. Preparation of Cells for Engineering

In some embodiments, preparation of the engineered cells includes one or more culture and/or preparation steps. The cells for introduction of the recombinant receptor (e.g., CAR) may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human T cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques.

For example, CD3+, CD28+ T cells can be positively selected using CD3/CD28 conjugated magnetic beads (e.g., DYNABEADS® M-450 CD3/CD28 T Cell Expander, MACSiBeads™, etc.).

In some embodiments, isolation is carried out by enrichment for a particular cell population by positive selection, and/or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (markerhigh) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase certain features, such as to improve long-term survival, expansion, and/or engraftment following administration, which in some aspects is particularly robust in such sub-populations (see Terakura et al. (2012) Blood. 1:72-82; Wang et al. (2012) J Immunother. 35(9):689-701). In some embodiments, combining TCM-enriched CD8+ T cells and CD4+ T cells further enhances response.

In embodiments, memory T cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA, and positive selection based on a marker characteristic of central memory T cells, such as CD62L or CCR7, where the positive and negative selections are carried out in either order.

CD4+ T helper cells are sorted into naïve, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO−.

In one example, to enrich for CD4+ cells by negative selection, a monoclonal antibody cocktail typically includes antibodies to CD14, CD20, CD11 b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher© Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynabeads® or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084, are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS®) (Miltenyi Biotec, Auburn, CA). Magnetic Activated Cell Sorting (MACS®) systems are capable of high-purity selection of cells having magnetized particles attached thereto. In certain embodiments, MACS® operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS® system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS® system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy® system (Miltenyi Biotec). The CliniMACS Prodigy® system in some aspects is equipped with a cell processing unity that permits automated washing and fractionation of cells by centrifugation. The CliniMACS Prodigy® system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy® system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope (see, e.g., Klebanoff et al. (2012) J Immunother. 35(9): 651-660, Terakura et al. (2012) Blood. 1:72-82, and Wang et al. (2012) J Immunother. 35(9):689-701).

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° C. per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In some embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

C. Engineered Cells, Vectors and Compositions for Multi-Targeting

Also provided are cells such as engineered cells that can bind to multiple antigens. In some embodiments, improved selectivity and specificity is achieved through strategies targeting multiple antigens. Such strategies generally involve multiple antigen-binding domains, which typically are present on distinct genetically engineered antigen receptors and specifically bind to distinct antigens. In some embodiments, the cells are engineered with the ability to bind more than one antigen. For example, in some embodiments, the cells are engineered to express multispecific binding molecules. In some embodiments, the cells express multiple binding molecules, antigen-binding domains, each of which can target one antigen or multiple antigens, e.g., one binding domain that targets GPRC5D, such as any described herein, and another binding domain that targets another antigen, such as a tumor antigen, e.g., BCMA. Exemplary GPRC5D-binding domains are described herein and in WO 2016/090312, WO 2016/090329, WO 2018/017786, WO2020148677, WO2019154890, WO2021018859, WO2021018925, and WO2018147245. Exemplary BCMA-binding domains are described herein and in WO 2013/154760, WO 2015/052538, WO 2015/090229, WO 2015/092024, WO 2015/158671, WO 2016/014565, WO 2016/014789, WO 2016/094304, WO 2016/166630, WO 2017/021450, WO 2017/083511, WO 2017/130223, WO 2017/211900, WO 2018/085690, WO 2018/028647.

In some aspects, the antigen receptor comprises a plurality of binding domains, which bind to different antigens, each expressed in or on the disease or condition to be targeted with the cells or tissues or cells thereof. Such features can in some aspects address or reduce the likelihood of off-target effects and/or increase response. For example, where a single antigen expressed in a disease or condition is also expressed on or in non-diseased or normal cells, such multi-targeting approaches can provide selectivity for desired cell types by requiring binding via multiple antigen receptors in order to activate the cell or induce a particular effector function. In some embodiments, a plurality of cells can be engineered to express a recombinant receptor (e.g., a CAR) comprising one or more different binding domains, e.g., a GPRC5D-binding domain and a BCMA-binding domain.

Also provided are multispecific cells or compositions, such as those containing one or more of any of the recombinant receptors or cells provided herein. In some aspects, the multispecific cells such as cells containing a cell surface protein including the GPRC5D-binding domain or portion thereof and the BCMA-binding domain or a portion thereof. In some embodiments, provided are compositions of cells that express recombinant receptors, wherein one or more of the binding domains binds and/or targets GPRC5D. In some embodiments, provided are compositions of cells that express recombinant receptors, wherein one or more of the binding domains binds and/or targets BCMA. In some embodiments, the bispecific recombinant receptors or cells or compositions expressing the same target one or more epitopes on GPRC5D and one or more epitopes on BCMA.

In some embodiments, provided are composition of cells, wherein cells within the composition expresses a bispecific recombinant receptor, e.g. a CAR. In some embodiments, the cell comprises (and in some cases has been transformed or transfected or transduced with) one or more vectors or constructs comprising one or more nucleic acid that encodes one or more amino acid sequence comprising one or more antibodies and/or portions thereof, e.g., antigen-binding fragments thereof. In some embodiments, one or more such cells are provided. In some embodiments, a composition containing one or more such cells is provided. In some embodiments, cells within the composition express a bispecific recombinant receptor, e.g., CAR. In some aspects, the provided embodiments include multi-targeting strategies that target GPRC5D and BCMA.

In some embodiments, GPRC5D and BCMA are expressed or suspected of being expressed on the cell, tissue, or disease or condition being targeted, such as on the cancer cell. In some aspects, the cell, tissue, disease or condition is multiple myeloma or a multiple myeloma cell.

In some embodiments, the BCMA-binding domain is specific for BCMA, e.g. human BCMA and the GPRC5D-binding domain is specific for GPRC5D, e.g., human GPRC5D. Chimeric antigen receptors containing anti-BCMA antibodies, including mouse anti-human BCMA antibodies and human anti-human antibodies, and cells expressing such chimeric receptors have been previously described. See Carpenter et al., Clin Cancer Res., 2013, 19(8):2048-2060, WO 2016/090320, WO2016090327, WO2010104949A2 and WO2017173256. In some embodiments, the anti-BCMA CAR contains an antigen-binding domain, such as an scFv, containing a variable heavy (VH) and/or a variable light (VL) region derived from an antibody described in WO 2016/090320 or WO2016090327. Chimeric antigen receptors containing anti-GPRC5D antibodies, including human anti-human antibodies, and cells expressing such chimeric receptors have been previously described. See WO 2016/090312, WO 2016/090329, WO 2018/017786, WO2020148677, WO2019154890, WO2021018859, WO2021018925, or WO2018147245.

Among provided BCMA-binding domain is a binding domain in which the antibody or antigen-binding fragment contains a V_{H} region comprising the sequence set forth in SEQ ID NO: 15 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:15; and contains a V_{L} region comprising the sequence set forth in SEQ ID NO:16 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:16. In some embodiments, the antibody or antigen-binding fragment of the provided CAR contains a V_{H} region that has a CDRH1, a CDRH2 and a CDRH3 comprising the amino acid sequence of SEQ ID NOS: 9, 10, and 11, respectively and a V_{L} region that has a CDRL1, a CDRL2 and a CDRL3 comprising the amino acid sequence of SEQ ID NOS: 12, 13, and 14, respectively. In some embodiments, the V_{H} region comprises the sequence set forth in SEQ ID NO:15 and the V_{L} region comprises the sequence set forth in SEQ ID NO: 16. In some embodiments, the V_{H} region and the V_{L} region are connected by a linker. In some embodiments, the linker comprises the amino acid sequence set forth in SEQ ID NO: 17. In some embodiments, the antibody or antigen-binding fragment is a single-chain antibody fragment, such as an scFv. In some embodiments, the scFv comprises the sequence of amino acids set forth in SEQ ID NO:47 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:47. In some embodiments, the scFv comprises the sequence of amino acids set forth in SEQ ID NO:47. In some embodiments, the scFv comprises the sequence of amino acids set forth in SEQ ID NO:48 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:48. In some embodiments, the scFv comprises the sequence of amino acids set forth in SEQ ID NO:48.

Among a provided GPRC5D-binding domain is a binding domain in which the antibody or antigen-binding fragment contains a V_{H} region comprising the sequence set forth in SEQ ID NO: 7 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:7; and contains a V_{L} region comprising the sequence set forth in SEQ ID NO:8 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:8. In some embodiments, the antibody or antigen-binding fragment of the provided CAR contains a V_{H} region that has a CDRH1, a CDRH2 and a CDRH3 comprising the amino acid sequence of SEQ ID NOS: 1, 2, and 3, respectively and a V_{L} region that has a CDRL1, a CDRL2 and a CDRL3 comprising the amino acid sequence of SEQ ID NOS: 4, 5, and 6, respectively. In some embodiments, the V_{H} region comprises the sequence set forth in SEQ ID NO:7 and the V_{L} region comprises the sequence set forth in SEQ ID NO:8. In some embodiments, the V_{H} region and the V_{L} region are connected by a linker. In some embodiments, the linker comprises the amino acid sequence set forth in SEQ ID NO: 17. In some embodiments, the antibody or antigen-binding fragment is a single-chain antibody fragment, such as an scFv. In some embodiments, the scFv comprises the sequence of amino acids set forth in SEQ ID NO:45 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:45. In some embodiments, the scFv comprises the sequence of amino acids set forth in SEQ ID NO:45. In some embodiments, the scFv comprises the sequence of amino acids set forth in SEQ ID NO:46 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:46. In some embodiments, the scFv comprises the sequence of amino acids set forth in SEQ ID NO:46.

Among the provided bispecific CARs is a CAR comprising the extracellular binding domain sequence set forth in any one of SEQ ID NOS: 77-90. In some embodiments, the extracellular binding domain comprises the amino acid sequence set forth in SEQ ID NO:77. In some embodiments, the extracellular binding domain comprises the amino acid sequence set forth in SEQ ID NO:78. In some embodiments, the extracellular binding domain comprises the amino acid sequence set forth in SEQ ID NO:79. In some embodiments, the extracellular binding domain comprises the amino acid sequence set forth in SEQ ID NO:80. In some embodiments, the extracellular binding domain comprises the amino acid sequence set forth in SEQ ID NO:81. In some embodiments, the extracellular binding domain comprises the amino acid sequence set forth in SEQ ID NO:82. In some embodiments, the extracellular binding domain comprises the amino acid sequence set forth in SEQ ID NO:83. In some embodiments, the extracellular binding domain comprises the amino acid sequence set forth in SEQ ID NO:84. In some embodiments, the extracellular binding domain comprises the amino acid sequence set forth in SEQ ID NO:85. In some embodiments, the extracellular binding domain comprises the amino acid sequence set forth in SEQ ID NO:86. In some embodiments, the extracellular binding domain comprises the amino acid sequence set forth in SEQ ID NO:87. In some embodiments, the extracellular binding domain comprises the amino acid sequence set forth in SEQ ID NO:88. In some embodiments, the extracellular binding domain comprises the amino acid sequence set forth in SEQ ID NO:89. In some embodiments, the extracellular binding domain comprises the amino acid sequence set forth in SEQ ID NO:90.

Among the provided bispecific CARs is a CAR comprising the sequence set forth in any one of SEQ ID NOS:31-44. In some embodiments, the CAR comprises the amino acid sequence set forth in SEQ ID NO:31. In some embodiments, the CAR comprises the amino acid sequence set forth in SEQ ID NO:32. In some embodiments, the CAR comprises the amino acid sequence set forth in SEQ ID NO:33. In some embodiments, the CAR comprises the amino acid sequence set forth in SEQ ID NO:34. In some embodiments, the CAR comprises the amino acid sequence set forth in SEQ ID NO:35. In some embodiments, the CAR comprises the amino acid sequence set forth in SEQ ID NO:36. In some embodiments, the CAR comprises the amino acid sequence set forth in SEQ ID NO:37. In some embodiments, the CAR comprises the amino acid sequence set forth in SEQ ID NO:38. In some embodiments, the CAR comprises the amino acid sequence set forth in SEQ ID NO:39. In some embodiments, the CAR comprises the amino acid sequence set forth in SEQ ID NO:40. In some embodiments, the CAR comprises the amino acid sequence set forth in SEQ ID NO:41. In some embodiments, the CAR comprises the amino acid sequence set forth in SEQ ID NO:42. In some embodiments, the CAR comprises the amino acid sequence set forth in SEQ ID NO:43. In some embodiments, the CAR comprises the amino acid sequence set forth in SEQ ID NO:44.

### IV. Pharmaceutical Compositions

Also provided are compositions including the bispecific recombinant receptors (e.g. bispecific CARs targeting GPRC5D and BCMA), and engineered cells, including pharmaceutical compositions and formulations. Among such compositions are those that include engineered cells, such as a plurality of engineered cells, expressing the provided bispecific recombinant receptors (e.g., CARs). In some embodiments, provided compositions include engineered cells, expressing the provided CARs that bind GPRC5D and BCMA, such as those comprising a GPRC5D-binding domain and a BCMA-binding domain.

Provided are pharmaceutical formulations comprising a bispecific recombinant receptor (CAR) that binds GPRC5D and BCMA and engineered cells expressing said receptors, a plurality of engineered cells expressing said receptors and/or additional agents for combination treatment or therapy. The pharmaceutical compositions and formulations generally include one or more optional pharmaceutically acceptable carrier(s) or excipient(s). In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell, binding molecule, and/or antibody, and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., bendamustine, asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the host cells (e.g., T-cells or NK cells) to a particular tissue. Many methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and 5,019,369.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains the binding molecules and/or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of genetically engineered cells containing the binding molecules, a subject is administered the range of about one million to about 100 billion cells, such as, e.g., 1 million to about 50 billion cells (e.g., about 5 million cells, about 25 million cells, about 75 million cells, about 500 million cells, about 1 billion cells, about 2 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 50 million cells, about 60 million cells, about 70 million cells, about 80 million cells, about 90 million cells, about 100 million cells, about 200 million cells, about 300 million cells, about 400 million cells, about 600 million cells, about 700 million cells, about 1.5 billion cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 150 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject. In some aspects, in the context of genetically engineered cells expressing the binding molecules, e.g., CAR, a composition can contain at least the number of cells for administration for a dose of cell therapy, such as about or at least a number of cells described herein for administration. In some embodiments, the number of cells is the number of such cells that are viable cells.

In some embodiments, the percentage of viable cells in the composition is greater than 90%. In some embodiments, the percentage of viable cells in the composition is greater than 95%. In some embodiments, the percentage of viable cells in the composition is greater than 96%. In some embodiments, the percentage of viable cells in the composition is greater than 97%. In some embodiments, the percentage of viable cells in the composition is greater than 98%. In some embodiments, the percentage of viable cells in the composition is greater than 99%.

The may be administered using standard administration techniques, formulations, and/or devices. Provided are formulations and devices, such as syringes and vials, for storage and administration of the compositions. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, intracranial, intrathoracic, and intraperitoneal administration. In some embodiments, the cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the binding molecule in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

Also provided are pharmaceutical compositions for combination therapy. Any of the additional agents for combination therapy described herein, can be prepared and administered as one or more pharmaceutical compositions, with the bispecific recombinant receptor (e.g., chimeric antigen receptor) and/or engineered cells expressing said molecules (e.g., recombinant receptor) described herein. The combination therapy can be administered in one or more pharmaceutical compositions, e.g., where the binding domains, recombinant receptors and/or cells are in the same pharmaceutical composition as the additional agent, or in separate pharmaceutical compositions. In some embodiments, each of the pharmaceutical composition is formulated in a suitable formulation according to the particular binding molecule, recombinant receptor, cell, e.g., engineered cell, and/or additional agent, and the particular dosage regimen and/or method of delivery

### V. Methods and Uses

Also provided are methods of using and uses of the GPRC5D- and BCMA-targeted recombinant receptors, engineered cells, and pharmaceutical compositions and formulations thereof, such as in the treatment of diseases, conditions, and disorders in which GPRC5D and/or BCMA is expressed, or detection, diagnostic, and prognostic methods. Among such methods and uses are those that involve administering to a subject engineered cells, such as a plurality of engineered cells, expressing the provided bispecific recombinant receptors (e.g., CARs). Also provided are methods of combination therapy and/or treatment.

A. Therapeutic and Prophylactic Methods and Uses

Also provided are methods of administering and uses of, such as therapeutic and prophylactic uses of, the bispecific recombinant receptors (e.g., CARs), engineered cells expressing the recombinant receptors (e.g., CARs), plurality of engineered cells expressing the receptors, and/or compositions comprising the same. Such methods and uses include therapeutic methods and uses, for example, involving administration of the molecules (e.g., recombinant receptors), cells (e.g., engineered cells), or compositions containing the same, to a subject having a disease, condition, or disorder associated with GPRC5D and/or BCMA such as a disease, condition, or disorder associated with GPRC5D and/or BCMA expression, and/or in which cells or tissues express, e.g., specifically express, GPRC5D and/or BCMA. Such methods and uses include therapeutic methods and uses, for example, involving administration of the molecules (e.g., recombinant receptors), cells (e.g., engineered cells), or compositions containing the same, to a subject having a disease, condition, or disorder associated with GPRC5D such as a disease, condition, or disorder associated with GPRC5D expression, and/or in which cells or tissues express, e.g., specifically express, GPRC5D. Such methods and uses include therapeutic methods and uses, for example, involving administration of the molecules (e.g., recombinant receptors), cells (e.g., engineered cells), or compositions containing the same, to a subject having a disease, condition, or disorder associated with BCMA such as a disease, condition, or disorder associated with BCMA expression, and/or in which cells or tissues express, e.g., specifically express, BCMA. Such methods and uses include therapeutic methods and uses, for example, involving administration of the molecules (e.g., recombinant receptors), cells (e.g., engineered cells), or compositions containing the same, to a subject having a disease, condition, or disorder associated with GPRC5D and BCMA such as a disease, condition, or disorder associated with GPRC5D and BCMA expression, and/or in which cells or tissues express, e.g., specifically express, GPRC5D and BCMA.

In some embodiments, the molecule, cell, and/or composition is/are administered in an effective amount to effect treatment of the disease or disorder. Provided herein are uses of the recombinant receptors (e.g., CARs), and cells (e.g., engineered cells) in such methods and treatments, and in the preparation of a medicament in order to carry out such therapeutic methods. In some embodiments, the methods are carried out by administering the binding molecules or cells, or compositions comprising the same, to the subject having, having had, or suspected of having the disease or condition. In some embodiments, the methods thereby treat the disease or condition or disorder in the subject. Also provided herein are use of any of the compositions, such as pharmaceutical compositions provided herein, for the treatment of a disease or disorder associated with GPRC5D and/or BCMA, such as use in a treatment regimen. Also provided herein are use of any of the compositions, such as pharmaceutical compositions provided herein, for the treatment of a disease or disorder associated with GPRC5D, such as use in a treatment regimen. Also provided herein are use of any of the compositions, such as pharmaceutical compositions provided herein, for the treatment of a disease or disorder associated with BCMA, such as use in a treatment regimen. Also provided herein are use of any of the compositions, such as pharmaceutical compositions provided herein, for the treatment of a disease or disorder associated with GPRC5D and BCMA, such as use in a treatment regimen.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or subject being treated. As sufficient or significant delay can, in effect, encompass prevention, in that the subject does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided molecules and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, an antibody or composition or cell which suppresses tumor growth reduces the rate of growth of the tumor compared to the rate of growth of the tumor in the absence of the antibody or composition or cell.

An “effective amount” of an agent, e.g., a pharmaceutical formulation, binding molecule, antibody, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation, binding molecule, antibody, cells, or composition refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the molecules, antibodies, cells, and/or compositions at effective amounts, e.g., therapeutically effective amounts.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

As used herein, a “subject” or an “individual” is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject is human.

Methods for administration of cells for adoptive cell therapy are known and may be used in connection with the provided methods and compositions. For example, adoptive T cell therapy methods are described, e.g., in US Pat. App. Pub. No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

Among the diseases to be treated is any disease or disorder associated with GPRC5D and/or BCMA or any disease or disorder in which GPRC5D and/or BCMA is specifically expressed and/or in which GPRC5D and/or BCMA has been targeted for treatment (also referred to herein interchangeably as a “GPRC5D-associated disease or disorder” or a “BCMA-associated disease or disorder”). Cancers associated with GPRC5D and/or BCMA expression include hematologic malignancies such as myeloma, e.g., multiple myeloma. In some embodiments, the disease or disorder associated with GPRC5D and/or BCMA is a B cell-related disorder or malignancy. In some embodiments the disease or disorder associated with GPRC5D and/or BCMA is multiple myeloma or Waldenstrom's Macroglobulinemia. In certain embodiments, the disease or disorder is multiple myeloma.

Among the diseases to be treated is any disease or disorder associated with GPRC5D or any disease or disorder in which GPRC5D is specifically expressed and/or in which GPRC5D has been targeted for treatment (also referred to herein interchangeably as a “GPRC5D-associated disease or disorder”). Cancers associated with GPRC5D expression include hematologic malignancies such as myeloma, e.g., multiple myeloma. In some embodiments, the disease or disorder associated with GPRC5D is a B cell-related disorder or malignancy. In some embodiments the disease or disorder associated with GPRC5D is multiple myeloma or Waldenstrom's Macroglobulinemia. In certain embodiments, the disease or disorder is multiple myeloma.

Among the diseases to be treated is any disease or disorder associated with BCMA or any disease or disorder in which BCMA is specifically expressed and/or in which BCMA has been targeted for treatment (also referred to herein interchangeably as a “BCMA-associated disease or disorder”). Cancers associated with BCMA expression include hematologic malignancies such as myeloma, e.g., multiple myeloma. In some embodiments, the disease or disorder associated with BCMA is a B cell-related disorder or malignancy. In some embodiments the disease or disorder associated with BCMA is multiple myeloma or Waldenstrom's Macroglobulinemia. In certain embodiments, the disease or disorder is multiple myeloma.

Among the diseases to be treated is any disease or disorder associated with GPRC5D and BCMA or any disease or disorder in which GPRC5D and BCMA is specifically expressed and/or in which GPRC5D and BCMA has been targeted for treatment (also referred to herein interchangeably as a “GPRC5D- and BCMA-associated disease or disorder”). Cancers associated with GPRC5D and BCMA expression include hematologic malignancies such as myeloma, e.g., multiple myeloma. In some embodiments, the disease or disorder associated with GPRC5D and BCMA is a B cell-related disorder or malignancy. In some embodiments the disease or disorder associated with GPRC5D and BCMA is multiple myeloma or Waldenstrom's Macroglobulinemia. In certain embodiments, the disease or disorder is multiple myeloma.

In some embodiments, the subject is an adult subject. In some embodiments, the subject is ≥18 years of age. In some embodiments, the subject to be treated is human.

In some embodiments, the disease or disorder is associated with expression of GPRC5D and BCMA. In some embodiments, cells of the disease are suspected of expressing both antigens. In some embodiments, one or both of the antigens is susceptible to antigen loss, in that some cells of the disease may no longer express both antigens. Thus, in some embodiments, a dual-targeting approach, targeting both GPRC5D and BCMA, may be advantageous.

In some embodiments, the methods may identify a subject who has, is suspected to have, or is at risk for developing a GPRC5D-associated and/or BCMA-associated disease or disorder. Hence, provided are methods for identifying subjects with diseases or disorders associated with GPRC5D and/or BCMA expression and selecting them for treatment with a provided bispecific recombinant receptors (e.g., CARs), and/or engineered cells expressing the recombinant receptors.

In some embodiments, the methods may identify a subject who has, is suspected to have, or is at risk for developing a GPRC5D-associated disease or disorder. Hence, provided are methods for identifying subjects with diseases or disorders associated with GPRC5D expression and selecting them for treatment with a provided bispecific recombinant receptors (e.g., CARs), and/or engineered cells expressing the recombinant receptors.

In some embodiments, the methods may identify a subject who has, is suspected to have, or is at risk for developing a BCMA-associated disease or disorder. Hence, provided are methods for identifying subjects with diseases or disorders associated with BCMA expression and selecting them for treatment with a provided bispecific recombinant receptors (e.g., CARs), and/or engineered cells expressing the recombinant receptors.

For example, a subject may be screened for the presence of a disease or disorder associated with elevated GPRC5D and/or BCMA expression, such as a GPRC5D- and/or BCMA-expressing cancer. In some embodiments, the methods include screening for or detecting the presence of a GPRC5D- and/or BCMA-associated disease, e.g., a tumor. Thus, in some aspects, a sample may be obtained from a patient suspected of having a disease or disorder associated with elevated GPRC5D and/or BCMA expression and assayed for the expression level of GPRC5D and/or BCMA. In some aspects, a subject who tests positive for a GPRC5D- and/or BCMA-associated disease or disorder may be selected for treatment by the present methods, and may be administered a therapeutically effective amount of a composition comprising cells expressing a recombinant receptor (e.g., CAR) comprising a GPRC5D-binding domain and a BCMA-binding domain, or a pharmaceutical composition thereof as described herein. In some aspects, a subject who tests positive for a GPRC5D- and/or BCMA-associated disease or disorder may be selected for treatment by the present methods, and may be administered a therapeutically effective amount of a composition comprising cells expressing a recombinant receptor (e.g., CAR) comprising a GPRC5D-binding domain and a BCMA-binding domain, cells expressing a recombinant receptor comprising a GPRC5D-binding domain and a BCMA-binding domain, or a pharmaceutical composition thereof as described herein.

For example, a subject may be screened for the presence of a disease or disorder associated with elevated GPRC5D expression, such as a GPRC5D-expressing cancer. In some embodiments, the methods include screening for or detecting the presence of a GPRC5D-associated disease, e.g., a tumor. Thus, in some aspects, a sample may be obtained from a patient suspected of having a disease or disorder associated with elevated GPRC5D expression and assayed for the expression level of GPRC5D. In some aspects, a subject who tests positive for a GPRC5D-associated disease or disorder may be selected for treatment by the present methods, and may be administered a therapeutically effective amount of a recombinant receptor (e.g., CAR) comprising a GPRC5D-binding domain, cells containing a recombinant receptor, or a pharmaceutical composition thereof as described herein.

For example, a subject may be screened for the presence of a disease or disorder associated with elevated BCMA expression, such as a BCMA-expressing cancer. In some embodiments, the methods include screening for or detecting the presence of a BCMA-associated disease, e.g., a tumor. Thus, in some aspects, a sample may be obtained from a patient suspected of having a disease or disorder associated with elevated BCMA expression and assayed for the expression level of BCMA. In some aspects, a subject who tests positive for a BCMA-associated disease or disorder may be selected for treatment by the present methods, and may be administered a therapeutically effective amount of a recombinant receptor (e.g., CAR) comprising a BCMA-binding domain, cells containing a recombinant receptor, or a pharmaceutical composition thereof as described herein.

For example, a subject may be screened for the presence of a disease or disorder associated with elevated GPRC5D and BCMA expression, such as a GPRC5D- and BCMA-expressing cancer. In some embodiments, the methods include screening for or detecting the presence of a GPRC5D- and associated disease, e.g., a tumor. Thus, in some aspects, a sample may be obtained from a patient suspected of having a disease or disorder associated with elevated GPRC5D and BCMA expression and assayed for the expression level of GPRC5D and BCMA. In some aspects, a subject who tests positive for a GPRC5D- and BCMA-associated disease or disorder may be selected for treatment by the present methods, and may be administered a therapeutically effective amount of a composition comprising cells expressing a recombinant receptor (e.g., CAR) comprising a GPRC5D-binding domain and a BCMA-binding domain, or a pharmaceutical composition thereof as described herein. In some aspects, a subject who tests positive for a GPRC5D- and BCMA-associated disease or disorder may be selected for treatment by the present methods, and may be administered a therapeutically effective amount of a composition comprising cells expressing a recombinant receptor (e.g., CAR) comprising a GPRC5D-binding domain and a BCMA-binding domain, cells expressing a recombinant receptor comprising a GPRC5D-binding domain and a BCMA-binding domain, or a pharmaceutical composition thereof as described herein.

In some embodiments, the subject has received a prior therapy that is a GPRC5D CAR therapy or other GPRC5D-targeted therapy. In some embodiments, the subject is refractory to or has relapsed following such GPRC5D CAR therapy or other GPRC5D-targeted therapy.

In some embodiments, the subject has persistent or relapsed disease, e.g., following treatment with a GPRC5D-specific antibody and/or cells expressing a GPRC5D-targeting chimeric receptor and/or other therapy, including chemotherapy, radiation, and/or hematopoietic stem cell transplantation (HSCT), e.g., allogenic HSCT or autologous HSCT. In some embodiments, the administration effectively treats the subject despite the subject having become resistant to another GPRC5D-targeted therapy. In some embodiments, the subject has persistent or relapsed disease, e.g., following treatment with a BCMA-specific antibody and/or cells expressing a BCMA-targeting chimeric receptor and/or other therapy, including chemotherapy, radiation, and/or hematopoietic stem cell transplantation (HSCT), e.g., allogenic HSCT or autologous HSCT. In some embodiments, the administration effectively treats the subject despite the subject having become resistant to another BCMA-targeted therapy. In some embodiments, the subject has not relapsed but is determined to be at risk for relapse, such as at a high risk of relapse, and thus the compound or composition is administered prophylactically, e.g., to reduce the likelihood of or prevent relapse.

In some embodiments, the subject has received a prior therapy that is a BCMA CAR therapy or other BCMA-targeted therapy. In some embodiments, the subject is refractory to or has relapsed following such BCMA CAR therapy or other BCMA-targeted therapy. In some cases, the subject is refractory to or has relapsed due to BCMA antigen-negative tumor cells and/or BCMA antigen/epitope loss following therapy.

In some embodiments, the subject has persistent or relapsed disease following treatment with another therapy, such as treatment with a BCMA-specific antibody, BCMA-targeting receptor, and/or cells expressing a BCMA-targeting chimeric receptor. In some embodiments, the administration effectively treats the subject despite the subject having become resistant to another therapy, such as a BCMA-targeted therapy. In some embodiments, the subject has not relapsed but is determined to be at risk for relapse, such as at a high risk of relapse, and thus the compound or composition is administered prophylactically, e.g., to reduce the likelihood of or prevent relapse.

In some embodiments, the subject is one that is eligible for a transplant, such as is eligible for a hematopoietic stem cell transplantation (HSCT), e.g., allogenic HSCT or autologous HSCT. In some of such embodiments, the subject has not previously received a transplant, despite being eligible, prior to administration of the bispecific recombinant receptors (e.g., CARs), engineered cells expressing the recombinant receptors (e.g., CARs), plurality of engineered cells expressing the receptors, and/or compositions comprising the same, as provided herein.

In some embodiments, the subject is one that is not eligible for a transplant, such as is not eligible for a hematopoietic stem cell transplantation (HSCT), e.g., allogenic HSCT or autologous HSCT. In some of such embodiments, such a subject is administered the bispecific recombinant receptors (e.g., CARs), engineered cells expressing the recombinant receptors (e.g., CARs), plurality of engineered cells expressing the receptors, and/or compositions comprising the same, according to the provided embodiments herein.

In some embodiments, prior to the initiation of administration of the engineered cells, the subject has received one or more prior therapies. In some embodiments, the subject has received at least 1, 2, 3, 4, 5, 6, 7, 8, 9, 10, 11, 12, 13, 14, 15, 16, 17, 18, 19 or 20 or more prior therapies. In some embodiments, the subject has received at least 3, 4, 5, 6, 7, 8, 9, 10 or more prior therapies. In some embodiments, the subject has received at least 3 prior therapies. In some embodiments, the subject has received at least 3 prior anti-myeloma therapies. In some embodiments, the subject has received at least 1, but no more than 3, prior therapies. In some embodiments, the subject has received at least 1, but no more than 3, prior anti-myeloma therapies.

In some embodiments, the disease or condition is multiple myeloma. In some embodiments, the subject has measurable disease defined as meeting at least one of the criteria: i) Serum M-protein≥0.5 g/dL by serum protein electrophoresis (SPEP); ii) Urine M-protein≥200 mg/24 hour by urine protein electrophoresis (UPEP); iii) involved serum free light chain (sFLC) level≥10 mg/dL with abnormal 1× ratio; or iv) for subjects with immunoglobulin class A (IgA) myeloma whose disease can only be reliably measured by quantitative immunoglobulin measurement, a serum IgA level≥0.5 g/dL.

In some embodiments, the subject has an Eastern Cooperative Oncology Group (ECOG) performance status 0 or 1.

In some embodiments, the subject has relapsed after or been refractory to one or more prior therapies for treating multiple myeloma in the subject, i.e. the subject has relapsed/refractory multiple myeloma (RRMM). In some embodiments, the subject has confirmed progressive disease (e.g., by IMWG criteria) on or within 12 months from the last dose of completing treatment with the last anti-myeloma treatment regimen. In some embodiments, the subject has progressive disease such as determined with in the 6 months prior to screening for treatment with the provided engineered cells and who are subsequently determined to be refractory or non-responsive to the most recent anti-myeloma treatment regimen. In some embodiments, the anti-myeloma treatment regimen is selected from an autologous stem cell transplant (ASCT), such an autologous hematopoietic stem cell transplantation (HSCT); an immunomodulatory agent (e.g., thalidomide, lenalidomide or pomalidomide); a proteasome inhibitor (e.g., bortezomib, carfilzomib or ixazomib); and an anti-CD38 antibody (e.g, daratumumab). In some embodiments, if the prior therapy was a cell therapy, such as a CAR-T cell therapy, a subject may be selected for treatment beyond 12 months from receiving the last infusion of the cell therapy.

In some aspects, the subject has relapsed following, or has been refractory to, one or more of, for example, each, individually, of the one or more prior therapies. In some aspects, the prior therapies include treatment with autologous stem cell transplant (ASCT), such an autologous hematopoietic stem cell transplantation (HSCT); an immunomodulatory agent; a proteasome inhibitor; and an anti-CD38 antibody; unless the subject was not a candidate for or was contraindicated for one or more of the therapies. In some embodiments, the immunomodulatory agent is selected from among thalidomide, lenalidomide or pomalidomide. In some embodiments, the proteasome inhibitor is selected from among bortezomib, carfilzomib or ixazomib. In some embodiments, the anti-CD38 antibody is or comprises daratumumab. In some embodiments, the subject must have undergone at least 2 consecutive cycles of treatment for each regimen unless progressive disease was the best response to the regimen.

In some aspects, the subject has been treated with at least 3 prior treatment therapies. In some aspects, the subject has previously received each of the following therapies: (1) a prior therapy that included at least 1 complete cycle of treatment (unless progressive disease was the best response to the regimen) of an immunomodulatory agent (e.g., thalidomide, lenalidomide, pomalidomide) and a proteasome inhibitor (e.g., bortezomib, carfilzomib, ixaxomib), either alone or in combination; (2) a prior therapy that included anti-CD38 antibody therapy (e.g., daratumumab), alone or in combination; and (3) a prior therapy that included autologous hematopoietic stem cell transplantation (HSCT), unless the participant was ineligible. In some embodiments, induction with or without HSCT and with or without maintenance therapy was considered 1 regimen.

In some embodiments, the subject has receive 1 to 3 prior anti-myeloma treatment regiment including a proteasome inhibitor (e.g. bortezomib, carfilzomib, ixaxomib) and an immunomodulatory agent (e.g., thalidomide, lenalidomide, pomalidomide). In such embodiments, induction with or without HSCT and with or without maintenance therapy is considered one regimen.

In some embodiments, the subject has received must have received a single immunomodulatory agent (eg, thalidomide, lenalidomide, pomalidomide) and a single proteasome inhibitor (eg, bortezomib, carfilzomib, ixazomib), either alone or in combination. In some embodiments, the subject has undergone at least 1 complete cycle of treatment unless progressive disease was the best response to the regimen. In such embodiments, induction with or without HSCT and with or without maintenance therapy is considered one regimen.

In some embodiments, the method can involve including or excluding particular subjects for therapy with the provided engineered cells (e.g., T cells) expressing the CARs based on particular criteria, diagnosis or indication. In some embodiments, at the time of administration of the dose of cells, the subject has not had active or history of plasma cell leukemia (PCL). In some embodiments, if the subject had active or a history of PCL at the time of administration, the subject can be excluded from being treated according to the provided methods. In some embodiments, if the subject develops a PCL, such as secondary PCL, at the time of administration, the subject can be excluded from being treated according to the provided methods. In some embodiments, the assessment for the criteria, diagnosis or indication can be performed at the time of screening the subjects for eligibility or suitability of treatment according to the provided methods, at various steps of the treatment regimen, at the time of receiving lymphodepleting therapy, and/or at or immediately prior to the initiation of administration of the engineered cells or composition thereof.

In some embodiments, the method can involve including or excluding particular subjects for therapy with the provided engineered cells (e.g., T cells) expressing the CAR, based on particular criteria, diagnosis or indication. In some embodiments, at the time of administration of the dose of cells, the subject has not had active or history of plasma cell leukemia (PCL). In some embodiments, if the subject had active or a history of PCL at the time of administration, the subject can be excluded from being treated according to the provided methods. In some embodiments, if the subject develops a PCL, such as secondary PCL, at the time of administration, the subject can be excluded from being treated according to the provided methods. In some embodiments, the assessment for the criteria, diagnosis or indication can be performed at the time of screening the subjects for eligibility or suitability of treatment according to the provided methods, at various steps of the treatment regimen, at the time of receiving lymphodepleting therapy, and/or at or immediately prior to the initiation of administration of the engineered cells or composition thereof.

In some embodiments, the treatment does not induce an immune response by the subject to the therapy, and/or does not induce such a response to a degree that prevents effective treatment of the disease or condition. In some aspects, the degree of immunogenicity and/or graft versus host response is less than that observed with a different but comparable treatment. For example, in the case of adoptive cell therapy using cells expressing CARs including the provided BCMA- and GPRC5D-binding domains, the degree of immunogenicity in some embodiments is reduced compared to CARs including a different antibody that binds to a similar, e.g., overlapping epitope and/or that competes for binding to GPRC5D or BCMA with the antibody, such as a mouse or monkey or rabbit or humanized antibody.

In some embodiments, the methods include adoptive cell therapy, whereby genetically engineered cells expressing the provided recombinant receptors comprising a GPRC5D-binding domain and a BCMA-binding domain (e.g., CARs comprising anti-GPRC5D antibody or antigen-binding fragment thereof and anti-BCMA antibody or antigen-binding fragment thereof) are administered to subjects. Such administration can promote activation of the cells (e.g., T cell activation) in a GPRC5D- and/or BCMA-targeted manner, such that the cells of the disease or disorder are targeted for destruction.

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition. In some aspects, the methods thereby treat, e.g., ameliorate one or more symptom of the disease or condition, such as by lessening tumor burden in a GPRC5D- and/or BCMA-expressing cancer. In some aspects, the methods thereby treat, e.g., ameliorate one or more symptom of the disease or condition, such as by lessening tumor burden in a GPRC5D-expressing cancer. In some aspects, the methods thereby treat, e.g., ameliorate one or more symptom of the disease or condition, such as by lessening tumor burden in a BCMA-expressing cancer. In some aspects, the methods thereby treat, e.g., ameliorate one or more symptom of the disease or condition, such as by lessening tumor burden in a GPRC5D- and BCMA-expressing cancer.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered, is a primate, such as a human. In some embodiments, the subject, to whom the cells, cell populations, or compositions are administered, is a non-human primate. In some embodiments, the non-human primate is a monkey (e.g., cynomolgus monkey) or an ape. The subject can be male or female and can be any suitable age, including infant, juvenile, adolescent, adult, and geriatric subjects. In some embodiments, the subject is a non-primate mammal, such as a rodent (e.g., mouse, rat, etc.). In some examples, the patient or subject is a validated animal model for disease, adoptive cell therapy, and/or for assessing toxic outcomes such as cytokine release syndrome (CRS).

The bispecific recombinant receptors (e.g., CARs) and cells expressing the same, can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, subconjunctival injection, sub-Tenon's injection, retrobulbar injection, peribulbar injection, or posterior juxtascleral delivery. The bispecific recombinant receptors (e.g., CARs) and cells expressing the same, can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, intracranial, intrathoracic, or subcutaneous administration. Dosing and administration may depend in part on whether the administration is brief or chronic. Various dosing schedules include but are not limited to single or multiple administrations over various time-points, bolus administration, and pulse infusion.

For the prevention or treatment of disease, the appropriate dosage of the binding molecule, recombinant receptor or cell may depend on the type of disease to be treated, the type of binding molecule or recombinant receptor, the severity and course of the disease, whether the binding molecule or recombinant receptor is administered for preventive or therapeutic purposes, previous therapy, the patient's clinical history and response to the recombinant receptor or cell, and the discretion of the attending physician. The compositions and molecules and cells are in some embodiments suitably administered to the patient at one time or over a series of treatments.

In some embodiments, the dose and/or frequency of administration is/are determined based on efficacy and/or response. In some embodiments, efficacy is determined by evaluating disease status. Exemplary methods for assessing disease status include: measurement of M protein in biological fluids, such as blood and/or urine, by electrophoresis and immunofixation; quantification of sFLC (κ and λ) in blood; skeletal survey; and imaging by positron emission tomography (PET)/computed tomography (CT) in subjects with extramedullary disease. In some embodiments, disease status can be evaluated by bone marrow examination.

In some examples, dose and/or frequency of administration is determined by the expansion and persistence of the recombinant receptor or cell in the blood and/or bone marrow. In some embodiments, dose and/or frequency of administration is determined based on the antitumor activity of the recombinant receptor or engineered cell. In some embodiments antitumor activity is determined by the overall response rate (ORR) and/or International Myeloma Working Group (IMWG) Uniform Response Criteria (see Kumar et al. (2016) Lancet Oncol 17(8):e328-346). In some embodiments, response is evaluated using minimal residual disease (MRD) assessment. In some embodiments, MRD can be assessed by methods such as flow cytometry and high-throughput sequencing, e.g., deep sequencing. In some embodiments, response is evaluated based on the duration of response following administration of the recombinant receptor or cells. In some examples, dose and/or frequency of administration can be based on toxicity. In some embodiments, dose and/or frequency can be determined based on health-related quality of life (HRQoL) of the subject to which the recombinant receptor and/or cells is/are administered. In some embodiments, dose and/or frequency of administration can be changed, i.e., increased or decreased, based on any of the above criteria.

In some embodiments, the disease or disorder to be treated is multiple myeloma. In some embodiments, measurable disease criteria for multiple myeloma can include (1) serum M-protein 1 g/dL or greater; (2) Urine M-protein 200 mg or greater/24 hour; (3) involved serum free light chain (sFLC) level 10 mg/dL or greater, with abnormal κ to λ ratio. In some cases, light chain disease is acceptable only for subjects without measurable disease in the serum or urine.

In some embodiments, the Eastern Cooperative Oncology Group (ECOG) performance status indicator can be used to assess or select subjects for treatment, e.g., subjects who have had poor performance from prior therapies (see, e.g., Oken et al. (1982) Am J Clin Oncol. 5:649-655). The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of selfcare; see e.g., Sorensen et al., (1993) Br J Cancer 67(4) 773-775. In some embodiments, the subject that are to be administered according to the methods or treatment regimen provided herein include those with an ECOG performance status of 0 or 1.

In some embodiments, the administration can treat the subject despite the subject having become resistant to another therapy. In some embodiments, when administered to subjects according to the embodiments described herein, the dose or the composition is capable of achieving objective response (OR), in at least 50%, 60%, 70%, 80%, 90%, or 95% of subjects that were administered. In some embodiments, OR includes subjects who achieve stringent complete response (sCR), complete response (CR), very good partial response (VGPR), partial response (PR) and minimal response (MR). In some embodiments, when administered to subjects according to the embodiments described herein, the dose or the composition is capable of achieving stringent complete response (sCR), complete response (CR), very good partial response (VGPR) or partial response (PR), in at least 50%, 60%, 70%, 80%, or 85% of subjects that were administered. In some embodiments, when administered to subjects according to the embodiments described herein, the dose or the composition is capable of achieving stringent complete response (sCR) or complete response (CR) at least 20%, 30%, 40% 50%, 60% or 70% of subjects that were administered.

In some embodiments, toxicity and/or side-effects of treatment can be monitored and used to adjust dose and/or frequency of administration of the recombinant receptor, e.g., CAR, cells, and or compositions. For example, adverse events and laboratory abnormalities can be monitored and used to adjust dose and/or frequency of administration. Adverse events include infusion reactions, cytokine release syndrome (CRS), neurotoxicity, macrophage activation syndrome, and tumor lysis syndrome (TLS). Any of such events can establish dose-limiting toxicities and warrant decrease in dose and/or a termination of treatment. Other side effects or adverse events which can be used as a guideline for establishing dose and/or frequency of administration include non-hematologic adverse events, which include but are not limited to fatigue, fever or febrile neutropenia, increase in transaminases for a set duration (e.g., less than or equal to 2 weeks or less than or equal to 7 days), headache, bone pain, hypotension, hypoxia, chills, diarrhea, nausea/vomiting, neurotoxicity (e.g., confusion, aphasia, seizures, convulsions, lethargy, and/or altered mental status), disseminated intravascular coagulation, other asymptomatic non-hematological clinical laboratory abnormalities, such as electrolyte abnormalities. Other side effects or adverse events which can be used as a guideline for establishing dose and/or frequency of administration include hematologic adverse events, which include but are not limited to neutropenia, leukopenia, thrombocytopenia, animal, and/or B-cell aplasia and hypogammaglobinemia.

In some embodiments, treatment according to the provided methods can result in a lower rate and/or lower degree of toxicity, toxic outcome or symptom, toxicity-promoting profile, factor, or property, such as a symptom or outcome associated with or indicative of cytokine release syndrome (CRS) or neurotoxicity, such as severe CRS or severe neurotoxicity, for example, compared to administration of other therapies.

In certain embodiments, in the context of genetically engineered cells containing the binding molecules or recombinant receptors, a subject is administered the range of about one million to about 100 billion cells and/or that amount of cells per kilogram of body weight, such as, e.g., about 1 million to about 50 billion cells (e.g., about 5 million cells, about 10 million, about 12.5 million, about 15 million, about 20 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 10 million cells, about 12.5 million cells, about 15 million cells, 20 million cells, about 25 million cells, about 30 million cells, about 40 million cells, about 50 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 150 million cells, about 250 million cells, about 300 million cells, about 350 million cells, about 450 million cells, about 500 million cells, about 600 million cells, about 650 million cells, about 800 million cells, about 900 million cells, about 1 billion cells, about 1.2 billion cells, about 3 billion cells, about 30 billion cells, about 45 billion cells, or about 50 billion cells) or any value in between these ranges and/or per kilogram of body weight. Again, dosages may vary depending on attributes particular to the disease or disorder and/or patient and/or other treatments. In some embodiments, the number of cells is the number of such cells that are viable cells.

In some embodiments, the methods comprise administering a dose of the engineered cells or a composition comprising a dose of the engineered cells. In some embodiments, the engineered cells or compositions containing engineered cells can be used in a treatment regimen, wherein the treatment regimen comprises administering a dose of the engineered cells or a composition comprising a dose of the engineered cells. In some embodiments, the dose can contain, for example, a particular number or range of recombinant receptor-expressing T cells, total T cells, or total peripheral blood mononuclear cells (PBMCs), such as any number of such cells described herein. In some embodiments, a composition containing a dose of the cells can be administered. In some aspects, the number, amount or proportion of CAR-expressing cells in a cell population or a cell composition can be assessed by detection of a surrogate marker, e.g., by flow cytometry or other means, or by detecting binding of a labelled molecule, such as a labelled antigen, that can specifically bind to the binding molecules or receptors provided herein.

In some embodiments, for example, where the subject is a human, the dose includes more than about 1×10⁶ total recombinant receptor (e.g., CAR)-expressing cells, T cells, or peripheral blood mononuclear cells (PBMCs) and fewer than about 2×10⁹ total recombinant receptor (e.g., CAR)-expressing cells, T cells, or peripheral blood mononuclear cells (PBMCs), e.g., in the range of about 2.5×10⁷ to about 1.2×10⁹ such cells, such as 2.5×10⁷, 5×10⁷, 1.5×10⁸, 3×10⁸, 4.5×10⁸, 8×10⁸, or 1.2×10⁹ total such cells, or the range between any two of the foregoing values. In some embodiments, for example, where the subject is a human, the dose includes more than about 1×10⁶ total recombinant receptor (e.g., CAR)-expressing cells, T cells, or peripheral blood mononuclear cells (PBMCs) and fewer than about 2×10⁹ total recombinant receptor (e.g., CAR)-expressing cells, T cells, or peripheral blood mononuclear cells (PBMCs), e.g., in the range of about 1.0×10⁷ to about 1.2×10⁹ such cells, such as 1.0×10⁷, 1.25×10⁷, 1.5×10⁷, 2.0×10⁷, 2.5×10⁷, 5×10⁷, 7.5×10⁷, 1.5×10⁸, 2.25×10⁸, 3×10⁸, 4.5×10⁸, 6.0×10⁸, 8×10⁸, or 1.2×10⁹ total such cells, or the range between any two of the foregoing values. In some embodiments, for example, where the subject is a human, the dose includes more than about 1×10⁶ total recombinant receptor (e.g., CAR)-expressing cells, T cells, or peripheral blood mononuclear cells (PBMCs) and fewer than about 2×10⁹ total recombinant receptor (e.g., CAR)-expressing cells, T cells, or peripheral blood mononuclear cells (PBMCs), e.g., in the range of about 1.0×10⁷ to about 6.5×10⁸ such cells, about 1.5×10⁷ to about 6.0×10⁸ such cells, about 1.5×10⁷ to about 6.5×10⁸ such cells, about 2.5×10⁷ to about 6.0×10⁸ such cells, or about 5.0×10⁷ to about 6.0×10⁸ such cells. In some embodiments, the number of cells is the number of such cells that are viable cells.

In some embodiments, the dose of genetically engineered cells comprises between at or about 2.5×10⁷ CAR-expressing T cells, total T cells, or total peripheral blood mononuclear cells (PBMCs), and at or about 1.2×10⁹ CAR-expressing T cells, total T cells, or total PBMCs, between at or about 5.0×10⁷ CAR-expressing T cells and at or about 4.5×10⁸ CAR-expressing T cells, total T cells, or total peripheral blood mononuclear cells (PBMCs), between at or about 1.5×10⁸ CAR-expressing T cells and at or about 3.0×10⁸ CAR-expressing T cells, total T cells, or total PBMCs, each inclusive. In some embodiments, the number is with reference to the total number of CD3+ or CD8+, in some cases also CAR-expressing (e.g. CAR+) cells. In some embodiments, the dose comprises a number of cell from or from about 2.5×10⁷ to or to about 1.2×10⁹ CD3+ or CD8+ total T cells or CD3+ or CD8+ CAR-expressing cells, from or from about 5.0×10⁷ to or to about 4.5×10⁸ CD3+ or CD8+ total T cells or CD3+ or CD8+ CAR-expressing cells, or from or from about 1.5×10⁸ to or to about 3.0×10⁸ CD3+ or CD8+ total T cells or CD3+ or CD8+ CAR-expressing cells, each inclusive. In some embodiments, the number of cells is the number of such cells that are viable cells.

In some embodiments, the dose of genetically engineered cells comprises between at or about 1.0×10⁷ CAR-expressing T cells, total T cells, or total peripheral blood mononuclear cells (PBMCs), and at or about 1.2×10⁹ CAR-expressing T cells, total T cells, or total PBMCs, between at or about 2.0×10⁷ CAR-expressing T cells and at or about 4.5×10⁸ CAR-expressing T cells, total T cells, or total peripheral blood mononuclear cells (PBMCs), between at or about 1.5×10⁸ CAR-expressing T cells and at or about 3.0×10⁸ CAR-expressing T cells, total T cells, or total PBMCs, each inclusive. In some embodiments, the number is with reference to the total number of CD3+ or CD8+, in some cases also CAR-expressing (e.g. CAR+) cells. In some embodiments, the dose comprises a number of cell from or from about 1.0×10⁷ to or to about 1.2×10⁹ CD3+ or CD8+ total T cells or CD3+ or CD8+ CAR-expressing cells, from or from about 1.5×10⁷ to or to about 1.2×10⁹ CD3+ or CD8+ total T cells or CD3+ or CD8+ CAR-expressing cells, from or from about 2.5×10⁷ to or to about 1.2×10⁹ CD3+ or CD8+ total T cells or CD3+ or CD8+ CAR-expressing cells, from or from about 1.5×10⁷ to or to about 8.0×10⁸ CD3+ or CD8+ total T cells or CD3+ or CD8+ CAR-expressing cells, from or from about 2.5×10⁷ to or to about 8.0×10⁸ CD3+ or CD8+ total T cells or CD3+ or CD8+ CAR-expressing cells, from or from about 1.5×10⁷ to or to about 6.0×10⁸ CD3+ or CD8+ total T cells or CD3+ or CD8+ CAR-expressing cells, from or from about 2.5×10⁷ to or to about 6.0×10⁸ CD3+ or CD8+ total T cells or CD3+ or CD8+ CAR-expressing cells, from or from about 5.0×10⁷ to or to about 6.0×10⁸ CD3+ or CD8+ total T cells or CD3+ or CD8+ CAR-expressing cells, from or from about 5.0×10⁷ to or to about 4.5×10⁸ CD3+ or CD8+ total T cells or CD3+ or CD8+ CAR-expressing cells, or from or from about 1.5×10⁸ to or to about 3.0×10⁸ CD3+ or CD8+ total T cells or CD3+ or CD8+ CAR-expressing cells, each inclusive. In some embodiments, the number of cells is the number of such cells that are viable cells.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between at or about 1×10⁶ and at or about 2×10⁹ total recombinant receptor (e.g., CAR)-expressing CD8+ cells, e.g., in the range of at or about 5×10⁷ to at or about 4.5×10⁸ such cells, such as at or about 2.5×10⁷, at or about 5×10⁷, at or about 1.5×10⁸, at or about 3×10⁸, at or about 4.5×10⁸, at or about 8×10⁸, or at or about 1.2×10⁹ total such cells, or the range between any two of the foregoing values. In some embodiments, the number of cells is the number of such cells that are viable cells.

In some embodiments, for example, where the subject is human, the CD8+ T cells of the dose, including in a dose including CD4+ and CD8+ T cells, includes between at or about 1×10⁶ and at or about 2×10⁹ total recombinant receptor (e.g., CAR)-expressing CD8+ cells, e.g., in the range of at or about 1×10⁷ to at or about 4.5×10⁸ such cells, such as at or about 1.0×10⁷, at or about 1.25×10⁷, at or about 1.5×10⁷, at or about 2.0×10⁷, at or about 2.5×10⁷, at or about 5×10⁷, at or about 7.5×10⁷, at or about 1.5×10⁸, at or about 3×10⁸, at or about 4.5×10⁸, at or about 6.0×10⁸, at or about 8×10⁸, or at or about 1.2×10⁹ total such cells, or the range between any two of the foregoing values. In some embodiments, the number of cells is the number of such cells that are viable cells.

In some embodiments, the methods and uses involve administering a dose of CAR-expressing T cells that is from or from about 1.0×10⁷ CAR-expressing to T cells to about 1.0×10⁹ CAR-expressing to T cells. In some embodiments, the dose is from or from about 2.5×10⁷ CAR-expressing to T cells to about 5×10⁸ CAR-expressing to T cells, such as from or from about 2.5×10⁷ CAR-expressing to T cells to about 4.5×10⁸ CAR-expressing to T cells, from or from about 2.5×10⁷ CAR-expressing to T cells to about 3×10⁸ CAR-expressing to T cells, from or from about 2.5×10⁷ CAR-expressing to T cells to about 1.5×10⁸ CAR-expressing to T cells, from or from about 2.5×10⁷ CAR-expressing to T cells to about 7.5×10⁷ CAR-expressing to T cells, from or from about 7.5×10⁷ CAR-expressing to T cells to about 4.5×10⁸ CAR-expressing to T cells, from or from about 7.5×10⁷ CAR-expressing to T cells to about 3×10⁸ CAR-expressing to T cells, from or from about 7.5×10⁷ CAR-expressing to T cells to about 1.5×10⁸ CAR-expressing to T cells, from or from about 1.5×10⁸ CAR-expressing to T cells to about 4.5×10⁸ CAR-expressing to T cells, from or from about 1.5×10⁸ CAR-expressing to T cells to about 3×10⁸ CAR-expressing to T cells, or from or from about 3×10⁸ CAR-expressing to T cells to about 4.5×10⁸ CAR-expressing to T cells. In some embodiments, the number of cells is the number of such cells that are viable cells. In some embodiments, the T cells of the dose include CD4+ and CD8+ T cells.

In some embodiments, exemplary doses include about 5.0×10⁷, 1.5×10⁸, 3.0×10⁸ or 4.5×10⁸ CAR-expressing T cells. In some embodiments, exemplary doses include about 1.0×10⁷, 1.25×10⁷, 1.5×10⁷, 1.75×10⁷, 2.0×10⁷, 2.5×10⁷, 3.0×10⁷, 3.5×10⁷, 4.0×10⁷, 4.5×10⁷, 5.0×10⁷, 7.5×10⁷, 1.5×10⁸, 2.25×10⁸, 3.0×10⁸, 4.5×10⁸, or 6.0×10⁸ CAR-expressing T cells. In some embodiments, the dose comprises about 1.0×10⁷ CAR-expressing T cells. In some embodiments, the dose comprises about 1.25×10⁷ CAR-expressing T cells. In some embodiments, the dose comprises about 1.5×10⁷ CAR-expressing T cells. In some embodiments, the dose comprises about 1.75×10⁷ CAR-expressing T cells. In some embodiments, the dose comprises about 2.0×10⁷ CAR-expressing T cells. In some embodiments, the dose comprises about 2.5×10⁷ CAR-expressing T cells. In some embodiments, the dose comprises about 3.0×10⁷ CAR-expressing T cells. In some embodiments, the dose comprises about 3.5×10⁷ CAR-expressing T cells. In some embodiments, the dose comprises about 4.0×10⁷ CAR-expressing T cells. In some embodiments, the dose comprises about 4.5×10⁷ CAR-expressing T cells. In some embodiments, the dose comprises about 5.0×10⁷ CAR-expressing T cells. In some embodiments, the dose comprises about 6.0×10⁷ CAR-expressing T cells. In some embodiments, the dose comprises about 7.0×10⁷ CAR-expressing T cells. In some embodiments, the dose comprises about 7.5×10⁷. In some embodiments, the dose comprises about 8.0×10⁷ CAR-expressing T cells. In some embodiments, the dose comprises about 9.0×10⁷ CAR-expressing T cells. In some embodiments, the dose comprises about 1.0×10⁸ CAR-expressing T cells. In some embodiments, the dose comprises about 1.25×10⁸ CAR-expressing T cells. In some embodiments, the dose comprises about 1.5×10⁸ CAR-expressing T cells. In some embodiments, the dose comprises about 1.75×10⁸ CAR-expressing T cells. In some embodiments, the dose comprises about 2.0×10⁸ CAR-expressing T cells. In some embodiments, the dose comprises about 2.25×10⁸ CAR-expressing T cells. In some embodiments, the dose comprises about 2.5×10⁸ CAR-expressing T cells. In some embodiments, the dose comprises about 3.0×10⁸ CAR-expressing T cells. In some embodiments, the dose comprises about 3.5×10⁸ CAR-expressing T cells. In some embodiments, the dose comprises about 4.0×10⁸ CAR-expressing T cells. In some embodiments, the dose comprises about 4.5×10⁸ CAR-expressing T cells. In some embodiments, the number of cells is the number of such cells that are viable cells. In some embodiments, the T cells of the dose include CD4+ and CD8+ T cells.

In some embodiments, the dose comprises about 6.0×10⁸ CAR-expressing T cells. In some aspects, particular response to the treatment, e.g., according to the methods provided herein, can be assessed based on the International Myeloma Working Group (IMWG) Uniform Response Criteria (see Kumar et al. (2016) Lancet Oncol 17(8):e328-346). In some embodiments, exemplary doses to achieve particular outcomes, such as OR, includes about 1.0×10⁷ CAR-expressing T cells. In some embodiments, exemplary doses to achieve particular outcomes, such as OR, includes about 5.0×10⁷ CAR-expressing T cells. In some embodiments, exemplary doses to achieve particular outcomes, such as OR, includes about 1.0×10⁸ CAR-expressing T cells. In some embodiments, exemplary doses to achieve particular outcomes, such as OR, includes about 1.5×10⁸ CAR-expressing T cells.

In some embodiments, the dose of cells, e.g., recombinant receptor-expressing T cells, is administered to the subject as a single dose or is administered only one time within a period of two weeks, one month, three months, six months, 1 year or more. In some embodiments, the patient is administered multiple doses, and each of the doses or the total dose can be within any of the foregoing values.

In some embodiments, the engineered cells for administration or composition of engineered cells for administration, exhibits properties indicative of or consistent with cell health. In some embodiments, at or about or at least at or about 70, 75, 80, 85, or 90% CAR+ cells of such dose exhibit one or more properties or phenotypes indicative of cell health or biologically active CAR cell, such as absence expression of an apoptotic marker.

In particular embodiments, the phenotype is or includes an absence of apoptosis and/or an indication the cell is undergoing the apoptotic process. Apoptosis is a process of programmed cell death that includes a series of stereotyped morphological and biochemical events that lead to characteristic cell changes and death, including blebbing, cell shrinkage, nuclear fragmentation, chromatin condensation, chromosomal DNA fragmentation, and global mRNA decay. In some aspects, early stages of apoptosis can be indicated by activation of certain caspases, e.g., 2, 8, 9, and 10. In some aspects, middle to late stages of apoptosis are characterized by further loss of membrane integrity, chromatin condensation and DNA fragmentation, include biochemical events such as activation of caspases 3, 6, and 7.

In particular embodiments, the phenotype is negative expression of one or more factors associated with programmed cell death, for example pro-apoptotic factors known to initiate apoptosis, e.g., members of the death receptor pathway, activated members of the mitochondrial (intrinsic) pathway, such as Bel-2 family members, e.g., Bax, Bad, and Bid, and caspases. In certain embodiments, the phenotype is the absence of an indicator, e.g., an Annexin V molecule or by TUNEL staining, that will preferentially bind to cells undergoing apoptosis when incubated with or contacted to a cell composition. In some embodiments, the phenotype is or includes the expression of one or more markers that are indicative of an apoptotic state in the cell. In some embodiments, the phenotype is lack of expression and/or activation of a caspase, such as caspase 3. In some aspects, activation of caspase-3 is indicative of an increase or revival of apoptosis. In certain embodiments, caspase activation can be detected by known methods. In some embodiments, an antibody that binds specifically to an activated caspase (i.e., binds specifically to the cleaved polypeptide) can be used to detect caspase activation. In particular embodiments, the phenotype is or includes active caspase 3-. In some embodiments, the marker of apoptosis is a reagent that detects a feature in a cell that is associated with apoptosis. In certain embodiments, the reagent is an annexin V molecule.

In some embodiments, the compositions containing the engineered cells for administration contain a certain number or amount of cells that exhibit phenotypes indicative of or consistent with cell health. In some of any embodiments, less than about 25%, 20%, 15%, 10%, 9%, 8%, 7%, 6%, 5%, 4%, 3%, 2% or 1% of the CAR-expressing T cells in the dose of engineered T cells express a marker of apoptosis, optionally Annexin V or active Caspase 3. In some of any embodiments, less than 5%, 4%, 3%, 2% or 1% of the CAR-expressing T cells in the dose of engineered T cells express Annexin V or active Caspase 3.

In some embodiments the cells administered are immune cells engineered to express the provided CAR (i.e. the GPRC5D-binding and BCMA-bindingCAR). In some embodiments the immune cells are T cells. In some embodiments, the administered cells are CD4+ T cells. In some embodiments the administered cells are CD8+ T cells. In some embodiments, the administered cells are a combination of CD4+ and CD8+ T cells, such as CAR T cells. In some examples the ratio of CD4+ cells to CD8+ cells (CD4:CD8) is 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1.

In some embodiments, the engineered cells, or compositions containing the same, are administered as part of a combination treatment, such as simultaneously with or sequentially with, in any order, another therapeutic intervention, such as another antibody or engineered cell or receptor or agent, such as a cytotoxic or therapeutic agent.

The engineered cells, or compositions containing the same, in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after to the one or more additional therapeutic agents.

In some embodiments, the subject is administered a lymphodepleting therapy. Lymphodepleting chemotherapy is thought to improve engraftment and activity of recombinant receptor-expressing cells, such as CAR T cells. In some embodiments, lymphodepleting chemotherapy may enhance adoptively transferred tumor-specific T cells to proliferate in vivo through homeostatic proliferation (Grossman 2004, Stachel 2004). In some embodiments, chemotherapy may reduce or eliminate CD4+CD25+ regulatory T cells, which can suppress the function of tumor-targeted adoptively transferred T cells (Turk 2004). In some embodiments, lymphodepleting chemotherapy prior to adoptive T-cell therapy may enhance the expression of stromal cell-derived factor 1 (SDF-1) in the bone marrow, enhancing the homing of modified T cells to the primary tumor site through binding of SDF-1 with CXCR-4 expressed on the T-cell surface (Pinthus 2004). In some embodiments, lymphodepleting chemotherapy may further reduce the subject's tumor burden and potentially lower the risk and severity of CRS.

In some embodiments, lymphodepletion is performed on a subject, e.g., prior to administering engineered cells, e.g., CAR-expressing cells. In some embodiments, the lymphodepletion comprises administering one or more of melphalan, Cytoxan, cyclophosphamide, bendamustine, and/or fludarabine. In some embodiments, a lymphodepleting chemotherapy is administered to the subject prior to, concurrently with, or after administration (e.g., infusion) of engineered cells, e.g., CAR-expressing cells. In an example, the lymphodepleting chemotherapy is administered to the subject prior to administration of engineered cells, e.g., CAR-expressing cells. In some embodiments the lymphodepleting chemotherapy is administered 1 to 10 days prior to administration of engineered cells, such as 1, 2, 3, 4, 5, 6, 7, 8, 9, or 10 days prior to the initiation of administration of engineered cells, or at least 2 days prior, such as at least 3, 4, 5, 6, or 7 days prior, to the initiation of administration of engineered cell. In some embodiments, the subject is administered a preconditioning agent no more than 7 days prior, such as no more than 6, 5, 4, 3, or 2 days prior, to the initiation of administration of engineered cell. The number of days after lymphodepleting chemotherapy that the engineered ells are administered can be determined based on clinical or logistical circumstances. In some examples, dose adjustments or other changes to the lymphodepleting chemotherapy regimen can implemented due to a subject's health, such as the subject's underlying organ function, as determined by the treating physician.

In some embodiments, lymphodepleting chemotherapy comprises administration of a lymphodepleting agent, such as cyclophosphamide, fludarabine, or combinations thereof. In some embodiments, the subject is administered cyclophosphamide at a dose between or between about 20 mg/kg and 100 mg/kg body weight of the subject, such as between or between about 40 mg/kg and 80 mg/kg. In some aspects, the subject is administered about 60 mg/kg of cyclophosphamide. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m² body surface area of the subject, such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 2 to 4 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m² body surface area of the subject, such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 2 to 4 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered fludarabine at or about 30 mg/m², daily, and cyclophosphamide at or about 300 mg/m², daily, for 3 days.

In some embodiments, the lymphodepleting agent comprises bendamustine. In some embodiments, the subject is administered bendamustine at a dose between or between about 50 mg/m2 and 130 mg/m2 body surface area of the subject, such as between or between about 60 mg/m2 and 120 mg/m2, 70 mg/m2 and 110 mg/m2, 80 mg/m2 and 100 mg/m2, or 85 mg/m2 and 95 mg/m2, inclusive. In some instances, the subject is administered about 90 mg/m2 of bendamustine. In some embodiments, the bendamustine can be administered in a single dose or can be administered in a plurality of doses, such as given daily, every other day or every three days. In some embodiments, bendamustine is administered daily, such as for 1-5 days, for example, for 2 to 4 days, for example, for 1 to 3 days. In some instances, the subject is administered about 90 mg/m2 of bendamustine, daily for 2 days, prior to initiation of the cell therapy.

In some embodiments, antiemetic therapy, except dexamethasone or other steroids, may be given prior to lymphodepleting chemotherapy. In some embodiments, Mesna may be used for subjects with a history of hemorrhagic cystitis.

In some embodiments, the subject may receive a bridging therapy after leukapheresis and before lymphodepleting chemotherapy. A treating physician can determine if bridging therapy is necessary, for example for disease control, during manufacturing of the provided composition or cells. In some embodiments, bridging therapies do not include biological agents, such as antibodies (e.g., Daratumumab). In some embodiments, bridging therapies may not contain any experimental therapy. In some embodiments, bridging therapies are discontinued prior to initiation of lymphodepletion. In some embodiments, bridging therapies are discontinued 1 day, 2 days 3 days, 4 days, 5 days, 7 days, 10 days, 14 days, 21 days, 28 days, 45 days, or 60 days before lymphodepletion. In some embodiments, bridging therapy is discontinued at least 14 days before lymphodelpletion. In some embodiments, corticosteroid bridging therapy is discontinued at least 72 hours prior lymphodepletion. In some embodiments, subjects must have recovered from bridging therapy related toxicities to Grade≤2 (except for alopecia) prior to initiation of LD chemotherapy.

Once the cells are administered to a mammal (e.g., a human), the biological activity of the engineered cell populations and/or antibodies in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD 107a, IFNγ, IL-2, and TNF. In some aspects the biological activity is measured by assessing clinical outcome, such as reduction in tumor burden or load.

In certain embodiments, engineered cells are modified in any number of ways, such that their therapeutic or prophylactic efficacy is increased. For example, the engineered CAR or TCR expressed by the population in some embodiments are conjugated either directly or indirectly through a linker to a targeting moiety. The practice of conjugating compounds, e.g., the CAR or TCR, to targeting moieties is known in the art. See, for instance, Wadwa et al., J. Drug Targeting, 3(2):111 (1995), and U.S. Pat. No. 5,087,616.

B. Combination Therapy

Also provided are methods of combination therapy that include administration and uses, such as therapeutic and prophylactic uses, of the GPRC5D- and BCMA-binding recombinant receptors (e.g., bispecific CARs), engineered cells expressing the recombinant receptors (e.g., CARs), plurality of engineered cells expressing the receptors, and/or compositions comprising the same.

In some embodiments, the GPRC5D- and BCMA-binding recombinant receptor (e.g., bispecific CAR), and/or engineered cells expressing said molecules (e.g., recombinant receptor) described herein are administered as part of a combination treatment or combination therapy, such as simultaneously with, sequentially with, or intermittently with, in any order, one or more additional therapeutic intervention. In some embodiments, the one or more additional therapeutic intervention includes, for example, an antibody, an engineered cell, a receptor and/or an agent, such as a cell expressing a recombinant receptor, and/or cytotoxic or therapeutic agent, e.g., a chemotherapeutic agent. In some embodiments, the combination therapy includes administration of one or more additional agents, therapies and/or treatments, e.g., any of the additional agents, therapy and/or treatments described herein. In some embodiments, the combination therapy includes administration of one or more additional agents for treatment or therapy, such as an immunomodulatory agent, immune checkpoint inhibitor, adenosine pathway or adenosine receptor antagonist or agonist and kinase inhibitors. In some embodiments, the combination treatment or combination therapy includes an additional treatment, such as a surgical treatment, transplant, and/or radiation therapy. Also provided are methods of combination treatment or combination therapy that includes GPRC5D- and BCMA-binding recombinant receptors (e.g., bispecific CARs), cells and/or compositions described herein and one or more additional therapeutic interventions.

In some embodiments, the additional agent for combination treatment or combination therapy enhances, boosts and/or promotes the efficacy and/or safety of the therapeutic effect of binding molecules, recombinant receptors, cells and/or compositions. In some embodiments, the additional agent enhances or improves the efficacy, survival or persistence of the administered cells, e.g., cells expressing the binding molecule or a recombinant receptor. In some embodiments, the additional agent is selected from among a protein phosphatase inhibitor, a kinase inhibitor, a cytokine, an immunomodulator, or an agent that decreases the level or activity of a regulatory T (Treg) cell. In some embodiments, the additional agent enhances safety, by virtue of reducing or ameliorating adverse effects of the administered binding molecules, recombinant receptors, cells and/or compositions. In some embodiments, the additional agent can treat the same disease, condition or a comorbidity. In some embodiments, the additional agent can ameliorate, reduce or eliminate one or more toxicities, adverse effects or side effects that are associated with administration of the recombinant receptors, cells and/or compositions, e.g., CAR-expressing cells.

In some embodiments, pain management medication such as acetaminophen, or antihistamine, such as diphenhydramine can be administered prior to, during or after administration of the recombinant receptor, cell or composition provided herein, to ameliorate or reduce or eliminate minor side effects associated with treatment. In some examples, red blood cell and platelet transfusions, and/or colony-stimulating factors can be administered reduce or eliminate one or more toxicities, adverse effects or side effects that are associated with administration of the recombinant receptors, cells and/or compositions, e.g., CAR-expressing cells. In some embodiments, prophylactic or empiric anti-infective agents (e.g., trimethoprim/sulfamethoxazole for Pneumocystis pneumonia [PCP] prophylaxis, broad spectrum antibiotics, antifungals, or antiviral agents for febrile neutropenia) can be administered to treat side-effects resulting from treatment. In some examples, when necessary, prophylaxis may be provided to treat lymphopenia and/or neutropenia occurring as a result of treatment.

In some embodiments, the additional therapy, treatment or agent includes chemotherapy, radiation therapy, surgery, transplantation, adoptive cell therapy, antibodies, cytotoxic agents, chemotherapeutic agents, cytokines, growth inhibitory agents, anti-hormonal agents, kinase inhibitors, anti-angiogenic agents, cardioprotectants, immunostimulatory agents, immunosuppressive agents, immune checkpoint inhibitors, antibiotics, angiogenesis inhibitors, metabolic modulators or other therapeutic agents or any combination thereof. In some embodiments, the additional agent is a protein, a peptide, a nucleic acid, a small molecule agent, a cell, a toxin, a lipid, a carbohydrate or combinations thereof, or any other type of therapeutic agent, e.g., radiation. In some embodiments, the additional therapy, agent or treatment includes surgery, chemotherapy, radiation therapy, transplantation, administration of cells expressing a recombinant receptor, e.g., CAR, kinase inhibitor, immune checkpoint inhibitor, mTOR pathway inhibitor, immunosuppressive agents, immunomodulators, antibodies, immunoablative agents, antibodies and/or antigen binding fragments thereof, antibody conjugates, other antibody therapies, cytotoxins, steroids, cytokines, peptide vaccines, hormone therapy, antimetabolites, metabolic modulators, drugs that inhibit either the calcium dependent phosphatase calcineurin or the p70S6 kinase FK506) or inhibit the p70S6 kinase, alkylating agents, anthracyclines, vinca alkaloids, proteasome inhibitors, GITR agonists, protein tyrosine phosphatase inhibitors, protein kinase inhibitors, an oncolytic virus, and/or other types of immunotherapy. In some embodiments, the additional agent or treatment is bone marrow transplantation, T cell ablative therapy using chemotherapy agents such as, fludarabine, bendamustine, external-beam radiation therapy (XRT), cyclophosphamide, and/or antibody therapy.

In some embodiments, the cells, GPRC5D- and BCMA-binding recombinant receptors and/or compositions, e.g., CAR-expressing cells, are administered in combination with other engineered cells, e.g., other CAR-expressing cells. In some embodiments, the cells, GPRC5D- and BCMA-binding recombinant receptors and/or compositions, e.g., CAR-expressing cells, are administered in combination with an additional agent. In some embodiments, the cells, GPRC5D- and BCMA-binding recombinant receptors and/or compositions, e.g., CAR-expressing cells, are administered in combination with other engineered cells, e.g., other CAR-expressing cells, as well as in combination with an additional agent. In some embodiments, the additional agent is a kinase inhibitor, e.g., an inhibitor of Bruton's tyrosine kinase (Btk), e.g., ibrutinib. In some embodiments, the additional agent is an adenosine pathway or adenosine receptor antagonist or agonist. In some embodiments, the additional agent is an immunomodulator such as thalidomide or a thalidomide derivative (e.g., lenalidomide). In some embodiments, the additional agent is a gamma secretase inhibitor, such as a gamma secretase inhibitor that inhibits or reduces intramembrane cleavage of a target of a gamma secretase, e.g., BCMA, on a cell (such as a tumor/cancer cell). In some embodiments, the additional therapy, agent or treatment is a cytotoxic or chemotherapy agent, a biologic therapy (e.g., antibody, e.g., monoclonal antibody, or cellular therapy), or an inhibitor (e.g., kinase inhibitor).

In some embodiments, the additional agent is a chemotherapeutic agent. Exemplary chemotherapeutic agents include bendamustine, an anthracycline (e.g., doxorubicin, such as liposomal doxorubicin); a vinca alkaloid (e.g., vinblastine, vincristine, vindesine, vinorelbine); an alkylating agent (e.g., cyclophosphamide, decarbazine, melphalan, ifosfamide, temozolomide); an immune cell antibody (e.g., alemtuzumab, gemtuzumab, rituximab, tositumomab); an antimetabolite (including, e.g., folic acid antagonists, pyrimidine analogs, purine analogs and adenosine deaminase inhibitors such as fludarabine); a TNFR glucocorticoid induced TNFR related protein (GITR) agonist; a proteasome inhibitor (e.g., aclacinomycin A, gliotoxin or bortezomib); an immunomodulatory such as thalidomide or a thalidomide derivative (e.g., lenalidomide).

In some embodiments, the additional therapy or treatment is cell therapy, e.g., adoptive cell therapy. In some embodiments, the additional therapy includes administration of engineered cells, e.g., additional CAR-expressing cell. In some embodiments, the additional engineered cell is a CAR-expressing cell that expresses the same or different recombinant receptor as the engineered cells provided herein, cells. In some embodiments, the recombinant receptor, e.g., CAR, expressed on the additional engineered cell, recognizes a different antigen and/or epitope. In some embodiments, the recombinant receptor, e.g., CAR, expressed on the additional engineered cell, recognizes a different epitope of the same antigen as the recombinant receptors described herein, e.g., GPRC5D or BCMA. In some embodiments, the recombinant receptor, e.g., CAR, expressed on the additional engineered cell, recognizes a different antigen, e.g., a different tumor antigen or combination of antigens. For example, in some embodiments, the recombinant receptor, e.g., CAR, expressed on the additional engineered cell, targets cancer cells that express early lineage markers, e.g., cancer stem cells, while other CAR-expressing cells target cancer cells that express later lineage markers. In such embodiments, the additional engineered cell is administered prior to, concurrently with, or after administration (e.g., infusion) of the CAR-expressing cells described herein. In some embodiments, the additional engineered cell expresses an allogeneic CAR.

In some embodiments, the configurations of one or more of the CAR molecules comprise a primary intracellular signaling domain and two or more, e.g., 2, 3, 4, or 5 or more, costimulatory signaling domains. In some embodiments, the one or more of the CAR molecules may have the same or a different primary intracellular signaling domain, the same or different costimulatory signaling domains, or the same number or a different number of costimulatory signaling domains. In some embodiments, the one or more of the CAR molecules can be configured as a split CAR, in which one of the CAR molecules comprises an antigen binding domain and a costimulatory domain (e.g., 4-1BB), while the other CAR molecule comprises an antigen binding domain and a primary intracellular signaling domain (e.g., CD3 zeta).

In some embodiments, the additional agent is any of the cells engineered to express one or more of the GPRC5D- and BCMA-binding molecules and/or cells engineered to express additional binding molecules, e.g., recombinant receptors, e.g., CAR, that target a different antigen. In some embodiments, the additional agent includes any of the cells or plurality of cells described herein, e.g., in Section III. In some embodiments, the additional agent is a cell engineered to express a recombinant receptor, e.g., CAR, targeting a different epitope and/or antigen, e.g., a different antigen associated with a disease or condition. In some embodiments, the additional agent is a cell engineered to express a recombinant receptor, e.g., CAR, targeting a second or additional antigen expressed in multiple myeloma, e.g., CD38, CD138, CS-1, BAFF-R, TACI and/or FcRH5.

In some embodiments, the additional agent is an immunomodulatory agent. In some embodiments, the combination therapy includes an immunomodulatory agent that can stimulate, amplify and/or otherwise enhance an anti-tumor immune response, e.g., anti-tumor immune response from the administered engineered cells, such as by inhibiting immunosuppressive signaling or enhancing immunostimulant signaling. In some embodiments, the immunomodulatory agent is a peptide, protein or is a small molecule. In some embodiments, the protein can be a fusion protein or a recombinant protein. In some embodiments, the immunomodulatory agent binds to an immunologic target, such as a cell surface receptor expressed on immune cells, such a T cells, B cells or antigen-presenting cells. For example, in some embodiments, the immunomodulatory agent is an antibody or antigen-binding antibody fragment, a fusion protein, a small molecule or a polypeptide. In some embodiments, the recombinant receptors, cells and/or compositions are administered in combination with an additional agent that is an antibody or an antigen-binding fragment thereof, such as a monoclonal antibody.

In some embodiments, the immunomodulatory agent blocks, inhibits or counteracts a component of the immune checkpoint pathway. The immune system has multiple inhibitory pathways that are involved in maintaining self-tolerance and for modulating immune responses. Tumors can use certain immune-checkpoint pathways as a major mechanism of immune resistance, particularly against T cells that are specific for tumor antigens (Pardoll (2012) Nature Reviews Cancer 12:252-264), e.g., engineered cells such as CAR-expressing cells. Because many such immune checkpoints are initiated by ligand-receptor interactions, they can be readily blocked by antibodies against the ligands and/or their receptors.

Therefore, therapy with antagonistic molecules blocking an immune checkpoint pathway, such as small molecules, nucleic acid inhibitors (e.g., RNAi) or antibody molecules, are becoming promising avenues of immunotherapy for cancer and other diseases. In contrast to the majority of anti-cancer agents, checkpoint inhibitors do not necessarily target tumor cells directly, but rather target lymphocyte receptors or their ligands in order to enhance the endogenous antitumor activity of the immune system.

As used herein, the term “immune checkpoint inhibitor” refers to molecules that totally or partially reduce, inhibit, interfere with or modulate one or more checkpoint proteins. Checkpoint proteins regulate T-cell activation or function. These proteins are responsible for co-stimulatory or inhibitory interactions of T-cell responses. Immune checkpoint proteins regulate and maintain self-tolerance and the duration and amplitude of physiological immune responses. In some embodiments, the subject can be administered an additional agent that can enhance or boost the immune response, e.g., immune response effected by the GPRC5D- and BCMA-binding recombinant receptors, cells and/or compositions provided herein, against a disease or condition, e.g., a cancer, such as any described herein.

Immune checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors, ligands and/or receptor-ligand interaction. In some embodiments, modulation, enhancement and/or stimulation of particular receptors can overcome immune checkpoint pathway components. Illustrative immune checkpoint molecules that may be targeted for blocking, inhibition, modulation, enhancement and/or stimulation include, but are not limited to, PD-1 (CD279), PD-L1 (CD274, B7-H1), PDL2 (CD273, B7-DC), CTLA-4, LAG-3 (CD223), TIM-3, 4-1BB (CD137), 4-1BBL (CD137L), GITR (TNFRSF18, AITR), CD40, OX40 (CD134, TNFRSF4), CXCR2, tumor associated antigens (TAA), B7-H3, B7-H4, BTLA, HVEM, GAL9, B7H3, B7H4, VISTA, KIR, 2B4 (belongs to the CD2 family of molecules and is expressed on all NK, γδ, and memory CD8+(αβ) T cells), CD160 (also referred to as BY55), CGEN-15049, CEACAM (e.g., CEACAM-1, CEACAM-3 and/or CEACAM-5), TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and a transforming growth factor receptor (TGFR; e.g., TGFR beta). Immune checkpoint inhibitors include antibodies, or antigen binding fragments thereof, or other binding proteins, that bind to and block or inhibit and/or enhance or stimulate the activity of one or more of any of the said molecules.

Exemplary immune checkpoint inhibitors include Tremelimumab (CTLA-4 blocking antibody, also known as ticilimumab, CP-675,206), anti-OX40, PD-L1 monoclonal antibody (Anti-B7-H1; MEDI4736), MK-3475 (PD-1 blocker), nivolumab (anti-PD-1 antibody), CT-011 (anti-PD-1 antibody), BY55 monoclonal antibody, AMP224 (anti-PD-L1 antibody), BMS-936559 (anti-PD-L1 antibody), MPLDL3280A (anti-PD-L1 antibody), MSB0010718C (anti-PD-L1 antibody) and ipilimumab (anti-CTLA-4 antibody, also known as Yervoy®, MDX-010 and MDX-101). Exemplary immunomodulatory antibodies include, but are not limited to, Daclizumab (Zenapax), Bevacizumab (Avastin®), Basiliximab, Ipilimumab, Nivolumab, pembrolizumab, MPDL3280A, Pidilizumab (CT-011), MK-3475, BMS-936559, MPDL3280A (Atezolizumab), tremelimumab, IMP321, BMS-986016, LAG525, urelumab, PF-05082566, TRX518, MK-4166, dacetuzumab (SGN-40), lucatumumab (HCD122), SEA-CD40, CP-870, CP-893, MEDI6469, MEDI6383, MOXR0916, AMP-224, MSB0010718C (Avelumab), MEDI4736, PDR001, rHIgM12B7, Ulocuplumab, BKT140, Varlilumab (CDX-1127), ARGX-110, MGA271, lirilumab (BMS-986015, IPH2101), IPH2201, ARGX-115, Emactuzumab, CC-90002 and MNRP1685A or an antibody-binding fragment thereof. Other exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon gamma, CAS 951209-71-5, available from IRX Therapeutics).

Programmed cell death 1 (PD-1) is an immune checkpoint protein that is expressed in B cells, NK cells, and T cells (Shinohara et al., 1995, Genomics 23:704-6; Blank et al., 2007, Cancer Immunol Immunother 56:739-45; Finger et al., 1997, Gene 197:177-87; Pardoll (2012) Nature Reviews Cancer 12:252-264). The major role of PD-1 is to limit the activity of T cells in peripheral tissues during inflammation in response to infection, as well as to limit autoimmunity. PD-1 expression is induced in activated T cells and binding of PD-1 to one of its endogenous ligands acts to inhibit T-cell activation by inhibiting stimulatory kinases. PD-1 also acts to inhibit the TCR “stop signal”. PD-1 is highly expressed on Treg cells and may increase their proliferation in the presence of ligand (Pardoll (2012) Nature Reviews Cancer 12:252-264). Anti-PD 1 antibodies have been used for treatment of melanoma, non-small-cell lung cancer, bladder cancer, prostate cancer, colorectal cancer, head and neck cancer, triple-negative breast cancer, leukemia, lymphoma and renal cell cancer (Topalian et al., 2012, N Engl J Med 366:2443-54; Lipson et al., 2013, Clin Cancer Res 19:462-8; Berger et al., 2008, Clin Cancer Res 14:3044-51; Gildener-Leapman et al., 2013, Oral Oncol 49:1089-96; Menzies & Long, 2013, Ther Adv Med Oncol 5:278-85). Exemplary anti-PD-1 antibodies include nivolumab (Opdivo by BMS), pembrolizumab (Keytruda by Merck), pidilizumab (CT-011 by Cure Tech), lambrolizumab (MK-3475 by Merck), and AMP-224 (Merck), nivolumab (also referred to as Opdivo, BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are described in U.S. Pat. No. 8,008,449 and WO2006/121168. Pidilizumab (CT-011; Cure Tech) is a humanized IgG1k monoclonal antibody that binds to PD-1. Pidilizumab and other humanized anti-PD-1 monoclonal antibodies are described in WO2009/101611. Pembrolizumab (formerly known as lambrolizumab, and also referred to as Keytruda, MK03475; Merck) is a humanized IgG4 monoclonal antibody that binds to PD-1. Pembrolizumab and other humanized anti-PD-1 antibodies are described in U.S. Pat. No. 8,354,509 and WO2009/114335. Other anti-PD-1 antibodies include AMP 514 (Amplimmune), among others, e.g., anti-PD-1 antibodies described in U.S. Pat. No. 8,609,089, US 2010028330, US 20120114649 and/or US 20150210769. AMP-224 (B7-DCIg; Amplimmune; e.g., described in WO2010/027827 and WO2011/066342), is a PD-L2 Fc fusion soluble receptor that blocks the interaction between PD-1 and B7-H1.

PD-L1 (also known as CD274 and B7-H1) and PD-L2 (also known as CD273 and B7-DC) are ligands for PD-1, found on activated T cells, B cells, myeloid cells, macrophages, and some types of tumor cells. Anti-tumor therapies have focused on anti-PD-L1 antibodies. The complex of PD-1 and PD-L1 inhibits proliferation of CD8+ T cells and reduces the immune response (Topalian et al., 2012, N Engl J Med 366:2443-54; Brahmer et al., 2012, N Eng J Med 366:2455-65). Anti-PD-L1 antibodies have been used for treatment of non-small cell lung cancer, melanoma, colorectal cancer, renal-cell cancer, pancreatic cancer, gastric cancer, ovarian cancer, breast cancer, and hematologic malignancies (Brahmer et al., 2012, N Eng J Med 366:2455-65; Ott et al., 2013, Clin Cancer Res 19:5300-9; Radvanyi et al., 2013, Clin Cancer Res 19:5541; Menzies & Long, 2013, Ther Adv Med Oncol 5:278-85; Berger et al., 2008, Clin Cancer Res 14:13044-51). Exemplary anti-PD-L1 antibodies include MDX-1105 (Medarex), MED14736 (Medimmune) MPDL3280A (Genentech), BMS-935559 (Bristol-Myers Squibb) and MSB0010718C. MED14736 (Medimmune) is a human monoclonal antibody that binds to PD-L1, and inhibits interaction of the ligand with PD-1. MDPL3280A (Genentech/Roche) is a human Fe optimized IgG1 monoclonal antibody that binds to PD-L1. MDPL3280A and other human monoclonal antibodies to PD-L1 are described in U.S. Pat. No. 7,943,743 and U.S Publication No. 20120039906. Other anti-PD-L1 binding agents include YW243.55.S70 (see WO2010/077634) and MDX-1105 (also referred to as BMS-936559, and, e.g., anti-PD-L1 binding agents described in WO2007/005874).

Cytotoxic T-lymphocyte-associated antigen (CTLA-4), also known as CD152, is a co-inhibitory molecule that functions to regulate T-cell activation. CTLA-4 is a member of the immunoglobulin superfamily that is expressed exclusively on T-cells. CTLA-4 acts to inhibit T-cell activation and is reported to inhibit helper T-cell activity and enhance regulatory T-cell immunosuppressive activity. Although the precise mechanism of action of CTLA-4 remains under investigation, it has been suggested that it inhibits T cell activation by outcompeting CD28 in binding to CD80 and CD86, as well as actively delivering inhibitor signals to the T cell (Pardoll (2012) Nature Reviews Cancer 12:252-264). Anti-CTLA-4 antibodies have been used in clinical trials for the treatment of melanoma, prostate cancer, small cell lung cancer, non-small cell lung cancer (Robert & Ghiringhelli, 2009, Oncologist 14:848-61; Ott et al., 2013, Clin Cancer Res 19:5300; Weber, 2007, Oncologist 12:864-72; Wada et al., 2013, J Transl Med 11:89). A significant feature of anti-CTLA-4 is the kinetics of anti-tumor effect, with a lag period of up to 6 months after initial treatment required for physiologic response. In some cases, tumors may actually increase in size after treatment initiation, before a reduction is seen (Pardoll (2012) Nature Reviews Cancer 12:252-264). Exemplary anti-CTLA-4 antibodies include ipilimumab (Bristol-Myers Squibb) and tremelimumab (Pfizer). Ipilimumab has recently received FDA approval for treatment of metastatic melanoma (Wada et al., 2013, J Transl Med 11:89).

Lymphocyte activation gene-3 (LAG-3), also known as CD223, is another immune checkpoint protein. LAG-3 has been associated with the inhibition of lymphocyte activity and in some cases the induction of lymphocyte anergy. LAG-3 is expressed on various cells in the immune system including B cells, NK cells, and dendritic cells. LAG-3 is a natural ligand for the MHC class II receptor, which is substantially expressed on melanoma-infiltrating T cells including those endowed with potent immune-suppressive activity. Exemplary anti-LAG-3 antibodies include Relatlimab (BMS-986016) (Bristol-Myers Squib), which is a monoclonal antibody that targets LAG-3. IMP701 (Immutep) is an antagonist LAG-3 antibody and IMP731 (Immutep and GlaxoSmithKline) is a depleting LAG-3 antibody. Other LAG-3 inhibitors include IMP321 (Immutep), which is a recombinant fusion protein of a soluble portion of LAG-3 and Ig that binds to MHC class II molecules and activates antigen presenting cells (APC). Other antibodies are described, e.g., in WO2010/019570 and US 2015/0259420.

T-cell immunoglobulin domain and mucin domain-3 (TIM-3), initially identified on activated Th1 cells, has been shown to be a negative regulator of the immune response. Blockade of TIM-3 promotes T-cell mediated anti-tumor immunity and has anti-tumor activity in a range of mouse tumor models. Combinations of TIM-3 blockade with other immunotherapeutic agents such as TSR-042, anti-CD137 antibodies and others, can be additive or synergistic in increasing anti-tumor effects. TIM-3 expression has been associated with a number of different tumor types including melanoma, NSCLC and renal cancer, and additionally, expression of intratumoral TIM-3 has been shown to correlate with poor prognosis across a range of tumor types including NSCLC, cervical, and gastric cancers. Blockade of TIM-3 is also of interest in promoting increased immunity to a number of chronic viral diseases. TIM-3 has also been shown to interact with a number of ligands including galectin-9, phosphatidylserine and HMGB1, although which of these, if any, are relevant in regulation of anti-tumor responses is not clear at present. In some embodiments, antibodies, antibody fragments, small molecules, or peptide inhibitors that target TIM-3 can bind to the IgV domain of TIM-3 to inhibit interaction with its ligands. Exemplary antibodies and peptides that inhibit TIM-3 are described in US 2015/0218274, WO2013/006490 and US 2010/0247521. Other anti-TIM-3 antibodies include humanized versions of RMT3-23 (Ngiow et al., 2011, Cancer Res, 71:3540-3551), and clone 8B.2C12 (Monney et al., 2002, Nature, 415:536-541). Bi-specific antibodies that inhibit TIM-3 and PD-1 are described in US 2013/0156774.

In some embodiments, the additional agent is a CEACAM inhibitor (e.g., CEACAM-1, CEACAM-3, and/or CEACAM-5 inhibitor). In some embodiments, the inhibitor of CEACAM is an anti-CEACAM antibody molecule. Exemplary anti-CEACAM-1 antibodies are described in WO 2010/125571, WO 2013/082366 WO 2014/059251 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In some embodiments, the anti-CEACAM antibody binds to CEACAM-5 as described in, e.g., Zheng et al. PLoS One. (2011) 6(6): e21146), or cross reacts with CEACAM-1 and CEACAM-5 as described in, e.g., WO 2013/054331 and US 2014/0271618.

4-1BB, also known as CD137, is transmembrane glycoprotein belonging to the TNFR superfamily. 4-1BB receptors are present on activated T cells and B cells and monocytes. An exemplary anti-4-1BB antibody is urelumab (BMS-663513), which has potential immunostimulatory and antineoplastic activities.

Tumor necrosis factor receptor superfamily, member 4 (TNFRSF4), also known as OX40 and CD134, is another member of the TNFR superfamily. OX40 is not constitutively expressed on resting naïve T cells and acts as a secondary co-stimulatory immune checkpoint molecule. Exemplary anti-OX40 antibodies are MEDI6469 and MOXR0916 (RG7888, Genentech).

In some embodiments, the additional agent includes a molecule that decreases the regulatory T cell (Treg) population. Methods that decrease the number of (e.g., deplete) Treg cells are known in the art and include, e.g., CD25 depletion, cyclophosphamide administration, and modulating Glucocorticoid-induced TNFR family related gene (GITR) function. GITR is a member of the TNFR superfamily that is upregulated on activated T cells, which enhances the immune system. Reducing the number of Treg cells in a subject prior to apheresis or prior to administration of engineered cells, e.g., CAR-expressing cells, can reduce the number of unwanted immune cells (e.g., Tregs) in the tumor microenvironment and reduces the subject's risk of relapse. In some embodiments, the additional agent includes a molecule targeting GITR and/or modulating GITR functions, such as a GITR agonist and/or a GITR antibody that depletes regulatory T cells (Tregs). In some embodiments, the additional agent includes cyclophosphamide. In some embodiments, the GITR binding molecule and/or molecule modulating GITR function (e.g., GITR agonist and/or Treg depleting GITR antibodies) is administered prior to the engineered cells, e.g., CAR-expressing cells. For example, in some embodiments, the GITR agonist can be administered prior to apheresis of the cells. In some embodiments, cyclophosphamide is administered to the subject prior to administration (e.g., infusion or re-infusion) of the engineered cells, e.g., CAR-expressing cells or prior to apheresis of the cells. In some embodiments, cyclophosphamide and an anti-GITR antibody are administered to the subject prior to administration (e.g., infusion or re-infusion) of the engineered cells, e.g., CAR-expressing cells or prior to apheresis of the cells.

In some embodiments, the additional agent is a GITR agonist. Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No. 090505B 1, U.S. Pat. No. 8,586,023, PCT Publication Nos.: WO 2010/003118 and 2011/090754, or an anti-GITR antibody described, e.g., in U.S. Pat. No. 7,025,962, European Patent No. 1947183B 1, U.S. Pat. Nos. 7,812,135, 8,388,967, 8,591,886, European Patent No. EP 1866339, PCT Publication No. WO 2011/028683, PCT Publication No. WO 2013/039954, PCT Publication No. WO2005/007190, PCT Publication No. WO 2007/133822, PCT Publication No. WO2005/055808, PCT Publication No. WO 99/40196, PCT Publication No. WO 2001/03720, PCT Publication No. WO99/20758, PCT Publication No. WO2006/083289, PCT Publication No. WO 2005/115451, U.S. Pat. No. 7,618,632, and PCT Publication No. WO 2011/051726. An exemplary anti-GITR antibody is TRX518.

In some embodiments, the additional agent enhances tumor infiltration or transmigration of the administered cells, e.g., CAR-expressing cells. For example, in some embodiments, the additional agent stimulates CD40, such as CD40L, e.g., recombinant human CD40L. Cluster of differentiation 40 (CD40) is also a member of the TNFR superfamily. CD40 is a costimulatory protein found on antigen-presenting cells and mediates a broad variety of immune and inflammatory responses. CD40 is also expressed on some malignancies, where it promotes proliferation. Exemplary anti-CD40 antibodies are dacetuzumab (SGN-40), lucatumumab (Novartis, antagonist), SEA-CD40 (Seattle Genetics), and CP-870,893. In some embodiments, the additional agent that enhances tumor infiltration includes tyrosine kinase inhibitor sunitnib, heparanase, and/or chemokine receptors such as CCR2, CCR4, and CCR7.

In some embodiments, the additional agent includes thalidomide drugs or analogs thereof and/or derivatives thereof, such as lenalidomide, pomalidomide or apremilast. See, e.g., Bertilaccio et al., Blood (2013) 122:4171, Otahal et al., Oncoimmunology (2016) 5(4):e1115940; Fecteau et al., Blood (2014) 124(10):1637-1644 and Kuramitsu et al., Cancer Gene Therapy (2015) 22:487-495). Lenalidomide ((RS)-3-(4-Amino-1-oxo-1,3-dihydro-2H-isoindol-2-yl)piperidine-2,6-dione; also known as Revlimid) is a synthetic derivative of thalidomide, and has multiple immunomodulatory effects, including enforcement of immune synapse formation between T cell and antigen presenting cells (APCs). For example, in some cases, lenalidomide modulates T cell responses and results in increased interleukin (IL)-2 production in CD4+ and CD8+ T cells, induces the shift of T helper (Th) responses from Th2 to Th1, inhibits expansion of regulatory subset of T cells (Tregs), and improves functioning of immunological synapses in follicular lymphoma and chronic lymphocytic leukemia (CLL) (Otahal et al., Oncoimmunology (2016) 5(4):e1115940). Lenalidomide also has direct tumoricidal activity in patients with multiple myeloma (MM) and directly and indirectly modulates survival of CLL tumor cells by affecting supportive cells, such as nurse-like cells found in the microenvironment of lymphoid tissues. Lenalidomide also can enhance T-cell proliferation and interferon-γ production in response to activation of T cells via CD3 ligation or dendritic cell-mediated activation. Lenalidomide can also induce malignant B cells to express higher levels of immunostimulatory molecules such as CD80, CD86, HLA-DR, CD95, and CD40 (Fecteau et al., Blood (2014) 124(10):1637-1644). In some embodiments, lenalidomide is administered at a dosage of from about 1 mg to about 20 mg daily, e.g., from about 1 mg to about 10 mg, from about 2.5 mg to about 7.5 mg, from about 5 mg to about 15 mg, such as about 5 mg, 10 mg, 15 mg or 20 mg daily. In some embodiments, lenalidomide is administered at a dose of from about 10 μg/kg to 5 mg/kg, e.g., about 100 μg/kg to about 2 mg/kg, about 200 μg/kg to about 1 mg/kg, about 400 μg/kg to about 600 μg/kg, such as about 500 μg/kg. In some embodiments, rituximab is administered at a dosage of about 350-550 mg/m² (e.g., 350-375, 375-400, 400-425, 425-450, 450-475, or 475-500 mg/m²), e.g., intravenously. In some embodiments, lenalidomide is administered at a low dose.

In some embodiments, the additional agent is a B-cell inhibitor. In some embodiments, the additional agent is one or more B-cell inhibitors selected from among inhibitors of CD10, CD19, CD20, CD22, CD34, CD123, CD79a, CD79b, CD179b, FLT-3, or ROR1, or a combination thereof. In some embodiments, the B-cell inhibitor is an antibody (e.g., a mono- or bispecific antibody) or an antigen binding fragment thereof. In some embodiments, the additional agent is an engineered cell expressing recombinant receptors that target B-cell targets, e.g., CD10, CD19, CD20, CD22, CD34, CD123, CD79a, CD79b, CD179b, FLT-3, or ROR1.

In some embodiments, the additional agent is a CD20 inhibitor, e.g., an anti-CD20 antibody (e.g., an anti-CD20 mono- or bi-specific antibody) or a fragment thereof. Exemplary anti-CD20 antibodies include but are not limited to rituximab, ofatumumab, ocrelizumab (also known as GA101 or RO5072759), veltuzumab, obinutuzumab, TRU-015 (Trubion Pharmaceuticals), ocaratuzumab (also known as AME-133v or ocaratuzumab), and Pro131921 (Genentech). See, e.g., Lim et al. Haematologica. (2010) 95(1):135-43. In some embodiments, the anti-CD20 antibody comprises rituximab. Rituximab is a chimeric mouse/human monoclonal antibody IgG1 kappa that binds to CD20 and causes cytolysis of a CD20 expressing cell. In some embodiments, the additional agent includes rituximab. In some embodiments, the CD20 inhibitor is a small molecule.

In some embodiments, the additional agent is a CD22 inhibitor, e.g., an anti-CD22 antibody (e.g., an anti-CD22 mono- or bi-specific antibody) or a fragment thereof. Exemplary anti-CD22 antibodies include epratuzumab and RFB4. In some embodiments, the CD22 inhibitor is a small molecule. In some embodiments, the antibody is a monospecific antibody, optionally conjugated to a second agent such as a chemotherapeutic agent. For instance, in some embodiments, the antibody is an anti-CD22 monoclonal antibody-MMAE conjugate (e.g., DCDT2980S). In some embodiments, the antibody is an scFv of an anti-CD22 antibody, e.g., an scFv of antibody RFB4. In some embodiments, the scFv is fused to all of or a fragment of Pseudomonas exotoxin-A (e.g., BL22). In some embodiments, the scFv is fused to all of or a fragment of (e.g., a 38 kDa fragment of) Pseudomonas exotoxin-A (e.g., moxetumomab pasudotox). In some embodiments, the anti-CD22 antibody is an anti-CD19/CD22 bispecific antibody, optionally conjugated to a toxin. For instance, in some embodiments, the anti-CD22 antibody comprises an anti-CD19/CD22 bispecific portion, (e.g., two scFv ligands, recognizing human CD19 and CD22) optionally linked to all of or a portion of diphtheria toxin (DT), e.g., first 389 amino acids of diphtheria toxin (DT), DT 390, e.g., a ligand-directed toxin such as DT2219ARL). In some embodiments, the bispecific portion (e.g., anti-CD 19/anti-CD22) is linked to a toxin such as deglycosylated ricin A chain (e.g., Combotox).

In some embodiments, the immunomodulatory agent is a cytokine. In some embodiments, the immunomodulatory agent is a cytokine or is an agent that induces increased expression of a cytokine in the tumor microenvironment. Cytokines have important functions related to T cell expansion, differentiation, survival, and homeostasis. Cytokines that can be administered to the subject receiving the GPRC5D-binding recombinant receptors, cells and/or compositions provided herein include one or more of IL-2, IL-4, IL-7, IL-9, IL-15, IL-18, and IL-21. Cytokines that can be administered to the subject receiving the GPRC5D-binding recombinant receptors, BCMA-binding recombinant receptors, GPRC5D- and BCMA-binding recombinant receptors, cells and/or compositions provided herein include one or more of IL-2, IL-4, IL-7, IL-9, IL-15, IL-18, and IL-21. In some embodiments, the cytokine administered is IL-7, IL-15, or IL-21, or a combination thereof. In some embodiments, administration of the cytokine to the may improve certain aspects such as response or anti-tumor activity of the administered cells, e.g., CAR-expressing cells.

Cytokine may refer to proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines. For example, the immunomodulatory agent is a cytokine and the cytokine is IL-4, TNF-α, GM-CSF or IL-2.

In some embodiments, the additional agent includes an interleukin-15 (IL-15) polypeptide, an interleukin-15 receptor alpha (IL-15Rα) polypeptide, or combination thereof, e.g., hetIL-15 (Admune Therapeutics, LLC). hetIL-15 is a heterodimeric non-covalent complex of IL-15 and IL-15Rα. hetIL-15 is described in, e.g., U.S. Pat. No. 8,124,084, U.S. 2012/0177598, U.S. 2009/0082299, U.S. 2012/0141413, and U.S. 2011/0081311. In some embodiments, the immunomodulatory agent can contain one or more cytokines. For example, the interleukin can include leukocyte interleukin injection (Multikine), which is a combination of natural cytokines. In some embodiments, the immunomodulatory agent is a Toll-like receptor (TLR) agonist, an adjuvant or a cytokine.

In some embodiments, the additional agent is an agent that ameliorates or neutralizes one or more toxicities or side effects associated with the cell therapy. In some embodiments, the additional agent is selected from among a steroid (e.g., corticosteroid), an inhibitor of TNFα, and an inhibitor of IL-6. An example of a TNFα inhibitor is an anti-TNFα antibody molecule such as, infliximab, adalimumab, certolizumab pegol, and golimumab. Another example of a TNFα inhibitor is a fusion protein such as entanercept. Small molecule inhibitors of TNFα include, but are not limited to, xanthine derivatives (e.g. pentoxifylline) and bupropion. An example of an IL-6 inhibitor is an anti-IL-6 antibody molecule such as tocilizumab, sarilumab, elsilimomab, CNTO 328, ALD518/BMS-945429, CNTO 136, CPSI-2364, CDP6038, VX30, ARGX-109, FE301, and FM101. In some embodiments, the anti-IL-6 antibody molecule is tocilizumab. In some embodiments, the additional agent is an IL-1R inhibitor, such as anakinra.

In some embodiments, the additional agent is a modulator of adenosine levels and/or an adenosine pathway component. Adenosine can function as an immunomodulatory agent in the body. For example, adenosine and some adenosine analogs that non-selectively activate adenosine receptor subtypes decrease neutrophil production of inflammatory oxidative products (Cronstein et al., Ann. N.Y. Acad. Sci. 451:291, 1985; Roberts et al., Biochem. J., 227:669, 1985; Schrier et al., J. Immunol. 137:3284, 1986; Cronstein et al., Clinical Immunol. Immunopath. 42:76, 1987). In some cases, concentration of extracellular adenosine or adenosine analogs can increase in specific environments, e.g., tumor microenvironment (TME). In some cases, adenosine or adenosine analog signaling depends on hypoxia or factors involved in hypoxia or its regulation, e.g., hypoxia inducible factor (HIF). In some embodiments, increase in adenosine signaling can increase in intracellular cAMP and cAMP-dependent protein kinase that results in inhibition of proinflammatory cytokine production, and can lead to the synthesis of immunosuppressive molecules and development of Tregs (Sitkovsky et al., Cancer Immunol Res (2014) 2(7):598-605). In some embodiments, the additional agent can reduce or reverse immunosuppressive effects of adenosine, adenosine analogs and/or adenosine signaling. In some embodiments, the additional agent can reduce or reverse hypoxia-driven A2-adenosinergic T cell immunosuppression. In some embodiments, the additional agent is selected from among antagonists of adenosine receptors, extracellular adenosine-degrading agents, inhibitors of adenosine generation by CD39/CD73 ectoenzymes, and inhibitors of hypoxia-HIF-1α signaling. In some embodiments, the additional agent is an adenosine receptor antagonist or agonist.

Inhibition or reduction of extracellular adenosine or the adenosine receptor by virtue of an inhibitor of extracellular adenosine (such as an agent that prevents the formation of, degrades, renders inactive, and/or decreases extracellular adenosine), and/or an adenosine receptor inhibitor (such as an adenosine receptor antagonist) can enhance immune response, such as a macrophage, neutrophil, granulocyte, dendritic cell, T- and/or B cell-mediated response. In addition, inhibitors of the Gs protein mediated cAMP dependent intracellular pathway and inhibitors of the adenosine receptor-triggered Gi protein mediated intracellular pathways, can also increase acute and chronic inflammation.

In some embodiments, the additional agent is an adenosine receptor antagonist or agonist, e.g., an antagonist or agonist of one or more of the adenosine receptors A2a, A2b, A1, and A3. A1 and A3 inhibit, and A2a and A2b stimulate, respectively, adenylate cyclase activity. Certain adenosine receptors, such as A2a, A2b, and A3, can suppress or reduce the immune response during inflammation. Thus, antagonizing immunosuppressive adenosine receptors can augment, boost or enhance immune response, e.g., immune response from administered cells, e.g., CAR-expressing T cells. In some embodiments, the additional agent inhibits the production of extracellular adenosine and adenosine-triggered signaling through adenosine receptors. For example, enhancement of an immune response, local tissue inflammation, and targeted tissue destruction can be enhanced by inhibiting or reducing the adenosine-producing local tissue hypoxia; by degrading (or rendering inactive) accumulated extracellular adenosine; by preventing or decreasing expression of adenosine receptors on immune cells; and/or by inhibiting/antagonizing signaling by adenosine ligands through adenosine receptors.

An antagonist is any substance that tends to nullify the action of another, as an agent that binds to a cell receptor without eliciting a biological response. In some embodiments, the antagonist is a chemical compound that is an antagonist for an adenosine receptor, such as the A2a, A2b, or A3 receptor. In some embodiments, the antagonist is a peptide, or a pepidomimetic, that binds the adenosine receptor but does not trigger a Gi protein dependent intracellular pathway. Exemplary antagonists are described in U.S. Pat. Nos. 5,565,566; 5,545,627, 5,981,524; 5,861,405; 6,066,642; 6,326,390; 5,670,501; 6,117,998; 6,232,297; 5,786,360; 5,424,297; 6,313,131, 5,504,090; and 6,322,771.

In some embodiments, the additional agent is an A2 receptor (A2R) antagonist, such as an A2a antagonist. Exemplary A2R antagonists include KW6002 (istradefyline), SCH58261, caffeine, paraxanthine, 3,7-dimethyl-1-propargylxanthine (DMPX), 8-(m-chlorostyryl) caffeine (CSC), MSX-2, MSX-3, MSX-4, CGS-15943, ZM-241385, SCH-442416, preladenant, vipadenant (BII014), V2006, ST-1535, SYN-115, PSB-1115, ZM241365, FSPTP, and an inhibitory nucleic acid targeting A2R expression, e.g., siRNA or shRNA, or any antibodies or antigen-binding fragment thereof that targets an A2R. In some embodiments, the additional agent is an A2R antagonist described in, e.g., Ohta et al., Proc Natl Acad Sci USA (2006) 103:13132-13137; Jin et al., Cancer Res. (2010) 70(6):2245-2255; Leone et al., Computational and Structural Biotechnology Journal (2015) 13:265-272; Beavis et al., Proc Natl Acad Sci USA (2013) 110:14711-14716; and Pinna, A., Expert Opin Investig Drugs (2009) 18:1619-1631; Sitkovsky et al., Cancer Immunol Res (2014) 2(7):598-605; U.S. Pat. Nos. 8,080,554; 8,716,301; US 20140056922; WO2008/147482; U.S. Pat. No. 8,883,500; US 20140377240; WO02/055083; U.S. Pat. Nos. 7,141,575; 7,405,219; 8,883,500; 8,450,329 and 8,987,279).

In some embodiments, the antagonist is an antisense molecule, inhibitory nucleic acid molecule (e.g., small inhibitory RNA (siRNA)) or catalytic nucleic acid molecule (e.g. a ribozyme) that specifically binds mRNA encoding an adenosine receptor. In some embodiments, the antisense molecule, inhibitory nucleic acid molecule or catalytic nucleic acid molecule binds nucleic acids encoding A2a, A2b, or A3. In some embodiments, an antisense molecule, inhibitory nucleic acid molecule or catalytic nucleic acid targets biochemical pathways downstream of the adenosine receptor. For example, the antisense molecule or catalytic nucleic acid can inhibit an enzyme involved in the Gs protein- or Gi protein-dependent intracellular pathway. In some embodiments, the additional agent includes dominant negative mutant form of an adenosine receptor, such as A2a, A2b, or A3.

In some embodiments, the additional agent that inhibits extracellular adenosine includes agents that render extracellular adenosine non-functional (or decrease such function), such as a substance that modifies the structure of adenosine to inhibit the ability of adenosine to signal through adenosine receptors. In some embodiments, the additional agent is an extracellular adenosine-generating or adenosine-degrading enzyme, a modified form thereof or a modulator thereof. For example, in some embodiments, the additional agent is an enzyme (e.g. adenosine deaminase) or another catalytic molecule that selectively binds and destroys the adenosine, thereby abolishing or significantly decreasing the ability of endogenously formed adenosine to signal through adenosine receptors and terminate inflammation.

In some embodiments, the additional agent is an adenosine deaminase (ADA) or a modified form thereof, e.g., recombinant ADA and/or polyethylene glycol-modified ADA (ADA-PEG), which can inhibit local tissue accumulation of extracellular adenosine. ADA-PEG has been used in treatment of patients with ADA SCID (Hershfield (1995) Hum Mutat. 5:107). In some embodiments, an agent that inhibits extracellular adenosine includes agents that prevent or decrease formation of extracellular adenosine, and/or prevent or decrease the accumulation of extracellular adenosine, thereby abolishing, or substantially decreasing, the immunosuppressive effects of adenosine. In some embodiments, the additional agent specifically inhibits enzymes and proteins that are involved in regulation of synthesis and/or secretion of pro-inflammatory molecules, including modulators of nuclear transcription factors. Suppression of adenosine receptor expression or expression of the Gs protein- or Gi protein-dependent intracellular pathway, or the cAMP dependent intracellular pathway, can result in an increase/enhancement of immune response.

In some embodiments, the additional agent can target ectoenzymes that generate or produce extracellular adenosine. In some embodiments, the additional agent targets CD39 and CD73 ectoenzymes, which function in tandem to generate extracellular adenosine. CD39 (also called ectonucleoside triphosphate diphosphohydrolase) converts extracellular ATP (or ADP) to 5′AMP. Subsequently, CD73 (also called 5′nucleotidase) converts 5′AMP to adenosine. The activity of CD39 is reversible by the actions of NDP kinase and adenylate kinase, whereas the activity of CD73 is irreversible. CD39 and CD73 are expressed on tumor stromal cells, including endothelial cells and Tregs, and also on many cancer cells. For example, the expression of CD39 and CD73 on endothelial cells is increased under the hypoxic conditions of the tumor microenvironment. Tumor hypoxia can result from inadequate blood supply and disorganized tumor vasculature, impairing delivery of oxygen (Carroll and Ashcroft (2005), Expert. Rev. Mol. Med. 7(6):1-16). Hypoxia also inhibits adenylate kinase (AK), which converts adenosine to AMP, leading to very high extracellular adenosine concentration. Thus, adenosine is released at high concentrations in response to hypoxia, which is a condition that frequently occurs the tumor microenvironment (TME), in or around solid tumors. In some embodiments, the additional agent is one or more of anti-CD39 antibody or antigen binding fragment thereof, anti-CD73 antibody or antigen binding fragment thereof, e.g., MEDI9447 or TY/23, α-β-methylene-adenosine diphosphate (ADP), ARL 67156, POM-3, IPH52 (see, e.g., Allard et al. Clin Cancer Res (2013) 19(20):5626-5635; Hausler et al., Am J Transl Res (2014) 6(2):129-139; Zhang, B., Cancer Res. (2010) 70(16):6407-6411).

In some embodiments, the additional agent is an inhibitor of hypoxia inducible factor 1 alpha (HIF-1α) signaling. Exemplary inhibitors of HIF-1α include digoxin, acriflavine, sirtuin-7 and ganetespib.

In some embodiments, the additional agent includes a protein tyrosine phosphatase inhibitor, e.g., a protein tyrosine phosphatase inhibitor described herein. In some embodiments, the protein tyrosine phosphatase inhibitor is an SHP-1 inhibitor, e.g., an SHP-1 inhibitor described herein, such as, e.g., sodium stibogluconate. In some embodiments, the protein tyrosine phosphatase inhibitor is an SHP-2 inhibitor, e.g., an SHP-2 inhibitor described herein.

In some embodiments, the additional agent is a kinase inhibitor. Kinase inhibitors, such as a CDK4 kinase inhibitor, a BTK kinase inhibitor, a MNK kinase inhibitor, or a DGK kinase inhibitor, can regulate the constitutively active survival pathways that exist in tumor cells and/or modulate the function of immune cells. In some embodiments, the kinase inhibitor is a Bruton's tyrosine kinase (BTK) inhibitor, e.g., ibrutinib. In some embodiments, the kinase inhibitor is a phosphatidylinositol-4,5-bisphosphate 3-kinase (PI3K) inhibitor. In some embodiments, the kinase inhibitor is a CDK4 inhibitor, e.g., a CDK4/6 inhibitor. In some embodiments, the kinase inhibitor is an mTOR inhibitor, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor. In some embodiments, the kinase inhibitor is an MNK inhibitor, or a dual PI3K/mTOR inhibitor. In some embodiments, other exemplary kinase inhibitors include the AKT inhibitor perifosine, the mTOR inhibitor temsirolimus, the Src kinase inhibitors dasatinib and fostamatinib, the JAK2 inhibitors pacritinib and ruxolitinib, the PKCβ inhibitors enzastaurin and bryostatin, and the AAK inhibitor alisertib.

In some embodiments, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13. In some embodiments, the BTK inhibitor does not reduce or inhibit the kinase activity of interleukin-2-inducible kinase (ITK), and is selected from GDC-0834; RN-486; CGI-560; CGI-1764; HM-71224; CC-292; ONO-4059; CNX-774; and LFM-A13.

In some embodiments, the kinase inhibitor is a BTK inhibitor, e.g., ibrutinib (1-[(3R)-3-[4-Amino-3-(4-phenoxyphenyl)-1H-pyrazolo[3,4-d]pyrimidin-1-yl]piperidin-1-yl]prop-2-en-1-one; also known as PCI-32765). In some embodiments, the kinase inhibitor is a BTK inhibitor, e.g., ibrutinib (PCI-32765), and the ibrutinib is administered at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle, or daily for 28 day cycle. In some embodiments, 1, 2, 3, 4, 5, 6, 7, 8, 9, 10, 11, 12 or more cycles of ibrutinib are administered. In some embodiments, the BTK inhibitor is a BTK inhibitor described in International Application WO 2015/079417.

In some embodiments, the kinase inhibitor is a PI3K inhibitor. PI3K is central to the PI3K/Akt/mTOR pathway involved in cell cycle regulation and lymphoma survival. Exemplary PI3K inhibitor includes idelalisib (PI3K6 inhibitor). In some embodiments, the additional agent is idelalisib and rituximab.

In some embodiments, the additional agent is an inhibitor of mammalian target of rapamycin (mTOR). In some embodiments, the kinase inhibitor is an mTOR inhibitor selected from temsirolimus; ridaforolimus (also known as AP23573 and MK8669); everolimus (RAD001); rapamycin (AY22989); simapimod; AZD8055; PF04691502; SF1126; and XL765. In some embodiments, the additional agent is an inhibitor of mitogen-activated protein kinase (MAPK), such as vemurafenib, dabrafenib, and trametinib.

In some embodiments, the additional agent is an agent that regulates pro- or anti-apoptotic proteins. In some embodiments, the additional agent includes a B-cell lymphoma 2 (BCL-2) inhibitor (e.g., venetoclax, also called ABT-199 or GDC-0199; or ABT-737). Venetoclax is a small molecule (4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide) that inhibits the anti-apoptotic protein, BCL-2. Other agents that modulate pro- or anti-apoptotic protein include BCL-2 inhibitor ABT-737, navitoclax (ABT-263); Mcl-1 siRNA or Mcl-1 inhibitor retinoid N-(4-hydroxyphenyl) retinamide (4-HPR) for maximal efficacy. In some embodiments, the additional agent provides a pro-apoptotic stimuli, such as recombinant tumor necrosis factor-related apoptosis-inducing ligand (TRAIL), which can activate the apoptosis pathway by binding to TRAIL death receptors DR-4 and DR-5 on tumor cell surface, or TRAIL-R2 agonistic antibodies.

In some embodiments, the additional agent includes an indoleamine 2,3-dioxygenase (IDO) inhibitor. IDO is an enzyme that catalyzes the degradation of the amino acid, L-tryptophan, to kynurenine. Many cancers overexpress IDO, e.g., prostatic, colorectal, pancreatic, cervical, gastric, ovarian, head, and lung cancer. Plasmacytoid dendritic cells (pDCs), macrophages, and dendritic cells (DCs) can express IDO. In some aspects, a decrease in L-tryptophan (e.g., catalyzed by IDO) results in an immunosuppressive milieu by inducing T-cell anergy and apoptosis. Thus, in some aspects, an IDO inhibitor can enhance the efficacy of the GPRC5D-binding recombinant receptors, cells and/or compositions described herein, e.g., by decreasing the suppression or death of the administered CAR-expressing cell. Exemplary inhibitors of IDO include but are not limited to 1-methyl-tryptophan, indoximod (New Link Genetics) (see, e.g., Clinical Trial Identifier Nos. NCT01191216; NCT01792050), and INCB024360 (Incyte Corp.) (see, e.g., Clinical Trial Identifier Nos. NCT01604889; NCT01685255).

In some embodiments, the additional agent includes a cytotoxic agent, e.g., CPX-351 (Celator Pharmaceuticals), cytarabine, daunorubicin, vosaroxin (Sunesis Pharmaceuticals), sapacitabine (Cyclacel Pharmaceuticals), idarubicin, or mitoxantrone. In some embodiments, the additional agent includes a hypomethylating agent, e.g., a DNA methyltransferase inhibitor, e.g., azacitidine or decitabine.

In another embodiment, the additional therapy is transplantation, e.g., an allogeneic stem cell transplant.

In some embodiments, the additional agent is an oncolytic virus. In some embodiments, oncolytic viruses are capable of selectively replicating in and triggering the death of or slowing the growth of a cancer cell. In some cases, oncolytic viruses have no effect or a minimal effect on non-cancer cells. An oncolytic virus includes but is not limited to an oncolytic adenovirus, oncolytic Herpes Simplex Viruses, oncolytic retrovirus, oncolytic parvovirus, oncolytic vaccinia virus, oncolytic Sinbis virus, oncolytic influenza virus, or oncolytic RNA virus (e.g., oncolytic reovirus, oncolytic Newcastle Disease Virus (NDV), oncolytic measles virus, or oncolytic vesicular stomatitis virus (VSV)).

Other exemplary combination therapy, treatment and/or agents include anti-allergenic agents, anti-emetics, analgesics and adjunct therapies. In some embodiments, the additional agent includes cytoprotective agents, such as neuroprotectants, free-radical scavengers, cardioprotectors, anthracycline extravasation neutralizers and nutrients.

In some embodiments, an antibody used as an additional agent is conjugated or otherwise bound to a therapeutic agent, e.g., a chemotherapeutic agent (e.g., Cytoxan, fludarabine, histone deacetylase inhibitor, demethylating agent, peptide vaccine, anti-tumor antibiotic, tyrosine kinase inhibitor, alkylating agent, anti-microtubule or anti-mitotic agent), anti-allergic agent, anti-nausea agent (or anti-emetic), pain reliever, or cytoprotective agent described herein. In some embodiments, the additional agent is an antibody-drug conjugate.

In some embodiments, the additional agent can modulate, inhibit or stimulate particular factors at the DNA, RNA or protein levels, such as to enhance or boost certain aspects. In some embodiments, the additional agent can modulate the factors at the nucleic acid level, e.g., DNA or RNA, within the administered cells, e.g., cells engineered to express recombinant receptors, e.g., CAR. In some embodiments, an inhibitory nucleic acid, e.g., an inhibitory nucleic acid, e.g., a dsRNA, e.g., an siRNA or shRNA, or a clustered regularly interspaced short palindromic repeats (CRISPR), a transcription-activator like effector nuclease (TALEN), or a zinc finger endonuclease (ZFN), can be used to inhibit expression of an inhibitory molecule in the engineered cell, e.g., CAR-expressing cell. In some embodiments the inhibitor is an shRNA. In some embodiments, the inhibitory molecule is inhibited within the engineered cell, e.g., CAR-expressing cell. In some embodiments, a nucleic acid molecule that encodes a dsRNA molecule that inhibits expression of the molecule that modulates or regulates, e.g., inhibits, T-cell function is operably linked to a promoter, e.g., a HI- or a U6-derived promoter such that the dsRNA molecule that inhibits expression of the inhibitory molecule is expressed within the engineered cell, e.g., CAR-expressing cell. See, e.g., Brummelkamp T R, et al. (2002) Science 296: 550-553; Miyagishi M, et al. (2002) Nat. Biotechnol. 19: 497-500.

In some embodiments, the additional agent is capable of disrupting the gene encoding an inhibitory molecule, such as any immune checkpoint inhibitors described herein. In some embodiments, disruption is by deletion, e.g., deletion of an entire gene, exon, or region, and/or replacement with an exogenous sequence, and/or by mutation, e.g., frameshift or missense mutation, within the gene, typically within an exon of the gene. In some embodiments, the disruption results in a premature stop codon being incorporated into the gene, such that the inhibitory molecule is not expressed or is not expressed in a form that is capable of being expressed on the cells surface and/or capable of mediating cell signaling. The disruption is generally carried out at the DNA level. The disruption generally is permanent, irreversible, or not transient.

In some aspects, the disruption is carried out by gene editing, such as using a DNA binding protein or DNA-binding nucleic acid, which specifically binds to or hybridizes to the gene at a region targeted for disruption. In some aspects, the protein or nucleic acid is coupled to or complexed with a nuclease, such as in a chimeric or fusion protein. For example, in some embodiments, the disruption is effected using a fusion comprising a DNA-targeting protein and a nuclease, such as a Zinc Finger Nuclease (ZFN) or TAL-effector nuclease (TALEN), or an RNA-guided nuclease such as a clustered regularly interspersed short palindromic nucleic acid (CRISPR)-Cas system, such as CRISPR-Cas9 system, specific for the gene being disrupted. In some embodiments, methods of producing or generating genetically engineered cells, e.g., CAR-expressing cells, include introducing into a population of cells nucleic acid molecules encoding a genetically engineered antigen receptor (e.g. CAR) and nucleic acid molecules encoding an agent targeting an inhibitory molecule that is a gene editing nuclease, such as a fusion of a DNA-targeting protein and a nuclease such as a ZFN or a TALEN, or an RNA-guided nuclease such as of the CRISPR-Cas9 system, specific for an inhibitory molecule.

Any of the additional agents described herein can be prepared and administered as combination therapy with the GPRC5D- and BCMA-binding recombinant receptor (e.g., bispecific chimeric antigen receptor) and/or engineered cells expressing said molecules (e.g., recombinant receptor) described herein, such as in pharmaceutical compositions comprising one or more agents of the combination therapy and a pharmaceutically acceptable carrier, such as any described herein. In some embodiments, the GPRC5D- and BCMA-binding recombinant receptor (e.g., bispecific chimeric antigen receptor), engineered cells expressing said molecules (e.g., recombinant receptor), plurality of engineered cells expressing said molecules (e.g., recombinant receptor) can be administered simultaneously, concurrently or sequentially, in any order with the additional agents, therapy or treatment, wherein such administration provides therapeutically effective levels each of the agents in the body of the subject. In some embodiments, the additional agent can be co-administered with the GPRC5D- and BCMA-binding recombinant receptors, cells and/or compositions described herein, for example, as part of the same pharmaceutical composition or using the same method of delivery. In some embodiments, the additional agent is administered simultaneously with the GPRC5D- and BCMA-binding recombinant receptors, cells and/or compositions described herein, but in separate compositions. In some embodiments, the additional agent is an additional engineered cell, e.g., cell engineered to express a different recombinant receptor, and is administered in the same composition or in a separate composition. In some embodiments, the additional agent is incubated with the engineered cell, e.g., CAR-expressing cells, prior to administration of the cells.

In some examples, the one or more additional agents are administered subsequent to or prior to the administration of the GPRC5D- and BCMA-binding recombinant receptors, cells and/or compositions described herein, separated by a selected time period. In some examples, the time period is 1 day, 2 days, 3 days, 4 days, 5 days, 6 days, 1 week, 2 weeks, 3 weeks, 1 month, 2 months, or 3 months. In some examples, the one or more additional agents are administered multiple times and/or the GPRC5D- and BCMA-binding recombinant receptors, cells and/or compositions described herein, is administered multiple times. For example, in some embodiments, the additional agent is administered prior to the GPRC5D- and BCMA-binding recombinant receptors, cells and/or compositions described herein, e.g., two weeks, 12 days, 10 days, 8 days, one week, 6 days, 5 days, 4 days, 3 days, 2 days or 1 day before the administration.

The dose of the additional agent can be any therapeutically effective amount, e.g., any dose amount described herein, and the appropriate dosage of the additional agent may depend on the type of disease to be treated, the type, dose and/or frequency of the recombinant receptor, cell and/or composition administered, the severity and course of the disease, whether the recombinant receptor, cell and/or composition is administered for preventive or therapeutic purposes, previous therapy, the patient's clinical history and response to the recombinant receptor, cell and/or composition, and the discretion of the attending physician. The recombinant receptor, cell and/or composition and/or the additional agent and/or therapy can be administered to the patient at one time, repeated or administered over a series of treatments.

### VI. Articles of Manufacture or Kits

Also provided are articles of manufacture or kits containing the provided recombinant receptors (e.g., CARs), genetically engineered cells, and/or compositions comprising the same. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., a cancer, such as multiple myeloma). Alternatively, or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating the GPRC5D-expressing or GPRC5D-associated disease, disorder or condition in an individual. The label or package insert may indicate that the composition is used for treating the BCMA-expressing or BCMA-associated disease, disorder or condition in an individual. The label or a package insert, which is on or associated with the container, may indicate directions for reconstitution and/or use of the formulation. The label or package insert may further indicate that the formulation is useful or intended for subcutaneous, intravenous, or other modes of administration for treating or preventing a GPRC5D-expressing or GPRC5D-associated disease, disorder or condition in an individual. The label or package insert may further indicate that the formulation is useful or intended for subcutaneous, intravenous, or other modes of administration for treating or preventing a BCMA-expressing or BCMA-associated disease, disorder or condition in an individual.

The container in some embodiments holds a composition which is by itself or combined with another composition effective for treating, preventing and/or diagnosing the condition. The article of manufacture or kit may include (a) a first container with a composition contained therein (i.e., first medicament), wherein the composition includes the recombinant receptor (e.g., CAR or engineered cell containing the CAR); and (b) a second container with a composition contained therein (i.e., second medicament), wherein the composition includes a further agent, such as a cytotoxic or otherwise therapeutic agent, and which article or kit further comprises instructions on the label or package insert for treating the subject with the second medicament, in an effective amount.

### VII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, reference to a “corresponding form” of an antibody means that when comparing a property or activity of two antibodies, the property is compared using the same form of the antibody. For example, if it is stated that an antibody has greater activity compared to the activity of the corresponding form of a first antibody, that means that a particular form, such as an scFv of that antibody, has greater activity compared to the scFv form of the first antibody.

The term “Fe region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

The terms “full length antibody,” “intact antibody,” and “whole antibody” are used herein interchangeably to refer to an antibody having a structure substantially similar to a native antibody structure or having heavy chains that contain an Fc region as defined herein.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-GPRC5D antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The terms “polypeptide” and “protein” are used interchangeably to refer to a polymer of amino acid residues, and are not limited to a minimum length. Polypeptides, including the antibodies and antibody chains and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into a binding molecule, e.g., antibody, of interest and the products screened for a desired activity, e.g., retained/improved antigen binding, or decreased immunogenicity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative amino acid substitutions will involve exchanging a member of one of these classes for another class.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “composition” refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

### VIII. Exemplary Embodiments

Among the provided embodiments are:
1. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus:
   (i) one of the VH region and the VL region of the GPRC5D-binding domain, one of the VH region and the VL region of the BCMA-binding domain, the other of the VH region and the VL region of the BCMA-binding domain, and the other of the VH region and the VL region of the GPRC5D-binding domain; or
   (ii) one of the VH region and the VL region of the BCMA-binding domain, one of the VH region and the VL region of the GPRC5D-binding domain, the other of the VH region and the VL region of the GPRC5D-binding domain, and the other of the VH region and the VL region of the BCMA-binding domain;
(b) a spacer;
(c) a transmembrane domain; and
(d) an intracellular signaling domain.
2. The bispecific CAR of embodiment 1, wherein the extracellular domain comprises, in order from amino to carboxy terminus, one of the VH region and the VL region of the GPRC5D-binding domain, one of the VH region and the VL region of the BCMA-binding domain, the other of the VH region and the VL region of the BCMA-binding domain, and the other of the VH region and the VL region of the GPRC5D-binding domain.
3. The bispecific CAR of embodiment 1 or embodiment 2, wherein the extracellular domain comprises, in order from amino to carboxy terminus: the VH region of the GPRC5D-binding domain, the VH region of the BCMA-binding domain, the VL region of the BCMA-binding domain, and the VL region of the GPRC5D-binding domain.
4. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus: the VH region of the GPRC5D-binding domain, the VH region of the BCMA-binding domain, the VL region of the BCMA-binding domain, and the VL region of the GPRC5D-binding domain;
(b) a spacer;
(c) a transmembrane domain; and
(d) an intracellular signaling domain.
5. The bispecific CAR of embodiment 1 or embodiment 2, wherein the extracellular domain comprises, in order from amino to carboxy terminus: the VH region of the GPRC5D-binding domain, the VL region of the BCMA-binding domain, the VH region of the BCMA-binding domain, and the VL region of the GPRC5D-binding domain.
6. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus: the VH region of the GPRC5D-binding domain, the VL region of the BCMA-binding domain, the VH region of the BCMA-binding domain, and the VL region of the GPRC5D-binding domain;
(b) a spacer;
(c) a transmembrane domain; and
(d) an intracellular signaling domain.
7. The bispecific CAR of embodiment 1 or embodiment 2, wherein the extracellular domain comprises, in order from amino to carboxy terminus: the VL region of the GPRC5D-binding domain, the VH region of the BCMA-binding domain, the VL region of the BCMA-binding domain, and the VH region of the GPRC5D-binding domain.
8. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VL region of the GPRC5D-binding domain, the VH region of the BCMA-binding domain, the VL region of the BCMA-binding domain, and the VH region of the GPRC5D-binding domain;
(b) a spacer;
(c) a transmembrane domain; and
(d) an intracellular signaling domain.
9. The bispecific CAR of embodiment 1 or embodiment 2, wherein the extracellular domain comprises in order from amino to carboxy terminus: the VL region of the GPRC5D-binding domain, the VL region of the BCMA-binding domain, the VH region of the BCMA-binding domain, and the VH region of the GPRC5D-binding domain.
10. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VL region of the GPRC5D-binding domain, the VL region of the BCMA-binding domain, the VH region of the BCMA-binding domain, and the VH region of the GPRC5D-binding domain;
(b) a spacer;
(c) a transmembrane domain; and
(d) an intracellular signaling domain.
11. The bispecific CAR of embodiment 1, wherein the extracellular domain comprises, in order from amino to carboxy terminus, one of the VH region and the VL region of the BCMA-binding domain, one of the VH region and the VL region of the GPRC5D-binding domain, the other of the VH region and the VL region of the GPRC5D-binding domain, and the other of the VH region and the VL region of the BCMA-binding domain.
12. The bispecific CAR of embodiment 1 or embodiment 11, wherein the extracellular domain comprises in order from amino to carboxy terminus: the VH region of the BCMA-binding domain, the VH region of the GPRC5D-binding domain, the VL region of the GPRC5D-binding domain, and the VL region of the BCMA-binding domain.
13. A bispecific chimeric antigen receptor (CAR), comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VH region of the BCMA-binding domain, the VH region of the GPRC5D-binding domain, the VL region of the GPRC5D-binding domain, and the VL region of the BCMA-binding domain;
(b) a spacer;
(c) a transmembrane domain; and
(d) an intracellular signaling domain.
14. The bispecific CAR of embodiment 1 or embodiment 11, wherein the extracellular domain comprises in order from amino to carboxy terminus: the VH region of the BCMA-binding domain, the VL region of the GPRC5D-binding domain, the VH region of the GPRC5D-binding domain, and the VL region of the BCMA-binding domain.
15. A bispecific chimeric antigen receptor (CAR), comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VH region of the BCMA-binding domain, the VL region of the GPRC5D-binding domain, the VH region of the GPRC5D-binding domain, and the VL region of the BCMA-binding domain;
(b) a spacer;
(c) a transmembrane domain; and
(d) an intracellular signaling domain.
16. The bispecific CAR of embodiment 1 or embodiment 11, wherein the extracellular domain comprises in order from amino to carboxy terminus: the VL region of the BCMA-binding domain, the VH region of the GPRC5D-binding domain, the VL region of the GPRC5D-binding domain, and the VH region of the BCMA-binding domain.
17. A bispecific chimeric antigen receptor comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VL region of the BCMA-binding domain, the VH region of the GPRC5D-binding domain, the VL region of the GPRC5D-binding domain, and the VH region of the BCMA-binding domain;
(b) a spacer;
(c) a transmembrane domain; and
(d) an intracellular signaling domain.
18. The bispecific CAR of embodiment 1 or embodiment 11, wherein the extracellular domain comprises in order from amino to carboxy terminus: the VL region of the BCMA-binding domain, the VL region of the GPRC5D-binding domain, the VH region of the GPRC5D-binding domain, and the VH region of the BCMA-binding domain.
19. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VL region of the BCMA-binding domain, the VL region of the GPRC5D-binding domain, the VH region of the GPRC5D-binding domain, and the VH region of the BCMA-binding domain;
(b) a spacer;
(c) a transmembrane domain; and
(d) an intracellular signaling domain.
20. The bispecific CAR of any one of embodiments 1-19, wherein (a) the VH region or the VL region of the GPRC5D-binding domain; and (b) the VH region or the VL region of the BCMA-binding domain are joined by a linker.
21. The bispecific CAR of embodiment 20, wherein the linker is a flexible peptide linker.
22. The bispecific CAR of embodiment 20 or embodiment 21, wherein the linker is 4 to 12 amino acids in length.
23. The bispecific CAR of any of embodiments 20-22, wherein the linker is or comprises the amino acid sequence set forth in SEQ ID NO:19, SEQ ID NO:21, or SEQ ID NO:22.
24. The bispecific CAR of any one of embodiments 1-23, wherein:
(a) the VH region and the VL region of the GPRC5D-binding domain are joined by a linker; or (b) the VH region and the VL region of the BCMA-binding domain are joined by a linker.
25. The bispecific CAR of embodiment 24, wherein the linker comprises the amino acid sequence set forth in SEQ ID NO:17 or SEQ ID NO:18.
26. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus:
   (i) the VH region of the GPRC5D-binding domain;
   (ii) the linker set forth in SEQ ID NO:21;
   (iii) the VL region of the BCMA-binding domain;
   (iv) the linker set forth in SEQ ID NO:17;
   (v) the VH region of the BCMA-binding domain;
   (vi) the linker set forth in SEQ ID NO:21; and
   (vii) the VL region of the GPRC5D-binding domain;
(b) a spacer;
(c) a transmembrane domain; and
(d) an intracellular signaling domain.
27. A bispecific chimeric antigen receptor (CAR) comprising:
(a) an extracellular domain comprising (i) a GPRC5D-binding domain that binds to GPRC5D comprising a heavy chain variable (VH) region and a light chain variable (VL) region; and (ii) a BCMA-binding domain that binds to BCMA comprising a VH region and a VL region, wherein the extracellular domain comprises, in order from amino to carboxy terminus: one of the VH region and the VL region of the BCMA-binding domain; the other of the VH region and the VL region of the BCMA-binding domain; one of the VH region and the VL region of the GPRC5D-binding domain; and the other of the VH region and the VL region of the GPRC5D-binding domain;
(b) a spacer;
(c) a transmembrane domain; and
(d) an intracellular signaling domain.
28. A bispecific chimeric antigen receptor (CAR) comprising:
(a) an extracellular domain comprising (i) a GPRC5D-binding domain that binds to GPRC5D comprising a heavy chain variable (VH) region and a light chain variable (VL) region; and (ii) a BCMA-binding domain that binds to BCMA comprising a VH region and a VL region, wherein the extracellular domain comprises, in order from amino to carboxy terminus: the VL region of the GPRC5D-binding domain; the VH region of the GPRC5D-binding domain; one of the VH region and the VL region of the BCMA-binding domain; and the other of the VH and the VL region of the BCMA-binding domain;
(b) a spacer;
(c) a transmembrane domain; and
(d) an intracellular signaling domain.
29. The bispecific CAR of embodiment 27 or embodiment 28, wherein the GPRC5D-binding region and the BCMA-binding region are joined by a linker.
30. The bispecific CAR of embodiment 29, wherein the linker is a flexible peptide linker.
31. The bispecific CAR of embodiment 29 or embodiment 30, wherein the linker is 4 to 12 amino acids in length.
32. The bispecific CAR of any one of embodiments 29-31, wherein the linker comprises the amino acid sequence set forth in SEQ ID NO:19, SEQ ID NO:21, or SEQ ID NO:24.
33. The bispecific CAR of any one of embodiments 27-32, wherein the VH region and the VL region of the BCMA-binding domain are joined by a linker comprising the amino acid sequence set forth in SEQ ID NO:17.
34. A bispecific chimeric antigen receptor (CAR) comprising:
(a) an extracellular domain comprising (i) a GPRC5D-binding domain that binds to GPRC5D comprising a heavy chain variable (VH) region and a light chain variable (VL) region; and (ii) a BCMA-binding domain that binds to BCMA comprising a VH region and a VL region, wherein the extracellular domain comprises in order from amino to carboxy terminus: the VH region of the GPRC5D-binding domain; the VL region of the GPRC5D-binding domain; one of the VH region and the VL region of the BCMA-binding domain; and the other of the VH and the VL region of the BCMA-binding domain;
(b) a spacer;
(c) a transmembrane domain; and
(d) an intracellular signaling domain,
wherein the GPRC5D-binding domain and the BCMA-binding domain are joined by a linker comprising the sequence set forth in SEQ ID NO:19 or SEQ ID NO:21.
35. The bispecific CAR of any one of embodiments 1-34, wherein the VH region of the GPRC5D-binding domain comprises a CDR-1, a CDR-2, and a CDR-3 comprising the amino acid sequences set forth in SEQ ID NO:1, SEQ ID NO:2, and SEQ ID NO:3, respectively.
36. The bispecific CAR of any one of embodiments 1-35, wherein the VL region of the GPRC5D-binding domain comprises a CDR-1, a CDR-2, and a CDR-3 comprising the amino acid sequences set forth in SEQ ID NO:4, SEQ ID NO:5, and SEQ ID NO:6, respectively.
37. The bispecific CAR of any one of embodiments 1-36, wherein the VH region of the GPRC5D-binding domain comprises a CDR-1, a CDR-2, and a CDR-3 comprising the amino acid sequences set forth in SEQ ID NO:1, SEQ ID NO:2, and SEQ ID NO:3, respectively; and the VL region of the GPRC5D-binding domain comprises a CDR-1, a CDR-2, and a CDR-3 comprising the amino acid sequences set forth in SEQ ID NO:4, SEQ ID NO:5, and SEQ ID NO:6, respectively.
38. The bispecific CAR of any one of embodiments 1-37, wherein the V_{H} region of the GPRC5D-binding domain an amino acid sequence having at least about 90% sequence identity to the amino acid sequence set forth in SEQ ID NO:7.
39. The bispecific CAR of any one of embodiments 1-38, wherein the VL region of the GPRC5D-binding domain comprises an amino acid sequence having at least about 90% sequence identity to the amino acid sequence set forth in SEQ ID NO:8.
40. The bispecific CAR of any one of embodiments 1-39, wherein the VH region of the GPRC5D-binding domain comprises an amino acid sequence having at least about 90% sequence identity to the amino acid sequence set forth in SEQ ID NO:7; and the VL region of the GPRC5D-binding domain comprises an amino acid sequence having at least about 90% sequence identity to the amino acid sequence set forth in SEQ ID NO:8.
41. The bispecific CAR of any one of embodiments 1-40, wherein the VH region of the GPRC5D-binding domain comprises the amino acid sequence set forth in SEQ ID NO:7.
42. The bispecific CAR of any one of embodiments 1-41, wherein the VL region of the GPRC5D-binding domain comprises the amino acid sequence set forth in SEQ ID NO:8.
43. The bispecific CAR of any one of embodiments 1-42, wherein the VH region of the GPRC5D-binding domain comprises the amino acid sequence set forth in SEQ ID NO:7; and the VL region of the GPRC5D-binding domain comprises the amino acid sequence set forth in SEQ ID NO:8.
44. The bispecific CAR of any one of embodiments 1-43, wherein the VH region of the BCMA-binding domain comprises a CDR-1, a CDR-2, and a CDR-3 comprising the amino acid sequences set forth in SEQ ID NO:9, SEQ ID NO:10, and SEQ ID NO:11, respectively.
45. The bispecific CAR of any one of embodiments 1-44, wherein the VL region of the BCMA-binding domain comprises a CDR-1, a CDR-2, and a CDR-3 comprising the amino acid sequences set forth in SEQ ID NO:12, SEQ ID NO:13, and SEQ ID NO:14, respectively.
46. The bispecific CAR of any one of embodiments 1-45, wherein the VH region of the BCMA-binding domain comprises an amino acid sequence having at least about 90% sequence identity to the amino acid sequence set forth in SEQ ID NO:15.
47. The bispecific CAR of any one of embodiments 1-46, wherein the VL region of the BCMA-binding domain comprises an amino acid sequence having at least about 90% sequence identity to the amino acid sequence set forth in SEQ ID NO:16.
48. The bispecific CAR of any one of embodiments 1-47, wherein the VH region of the BCMA-binding domain comprises an amino acid sequence having at least about 90% sequence identity to the amino acid sequence set forth in SEQ ID NO:15; and the VL region of the BCMA-binding domain comprises an amino acid sequence having at least about 90% sequence identity to the amino acid sequence set forth in SEQ ID NO:16.
49. The bispecific CAR of any one of embodiments 1-48, wherein the VH region of the BCMA-binding domain comprises the amino acid sequence set forth in SEQ ID NO:15.
50. The bispecific CAR of any one of embodiments 1-49, wherein the VL region of the BCMA-binding domain comprises the amino acid sequence set forth in SEQ ID NO:16.
51. The bispecific CAR of any one of embodiments 1-50, wherein the VH region of the BCMA-binding domain comprises the amino acid sequence set forth in SEQ ID NO:15; and the VL region of the BCMA-binding domain comprises the amino acid sequence set forth in SEQ ID NO:16.
52. The bispecific CAR of any one of embodiments 1, 20-25, and 35-51, wherein the extracellular binding domain comprises the amino acid sequence set forth in any one of SEQ ID NO:77, 78, 79, and 80.
53. The bispecific CAR of any one of embodiments 1, 20-25, and 35-51, wherein the extracellular binding domain comprises the amino acid sequence set forth in SEQ ID NO:81, 82, 83, 84, 85, 86, 87, 88, 89, and 90.
54. The bispecific CAR of any one of embodiments 1, 2, 5, 6, 20-26, 35-51 and 53, wherein the extracellular binding domain comprises the amino acid sequence set forth in SEQ ID NO: 83.
55. The bispecific CAR of any one of embodiments 1, 2, 7, 8, 20-25, 35-51 and 53, wherein the extracellular binding domain comprises the amino acid sequence set forth in SEQ ID NO: 84.
56. The bispecific CAR of any one of embodiments 1, 2, 5, 6, 20-25, 35-51 and 53, wherein the extracellular binding domain comprises the amino acid sequence set forth in SEQ ID NO: 87.
57. The bispecific CAR of any one of embodiments 1, 11, 14, 15, 20-25, 35-51 and 53, wherein the extracellular binding domain comprises the amino acid sequence set forth in SEQ ID NO: 81.
58. The bispecific CAR of any one of embodiments 1, 11, 16, 17, 20-25, 35-51 and 53, wherein the extracellular binding domain comprises the amino acid sequence set forth in SEQ ID NO: 85.
59. The bispecific CAR of any one of embodiments 1, 11, 18-25, 35-51 and 53, wherein the extracellular binding domain comprises the amino acid sequence set forth in SEQ ID NO: 86.
60. The bispecific CAR of any one of embodiments 1, 11, 16, 17, 20-25, 35-51 and 53, wherein the extracellular binding domain comprises the amino acid sequence set forth in SEQ ID NO: 90.
61. The bispecific CAR of any one of embodiments 1-60, wherein the spacer comprises at least a portion of an immunoglobulin or a variant thereof.
62. The bispecific CAR of any one of embodiments 1-61, wherein the spacer comprises a hinge region of an immunoglobulin or a variant thereof.
63. The bispecific CAR of embodiment 62, wherein the hinge region of an immunoglobulin is an IgG4 hinge region, optionally a human IgG4 hinge region, or a variant thereof.
64. The bispecific CAR of any one of embodiments 1-63, wherein the spacer is less than at or about 15 amino acids in length.
65. The bispecific CAR of any one of embodiments 1-64, wherein the spacer is between 12 and 15 amino acids in length.
66. The bispecific CAR of any one of embodiments 1-65, wherein the spacer comprises the amino acid sequence set forth in SEQ ID NO:25, or an amino acid sequence having at least about 90% sequence identity to the amino acid sequence set forth in SEQ ID NO:25.
67. The bispecific CAR of any one of embodiments 1-64, wherein the spacer is between 200 and 250 amino acids in length, optionally between 220 and 240 amino acids in length.
68. The bispecific CAR of any one of embodiment 1-64 and 67, wherein the spacer comprises a hinge region of an immunoglobulin, a CH2 region of an immunoglobulin or a chimeric CH2 region of two different immunoglobulins, and a CH3 region of an immunoglobulin.
69. The bispecific CAR of any one of embodiments 1-64, 67, and 68, wherein the spacer comprises IgG4 hinge region or a variant thereof, a chimeric CH2 region comprising a portion of an IgG4 CH2 and a portion of an IgG2 CH2 (IgG2/4 CH2 region), and an IgG4 C_{H}3 region.
70. The bispecific CAR of any one of embodiments 1-64 and 67-69, wherein the spacer comprises the amino acid sequence set forth in SEQ ID NO:27, or an amino acid sequence having at least about 90% sequence identity to the amino acid sequence set forth in SEQ ID NO:27.
71. The bispecific CAR of any one of embodiments 1-70, wherein the transmembrane domain is or comprises a transmembrane domain from CD4, CD28, or CD8, optionally from human CD4, human CD28 or human CD8.
72. The bispecific CAR of any one of embodiments 1-71, wherein the transmembrane domain is or comprises a transmembrane domain from human CD28.
73. The bispecific CAR of any one of embodiments 1-72, wherein the transmembrane domain comprises the amino acid sequence set forth in SEQ ID NO:28, or an amino acid sequence having at least about 90% sequence identity to the amino acid sequence set forth in SEQ ID NO:28.
74. The bispecific CAR of any one of embodiments 1-73, wherein the intracellular signaling domain is a domain from a T cell receptor (TCR) component or comprises an immunoreceptor tyrosine-based activation motif (ITAM).
75. The bispecific CAR of any one of embodiments 1-74, wherein the intracellular signaling domain comprises a cytoplasmic signaling domain of a CD3-zeta chain, optionally a human CD3-zeta chain.
76. The bispecific CAR of any one of embodiments 1-75, wherein the intracellular signaling domain comprises the amino acid sequence set forth in SEQ ID NO:30, or an amino acid sequence having at least about 90% sequence identity to the amino acid sequence set forth in SEQ ID NO:30.
77. The bispecific CAR of any one of embodiments 1-76, wherein the intracellular signaling domain further comprises a costimulatory signaling region.
78. The bispecific CAR of embodiment 77, wherein the costimulatory signaling region is located between the transmembrane region and the intracellular signaling domain.
79. The bispecific CAR of embodiment 77 or embodiment 78, wherein the costimulatory signaling region comprises an intracellular signaling domain of a T cell costimulatory molecule or a signaling portion thereof.
80. The bispecific CAR of any one of embodiments 77-79, wherein the costimulatory signaling region comprises an intracellular signaling domain of CD28, 4-1BB, or ICOS, or a signaling portion thereof, optionally human CD28, human 4-1BB, or human ICOS.
81. The bispecific CAR of any one of embodiments 77-80, wherein the costimulatory signaling region comprises an intracellular signaling domain of 4-1BB or a signaling portion thereof, optionally human 4-1BB.
82. The bispecific CAR of any one of embodiments 68-72, wherein the costimulatory signaling region comprises the amino acid sequence set forth in SEQ ID NO:29, or an amino acid sequence having at least about 90% sequence identity to the amino acid sequence set forth in SEQ ID NO:29.
83. The bispecific CAR of any one of embodiments 1-82, wherein the CAR comprises the amino acid sequence that has at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 98% sequence identity to any one of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, or SEQ ID NO:44.
84. The bispecific CAR of any one of embodiments 1-83, wherein the CAR comprises the amino acid sequence set forth in SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, or SEQ ID NO:44.
85. The bispecific CAR of embodiment 84, wherein the CAR embodiments the amino acid sequence set forth in SEQ ID NO:37.
86. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus:
(i) the VH region of the GPRC5D-binding domain comprising a CDR-1, a CDR-2, and a CDR-3 comprising the amino acid sequences set forth in SEQ ID NO:1, SEQ ID NO:2, and SEQ ID NO:3, respectively;
(ii) the linker set forth in SEQ ID NO:21;
(iii) the VL region of the BCMA-binding domain comprising a CDR-1, a CDR-2, and a CDR-3 comprising the amino acid sequences set forth in SEQ ID NO:12, SEQ ID NO:13, and SEQ ID NO:14, respectively;
(iv) the linker set forth in SEQ ID NO:17;
(v) the VH region of the BCMA-binding domain comprising a CDR-1, a CDR-2, and a CDR-3 comprising the amino acid sequences set forth in SEQ ID NO:9, SEQ ID NO:10, and SEQ ID NO:11, respectively;
(vi) the linker set forth in SEQ ID NO:21; and
(vii) the VL region of the GPRC5D-binding domain comprising a CDR-1, a CDR-2, and a CDR-3 comprising the amino acid sequences set forth in SEQ ID NO:4, SEQ ID NO:5, and SEQ ID NO:6, respectively;
(b) a spacer comprising the amino acid sequence set forth in SEQ ID NO:27;
(c) a transmembrane domain comprising the amino acid sequence set forth in SEQ ID NO:28; and
(d) an intracellular signaling domain comprising the amino acid sequences set forth in SEQ ID NOS:29 and 30.
87. The bispecific CAR of embodiment 86, wherein the extracellular binding domain comprises the amino acid sequence set forth in SEQ ID NO: 83.
88. The bispecific CAR of embodiment 86 or embodiment 87, wherein the CAR comprises the amino acid sequence set forth in SEQ ID NO:37.
89. The bispecific CAR of any of embodiments 76-88, which is encoded by the nucleotide sequence set forth in SEQ ID NO:119.
90. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus:
(i) the VL region of the BCMA-binding domain comprising a CDR-1, a CDR-2, and a CDR-3 comprising the amino acid sequences set forth in SEQ ID NO:12, SEQ ID NO:13, and SEQ ID NO:14, respectively;
(ii) the linker set forth in SEQ ID NO:21;
(vii) the VL region of the GPRC5D-binding domain comprising a CDR-1, a CDR-2, and a CDR-3 comprising the amino acid sequences set forth in SEQ ID NO:4, SEQ ID NO:5, and SEQ ID NO:6, respectively;
(iv) the linker set forth in SEQ ID NO:17;
(i) the VH region of the GPRC5D-binding domain comprising a CDR-1, a CDR-2, and a CDR-3 comprising the amino acid sequences set forth in SEQ ID NO:1, SEQ ID NO:2, and SEQ ID NO:3, respectively;
(vi) the linker set forth in SEQ ID NO:21; and
(v) the VH region of the BCMA-binding domain comprising a CDR-1, a CDR-2, and a CDR-3 comprising the amino acid sequences set forth in SEQ ID NO:9, SEQ ID NO:10, and SEQ ID NO:11, respectively;
(b) a spacer comprising the amino acid sequence set forth in SEQ ID NO:27;
(c) a transmembrane domain comprising the amino acid sequence set forth in SEQ ID NO:28; and
(d) an intracellular signaling domain comprising the amino acid sequences set forth in SEQ ID NOS:29 and 30.
91. The bispecific CAR of embodiment 90, wherein the extracellular binding domain comprises the amino acid sequence set forth in SEQ ID NO: 86.
92. The bispecific CAR of embodiment 90 or embodiment 91, wherein the CAR comprises the amino acid sequence set forth in SEQ ID NO:40.
93. The bispecific CAR of any one of embodiments 90-92, which is encoded by the polynucleotide sequence set forth in SEQ ID NO:120.
94. A polynucleotide encoding the CAR of any one of embodiments 1-88 and 90.
95. The polynucleotide of embodiment 94, comprising the nucleotide sequence set forth in any one of SEQ ID NOS:105-120.
96. A polynucleotide comprising the nucleotide sequence set forth in any one of SEQ ID NOS:105-120.
97. The polynucleotide of any of embodiments 94-96, wherein the polynucleotide is optimized by splice site elimination.
98. The polynucleotide of any of embodiments 94-97, wherein the polynucleotide is codon-optimized for expression in a human cell.
99. The polynucleotide of any of embodiments 94-98, comprising the nucleotide sequence set forth in SEQ ID NO:119 or SEQ ID NO:120.
100. The polynucleotide of any of embodiments 94-99, comprising the nucleotide sequence set forth in SEQ ID NO:119.
101. The polynucleotide of any of embodiments 94-100, comprising the nucleotide sequence set forth in SEQ ID NO:120.
102. A vector comprising the polynucleotide of any one of embodiments 94-101.
103. The vector of embodiment 102, which is a viral vector.
104. The vector of embodiment 102 or embodiment 103, which is a retroviral vector.
105. The vector of any one of embodiments 102-104, which is a lentiviral vector or an adeno-associated viral (AAV) vector.
106. A cell comprising the CAR of any one of embodiments 1-93.
107. A cell comprising the polynucleotide of any one of embodiments 90-101 or the vector or any one of embodiments 102-105.
108. The cell of embodiment 106 or embodiment 107, wherein the cell is an immune cell.
109. The cell of any one of embodiments 106-108, wherein the cell is a lymphocyte.
110. The cell of any one of embodiments 106-109, wherein the cell is a NK cell or a T cell.
111. The cell of any one of embodiments 106-110, wherein the cell is a T cell.
112. The cell of embodiment 111, wherein the T cell is a CD4+ T cell or a CD8+ T cell.
113. The cell of embodiment 111 or embodiment 112, wherein the T cell is a primary T cell.
114. The cell of any one of embodiment 106 or embodiment 107, wherein the cell is a stem cell.
115. The cell of any embodiment 114, wherein the stem cell is a multipotent and pluripotent stem cell.
116. The cell of embodiment 114 or embodiment 115, wherein the stem cell is an induced pluripotent stem cell (iPSC).
117. The cell of any one of embodiments 106-112, wherein the cell has been differentiated from an induced pluripotent stem cell.
118. The cell of any one of embodiments 106-117, wherein the cell is an allogeneic cell.
119. The cell of any one of embodiments 106-118, wherein the cell is engineered to be hypoimmune.
120. The cell of any one of embodiments 98-119, wherein the cell exhibits cytotoxic activity against GPRC5D+ cells, BCMA+ cells, or GPRC5D+/BCMA+ cells.
121. A composition comprising a plurality of the cell of any one of embodiments 10⁶-120.
122. The composition of embodiment 121, further comprising a pharmaceutically acceptable excipient.
123. A pharmaceutical composition comprising a plurality of the cell of any one of embodiments 106-120, and a pharmaceutically acceptable excipient.
124. The composition of any one of embodiments 121-123, wherein the composition comprises CD4+ T cells and CD8+ T cells.
125. The composition of embodiment 124, wherein the composition comprises a ratio of CD4+ T cells to CD8+ T cells that is between about 1:3 and about 3:1, optionally between about 1:2 and about 2:1, further optionally about 1:1.
126. The composition of embodiment 124 or embodiment 125, wherein the composition comprises a ratio of CD4+ T cells to CD8+ T cells that is between about 1:3 and about 3:1.
127. The composition of any one of embodiments 124-126, wherein the composition comprises a ratio of CD4+ T cells to CD8+ T cells that is about 1:1.
128. The composition of any one of embodiments 121-127, wherein greater than about 90%, greater than about 95% or greater than about 99% of cells in the composition are CD3+ T cells.
129. The composition of any one of embodiments 121-128, wherein at least about 40%, at least about 50%, at least about 60%, at least about 70%, at least about 80%, or at least about 90% of cells in the composition express the CAR.
130. The composition of any one of embodiments 121-129, wherein, among a plurality of the cells in the composition expressing the CAR, less than about 10%, about 9%, about 8%, about 7%, about 5%, about 4%, about 3%, about 2%, or about 1% of the cells exhibit tonic signaling.
131. The composition of any one of embodiments 121-130, wherein the composition comprises between about 1.0×10⁷ CAR-expressing T cells and 1.2×10⁹ CAR-expressing T cells, between about 1.0×10⁷ CAR-expressing T cells and 6.5×10⁸ CAR-expressing T cells, between about 1.5×10⁷ CAR-expressing T cells and 6.5×10⁸ CAR-expressing T cells, between about 1.5×10⁷ CAR-expressing T cells and 6.0×10⁸ CAR-expressing T cells, between about 2.5×10⁷ CAR-expressing T cells and 6.0×10⁸ CAR-expressing T cells, between about 5.0×10⁷ CAR-expressing T cells and 6.0×10⁸ CAR-expressing T cells, between about 1.25×10⁷ CAR-expressing T cells and 1.2×10⁹ CAR-expressing T cells, between about 1.5×10⁷ CAR-expressing T cells and 1.2×10⁹ CAR-expressing T cells, between about 5.0×10⁷ CAR-expressing T cells and 4.5×10⁸ CAR-expressing T cells, or between about 1.5×10⁸ CAR-expressing T cells and 3.0×10⁸ CAR-expressing T cells, each inclusive.
132. The composition of any one of embodiments 121-131, wherein the composition comprises at or about 1.5×10⁷, at or about 2.5×10⁷, at or about 5.0×10⁷, at or about 7.5×10⁷, at or about 1.0×10⁸, at or about 1.25×10⁸, at or about 1.5×10⁸, at or about 1.75×10⁸, at or about 2×10⁸, at or about 2.25×10⁸, at or about 2.5×10⁸, at or about 3.0×10⁸, at or about 3.5×10⁸, at or about 4×10⁸, at or about 4.5×10⁸, at or about 6.0×10⁸, at or about 8.0×10⁸, or at or about 1.2×10⁹ CAR-expressing T cells.
133. A method of treating a disease or condition comprising administering the cell of any one of embodiments 10⁶-120 or the composition of any one of embodiments 121-132 to a subject.
134. The method of embodiment 133, wherein the cell is administered to the subject at a dose of from at or about 1×10⁷ CAR-expressing T cells and 1×10⁹ CAR-expressing T cells 135. The method of embodiment 133, wherein the cell is administered to the subject at a dose of from or from about 2.5×10⁷ CAR-expressing T cells to about 4.5×10⁸ CAR-expressing T cells.
136. The method of any one of embodiments 133-135, wherein the cell is administered to the subject at a dose of or about 2.5×10⁷ CAR-expressing T cells.
137. The method of any one of embodiments 133-135, wherein the cell is administered to the subject at a dose of or about 7.5×10⁷ CAR-expressing T cells.
138. The method of any one of embodiments 133-135, wherein the cell is administered to the subject at a dose of or about 1.5×10⁸ CAR-expressing T cells.
139. The method of any one of embodiments 133-135, wherein the cell is administered to the subject at a dose of or about 3.0×10⁸ CAR-expressing T cells.
140. The method of any one of embodiments 133-135, wherein the cell is administered to the subject at a dose of or about 4.5×10⁸ CAR-expressing T cells.
141. The method of any one of embodiments 133-140, further comprising administering a lymphodepleting therapy to the subject prior to administration of the dose of the CAR-expressing T cells.
142. The method of any one of embodiments 133-141, wherein the lymphodepleting therapy is completed within about 7 days prior to initiation of the administration of the dose of the CAR-expressing T cells.
143. The method of any one of embodiments 133-142, wherein the administration of the lymphodepleting therapy is completed within about 2 to 7 days prior to initiation of the administration of the dose of engineered T cells.
144. The method of any one of embodiments 133-143, wherein the lymphodepleting therapy comprises the administration of fludarabine and/or cyclophosphamide.
145. The method of any one of embodiments 133-144, wherein the lymphodepleting therapy comprises the administration of fludarabine and cyclophosphamide.
146. The method of any one of embodiments 133-145, wherein the lymphodepleting therapy comprises administration of cyclophosphamide at or about 200-400 mg/m² inclusive daily.
147. The method of any one of embodiments 133-146, wherein the lyphodepleting therapy comprises administration of cyclophosphamide at or about 300 mg/m² daily.
148. The method of any one of embodiments 133-145, wherein the lyphodepleting therapy comprises administration of fludarabine at or about 20-40 mg/m² inclusive daily.
149. The method of any one of embodiments 133-146 and 148, wherein the lyphodepleting therapy comprises administration of fludarabine at or about 30 mg/m² daily.
150. The method of any one of embodiments 133-149, wherein the lyphodepleting therapy comprises administration of fludarabine and cyclophosphamide for 2-4 days.
151. The method of any one of embodiments 133-150, wherein the lyphodepleting therapy comprises administration of fludarabine and cyclophosphamide for 3 days.
152. The method of any one of embodiments 133-143 wherein the lymphodepleting therapy comprises the administration of bendamustine.
153. The method of any one of embodiments 133-143 and 152, wherein the lymphodepleting therapy comprises administration of bendamustine at or about 50-130 mg/m² inclusive daily.
154. The method of any one of embodiments 133-143, 152, and 153, wherein the lyphodepleting therapy comprises administration of bendamustine at or about 90 mg/m² daily.
155. The method of any one of embodiments 133-143 and 152-154, wherein the lyphodepleting therapy comprises administration of bendamustine for 1-3 days.
156. The method of any one of embodiments 133-143 and 152-155, wherein the lyphodepleting therapy comprises administration of bendamustine for 2 days.
157. The method of any one of embodiments 133-156, wherein the disease or condition is a cancer, optionally a plasma cell malignancy.
158. The method of any one of embodiments 133-157, wherein the disease or condition is a BCMA-expressing cancer and/or a GPRC5D-expressing cancer.
159. The method of any one of embodiments 133-158, wherein the disease or condition is a multiple myeloma.
160. The method of any one of embodiments 133-159, wherein the disease or condition is a relapsed/refractory multiple myeloma (RRMM).
161. The method of any one of embodiments 133-160, wherein the subject has received one or more prior therapies.
162. The method of any one of embodiments 133-161, wherein, the subject has received at least 1, but no more than 3, prior therapies.
163. The method of embodiment 161 or embodiment 162, wherein the prior therapies are an proteasome inhibitor, an immumodulatory agent, an anti-CD38 antibody, a prior therapy that included autologous hematopoietic stem cell transplantation (HSCT), or a combination of any of the foregoing.
164. Use of the cell of any one of embodiments 10⁶-120 or the composition of any one of embodiments 121-132 for manufacture of a medicament for treating a disease or condition in a subject.
165. Use of the cell of any one of embodiments 10⁶-120 or the composition of any one of embodiments 121-132 for treatment of a disease or condition in a subject.
166. The use of embodiment 164 or embodiment 165, wherein the disease or condition is a cancer, optionally a plasma cell malignancy.
167. The use of any one of embodiments 164-166, wherein the disease or condition is a BCMA-expressing cancer and/or a GPRC5D-expressing cancer.
168. The use of any one of embodiments 164-167, wherein the disease or condition is a multiple myeloma.
169. The use of any one of embodiments 164-168, wherein the disease or condition is a relapsed/refractory multiple myeloma (RRMM).
170. The use of any one of embodiments 164-169, wherein the subject has received one or more prior therapies.
171. The use of any one of embodiments 164-169, wherein, the subject has received at least 1, but no more than 3, prior therapies.
172. The use of embodiment 170 or embodiment 171, wherein the prior therapies are an proteasome inhibitor, an immumodulatory agent, an anti-CD38 antibody, a prior therapy that included autologous hematopoietic stem cell transplantation (HSCT), or a combination of any of the foregoing.
173. The cell of any one of embodiments 106-120 or the composition of any one of embodiments 121-132 for treatment of a disease or condition in a subject.
174. The cell or composition for use of embodiment 173, wherein the disease or condition is a cancer, optionally a plasma cell malignancy.
175. The cell or composition for use of embodiment 173 or embodiment 174, wherein the disease or condition is a BCMA-expressing cancer and/or a GPRC5D-expressing cancer.
176. The cell or composition for use of any one of embodiments 173-175, wherein the disease or condition is a multiple myeloma.
177. The cell or composition for use of any one of embodiments 173-176, wherein the disease or condition is a relapsed/refractory multiple myeloma (RRMM).
178. The cell or composition of any one of embodiments 173-177, wherein the subject has received one or more prior therapies.
179. The cell or composition of any one of embodiments 173-178, wherein, the subject has received at least 1, but no more than 3, prior therapies.
180. The cell or composition of embodiment 178 or embodiment 179, wherein the prior therapies are an proteasome inhibitor, an immumodulatory agent, an anti-CD38 antibody, a prior therapy that included autologous hematopoietic stem cell transplantation (HSCT), or a combination of any of the foregoing.
181. A kit comprising the CAR of any one of embodiments 1-93, the polynucleotide of any one of embodiments 94-101, the vector of any one of embodiments 102-105, the cell of any one of embodiments 10⁶-120, or the composition of any one of embodiments 121-132, and instructions for use, optionally wherein the instructions are for administering the CAR, the cell, or the composition.
182. The kit of embodiment 181, wherein the instructions specify administering the CAR, the cell, or the composition to a subject having a disease or disorder.
183. An article of manufacture comprising the CAR of any one of embodiments 1-93, the polynucleotide of any one of embodiments 94-101, the vector of any one of embodiments 102-105, the cell of any one of embodiments 106-120, the composition of any one of embodiments 121-132, or the kit of embodiment 181 or embodiment 182.

### IX. Examples

The following examples are included for illustrative purposes only and are not intended to limit the scope of the invention.

### Example 1: Assessment of Human GPRC5D and Human BCMA Expression on Tumor Cell Lines

Protein expression of human GPRC5D and human BCMA was evaluated on parental MM.1 S, OPM-2, and RPMI-8226 tumor cell lines by flow cytometry with anti-GPRC5D and anti-BCMA antibodies, respectively. As controls, the parental MM.1S and OPM-2 cell lines were also knocked out for GPRC5D (MM.1S GPRC5D KO and OPM-2 GPRC5D KO) or BCMA (MM.1S BCMA KO and OPM-2 BCMA KO). Expression of GPRC5D and BCMA was defined as the fluorescent signal above the fluorescent signal of the fluorescent minus one (FMO) control.

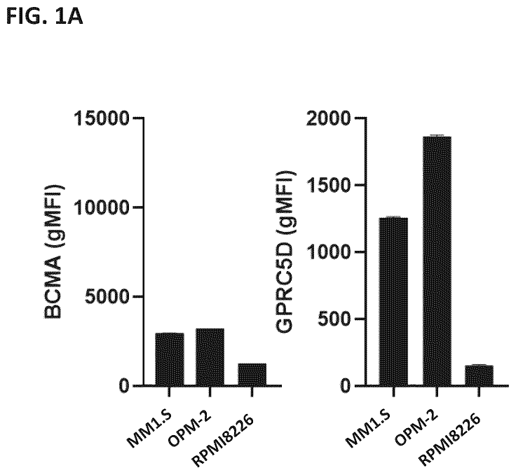

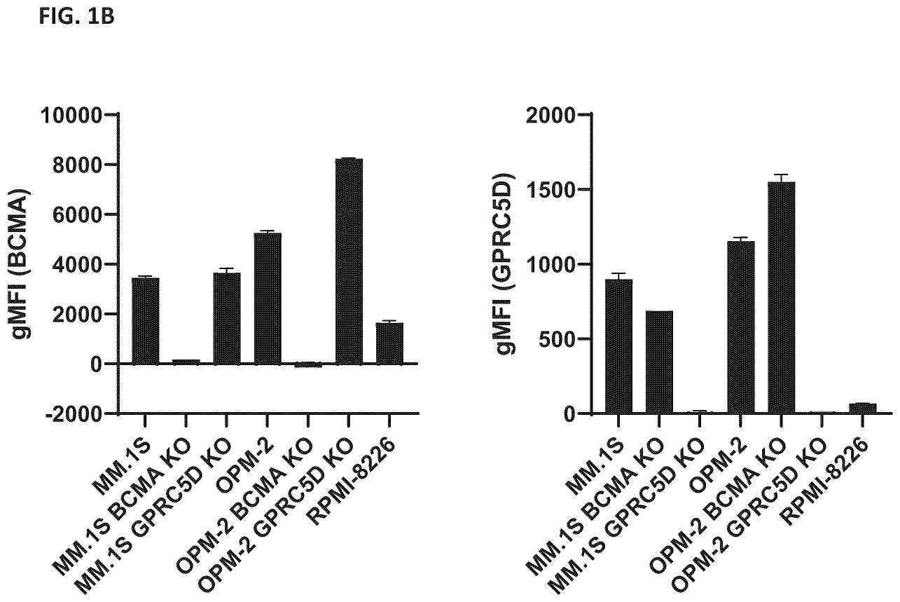

Expression of both BCMA and GPRC5D was confirmed in MM.1S, OPM-2 and RPMI-8226 parental cell lines (FIG. 1A). MM.1S BCMA KO and OPM-2 BCMA KO cell lines were observed to express GPRC5D but did not express detectable levels of BCMA. Conversely, MM.1S GPRC5D KO and OPM-2 GPRC5D KO cell lines were observed to express BCMA but did not express detectable levels of GPRC5D (FIG. 1B).

### Example 2: Generation of BCMA×GPRC5D Tandem Chimeric Antigen Receptors (CARs)

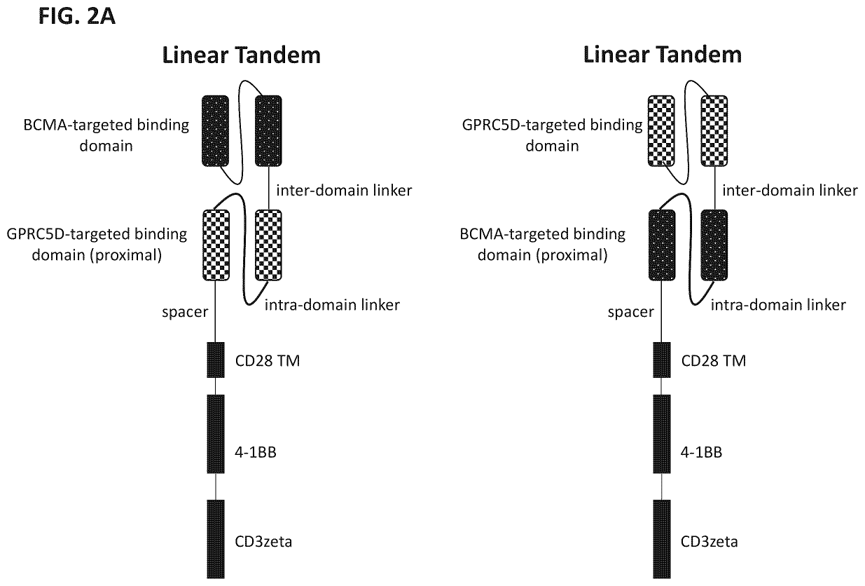

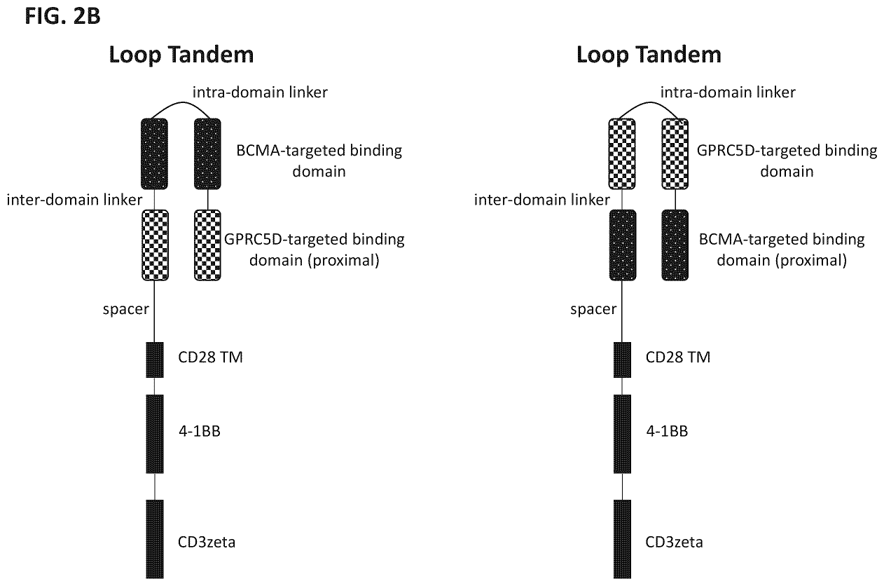

123 bispecific, tandem chimeric antigen receptors (CARs) were engineered, each incorporating an extracellular antigen-binding domain having (1) a BCMA-targeted binding domain comprising the variable heavy chain (V_{H}) and the variable light chain (V_{L}) sequences set forth in SEQ ID NOS: 15 and 16, respectively, and (2) a GPRC5D-targeted binding domain comprising the variable heavy chain (V_{H}) and the variable light chain (V_{L}) sequences set forth in SEQ ID NOS: 7 and 8, respectively. The extracellular antigen-binding domains were generated to have various structural configurations, including different combinations of: arrangement of the two binding domains in either linear tandem or loop tandem format; order of the V_{H} and the V_{L} of each binding domain, from N- to C-terminus; linkers between the V_{H} and the V_{L} of the same binding domain (“intra-domain linker,” e.g., SEQ ID NO: 17 or 18); linkers between the different binding domains (“inter-domain linker,” e.g. SEQ ID NO: 19, 21, 22, or 24); and arrangement of either the BCMA-targeted binding domain or the GPRC5D-targeted binding domain proximal to the cellular membrane. In the linear tandem format, the variable heavy chain (V_{H}) and the variable light chain (V_{L}) of the BCMA-targeted binding domain were arranged in linear order, followed by the V_{H} and V_{L} of the GPRC5D-targeted binding domain arranged in linear order, or vice versa. In the loop tandem format, the V_{H} and V_{L} of one of the binding domains were separated by the V_{H} and V_{L} of the other of the binding domains. Exemplary loop and linear tandem configurations of the extracellular antigen-binding domains are shown in (FIG. 2).

Each generated tandem CAR construct contained the extracellular antigen-binding domain having both the BCMA- and GPRC5D-targeted binding domains as described above; an immunoglobulin derived spacer domain (e.g., SEQ ID NO: 27); a human CD28-derived transmembrane domain (e.g., SEQ ID NO: 28); a human 4-1BB-derived intracellular signaling domain (e.g., SEQ ID NO: 29); and a human CD3zeta-derived intracellular signaling domain (e.g., SEQ ID NO: 30).

For transduction of primary human T cells, as described in studies below, CD4 and CD8 T cells were isolated from the peripheral blood of healthy human adult donors, and combined at a ratio of 1:1. The combined cell population was stimulated with anti-CD3/anti-CD28 antibody-conjugated beads in the presence of recombinant IL-2, IL-7, and IL-15 prior to transduction with lentivirus comprising a CAR described herein. CD4 and CD8 T cells from the same donors were mock-processed in parallel without lentivirus, to serve as controls. Following transduction, the mock-processed and CAR T cells were optionally expanded, and then harvested and cryopreserved before downstream use.

### Example 3: Assessment of Antigen-Independent (Tonic) and Antigen-Dependent Signaling by BCMA×GPRC5D Tandem Chimeric Antigen Receptors (CARs)

A stable Jurkat T cell reporter line was generated to contain a red fluorescent reporter protein knocked into the Nur77 locus. The Jurkat Nur77 reporter cells (Nurkat) were stably transduced with the 123 different BCMA×GPRC5D tandem CAR constructs described in Example 2.

As controls, singly targeting CARs against BCMA (BCMA CAR; e.g., SEQ ID NO:101) or GPRC5D (GPRC5D CAR; e.g., SEQ ID NO:102) were generated to contain extracellular antigen binding domains having the same V_{H} and the V_{L} amino acid sequences of the BCMA- or GPRC5D-targeted binding domain as the bispecific tandem CARs generated in Example 2, as well as the same spacer domain, CD28 transmembrane domain, and 4-1BB and CD3zeta intracellular signaling domains. As a further control, a bispecific, bicistronic CAR targeting BCMA and GPRC5D (“bicistronic”) was generated to contain: (1) an anti-GPRC5D scFv (e.g. SEQ ID NO: 46); an immunoglobulin hinge-CH2-CH3 (e.g. SEQ ID NO: 27); a human CD28-derived transmembrane domain (e.g. SEQ ID NO: 28); a human 4-1BB-derived intracellular signaling domain (e.g. SEQ ID NO: 29); and a human CD3zeta-derived intracellular signaling domain (e.g. SEQ ID NO: 30); and (2) an anti-BCMA scFv (e.g. SEQ ID NO: 47); an immunoglobulin hinge-CH2-CH3 (e.g. SEQ ID NO: 27); a human CD28-derived transmembrane domain (e.g. SEQ ID NO: 28); a human 4-1BB-derived intracellular signaling domain (e.g. SEQ ID NO: 29); and a human CD3zeta-derived intracellular signaling domain (e.g. SEQ ID NO: 30). The nucleotide sequences encoding (1) and (2) of the bicistronic CAR were separated by a nucleotide sequence encoding a downstream ribosome skip element (such as a self-cleaving T2A peptide; e.g., SEQ ID NO: 103 or 104, encoding SEQ ID NO: 69).

1×10⁵ Nurkat cells were plated in a 96-well round bottom plate with 100 μl of lentivirus encoding CAR and R10 cell culture medium and centrifuged at 1000 g for 30 minutes at 30° C. After centrifugation, supernatant was removed, and cells were re-suspended and cultured in cell culture medium. Cells were subsequently removed from cell culture, plated in a 96-well round bottom plate, washed in cell staining buffer, centrifuged at 1500 g for 15 seconds, and supernatant was aspirated. Cells were then incubated with an anti-IgG4 antibody that binds the spacer of the CAR constructs and LIVE/DEAD™ Near-IR dead cell stain. Cells were washed then resuspended in cell staining buffer to determine expression of the singly targeting, tandem, and bicistronic CARs on Nurkat reporter cell lines by flow cytometry. Results of this assay demonstrated that the incorporation of the tandem CAR constructs into the Nurkat reporter cell line was successful for all 123 tandem constructs generated, with frequency of CAR expression ranging from 17-100% of cells.

Antigen-independent (tonic) or dependent signaling of Nurkat reporter cells transduced with the CARs was assessed by co-culturing the CAR-transduced Nurkat cells alone or with multiple myeloma cell lines that were BCMA+/GPRC5D+(parental lines MM.1S and OPM-2), BCMA−/GPRC5D+(MM.1S BCMA KO, OPM-2 BCMA KO) or BCMA+/GPRC5D− (MM.1S GPRC5D KO, OPM-2 GPRC5D KO). To assist in distinguishing Nurkat reporter cells from target cells, reporter cells were labeled with CellTrace™ Violet (CTV). Labeled Nurkat reporter cells (2×10⁴; effectors) were cultured in duplicate at a 1:1 ratio with multiple myeloma cells (targets) in a 96-well round bottom plate for 20 hours at 37° C.

After incubation of reporter (effector) cells with multiple myeloma (target) cells, cells were washed two times with cell staining buffer and then stained with LIVE/DEAD™ Fixable Near-IR dead cell stain. After incubation, cells were washed twice in cell staining buffer and resuspended in 150 μL cell staining buffer for analysis of antigen-independent signaling by flow cytometry.

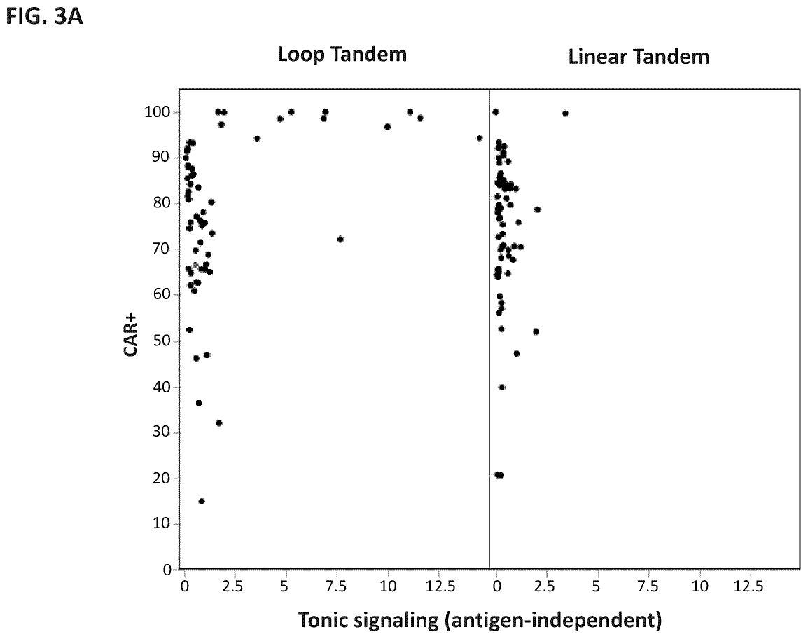

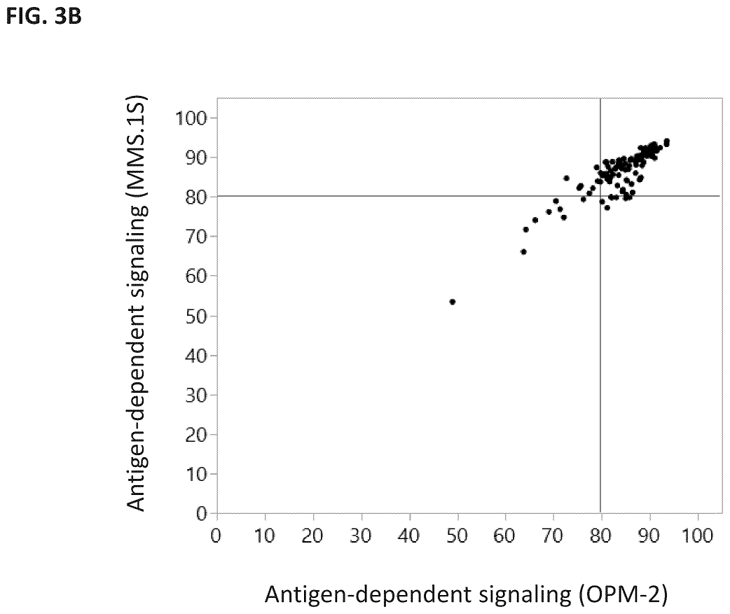

Among the Nurkat reporter cell lines transduced with the 123 different tandem CAR constructs, a range of 0.062-14.9% of CAR+ cells were observed to exhibit antigen-independent signaling, with 86 of 123 constructs exhibiting less than 5% tonic signaling (FIG. 3A).

Antigen-dependent activation of the 123 tandem CAR constructs was assessed by co-culturing transduced Nurkat reporter cells with BCMA+/GPRC5D+ parental cell lines MM.1S and OPM-2. Cells transduced with 12 of the 123 tandem constructs did not grow in culture and were excluded from further evaluation. 90 of the 111 remaining constructs showed≥80% antigen-dependent activation after co-culture with MM.1S and OPM-2 cell lines (FIG. 3B; see lines indicating 80%).

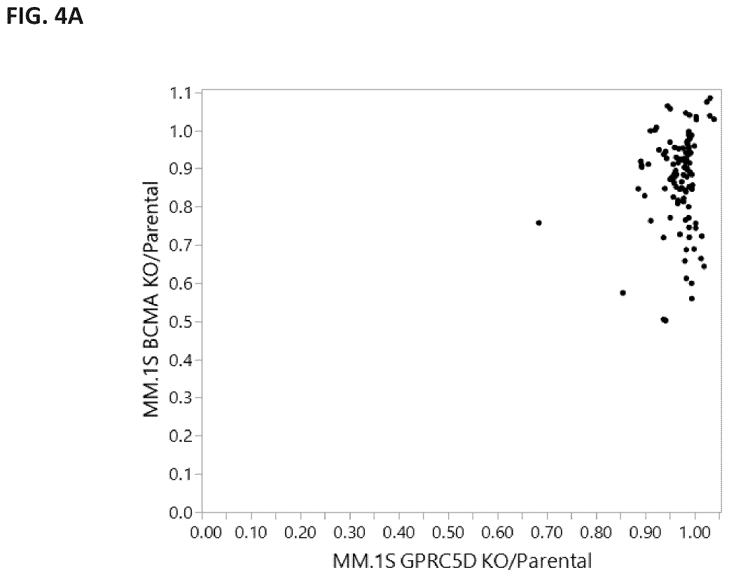

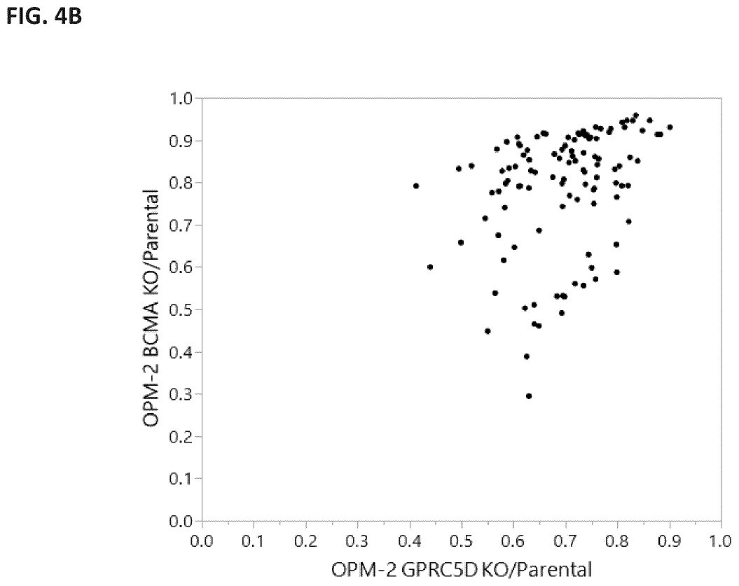

BCMA-dependent CAR activation was assessed by co-culturing transduced Nurkat reporter cells with MM.1S GPRC5D KO or OPM-2 GPRC5D KO cells lines, which express BCMA but not GPRC5D. GPRC5D-dependent CAR activation was assessed by co-culturing transduced Nurkat reporter cells with MM.1S BCMA KO or OPM-2 BCMA KO cell lines, which express GPRC5D but not BCMA. Activation through a single antigen was normalized to the parental cell lines (MM.1S or OPM-2) (FIGS. 4A and 4B). Top constructs were identified as those that exhibited≤5% antigen-independent signaling and ≥0.8 antigen-dependent activation after co-culture with MM.1S GPRC5D KO, MM.1S BCMA KO and OPM-2 BCMA KO. 27 constructs were selected for further analysis by ranking the normalized antigen-dependent activation after co-culture with OPM-2 GPRC5D KO cells with values ranging from 0.68-0.88.

### Example 4: In Vitro Activity of BCMA×GPRC5D Tandem CAR T Cells

Primary T cells from two healthy donors were transduced with one of the 27 tandem CARs as described in Example 3, or with the BCMA, GPRC5D, or bicistronic CAR as a control. In vitro activity of the 27 tandem constructs was carried out as described below to determine the top 14 constructs for further evaluation.

Cell viability and expression of the CARs was evaluated by flow cytometry. To determine viability, cells were removed from cryopreservation vials and resuspended in pre-warmed 2D25G cell culture medium and centrifuged at 300 g for 5 minutes. Cells were again resuspended in 1 mL of pre-warmed 2D25G and 20 μL were removed and mixed with 20 μL of ViaStain™ AOPI Staining Solution and counted on a Cellometer® Auto 2000 cell viability counter according to manufacturer's instructions. To determine the percentage of CAR+ cells in the T cell preparations, 5×10⁵ cells were removed and plated in a 96-well round bottom plate. Plates were centrifuged at 2000 rpm for 1 minute and supernatant was aspirated. Cell pellets were resuspended in 50 μL PBS containing LIVE/DEAD™ Fixable Near-IR dead cell stain. After incubation, cells were washed one time and then incubated with anti-CD4 and anti-CD8 antibodies to identify T cells, and anti-idiotype antibodies to identify the anti-BCMA and anti-GPRC5D binding domains. After incubation, cells were washed and further incubated with an anti-caspase3 antibody to identify dying cells.

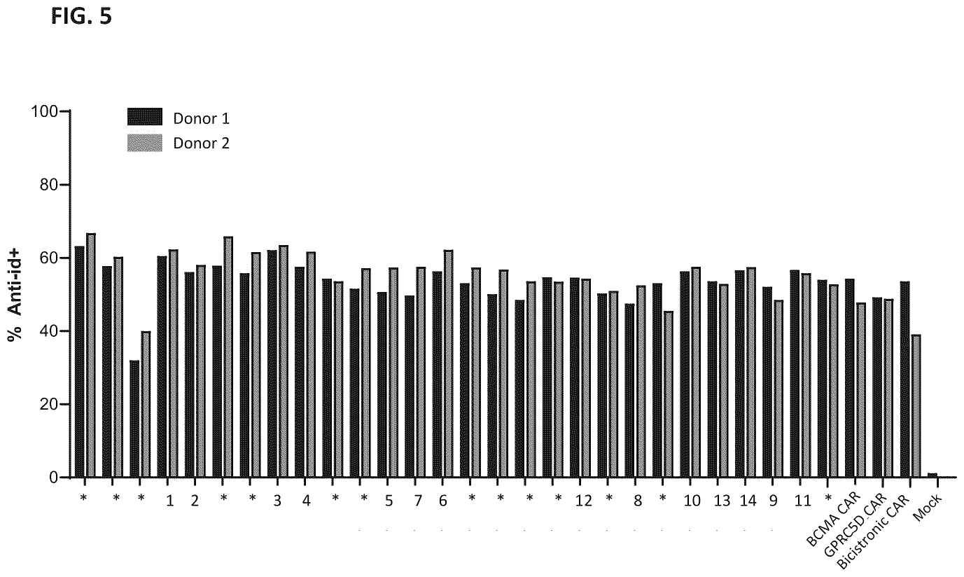

Surface expression of the CAR was detectable on T cells from two healthy donors for all tandem constructs tested (FIG. 5). The mean fluorescent intensity (MFI) of CAR expression was also similar across constructs in the two healthy donors.

Primary T cells transduced with one of the 27 different tandem CAR constructs were co-cultured with cell lines BCMA+/GPRC5D+(MM.1S, OPM-2, RPMI-8226), BCMA−/GPRC5D+(MM.1S BCMA KO, OPM-2 BCMA KO) and BCMA+/GPRC5D− (MM.1S GPRC5D KO, OPM-2 GPRC5D KO) to assess activation and proliferation. Before culturing, CAR T or mock-processed T cells were stained with CellTrace™ Violet (CTV) according to manufacturer's instructions. T cells and tumor cells were plated in duplicate in a 96-well flat bottom plate at an effector to target (E:T) ratio of 1:1. Cells were co-incubated in 50% 2D25G and 50% R10 cell culture medium for 72 hours at 37° C. After co-culturing, cells were stained with an anti-CD25 antibody to identify activated cells. To determine proliferation, cells were incubated with anti-CD4, anti-CD8, anti-CD27, anti-CCR7, anti-PD1, anti-CD25, and anti-idiotype antibodies against the anti-BCMA and anti-GPRC5D binding domains.

Bulk cytokines from the activation and proliferation assays were measured from supernatant after 24 hours of co-culture using MSD custom V-plex plates pre-coated with interferon gamma (IFNγ), interleukin-2 (IL-2), and tumor necrosis factor alpha (TNFα) antibodies, according to manufacturer's instructions. Values were normalized to the bicistronic CAR for each condition.

The ability of tandem BCMA×GPRC5D CAR T cells to lyse target cells was evaluated by co-culturing CAR T cells with BCMA+/GPRC5D+MM.1S, OPM-2, and RPMI-8226 tumor cells expressing NucLight Red (NLR), normalized to the performance of the bicistronic construct. CAR T or mock-processed T cells from healthy donors (effectors) and tumor cells (targets; 2×10⁴) were plated in duplicate in 96-well flat bottom plates coated with poly-d-lysine with 50% R10 and 50% 2D25G cell culture medium at E:T ratios of 1:2, 1:4, 1:8 for MM.1S and OPM-2 and 1:1, 1:2 and 1:4 for RPMI-8226. Total integrated intensity was determined by expression of NLR fluorescence and was monitored for 72 hours of culture. For determining target cell lysis, cellularity was plotted and the area under the curve (AUC) was calculated. Target-cell lysis was calculated using the following formula: % lysis=(AUC for CAR T cells÷AUC for mock-processed T cells)×100.

Values from each assay were ranked, and the top 14 constructs were selected for further analysis based on overall performance across assays. Of the 14 constructs selected, 4 were arranged in linear tandem format, each with the BCMA-targeted binding domain membrane proximal. The remaining 10 were arranged in loop tandem format. Of the loop tandem formatted constructs, 3 contained the GPRC5D-targeted binding domain as the membrane proximal binding domain, and 7 contained the BCMA-targeted binding domain as the membrane proximal binding domain. Diversity in inter-domain and intra-domain linkers was observed among the top 14 constructs. The components of the extracellular antigen binding domains of each of the top 14 tandem CAR constructs are provided in Table E1 below.

TABLE E1
Extracellular antigen-binding domain configuration (from N-terminus to C-terminus, as set forth
from left to right below)

| | Extra- | | | | | | | |
|---|---|---|---|---|---|---|---|---|
| | cellular | | | | | | | |
| | binding | | | | | | | |
| CAR | domain | | | | | | | |
| (SEQ | (SEQ | Component | | | | | | |
| ID NO) | ID NO) | (SEQ ID NO) | | | | | | |
| 1 (31) | 77 | GPRC5 | Intra-domain | GPRC5D | Inter-domain | BCMA | Intra-domain | BCMA |
| linear | | D V_{L} | Linker | V_{H} | Linker | V_{H} | Linker | V_{L} |
| | | (8) | (17) | (7) | (19) | (15) | (17) | (16) |
| 2 (32) | 78 | GPRC5 | Intra-domain | GPRC5D | Inter-domain | BCMA | Intra-domain | BCMA |
| linear | | D V_{H} | Linker | V_{L} | Linker | V_{H} | Linker | V_{L} |
| | | (7) | (17) | (8) | (21) | (15) | (17) | (16) |
| 3 (33) | 79 | GPRC5 | Intra-domain | GPRC5D | Inter-domain | BCMA | Intra-domain | BCMA |
| linear | | D V_{H} | Linker | V_{L} | Linker | V_{L} | Linker | V_{H} |
| | | (7) | (17) | (8) | (24) | (16) | (17) | (15) |
| 4 (34) | 80 | GPRC5 | Intra-domain | GPRC5D | Inter-domain | BCMA | Intra-domain | BCMA |
| linear | | D V_{L} | Linker | V_{H} | Linker | V_{H} | Linker | V_{L} |
| | | (8) | (17) | (7) | (24) | (15) | (17) | (16) |
| 5 (37) | 83 | GPRC5 | Inter-domain | BCMA V_{L} | Intra-domain | BCMA | Inter-domain | GPRC5 |
| loop | | D V_{H} | Linker | (16) | Linker | V_{H} | Linker | D V_{L} |
| | | (7) | (21) | | (17) | (15) | (21) | (7) |
| 6 (38) | 84 | GPRC5 | Inter-domain | BCMA V_{H} | Intra-domain | BCMA | Inter-domain | GPRC5 |
| loop | | D V_{L} | Linker | (15) | Linker | V_{L} | Linker | D V_{H} |
| | | (8) | (21) | | (17) | (16) | (21) | (7) |
| 7 (41) | 87 | GPRC5 | Inter-domain | BCMA V_{L} | Intra-domain | BCMA | Inter-domain | GPRC5 |
| loop | | D V_{H} | Linker | (16) | Linker | V_{H} | Linker | D V_{L} |
| | | (7) | (22) | | (17) | (15) | (22) | (8) |
| 8 (35) | 81 | BCMA | Inter-domain | GPRC5D | Intra-domain | GPRC5D | Inter-domain | BCMA |
| loop | | V_{H} | Linker | V_{L} | Linker | V_{H} | Linker | V_{L} |
| | | (15) | (19) | (8) | (17) | (7) | (19) | (16) |
| 9 (36) | 82 | BCMA | Inter-domain | GPRC5D | Intra-domain | GPRC5D | Inter-domain | BCMA |
| loop | | V_{L} | Linker | V_{H} | Linker | V_{L} | Linker | V_{H} |
| | | (16) | (19) | (7) | (18) | (8) | (19) | (15) |
| 10 (39) | 85 | BCMA | Inter-domain | GPRC5D | Intra-domain | GPRC5D | Inter-domain | BCMA |
| loop | | V_{L} | Linker | V_{H} | Linker | V_{L} | Linker | V_{H} |
| | | (16) | (21) | (7) | (17) | (8) | (21) | (15) |
| 11 (40) | 86 | BCMA | Inter-domain | GPRC5D | Intra-domain | GPRC5D | Inter-domain | BCMA |
| loop | | V_{L} | Linker | V_{L} | Linker | V_{H} | Linker | V_{H} |
| | | (16) | (21) | (8) | (17) | (7) | (21) | (15) |
| 12 (42) | 88 | BCMA | Inter-domain | GPRC5D | Intra-domain | GPRC5D | Inter-domain | BCMA |
| loop | | V_{H} | Linker | V_{H} | Linker | V_{L} | Linker | V_{L} |
| | | (15) | (22) | (7) | (17) | (8) | (22) | (16) |
| 13 (43) | 89 | BCMA | Inter-domain | GPRC5D | Intra-domain | GPRC5D | Inter-domain | BCMA |
| loop | | V_{L} | Linker | V_{H} | Linker | V_{L} | Linker | V_{H} |
| | | (16) | (22) | (7) | (17) | (8) | (22) | (15) |
| 14 (44) | 90 | BCMA | Inter-domain | GPRC5D | Intra-domain | GPRC5D | Inter-domain | BCMA |
| loop | | V_{L} | Linker | V_{H} | Linker | V_{L} | Linker | V_{H} |
| | | (16) | (22) | (7) | (18) | (8) | (22) | (15) |

The top 7 constructs selected for further analysis are shown in bold.

T cells transduced with the top 14 tandem CAR constructs were co-cultured with cell lines BCMA+/GPRC5D+(MM.1S, OPM-2, RPMI-8226), BCMA−/GPRC5D+(MM.1S BCMA KG, OPM-2 BCMA KO) and BCMA+/GPRC5D− (MM.1S GPRC5D KO, OPM-2 GPRC5D KO). Activation was assessed by expression of the activation marker CD25 after 72 hours of co-culture and cell lysis was evaluated as described above. In vitro activity of the 14 tandem constructs was carried out as described below to determine the top 7 constructs for in vivo evaluation.

Performance of the constructs in a serial re-stimulation assay was assessed by measuring proliferation, ability to lyse target cells, and cytokine production. Briefly, the ability of transduced T cells expressing the 14 different tandem CAR constructs to repeatedly kill tumor cells was evaluated by co-culturing the T cells with tumor cells for 21 days. 9×10⁴ CAR T cells were co-incubated with the MM.1S parental, MM.1S BCMA KO, or MM.1S GPRC5D KO cell line at an effector:target (E:T) ratio of 1:1. After 7 days, an aliquot of cells was removed from culture for analysis of CAR expression, cell number, cell viability, and CD25 expression.

Based on the results, an aliquot of 3×10⁴ CAR T cells was plated at an E:T ratio of 1:2 with the parental MM.1 S cell line expressing NLR for a cytotoxicity assay in duplicate. After 24 hours, 50 μL of supernatant was harvested for bulk cytokine analysis as described above. An aliquot of 9×10⁴ CAR T cells was plated at a 1:1 E:T ratio with tumor cells in a new 24-well plate to assess the ability of CAR T cells to proliferate after repeated stimulation. This procedure was repeated on day 14. On day 21, an aliquot of cells was removed from culture for analysis of CAR expression, cell number, cell viability and CD25 expression. An aliquot of 3×10⁴ CAR T cells was plated at an E:T ratio of 1:4 and 1:8 in duplicate with the parental MM.1S cell line expressing NLR for a cytotoxicity assay.

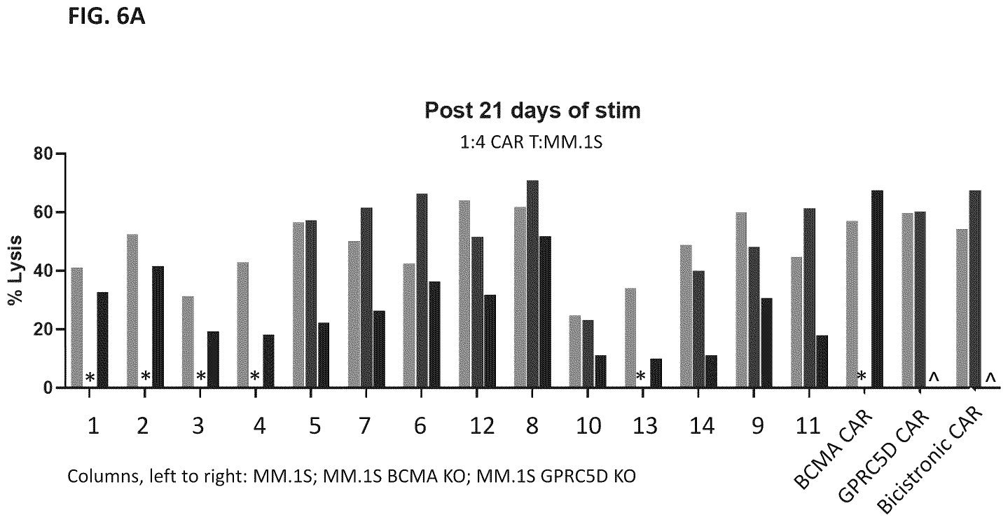

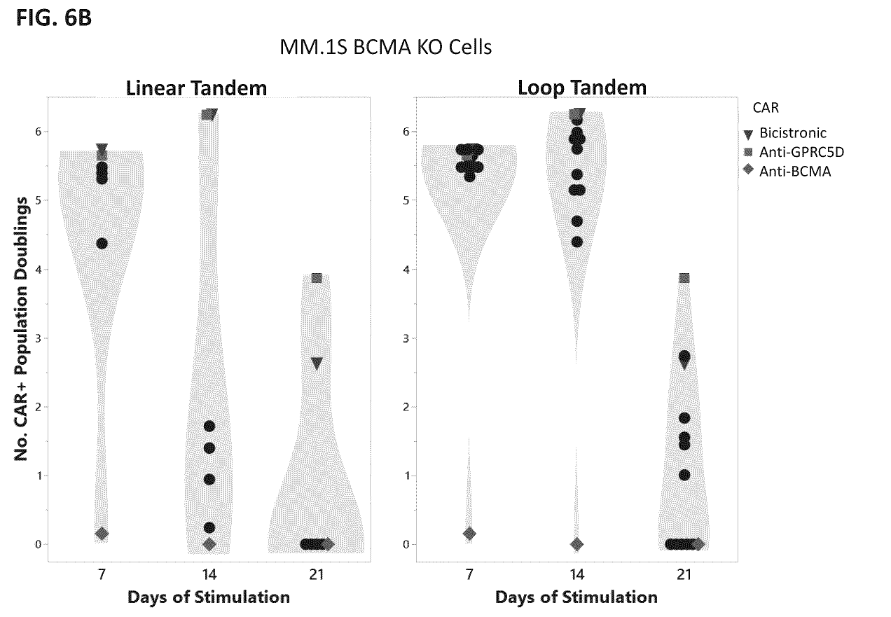

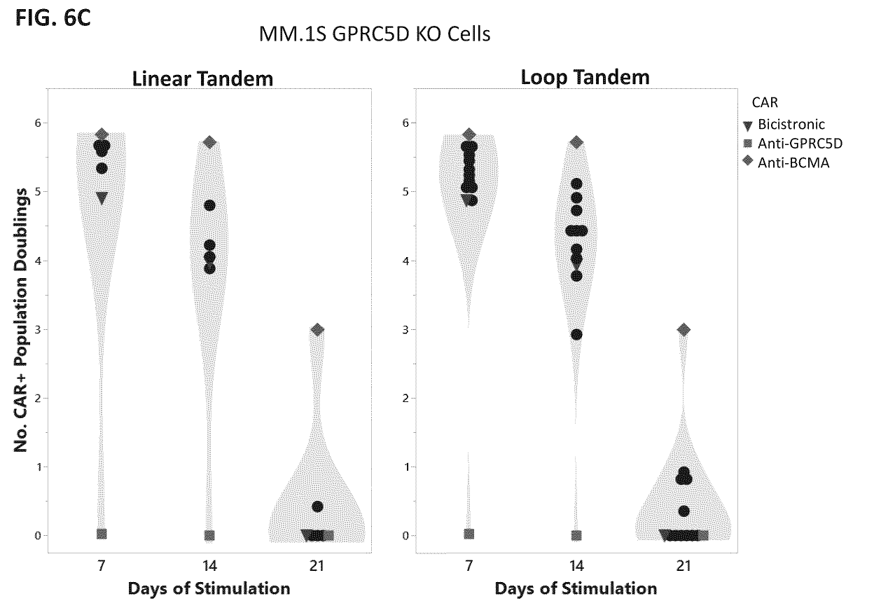

The majority of linear and loop tandem constructs proliferated and retained cytotoxic activity (FIG. 6A) against the MM.1S parental cell line throughout the duration of re-stimulation. Most of the tandem constructs retained cytolytic activity better than the bicistronic CAR in the absence of GPRC5D antigen; cells expressing the BCMA or GPRC5D single CAR also retained cytotoxicity (FIG. 6A). Cells expressing tandem constructs were observed to proliferate similarly or greater than cells expressing the bicistronic CAR in the absence of GPRC5D antigen, and cells expressing the BCMA or GPRC5D CAR proliferated well under all conditions (FIGS. 6B and 6C).

The values from each assay were ranked, and the top 7 constructs were selected based on overall performance across assays (construct numbers in bold in Table E1).

### Example 5: In Vivo Activity of BCMA×GPRC5D Tandem CAR T Cells in the MM.1S Mouse Model of Multiple Myeloma

T cells transduced with one of the top 7 tandem CAR constructs described in Example 4 were further assessed in vivo using the MM1.S disseminated multiple myeloma xenograft mouse model. The efficacy of the tandem CAR T cells was assessed by monitoring tumor burden by bioluminescent imaging (BLI), and compared to that of cells expressing the BCMA, GPRC5D, or bicistronic CAR.

MM.1S cells were genetically engineered to express green fluorescent protein (GFP), to facilitate cell sorting, and red-shifted Italian firefly luciferase (MM.1S-rFLuc), to facilitate in vivo BLI. On day −14, MM.1 S rFLuc cells were harvested during the exponential growth phase, washed, and resuspended in PBS for administration. NOD.Cg-Prkdc^{scid}IL-2rg^{tm1Wj1}/SzJ mice (NSG) were injected via lateral tail vein with 2×10⁶ MM1.S-rFLuc cells. The MM1.S-rFLuc cells were allowed to engraft and expand for 14 days (Table 1). On day −1, tumor burden was assessed in mice by BLI, and mice were assigned to treatment groups such that each group had an equal distribution of tumor burden. On day 1, cryopreserved transduced or mock-processed T cells were thawed, washed, and centrifuged at 250 g for 10 minutes. Cell pellets were resuspended in PBS, and Mice were intravenously injected with a low (0.5×10⁶) or high (2×10⁶) dose of CAR T cells expressing one of the top 7 tandem CAR constructs, the BCMA CAR, the GPRC5D CAR, the bicistronic CAR, or mock-processed T cells. An untreated group of mice was also included as a control. Disseminated tumor burden was measured over time by BLI 1-2 times per week.

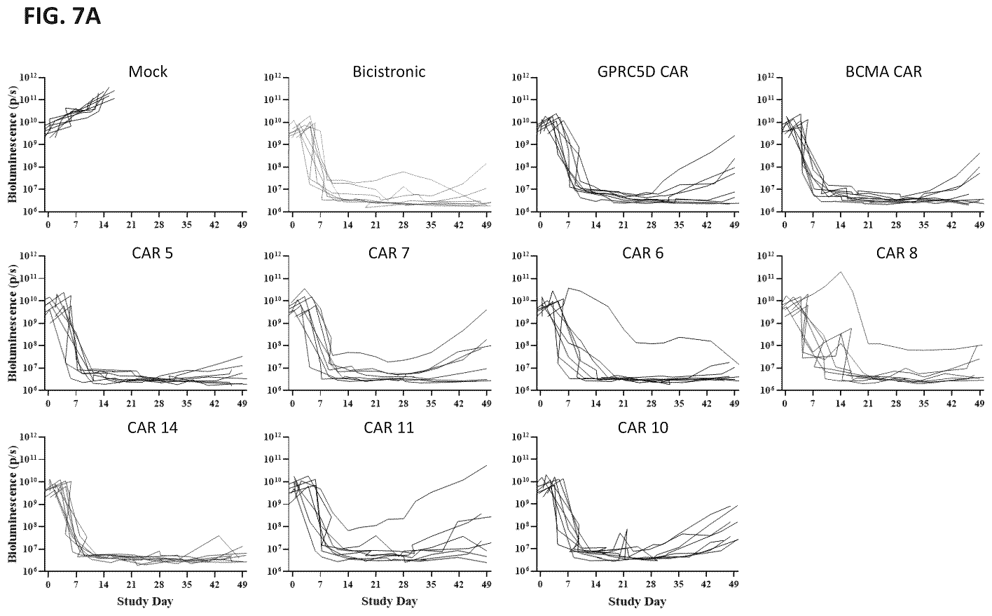

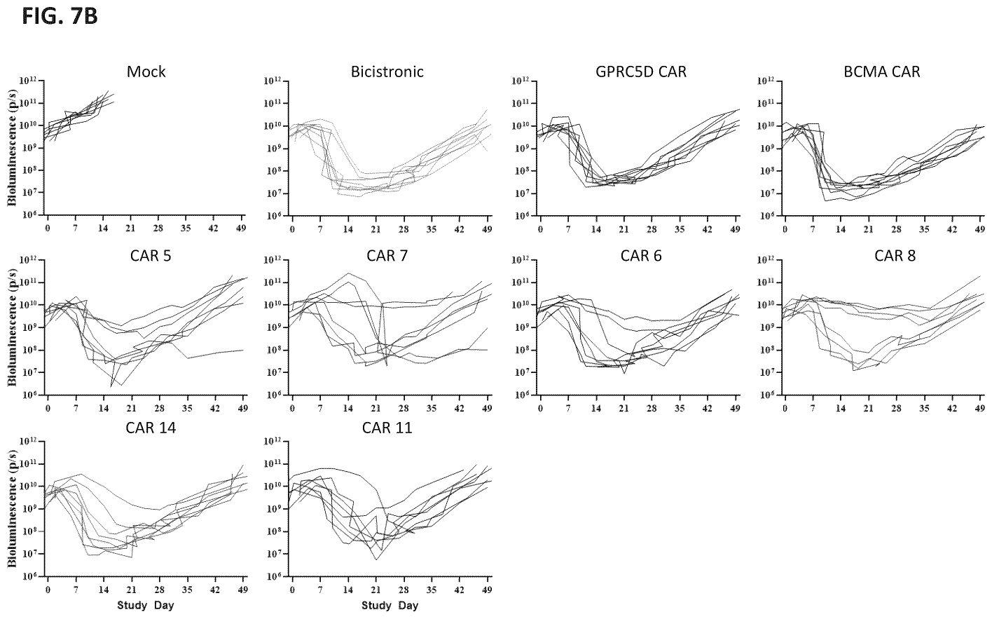

As shown in FIG. 7A, mice treated with the high dose of 2.0×10⁶ CART cells expressing any of the tandem CAR constructs exhibited robust tumor growth suppression. At the low dose of 0.5×10⁶ cells, all tandem CAR T cells demonstrated tumor growth regression and control relative to mock-processed T cells, although individual tumor growth curves were more variable and tumor inhibition more modest than at the higher dose (FIG. 7B). Mice treated with mock-processed T cells did not exhibit control of tumor growth and all mice succumbed to tumor progression before day 21. Anti-tumor efficacy of BCMA CAR or GPRC5D CAR T cells was noted at both doses, with regrowth occurring earlier at low dose than high dose in this 50-day study.

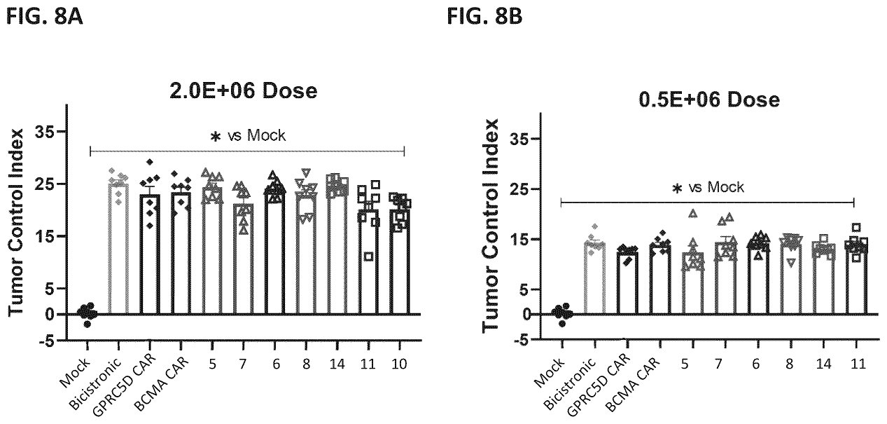

Tumor Control Index (TCI) analysis was performed on bioluminescent tumor growth curves following treatment at both dose levels. Higher TCI indicated lower tumor burden. At the high dose level (2.0×10⁶)(FIG. 8A), all bispecific tandem CAR constructs significantly improved tumor control relative to mock-processed T cells (minimum p≤0.05), similar to the bicistronic CAR, the GPRC5D CAR, and the BCMA CAR. At the low dose level (0.5×10⁶), although achieving lower TCI scores, reflecting reduced potency due to the lower dose given, all bispecific tandem CAR constructs, the bicistronic CAR, the GPRC5D CAR, and the BCMA CAR significantly improved tumor control relative to mock-processed T cells (minimum p<0.05) (FIG. 8B).

### Example 6: In Vivo Activity of BCMA×GPRC5D Tandem CAR T Cells in the RPMI8226 Mouse Model of Multiple Myeloma

T cells transduced with one of the top 7 tandem CAR constructs described in Example 4 were further assessed in vivo using another xenograft mouse model (RPMI8226) expressing relatively low levels of BCMA and GPRC5D. The preparation of the tumor cell line, its implantation into the mice, the preparation of the T cells (CAR and mock-processed) and the injection of T cells into the mice, and monitoring of tumor growth were conducted as described in Example 5.

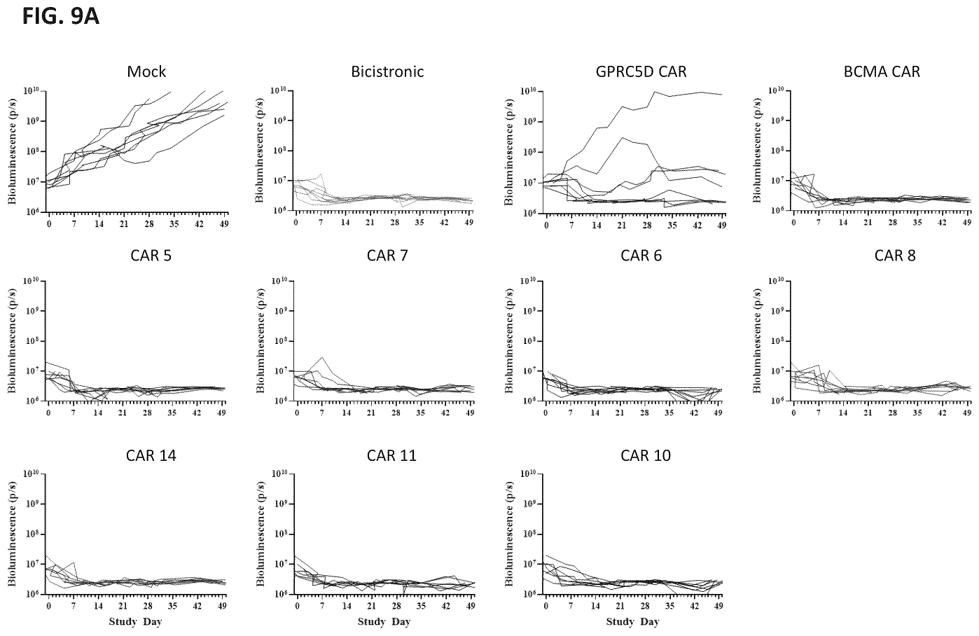

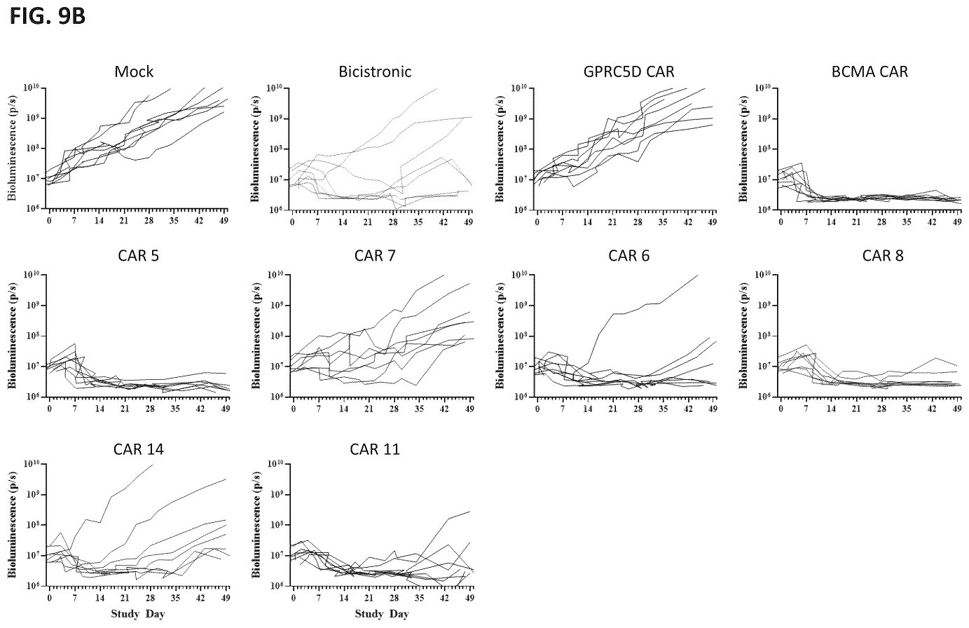

All of the tandem CAR T cells tested demonstrated potent anti-tumor efficacy in this model. As shown in FIG. 9A, mice treated with the high dose of 2.0×10⁶ CAR+ T cells expressing any of the tandem CAR constructs exhibited robust tumor clearance over the course of the study, reaching stable, minimal bioluminescence by day 14 post CAR T cell administration and remaining low through the duration of the study in all mice. At the low dose (0.5×10⁶), CAR T cells expressing tandem CAR constructs 5, 6, 8, and 11 exhibited strong tumor growth control, while tumor growth inhibition was more variable with CAR T cells expressing tandem CAR constructs 7 and 14 (FIG. 9B). Mice treated with mock-processed T cells did not exhibit control of tumor growth. The anti-tumor efficacy of T cells expressing the BCMA CAR was robust at both doses. By contrast, very low expression of GPRC5D by RPMI8226 cells resulted in failure to contain tumor growth by T cells expressing the GPRC5D CAR at the low dose level, with strong but incomplete tumor growth control by at high dose level.

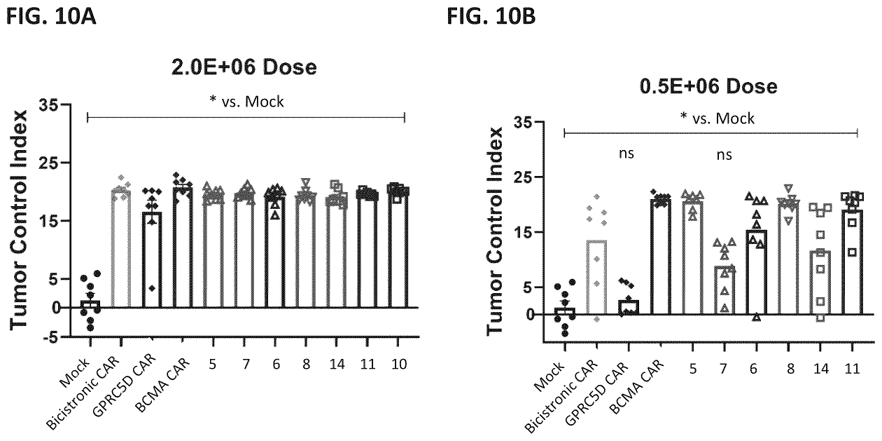

Tumor Control Index (TCI) analysis was performed as described in Example 5. At the high dose level, all tandem CAR constructs significantly improved tumor control relative to mock-processed T cells (minimum p<0.05), similar to the bicistronic CAR, the GPRC5D CAR, and the BCMA CAR (FIG. 10A). While the GPRC5D CAR, and the bispecific tandem CAR construct 7 demonstrated less tumor control at the low dose level, tandem CAR constructs 5, 6, 8, 14, and 11 significantly improved tumor control relative to mock-processed T cells (minimum p<0.05), as did the bicistronic CAR and the BCMA CAR (FIG. 10B).

### Example 7: In Vivo Activity of BCMA×GPRC5D Tandem CAR T Cells in an Antigen Heterogeneity Mouse Model of Multiple Myeloma

Based upon the assessments described in Examples 2-6, the top 2 tandem CAR constructs for further analysis were selected as constructs 5 and 11. Tandem CAR constructs 5 and 11 were further tested in vivo using a model of multiple myeloma antigen heterogeneity.

The MM.1S cell line was genetically engineered to express GFP and rFluc (MM1.S-rFLuc) as described in Example 5. On day −14, NOD.Cg-Prkdc^{scid}IL2rg^{tm1Wj1}/SzJ mice (NSG) were injected via tail vein with 4×10⁶ total (1) MM1.S-rFLuc parental cells (BCMA+, GPRC5D+) only; (2) MM1.S-rFLuc BCMA KO cells only; (3) MM1.S-rFLuc GPRC5D KO cells only, or (4) a cell mixture containing 33.3% MM1.S-rFLuc parental cells, 33.3% MM1.S-rFLuc BCMA KO cells, and 33.3% MM1.S-rFLuc GPRC5D KO cells (“mixed model”).

The anti-tumor efficacy of the top 2 tandem CAR constructs was tested in the mixed model of antigen heterogeneity. T cells expressing tandem CAR construct 5, tandem CAR construct 11, the BCMA CAR, or the GPRC5D CAR, or mock-processed T cells, were prepared as described in Example 5. Mice were distributed into treatment groups with equal distribution of tumor burden on day −1, and intravenously injected with 4×10⁶ CAR T cells on day 1. Untreated mice were also included as a control. Tumor burden was assessed two times per week over 28 days.

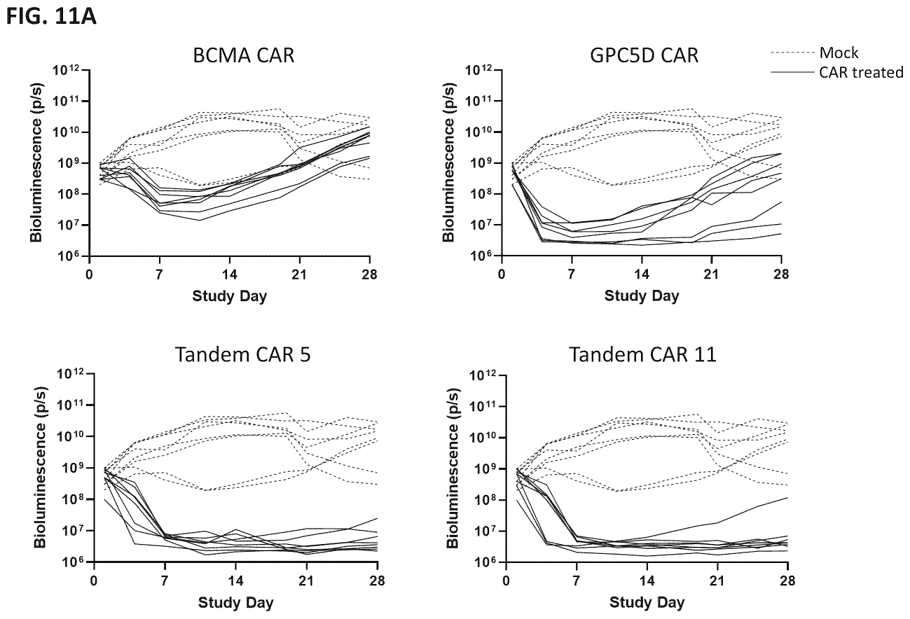

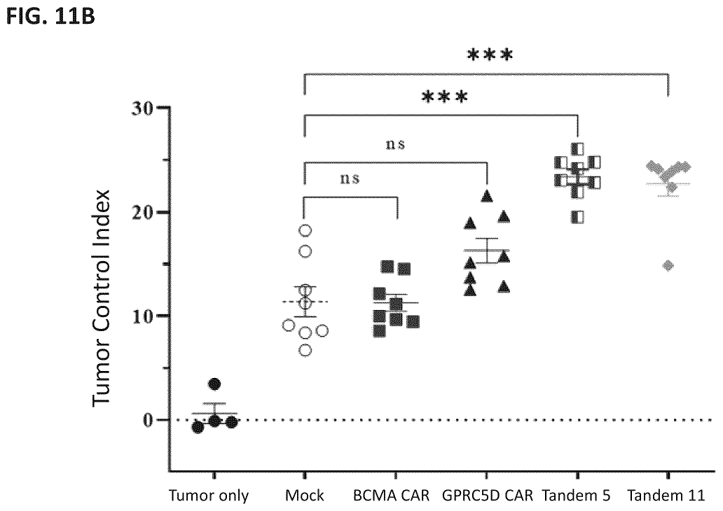

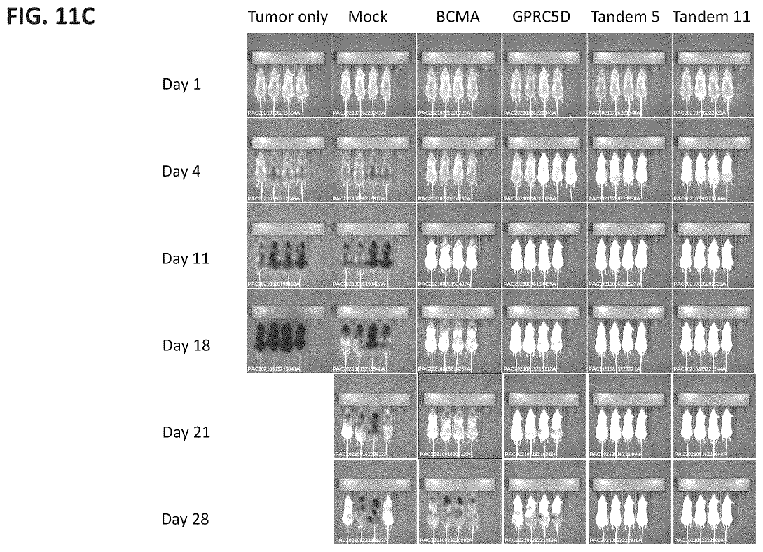

Tumor regression was observed in all treatment groups, but only T cells expressing the bispecific tandem CAR constructs were able to achieve deep and durable responses (FIG. 11A). Mock-processed T cells largely did not prevent MM1.S rFLuc tumor growth and disease progression.

Tumor Control Index (TCI) analysis was performed as described in Example 5. The deeper regression and sustained tumor growth inhibition observed in the bioluminescent curves were reflected in the calculated TCJ scores of tandem CAR constructs 5 and 11 (FIG. 11B). Cells expressing either tandem CAR construct were shown to significantly improve tumor control relative to mock-processed T cells at the 4.0×10⁶ dose used in this experiment. In contrast, while T cells expressing the BCMA CAR or the GPRC5D CAR both resulted in tumor regression in the first seven days post treatment, tumor re-growth was rapid and the overall impact reflected in TCI score was statistically insignificant relative to mock-processed T cells. BLI of representative untreated, mock-processed T cell-treated, and CAR T cell-treated tumor bearing mice demonstrated the enhanced efficacy of the dual targeting approach to drive deep and durable anti-tumor response in this model of multiple myeloma with BCMA and GPRC5D antigen heterogeneity (FIG. 11C). Mice treated with tandem CAR constructs 5 and 11 showed minimal tumor bioluminescence on Days 11, 18, 21, and 28 post-treatment, while untreated and mock-processed T cell-treated mice exhibited increasing levels of tumor bioluminescence throughout the 28-day period (untreated mice did not survive to Day 21) (FIG. 11C; increasing BLI shown by increased intensity of dark shading overlay). Mice treated with the BCMA CAR or the GPRC5D CAR both showed reduced bioluminescence compared to untreated mice and mice treated with mock-processed T cells, but their bioluminescence levels increased after Day 11 (FIG. 11C; reduced BLI shown by lack of or decreased intensity of dark shading overlay).

### Example 8: Further In Vitro Characterization of Tandem CAR 5

a. CAR Expression

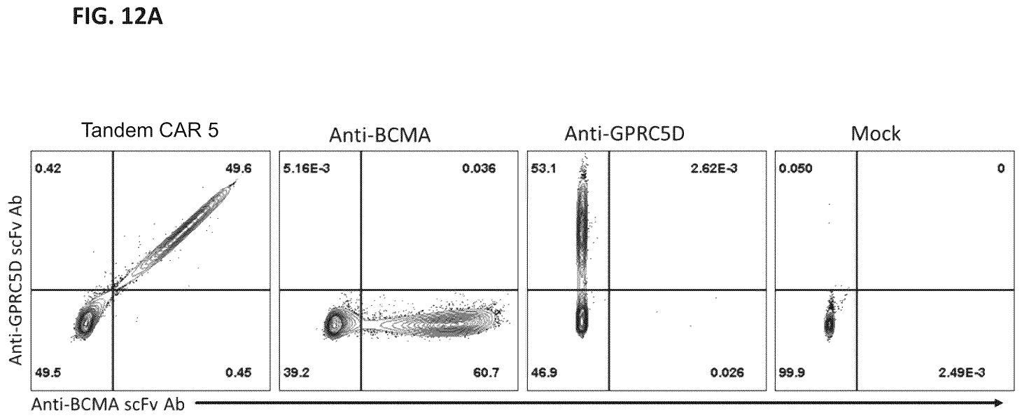

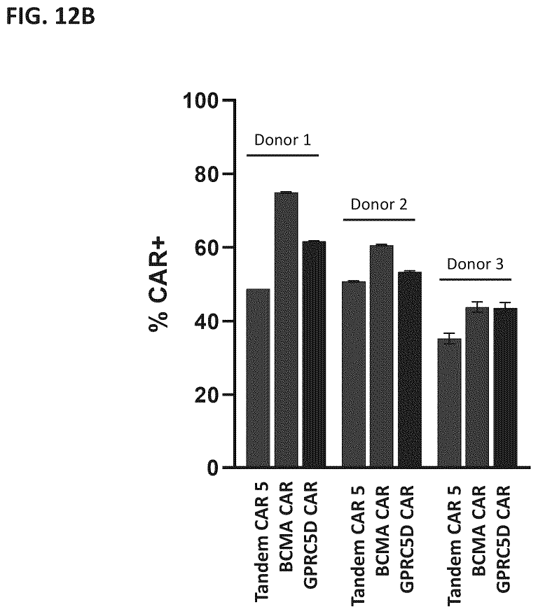

Tandem CAR 5, anti-BCMA CAR, and anti-GPRC5D CAR T cells, or mock-processed T cells, from three healthy human donors were analyzed for CAR expression by flow cytometry. Surface expression of the bispecific tandem CAR 5 construct (tandem CAR 5) was confirmed on T cells using anti-BCMA scFv- and anti-GPRC5D scFv-specific antibodies, each of which in previous studies was demonstrated to specifically and exclusively bind only the scFv to which it was raised and there is no cross-reactivity based on any common feature between the scFvs. Tandem CAR 5 showed expression of both the anti-GPRC5D and anti-BCMA scFvs with no meaningful expression of cells that expressed a single scFv observed (FIG. 12A). The anti-BCMA CAR T only bound the anti-BCMA scFv antibody, and the anti-GPRC5D CAR T only bound the anti-GPRC5D scFv antibody. None of the T cells that were mock-processed were positive for either of the scFv-specific antibodies. Surface expression of CAR was detected on T cells from all three healthy human donors (FIG. 12B).

b. Activation and Cytokine Production

To determine whether tandem CAR 5-expressing cells functionally respond through each scFv binding domain of the bispecific CAR construct, CAR function was assessed following co-culture with either control target cells or target cells expressing individual antigens. Briefly, 3.0×10⁴ T cells expressing tandem CAR 5 or mock transduced T cells were plated alone or with various 3.0×10⁴ target cells: BCMA/GPRC5D-double positive cell lines (MM.1S, OPM-2, KMS12-BM, NCI-H929, OPM-2, RPMI-8226), BCMA-negative/GPRC5D-positive (MM.1S BCMA KO, OPM-2 BCMA KO), BCMA− positive/GPRC5D-negative (MM.1S GPRC5D KO, OPM-2 GPRC5D KO) or BCMA− negative/GPRC5D-negative (Jurkat) tumor cell lines. Monospecific CAR T cells targeting BCMA or GPRC5D, as described in previous Examples, were also included for comparison. Proliferation was measured after 72 hours by the dilution of intracellular CellTrace™ Violet (CTV) measured by geometric mean fluorescence intensity (gMFI) and normalized to CAR T cells cultured without target cells (T cells alone), where a decrease in CTV represented the measurement of proliferation of T cells. T cell activation was measured by upregulation of surface expression of CD25 after 72 hours of co-culture with tumor cell lines. Pro-inflammatory cytokines IFNγ, IL-2, and TNFα were measured in bulk supernatant after 24 hours of co-culture using an electrochemiluminescence cytokine immune assay with V-Plex Custom MSD plates coated with antibodies for IFNγ, IL-2 and TNFα detection (Meso Scale Discovery, Rockville MD) and cytokine concentrations were interpolated from a standard curve and plotted using Prism software (GraphPad Software Inc., La Jolla CA).

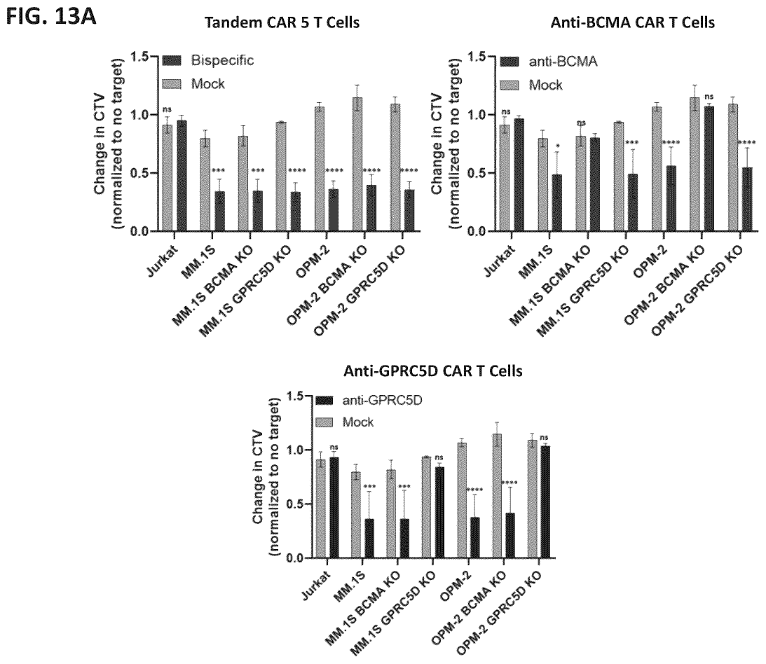

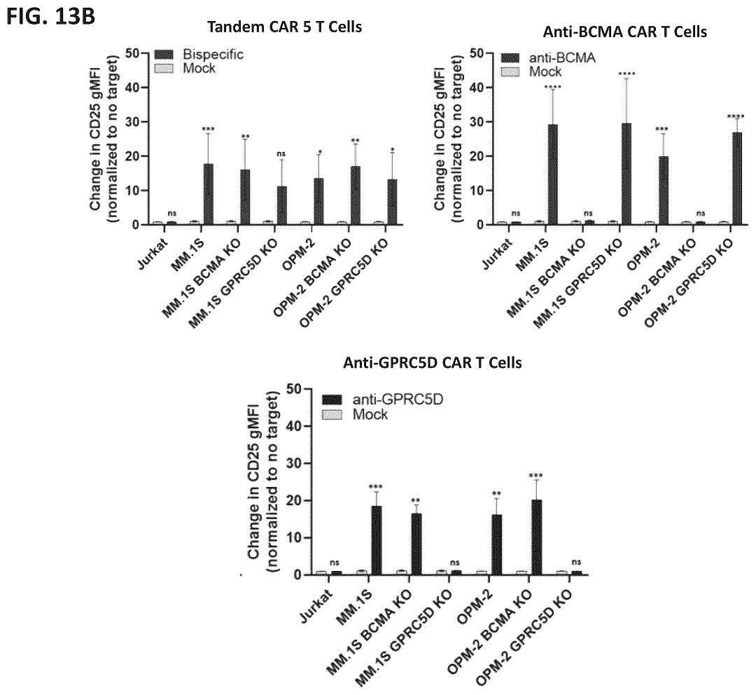

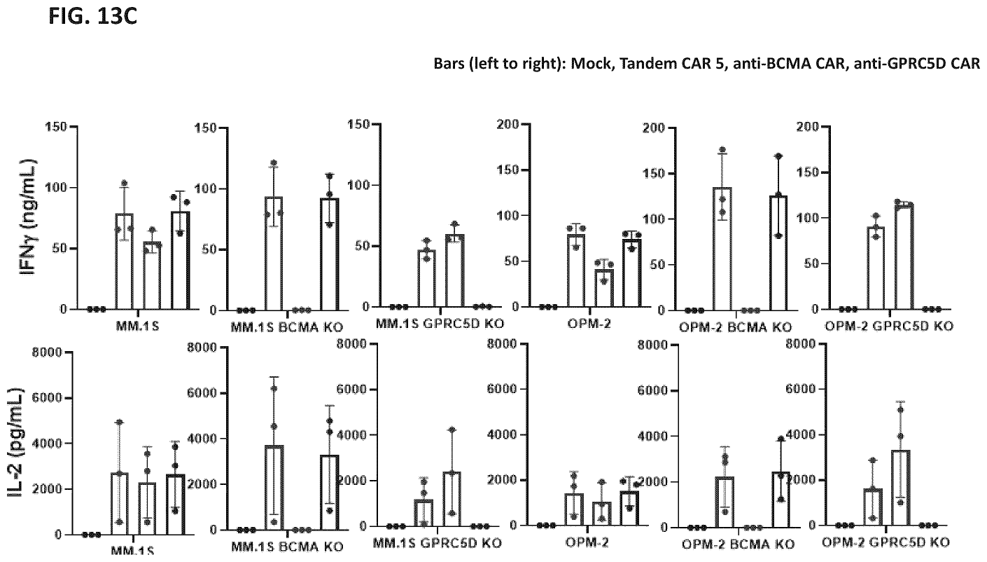

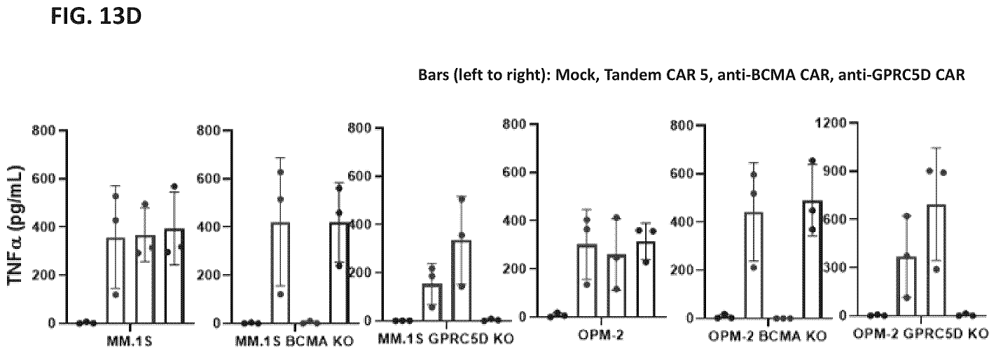

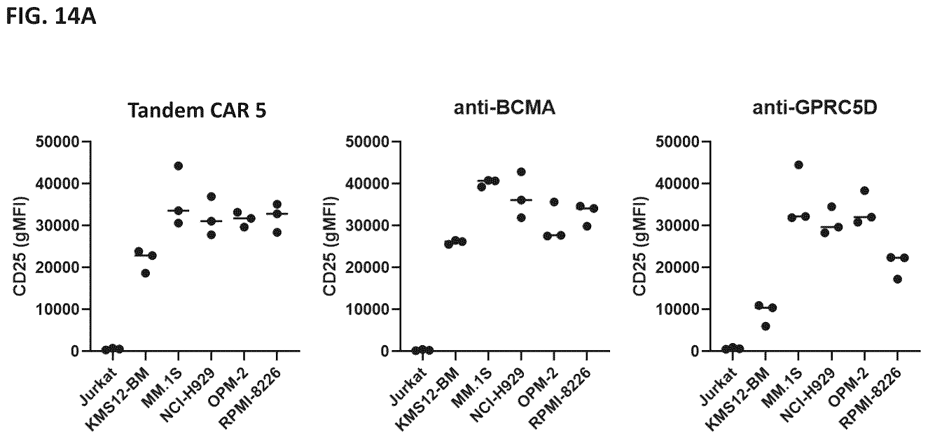

Tandem CAR 5, anti-BCMA CAR, and anti-GPRC5D CAR T cells co-cultured with BCMA-negative/GPRC5D-negative tumor cells (Jurkat) did not proliferate (FIG. 13A) or upregulate CD25 (FIG. 13B), indicating no non-specific activity. All three CAR T cells showed robust proliferation and CD25 upregulation when co-cultured with BCMA-positive/GPRC5D-positive tumor cell lines (MM.1S, OPM-2). However, only Tandem CAR 5 T cells proliferated and showed upregulation of CD25 after co-culture with both single antigen-positive cell lines. In contrast, anti-BCMA CAR T cells did not proliferate after co-culture with BCMA-negative/GPRC5D-positive tumor cell lines (MM.1S BCMA KO, OPM-2 BCMA KO). Similarly, anti-GPRC5D CAR T cells did not proliferate or show upregulation of CD25 after co-culture with BCMA-positive/GPRC5D-negative tumor cell lines (MM.1S GPRC5D KO, OPM-2 GPRC5D KO). These data confirm BCMA and GPRC5D-specific proliferation and activation of tandem CAR 5 CAR T cells, as well as the ability of tandem CAR 5 T cells to function in the presence of both BCMA and GPRC5D, or just a single antigen.

Similarly, tandem CAR 5 T cells secreted IFNγ (FIG. 13C), IL-2 (FIG. 13C), and TNFα (FIG. 13D) after co-culture with BCMA-positive, GPRC5D-positive, or dual-positive tumor cell lines. In contrast, the anti-BCMA and anti-GPRC5D CAR T were only functional when their cognate antigen was present (FIG. 13C-D). None of the CAR T cells secreted measurable cytokines when co-cultured with BCMA-negative/GPRC5D-negative tumor cells (Jurkat) further demonstrating antigen-specific activity of the CAR T cells.

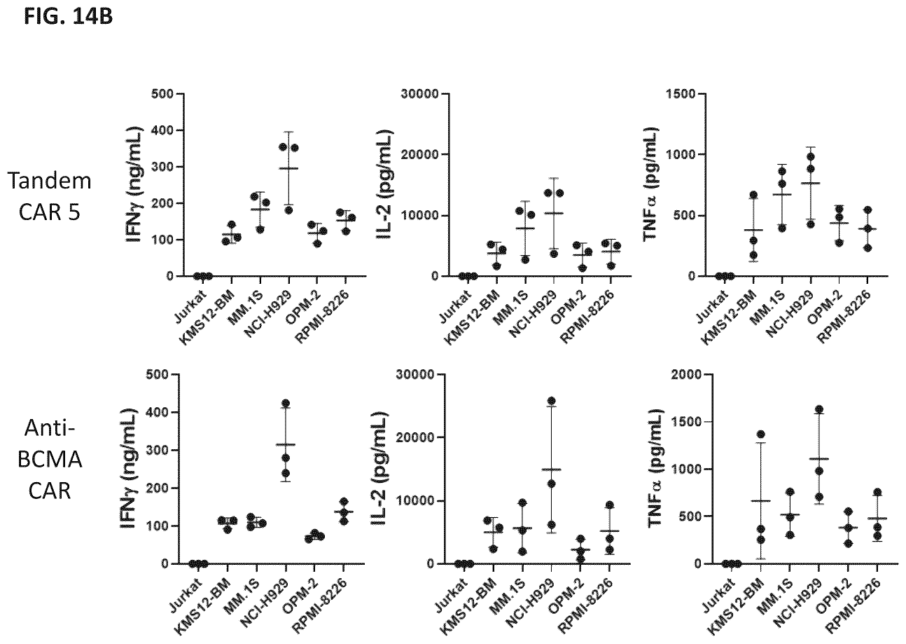

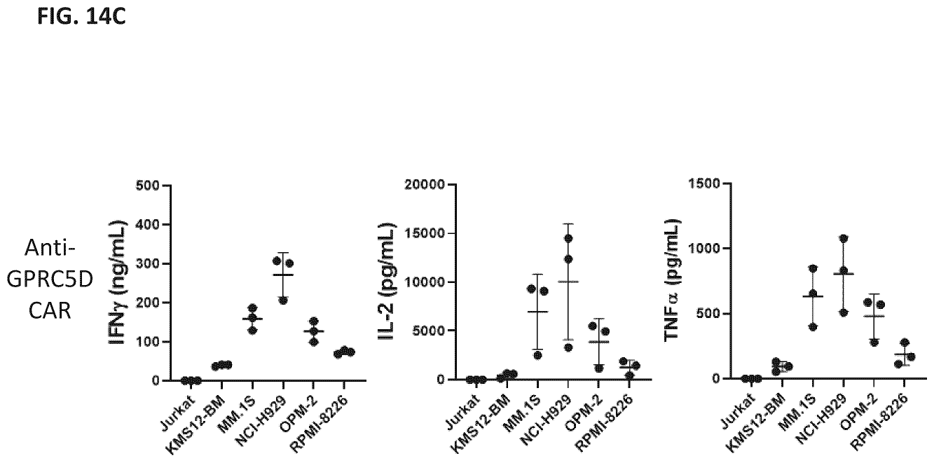

Activation and cytokine production of tandem CAR 5 T cells after co-culture with tumor cell lines with different expression levels of BCMA and GPRC5D was further assessed. None of the CAR T cells showed a change in CD25 expression after co-culture with Jurkat cells which do not express BCMA or GPRC5D. All CAR T cells tested showed an increase in CD25 expression after co-culture with MM cell lines (FIG. 14A). Tandem CAR 5 and anti-BCMA CAR T cells showed robust upregulation of CD25 after co-culture with all BCMA/GPRC5D positive cell lines. Anti-GPRC5D CAR T cells showed a broader range of CD25 upregulation with the least upregulation after co-culture with KMS12-BM and RPMI-8226, which have the lowest GPRC5D expression.

All CAR T cells secreted cytokines after co-culture with MM cell lines expressing BCMA and GPRC5D (FIG. 14B-C). Tandem CAR 5 and anti-BCMA CAR T cells showed robust cytokine production after co-culture with all MM cell lines tested (FIG. 14B). In contrast, the anti-GPRC5D CAR T secreted very low levels of IFNγ, IL-2, and TNFα after co-culture with KMS-12 BM and RPMI-8226, which have GPRC5D expression just above the level of detection (FIG. 14C).

c. Cytotoxic Activity

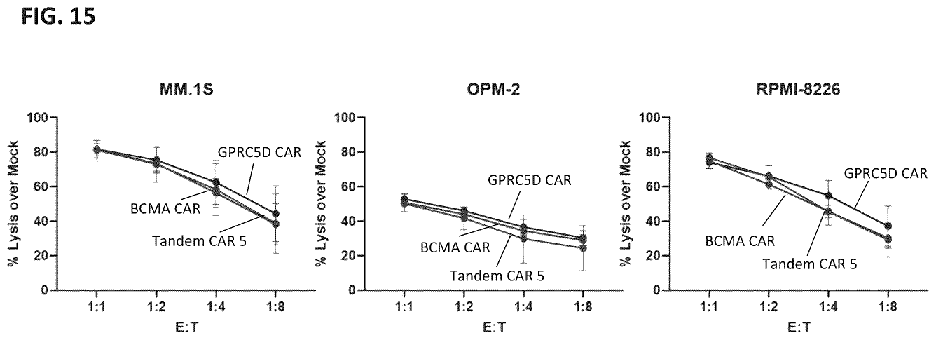

The cytotoxic activity of tandem CAR 5, anti-BCMA CAR and anti-GPRC5D CAR T cells against tumor cell lines expressing variable levels of BCMA and GPRC5D was evaluated. Specifically, tandem CAR 5, anti-BCMA CAR, anti-GPRC5D CAR T cells or mock-processed T cells (effectors) from three healthy human donors were plated in duplicate with BCMA/GPRC5D-positive tumor cell lines (targets) that expressed NucLight Red in 96-well flat bottom plates coated with poly-d-lysine at effector to target ratios of 1:1, 1:2, 1:4, 1:8 using 2.0×10⁴ tumor cells. Total integrated intensity was measured by monitoring NucLight Red fluorescence for 72 hours of culture using IncuCyte® S3 Live-Cell Analysis System with IncuCyte® software (Essen Biosciences, Inc., Ann Arbor, MI). For determining target-cell lysis, total integrated intensity was plotted and the area under the curve (AUC) calculated using Prism software (GraphPad Software Inc., La Jolla, CA). Specific lysis was calculated using the formula % lysis=(AUC for CAR T cells÷AUC for mock T cells)×100. All CAR T cells tested showed cytolytic activity against all three cell lines tested at all effector to target (E:T) ratios tested (FIG. 15).

### Example 9: Further In Vivo Characterization of Tandem Construct 5 in the MM.1S Mouse Model of Multiple Myeloma

NOD.Cg-PrkdcscidIL-2rgtm1Wj1/SzJ mice (NSG) were injected intravenously (IV) with 2.0×10⁶ MM.1S-rFluc multiple myeloma cells on Day −14. On Day −1, mice were randomized into groups (8 mice per group) to balance tumor burden measured by whole-body bioluminescence imaging (BLI). On Day 1, mice were injected IV with a single dose of 5.0×10⁵ (low) or 2.0×10⁶ (high) healthy donor-derived anti-BCMA CAR, anti-GPRC5D CAR, or tandem CAR 5 (anti-BCMA×anti-GPRC5D bispecific CAR) T cells. Control groups included mice that did not receive T cells (n=3 per group) or received mock-processed T cells derived from the same donor. Disseminated tumor growth was assessed in mice by imaging MM.1S firefly luciferase-positive bioluminescence ten minutes after intraperitoneal injection with 3 mg of D-Luciferin. All mice were imaged while under isoflurane anesthesia and tumor burden was measured twice weekly until Day 50 post CAR T cell treatment. Group comparisons of antitumor efficacy were performed with the modified Tumor Control Index (mTCI) method at low and high doses of CAR T cells. Mice were monitored for overall survival until Day 50 post-CAR T treatment. Blood samples from mice were obtained on Days 6, 13, 20, and 27 post-treatment in half the animals per group and alternating cages between time points.

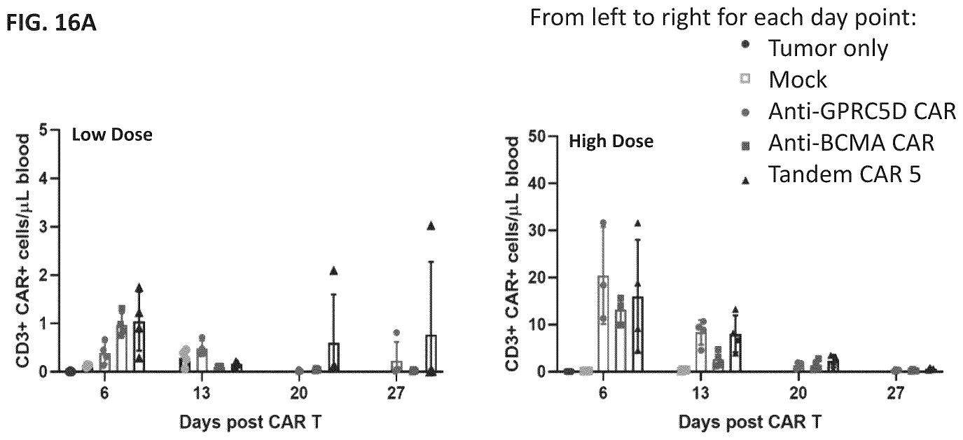

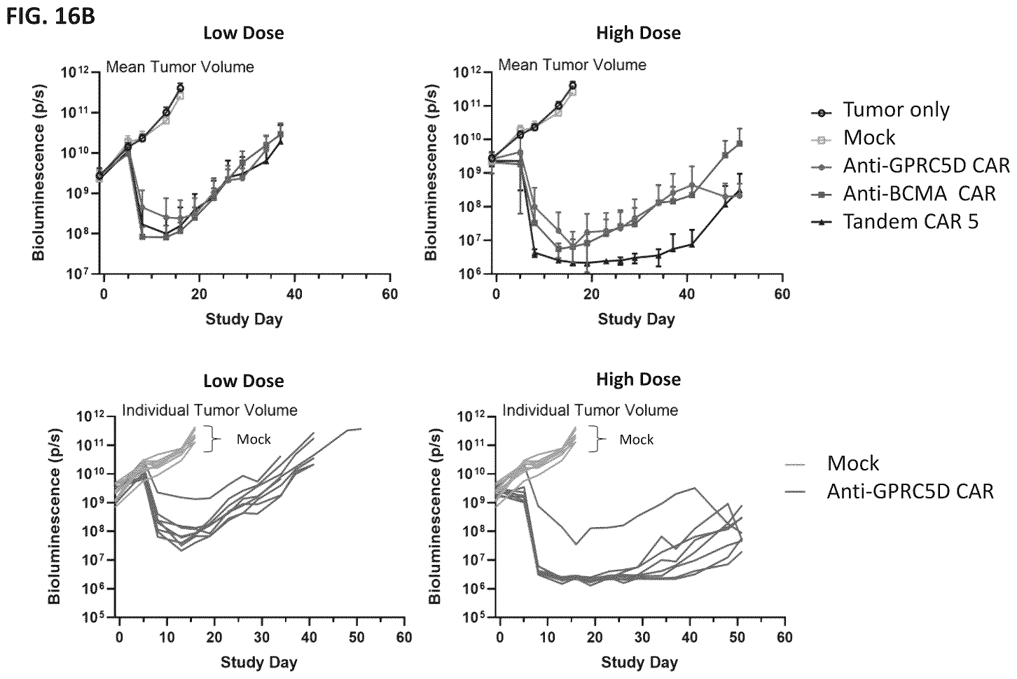

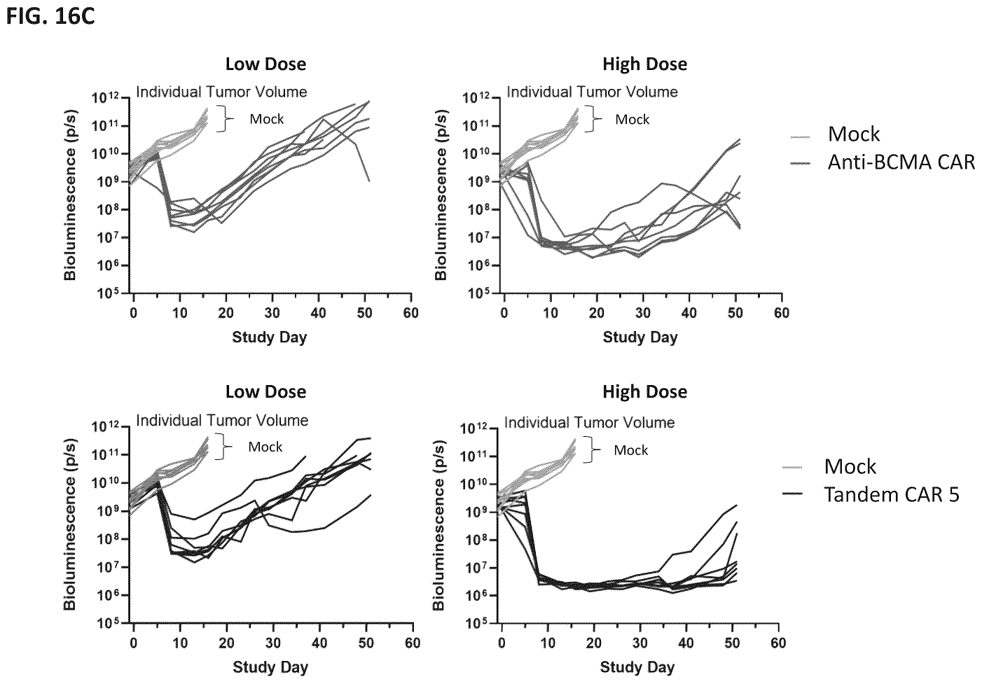

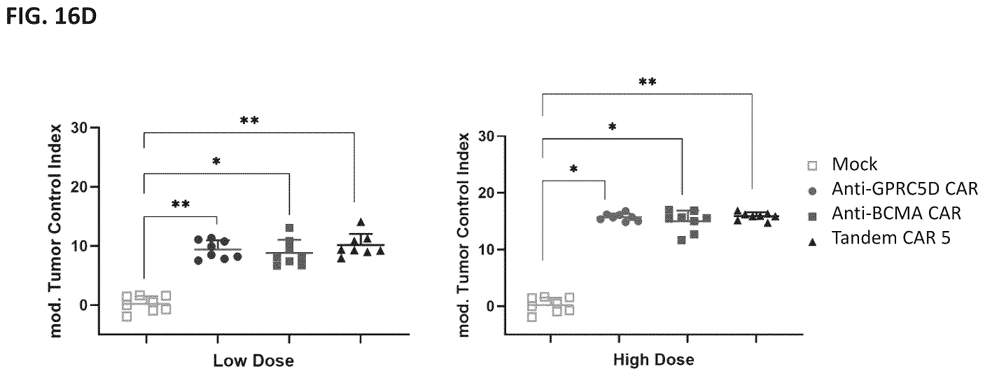

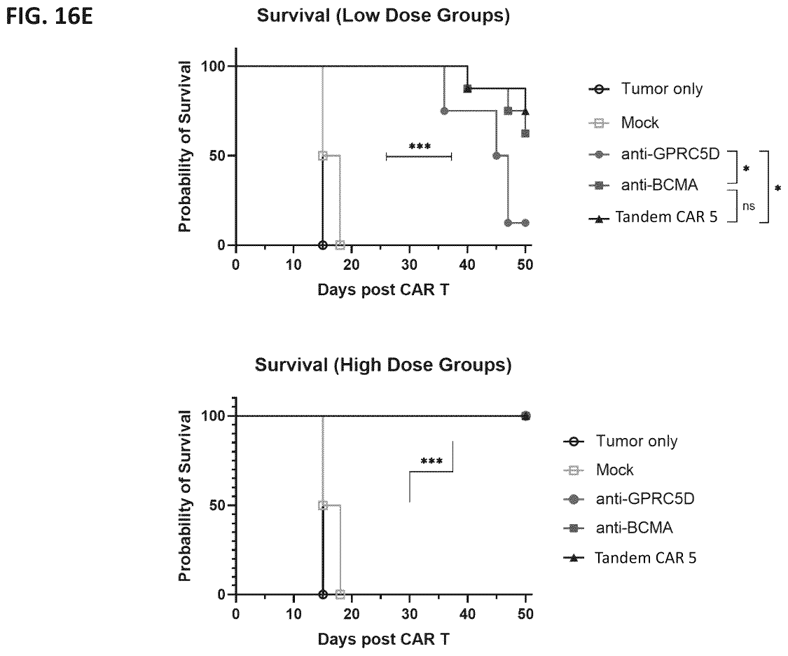

CAR T cell therapy with tandem CAR 5, anti-BCMA CAR, or anti-GPRC5D CAR T cells demonstrated dose-dependent tumor growth inhibition in NSG mice engrafted with disseminated MM.1S multiple myeloma, which express moderate levels of BCMA and GPRC5D. Circulating numbers of CAR+ human CD3+ T cells in peripheral blood of MM.1S xenograft mice were analyzed by multicolor flow cytometry. Peak expansion occurred at Day 6 post CAR T cell infusion for all groups (FIG. 16A). There was no statistically significant difference in antitumor efficacy (tumor volume and mTCI) between the CAR T treatment groups at either dose level (FIGS. 16B-D). However, both low and high doses of anti-BCMA, anti-GPRC5D, and tandem CAR 5 CAR T cells demonstrated superior antitumor activity compared to the mock-processed control group. Mice treated with 5×10⁵ (low dose) or 2×10⁶ (high dose) tandem CAR 5, anti-BCMA CAR, or anti-GPRC5D CAR T cells had a significant survival advantage over mice that received mock-processed T cells (p<0.001; log-rank [Mantel-Cox] test followed by BH post-test correction for multiple comparisons) (FIG. 16E). There was no survival advantage between any treatment group following high dose treatment, whereas low dose tandem CAR 5 and anti-BCMA CAR T cell treatment showed a statistically significant survival advantage (p<0.05) over low dose anti-GPRC5D CAR T cell treatment (FIG. 16E).

### Example 10: Further In Vivo Characterization of Tandem Construct 5 in the OPM-2 Mouse Model of Multiple Myeloma

NOD.Cg-Prkdc^{scid}IL-2rg^{tm1Wj1}/SzJ mice (NSG) were injected intravenously (IV) with 2.0×10⁶ OPM-2-rFluc multiple myeloma cells on Day −14. On Day −1, mice were randomized into groups (8 mice per group) to balance tumor burden measured by whole-body bioluminescence imaging (BLI). On Study Day 1, mice were injected IV with a single dose of 2.0×10⁶ (high) or 5.0×10⁵ (low) healthy donor-derived anti-BCMA, anti-GPRC5D, or tandem CAR 5 (bispecific anti-BCMA×anti-GPRC5D) CAR T cells. Control groups included mice that did not receive T cells (n=3 per group; tumor only) or received mock process T cells derived from the same donor. Disseminated tumor growth was assessed in mice by imaging OPM-2 firefly luciferase-positive bioluminescence ten minutes after intraperitoneal injection with 3 mg of D-Luciferin. All mice were imaged while under isoflurane anesthesia and tumor burden was measured twice-weekly until Day 50 post CAR T cell transfer. Mice were monitored for overall survival until Day 50 post-CAR T treatment. Group comparisons of antitumor efficacy were performed with the modified Tumor Control Index (mTCI) method at low and high doses of CAR T cells

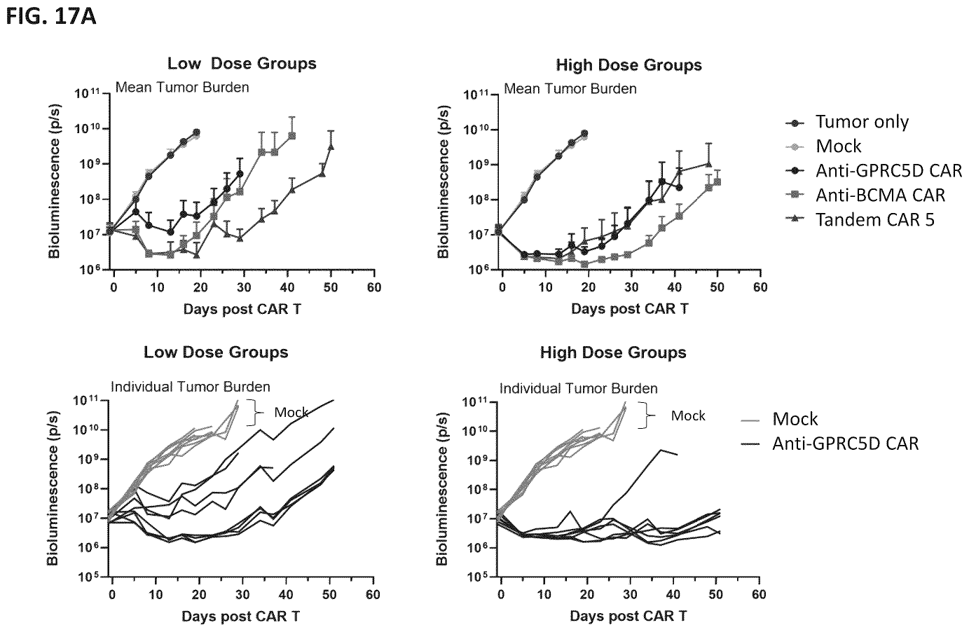

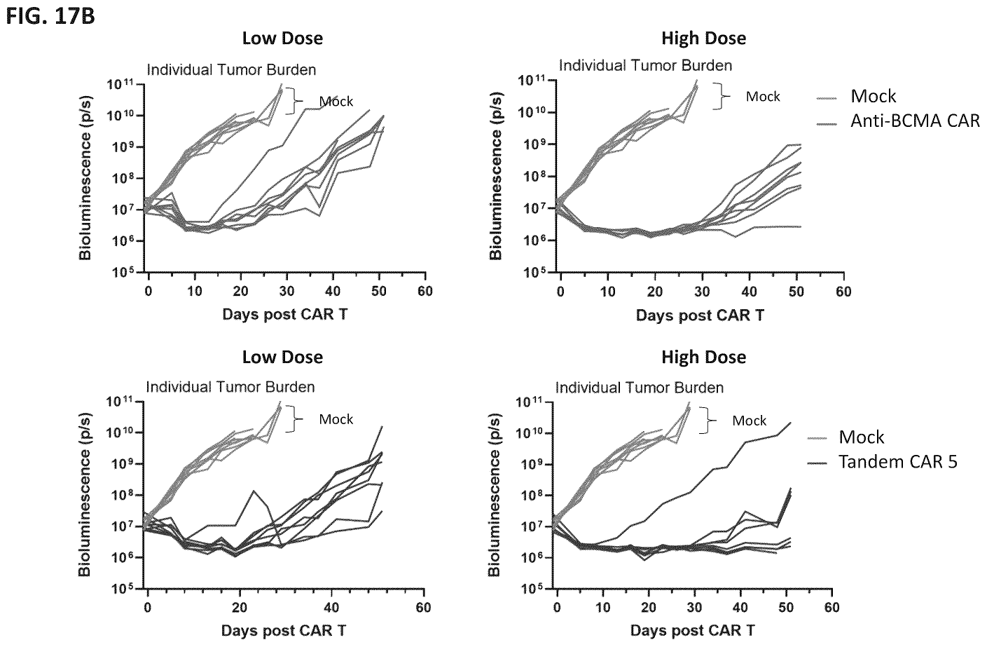

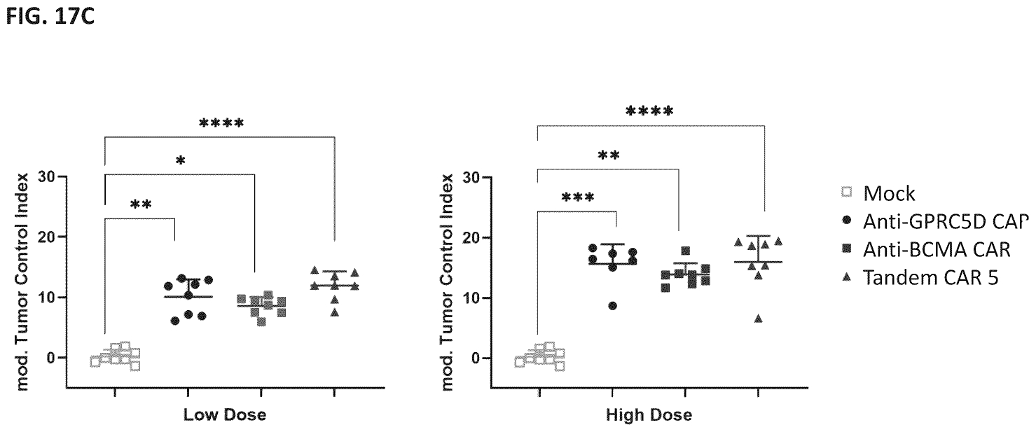

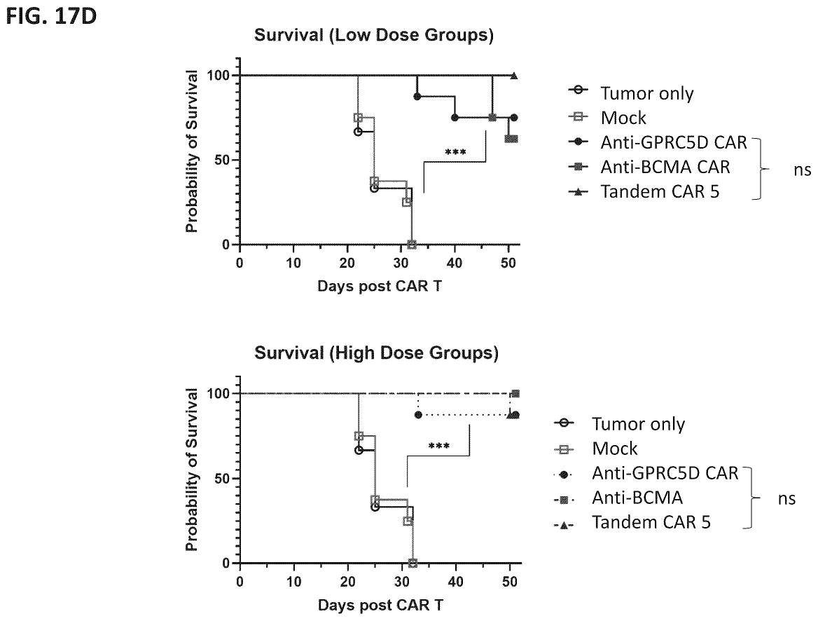

Tandem CAR 5 T cells demonstrated robust, dose-dependent antitumor activity that was superior to the mock process T cell control group (FIG. 17A-B). Tandem CAR 5 T cells also demonstrated antitumor efficacy equivalent to anti-BCMA or anti-GPRC5D CAR T treatment at both 5×10⁵ (low dose) and 2×10⁶ (high dose) dose levels (FIG. 17C). Mice treated with 5×10⁵ (low dose) and 2×10⁶ (high dose) tandem CAR 5, anti-BCMA, or anti-GPRC5D CAR T cells had a significant survival advantage over mice that received mock process T cells (p<0.001; log-rank [Mantel-Cox] test followed by BH post-test correction for multiple comparisons) (FIG. 17D). However, there was no statistically significant difference in survival between the three CAR T treatment groups at either dose level. Circulating tandem CAR 5 BCMA×GPRC5D CAR-positive (CAR+) human CD3+ T cells were detected in the peripheral blood of OPM-2 xenograft mice, but cell numbers were relatively low.

### Example 11: Administration of BCMA×GPRC5D CAR-Positive T Cells to Subjects with Relapsed and/or Refractory Multiple Myeloma (RRMM)

This study is performed to evaluate the exemplary BCMA×GPRC5D CAR-Positive T Cell therapy, tandem CAR 5 BCMA×GPRC5D CAR-positive T cells (e.g., SEQ ID NO: 37), in subjects with RRMM.

A group of human subjects (age≥18 years of age) are selected for administration of BCMA×GPRC5D CAR-positive T cells. The subjects are those with a history of RRMM (measurable multiple myeloma as per IMWG; Eastern Cooperative Oncology Group performance status of 0-1) who have been treated with at least 3 anti-myeloma treatment regimens. The subject must have previously received each of the following therapies: (1) a prior therapy that included at least 1 complete cycle of treatment (unless progressive disease was the best response to the regimen) of an immunomodulatory agent (e.g., thalidomide, lenalidomide, pomalidomide) and a proteasome inhibitor (e.g., bortezomib, carfilzomib, ixaxomib), either alone or in combination; (2) a prior therapy that included anti-CD38 antibody therapy (e.g., daratumumab), alone or in combination; and (3) a prior therapy that included autologous hematopoietic stem cell transplantation (HSCT), unless the participant was ineligible. For the prior therapies, induction with or without HSCT and with or without maintenance therapy was considered 1 regimen. The subjects must have confirmed progressive disease (measurable MM) as determined by IMWG criteria on or within 12 months (measured from the last dose) of completing treatment with the last anti-myeloma treatment regimen or have confirmed progressive disease within 6 months prior to screening for CAR-T cell treatment and who are subsequently determined to be refractory or non-responsive to their most recent anti-myeloma treatment regimen. However, subjects who have received as their last treatment a CAR T-cell therapy may be eligible beyond 12 months of their last treatment.

The subject is a subject who:
i) has no known active or history of CNS involvement of multiple myeloma;
ii) has no active or history of plasma cell leukemia, Waldenstrom's macroglobulinemia, POEMS (polyneuropathy, organomegaly, endocrinopathy, monoclonal protein, skin changes) syndrome, or clinically significant amyloidosis; and
iii) has no uncontrolled or active systemic fungal, bacterial, viral, or other infection despite appropriate anti-infective treatment at the time of leukapheresis, or within 7 days prior to starting LD chemotherapy, or within 7 days before CAR-T cell infusion.

Safety and tolerability are assessed, as well assessment of efficacy in the subjects by International Myeloma Working Group (IMWG) Uniform Response Criteria. The study also includes the characterization of the PK profile (cellular kinetics) of the BCMA×GPRC5D CAR-positive T cells. Safety and tolerability endpoints examined in the study include type, frequency, and severity of treatment emergent adverse events (AEs), serious adverse events (SAEs), and dose limited toxicities (DLTs) (in dose escalation). Endpoints of PK profile and response examined include Cmax, Tmax, AUC(0-28), persistence, expansion rate, and other relevant PK parameters of the CAR T cells in peripheral blood; and measures of clinical response. Measures of clinical response include overall response rate (ORR), complete response rate (CRR), very good partial response (VGPR) or better, progression free survival (PFS), overall survival (OS), time to response/time to complete response (TTR/TTCR), and/or duration of response/duration of complete response (DOR/DOCR).

A minimum of 3 and up to 15 subjects with relapsed/refractory multiple melanoma who had received at least 3 prior multiple myeloma treatment regimens are administered BCMA×GPRC5D CAR-positive T cells. Prior to CAR-T cell infusion, subjects are treated with lymphodepleting chemotherapy prior to infusion that is completed at least 48 hours and up to 9 days before CAR-T cell infusion. Subjects are treated either by intravenous administration of fludarabine (flu, 30 mg/m²/day) and cyclophosphamide (Cy, 300 mg/m²/day) for 3 days prior to CAR-T cell infusion (Days −5, −4, and −3), or by intravenous administration of bendamustine (90 mg/m²) for 2 days prior to CAR-T cell infusion (Days −4 and −3).

The subjects optionally may receive a bridging therapy (typically for ≤4 weeks) between the time of leukapheresis and the lymphodepleting therapy during the time the BCMA×GPRC5D CAR-positive T cells are being manufactured (e.g., engineered and expanded). Bridging therapy is discontinued at least 14 days prior to initiation of lymphodepleting chemotherapy, except corticosteroids which are discontinued at least 72 hours prior. The subject also is recovered from bridging therapy relates toxicities to Grade≤2 (except for alopecia) prior to initiation of lymphodepleting therapy.

Subjects are administered a single-dose IV infusion of the BCMA×GPRC5D CAR-positive T cells (e.g., SEQ ID NO: 37) on Day 1. The subjects are administered one of four escalating dose levels (DL): 75×10⁶, 150×10⁶, 300×10⁶, 450×10⁶ CAR T cells. An optional dose level (DL) (25×10⁶ CAR T cells) is utilized if the starting dose has unacceptable toxicity. Response is assessed per IMWG uniform criteria.

Subjects are monitored for safety (including serious and severity of adverse events), pharmacokinetics and pharmacodynamics, and clinical response through responses by International Myeloma Working Group (IMWG) Uniform Response Criteria (Kuman 2016). Determining clinical response to the treatment includes assessing the overall response rate (ORR; partial response or better), complete response rate (CRR; including proportion of patients with sCR or CR) and very good partial response rate or better (VGPRR; including proportion of patients achieving sCR, CR or VGPR). The estimated Cmax, tmax, AUC (0-28D, and other relevant PK parameters of CAR T cells in the peripheral blood are assessed.

All participants are observed for dose limiting toxicities (DLTs). DLT-evaluable participants were all participants who received conforming BCMA×GPRC5D CAR-positive T cells at the assigned dose level, and who either experienced a DLT or had been followed for a full evaluation period. The DLT-evaluable period is defined as Days 1 to 28 for a total evaluation period of 28 days following CAR T cell infusion (inclusive of Day 1). The severity of adverse events (AEs) are graded according to National Cancer Institute (NCI) Common Terminology Criteria for Adverse Events (CTCAE) v5.0 (National Cancer Institute. Common terminology criteria for adverse events (CTCAE). Version 5.0. Available at: https://ctep.cancer.gov/protocolDevelopment/electronic_applications/docs/CTCAE_v5_Quick_Reference_8.5x11.pdf), American Society for Transplantation and Cellular Therapy (ASTCT) Consensus Grading for CRS and Neurologic Toxicity Associated with Immune Effector Cells (Lee et al. Biol Blood Marrow Transplant., 2019; 25:625-38), and according to Cairo-Bishop Criteria (Cairo et al. Br J Haemat, 2004; 127:3-11) for tumor lysis syndrome (TLS). Additional information may also be collected according to Lee, 2014 Grading criteria (Lee et al., Blood, 2014; 124:188-195).

DLTs are defined as AEs occurring within 28-days following CAR T cell infusion that (a) are at least possibly attributed to CAR T cells and (b) meet any of the following criteria:
Grade 5 toxicity not related to disease progression and deemed suspected to be related to CAR T cell infusion;
Grade 4 CRS not improved to Grade 2 or lower≤72 hours;
Grade 3 or 4 ICANS or other NT not improved to Grade 2 or lower≤72 hours; or
Grade 3 or 4 toxicity involving vital organs (eg, cardiac, pulmonary) of any duration; all other
Grade 3 toxicities, not attributable to underlying disease or lymphodepleting chemotherapy, that do not resolve to ≤Grade 2 within 72 hours, except as follows:
   a. Confirmed Hy's law case;
   b. Grade 3 AST and/or ALT increase lasting≤14 days or Grade 4 AST and/or ALT increase lasting≤7 days;
   c. Certain treatment-emergent, isolated Grade 3 or 4, asymptomatic laboratory electrolyte abnormalities (ie, those occurring without clinical consequence) not directly related to vital organ toxicities (eg, hypomagnesemia, hyponatremia, hypernatremia, hypophosphatemia, hyperphosphatemia, hypocalcemia, hypercalcemia) that resolve, with or without intervention, to Grade≤2 in ≤7 days; these findings will be discussed and reviewed in consultation with the SRC for a final recommendation of DLT status; and
   d. Hematologic toxicities:
      1) Grade 4 neutropenia lasting beyond Day 42; or
      2) Grade 3 thrombocytopenia accompanied by clinically significant bleeding; or Grade 4 thrombocytopenia that does not resolve to Grade 3 or lower by Day 42 or accompanied by clinically significant bleeding.

An additional study is performed to evaluate the exemplary BCMA×GPRC5D CAR-Positive T Cell therapy, designated tandem CAR 5 BCMA×GPRC5D CAR-positive T cells (e.g., SEQ ID NO: 37), in subjects with RRMM who have been treated with at least 1, but not more than 3, anti-myeloma treatment regimens.

As above, the subjects are human subjects (age≥18 years of age) with a history of RRMM (measurable multiple myeloma as per IMWG; Eastern Cooperative Oncology Group performance status of 0-1). The subjects of this additional study must have received 1 to 3 prior anti-myeloma treatment therapies including a PI and IMiD. The subjects must have previously received a prior therapy that included at least 1 complete cycle of treatment (unless progressive disease was the best response to the regimen) of an immunomodulatory agent (e.g., thalidomide, lenalidomide, pomalidomide) and a proteasome inhibitor (e.g., bortezomib, carfilzomib, ixaxomib), either alone or in combination.

As above, the subjects must have confirmed progressive disease (measurable MM) as determined by IMWG criteria on or within 12 months (measured from the last dose) of completing treatment with the last anti-myeloma treatment regimen or have confirmed progressive disease within 6 months prior to screening for CAR-T cell treatment and who are subsequently determined to be refractory or non-responsive to their most recent anti-myeloma treatment regimen. However, subjects who have received as their last treatment a CAR T-cell therapy may be eligible beyond 12 months of their last treatment.

As above, subjects are treated with lymphodepleting chemotherapy and an optional bridging therapy prior to CAR-T cell infusion. Subjects are monitored for safety (including serious and severity of adverse events), pharmacokinetics and pharmacodynamics, and clinical response through responses by International Myeloma Working Group (IMWG) Uniform Response Criteria (Kuman 2016).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | ANNOTATION |
|---|---|---|
| 1 | SHSMN | CDRH1 |
| 2 | SISSDSTYTYYADSVKG | CDRH2 |
| 3 | SGGQWKYYDY | CDRH3 |
| 4 | QGDSLRSYYAS | CRDL1 |
| 5 | GKNNRPS | CDRL2 |
| 6 | NSRDSSGNPPVV | CDRL3 |
| 7 | QVQLVESGGGLVHPGGSLRLSCAASGFTFRSHSMNWVRQAPGKGLEWVSS | GPRC5D VH |
| | ISSDSTYTYYADSVKGRFTISRDNAKNSLYLQMNSLRAEDTAVYYCARSG | |
| | GQWKYYDYWGQGTLVTVSS | |
| 8 | SSELTQDPAVSVALGQTVRITCQGDSLRSYYASWYQQKPGQAPVLVIYGK | GPRC5D VL |
| | NNRPSGIPDRFSGSSSGNTASLTITGAQAEDEADYYCNSRDSSGNPPVVF | |
| | GGGTKLTVL | |
| 9 | DYYVY | CDRH1 |
| 10 | WINPNSGGTNYAQKFQG | CDRH2 |
| 11 | SQRDGYMDY | CDRH3 |
| 12 | TGTSSDVG | CRDL1 |
| 13 | EDSKRPS | CDRL2 |
| 14 | SSNTRSSTLV | CDRL3 |
| 15 | EVQLVQSGAEMKKPGASLKLSCKASGYTFIDYYVYWMRQAPGQGLESMGW | BCMA VH |
| | INPNSGGTNYAQKFQGRVTMTRDTSISTAYMELSRLRSDDTAMYYCARSQ | |
| | RDGYMDYWGQGTLVTVSS | |
| 16 | QSALTQPASVSASPGQSIAISCTGTSSDVGWYQQHPGKAPKLMIYEDSKR | BCMA VL |
| | PSGVSNRFSGSKSGNTASLTISGLQAEDEADYYCSSNTRSSTLVFGGGTK | |
| | LTVL | |
| 17 | GSRGGGGSGGGGSGGGGSLEMA | linker |
| 18 | GSTSGSGKPGSGEGSTKG | linker |
| 19 | EAAAK | linker |
| 20 | GGGS | 3GSlinker |
| 21 | GGGGS | 4GS linker |
| 22 | GGGGSGGGGS | linker |
| 23 | GGGGSGGGGSGGGGS | linker |
| 24 | GGGGSGGGGSGGGGSGGGGS | linker |
| 25 | ESKYGPPCPPCP | short spacer |
| 26 | ESKYGPPCPPCPGQPREPQVYTLPPSQEEMTKNQVSLTCLVKGFYPSDIA | medium |
| | VEWESNGQPENNYKTTPPVLDSDGSFFLYSRLTVDKSRWQEGNVFSCSVM | spacer (hinge- |
| | HEALHNHYTQKSLSLSLGK | CH3 119 aa) |
| 27 | ESKYGPPCPPCPAPPVAGPSVFLFPPKPKDTLMISRTPEVTCVVVDVSQE | long spacer |
| | DPEVQFNWYVDGVEVHNAKTKPREEQFQSTYRVVSVLTVLHQDWLNGKEY | (IgG4/IgG2 |
| | KCKVSNKGLPSSIEKTISKAKGQPREPQVYTLPPSQEEMTKNQVSLTCLV | hinge- |
| | KGFYPSDIAVEWESNGQPENNYKTTPPVLDSDGSFFLYSRLTVDKSRWQE | IgG2/IgG4 |
| | GNVFSCSVMHEALHNHYTQKSLSLSLGK | CH2- IgG4 |
| | | CH3 spacer; |
| | | 228 aa) |
| 28 | MFWVLVVVGGVLACYSLLVTVAFIIFWV | CD28 |
| | | transmembrane |
| | | domain |
| 29 | KRGRKKLLYIFKQPFMRPVQTTQEEDGCSCRFPEEEEGGCEL | 4-1BB- |
| | | derived |
| | | intracellular |
| | | co-signaling |
| | | sequence (aa) |
| 30 | RVKFSRSADAPAYQQGQNQLYNELNLGRREEYDVLDKRRGRDPEMGGKPR | CD3-zeta |
| | RKNPQEGLYNELQKDKMAEAYSEIGMKGERRRGKGHDGLYQGLSTATKDT | derived |
| | YDALHMQALPPR | intracellular |
| | | signaling |
| | | domain (aa) |
| 31 | SSELTQDPAVSVALGQTVRITCQGDSLRSYYASWYQQKPGQAPVLVIYGK | CAR 1 (aa) |
| | NNRPSGIPDRFSGSSSGNTASLTITGAQAEDEADYYCNSRDSSGNPPVVF | |
| | GGGTKLTVLGSRGGGGSGGGGSGGGGSLEMAQVQLVESGGGLVHPGGSLR | |
| | LSCAASGFTFRSHSMNWVRQAPGKGLEWVSSISSDSTYTYYADSVKGRFT | |
| | ISRDNAKNSLYLQMNSLRAEDTAVYYCARSGGQWKYYDYWGQGTLVTVSS | |
| | EAAAKEVQLVQSGAEMKKPGASLKLSCKASGYTFIDYYVYWMRQAPGQGL | |
| | ESMGWINPNSGGTNYAQKFQGRVTMTRDTSISTAYMELSRLRSDDTAMYY | |
| | CARSQRDGYMDYWGQGTLVTVSSGSRGGGGSGGGGSGGGGSLEMAQSALT | |
| | QPASVSASPGQSIAISCTGTSSDVGWYQQHPGKAPKLMIYEDSKRPSGVS | |
| | NRFSGSKSGNTASLTISGLQAEDEADYYCSSNTRSSTLVFGGGTKLTVLE | |
| | SKYGPPCPPCPAPPVAGPSVFLFPPKPKDTLMISRTPEVTCVVVDVSQED | |
| | PEVQFNWYVDGVEVHNAKTKPREEQFQSTYRVVSVLTVLHQDWLNGKEYK | |
| | CKVSNKGLPSSIEKTISKAKGQPREPQVYTLPPSQEEMTKNQVSLTCLVK | |
| | GFYPSDIAVEWESNGQPENNYKTTPPVLDSDGSFFLYSRLTVDKSRWQEG | |
| | NVFSCSVMHEALHNHYTQKSLSLSLGKMFWVLVVVGGVLACYSLLVTVAF | |
| | IIFWVKRGRKKLLYIFKQPFMRPVQTTQEEDGCSCRFPEEEEGGCELRVK | |
| | FSRSADAPAYQQGQNQLYNELNLGRREEYDVLDKRRGRDPEMGGKPRRKN | |
| | PQEGLYNELQKDKMAEAYSEIGMKGERRRGKGHDGLYQGLSTATKDTYDA | |
| | LHMQALPPR | |
| 32 | QVQLVESGGGLVHPGGSLRLSCAASGFTFRSHSMNWVRQAPGKGLEWVSS | CAR 2 (aa) |
| | ISSDSTYTYYADSVKGRFTISRDNAKNSLYLQMNSLRAEDTAVYYCARSG | |
| | GQWKYYDYWGQGTLVTVSSGSRGGGGSGGGGSGGGGSLEMASSELTQDPA | |
| | VSVALGQTVRITCQGDSLRSYYASWYQQKPGQAPVLVIYGKNNRPSGIPD | |
| | RFSGSSSGNTASLTITGAQAEDEADYYCNSRDSSGNPPVVFGGGTKLTVL | |
| | GGGGSEVQLVQSGAEMKKPGASLKLSCKASGYTFIDYYVYWMRQAPGQGL | |
| | ESMGWINPNSGGTNYAQKFQGRVTMTRDTSISTAYMELSRLRSDDTAMYY | |
| | CARSQRDGYMDYWGQGTLVTVSSGSRGGGGSGGGGSGGGGSLEMAQSALT | |
| | QPASVSASPGQSIAISCTGTSSDVGWYQQHPGKAPKLMIYEDSKRPSGVS | |
| | NRFSGSKSGNTASLTISGLQAEDEADYYCSSNTRSSTLVFGGGTKLTVLE | |
| | SKYGPPCPPCPAPPVAGPSVFLFPPKPKDTLMISRTPEVTCVVVDVSQED | |
| | PEVQFNWYVDGVEVHNAKTKPREEQFQSTYRVVSVLTVLHQDWLNGKEYK | |
| | CKVSNKGLPSSIEKTISKAKGQPREPQVYTLPPSQEEMTKNQVSLTCLVK | |
| | GFYPSDIAVEWESNGQPENNYKTTPPVLDSDGSFFLYSRLTVDKSRWQEG | |
| | NVFSCSVMHEALHNHYTQKSLSLSLGKMFWVLVVVGGVLACYSLLVTVAF | |
| | IIFWVKRGRKKLLYIFKQPFMRPVQTTQEEDGCSCRFPEEEEGGCELRVK | |
| | FSRSADAPAYQQGQNQLYNELNLGRREEYDVLDKRRGRDPEMGGKPRRKN | |
| | PQEGLYNELQKDKMAEAYSEIGMKGERRRGKGHDGLYQGLSTATKDTYDA | |
| | LHMQALPPR | |
| 33 | SSELTQDPAVSVALGQTVRITCQGDSLRSYYASWYQQKPGQAPVLVIYGK | CAR 3 (aa) |
| | NNRPSGIPDRFSGSSSGNTASLTITGAQAEDEADYYCNSRDSSGNPPVVF | |
| | GGGTKLTVLGSRGGGGSGGGGSGGGGSLEMAQVQLVESGGGLVHPGGSLR | |
| | LSCAASGFTFRSHSMNWVRQAPGKGLEWVSSISSDSTYTYYADSVKGRFT | |
| | ISRDNAKNSLYLQMNSLRAEDTAVYYCARSGGQWKYYDYWGQGTLVTVSS | |
| | GGGGSGGGGSGGGGSGGGGSEVQLVQSGAEMKKPGASLKLSCKASGYTFI | |
| | DYYVYWMRQAPGQGLESMGWINPNSGGTNYAQKFQGRVTMTRDTSISTAY | |
| | MELSRLRSDDTAMYYCARSQRDGYMDYWGQGTLVTVSSGSRGGGGSGGGG | |
| | SGGGGSLEMAQSALTQPASVSASPGQSIAISCTGTSSDVGWYQQHPGKAP | |
| | KLMIYEDSKRPSGVSNRFSGSKSGNTASLTISGLQAEDEADYYCSSNTRS | |
| | STLVFGGGTKLTVLESKYGPPCPPCPAPPVAGPSVFLFPPKPKDTLMISR | |
| | TPEVTCVVVDVSQEDPEVQFNWYVDGVEVHNAKTKPREEQFQSTYRVVSV | |
| | LTVLHQDWLNGKEYKCKVSNKGLPSSIEKTISKAKGQPREPQVYTLPPSQ | |
| | EEMTKNQVSLTCLVKGFYPSDIAVEWESNGQPENNYKTTPPVLDSDGSFF | |
| | LYSRLTVDKSRWQEGNVFSCSVMHEALHNHYTQKSLSLSLGKMFWVLVVV | |
| | GGVLACYSLLVTVAFIIFWVKRGRKKLLYIFKQPFMRPVQTTQEEDGCSC | |
| | RFPEEEEGGCELRVKFSRSADAPAYQQGQNQLYNELNLGRREEYDVLDKR | |
| | RGRDPEMGGKPRRKNPQEGLYNELQKDKMAEAYSEIGMKGERRRGKGHDG | |
| | LYQGLSTATKDTYDALHMQALPPR | |
| 34 | QVQLVESGGGLVHPGGSLRLSCAASGFTFRSHSMNWVRQAPGKGLEWVSS | CAR 4 (aa) |
| | ISSDSTYTYYADSVKGRFTISRDNAKNSLYLQMNSLRAEDTAVYYCARSG | |
| | GQWKYYDYWGQGTLVTVSSGSRGGGGSGGGGSGGGGSLEMASSELTQDPA | |
| | VSVALGQTVRITCQGDSLRSYYASWYQQKPGQAPVLVIYGKNNRPSGIPD | |
| | RFSGSSSGNTASLTITGAQAEDEADYYCNSRDSSGNPPVVFGGGTKLTVL | |
| | GGGGSGGGGSGGGGSGGGGSEVQLVQSGAEMKKPGASLKLSCKASGYTFI | |
| | DYYVYWMRQAPGQGLESMGWINPNSGGTNYAQKFQGRVTMTRDTSISTAY | |
| | MELSRLRSDDTAMYYCARSQRDGYMDYWGQGTLVTVSSGSRGGGGSGGGG | |
| | SGGGGSLEMAQSALTQPASVSASPGQSIAISCTGTSSDVGWYQQHPGKAP | |
| | KLMIYEDSKRPSGVSNRFSGSKSGNTASLTISGLQAEDEADYYCSSNTRS | |
| | STLVFGGGTKLTVLESKYGPPCPPCPAPPVAGPSVFLFPPKPKDTLMISR | |
| | TPEVTCVVVDVSQEDPEVQFNWYVDGVEVHNAKTKPREEQFQSTYRVVSV | |
| | LTVLHQDWLNGKEYKCKVSNKGLPSSIEKTISKAKGQPREPQVYTLPPSQ | |
| | EEMTKNQVSLTCLVKGFYPSDIAVEWESNGQPENNYKTTPPVLDSDGSFF | |
| | LYSRLTVDKSRWQEGNVFSCSVMHEALHNHYTQKSLSLSLGKMFWVLVVV | |
| | GGVLACYSLLVTVAFIIFWVKRGRKKLLYIFKQPFMRPVQTTQEEDGCSC | |
| | RFPEEEEGGCELRVKFSRSADAPAYQQGQNQLYNELNLGRREEYDVLDKR | |
| | RGRDPEMGGKPRRKNPQEGLYNELQKDKMAEAYSEIGMKGERRRGKGHDG | |
| | LYQGLSTATKDTYDALHMQALPPR | |
| 35 | EVQLVQSGAEMKKPGASLKLSCKASGYTFIDYYVYWMRQAPGQGLESMGW | CAR 8 (aa) |
| | INPNSGGTNYAQKFQGRVTMTRDTSISTAYMELSRLRSDDTAMYYCARSQ | |
| | RDGYMDYWGQGTLVTVSSEAAAKSSELTQDPAVSVALGQTVRITCQGDSL | |
| | RSYYASWYQQKPGQAPVLVIYGKNNRPSGIPDRFSGSSSGNTASLTITGA | |
| | QAEDEADYYCNSRDSSGNPPVVFGGGTKLTVLGSRGGGGSGGGGSGGGGS | |
| | LEMAQVQLVESGGGLVHPGGSLRLSCAASGFTFRSHSMNWVRQAPGKGLE | |
| | WVSSISSDSTYTYYADSVKGRFTISRDNAKNSLYLQMNSLRAEDTAVYYC | |
| | ARSGGQWKYYDYWGQGTLVTVSSEAAAKQSALTQPASVSASPGQSIAISC | |
| | TGTSSDVGWYQQHPGKAPKLMIYEDSKRPSGVSNRFSGSKSGNTASLTIS | |
| | GLQAEDEADYYCSSNTRSSTLVFGGGTKLTVLESKYGPPCPPCPAPPVAG | |
| | PSVFLFPPKPKDTLMISRTPEVTCVVVDVSQEDPEVQFNWYVDGVEVHNA | |
| | KTKPREEQFQSTYRVVSVLTVLHQDWLNGKEYKCKVSNKGLPSSIEKTIS | |
| | KAKGQPREPQVYTLPPSQEEMTKNQVSLTCLVKGFYPSDIAVEWESNGQP | |
| | ENNYKTTPPVLDSDGSFFLYSRLTVDKSRWQEGNVFSCSVMHEALHNHYT | |
| | QKSLSLSLGKMFWVLVVVGGVLACYSLLVTVAFIIFWVKRGRKKLLYIFK | |
| | QPFMRPVQTTQEEDGCSCRFPEEEEGGCELRVKFSRSADAPAYQQGQNQL | |
| | YNELNLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| 36 | QSALTQPASVSASPGQSIAISCTGTSSDVGWYQQHPGKAPKLMIYEDSKR | CAR 9 (aa) |
| | PSGVSNRFSGSKSGNTASLTISGLQAEDEADYYCSSNTRSSTLVFGGGTK | |
| | LTVLEAAAKQVQLVESGGGLVHPGGSLRLSCAASGFTFRSHSMNWVRQAP | |
| | GKGLEWVSSISSDSTYTYYADSVKGRFTISRDNAKNSLYLQMNSLRAEDT | |
| | AVYYCARSGGQWKYYDYWGQGTLVTVSSGSTSGSGKPGSGEGSTKGSSEL | |
| | TQDPAVSVALGQTVRITCQGDSLRSYYASWYQQKPGQAPVLVIYGKNNRP | |
| | SGIPDRFSGSSSGNTASLTITGAQAEDEADYYCNSRDSSGNPPVVFGGGT | |
| | KLTVLEAAAKEVQLVQSGAEMKKPGASLKLSCKASGYTFIDYYVYWMRQA | |
| | PGQGLESMGWINPNSGGTNYAQKFQGRVTMTRDTSISTAYMELSRLRSDD | |
| | TAMYYCARSQRDGYMDYWGQGTLVTVSSESKYGPPCPPCPAPPVAGPSVF | |
| | LFPPKPKDTLMISRTPEVTCVVVDVSQEDPEVQFNWYVDGVEVHNAKTKP | |
| | REEQFQSTYRVVSVLTVLHQDWLNGKEYKCKVSNKGLPSSIEKTISKAKG | |
| | QPREPQVYTLPPSQEEMTKNQVSLTCLVKGFYPSDIAVEWESNGQPENNY | |
| | KTTPPVLDSDGSFFLYSRLTVDKSRWQEGNVFSCSVMHEALHNHYTQKSL | |
| | SLSLGKMFWVLVVVGGVLACYSLLVTVAFIIFWVKRGRKKLLYIFKQPFM | |
| | RPVQTTQEEDGCSCRFPEEEEGGCELRVKFSRSADAPAYQQGQNQLYNEL | |
| | NLGRREEYDVLDKRRGRDPEMGGKPRRKNPQEGLYNELQKDKMAEAYSEI | |
| | GMKGERRRGKGHDGLYQGLSTATKDTYDALHMQALPPR | |
| 37 | QVQLVESGGGLVHPGGSLRLSCAASGFTFRSHSMNWVRQAPGKGLEWVSS | CAR 5 (aa) |
| | ISSDSTYTYYADSVKGRFTISRDNAKNSLYLQMNSLRAEDTAVYYCARSG | |
| | GQWKYYDYWGQGTLVTVSSGGGGSQSALTQPASVSASPGQSIAISCTGTS | |
| | SDVGWYQQHPGKAPKLMIYEDSKRPSGVSNRFSGSKSGNTASLTISGLQA | |
| | EDEADYYCSSNTRSSTLVFGGGTKLTVLGSRGGGGSGGGGSGGGGSLEMA | |
| | EVQLVQSGAEMKKPGASLKLSCKASGYTFIDYYVYWMRQAPGQGLESMGW | |
| | INPNSGGTNYAQKFQGRVTMTRDTSISTAYMELSRLRSDDTAMYYCARSQ | |
| | RDGYMDYWGQGTLVTVSSGGGGSSSELTQDPAVSVALGQTVRITCQGDSL | |
| | RSYYASWYQQKPGQAPVLVIYGKNNRPSGIPDRFSGSSSGNTASLTITGA | |
| | QAEDEADYYCNSRDSSGNPPVVFGGGTKLTVLESKYGPPCPPCPAPPVAG | |
| | PSVFLFPPKPKDTLMISRTPEVTCVVVDVSQEDPEVQFNWYVDGVEVHNA | |
| | KTKPREEQFQSTYRVVSVLTVLHQDWLNGKEYKCKVSNKGLPSSIEKTIS | |
| | KAKGQPREPQVYTLPPSQEEMTKNQVSLTCLVKGFYPSDIAVEWESNGQP | |
| | ENNYKTTPPVLDSDGSFFLYSRLTVDKSRWQEGNVFSCSVMHEALHNHYT | |
| | QKSLSLSLGKMFWVLVVVGGVLACYSLLVTVAFIIFWVKRGRKKLLYIFK | |
| | QPFMRPVQTTQEEDGCSCRFPEEEEGGCELRVKFSRSADAPAYQQGQNQL | |
| | YNELNLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| 38 | SSELTQDPAVSVALGQTVRITCQGDSLRSYYASWYQQKPGQAPVLVIYGK | CAR 6 (aa) |
| | NNRPSGIPDRFSGSSSGNTASLTITGAQAEDEADYYCNSRDSSGNPPVVF | |
| | GGGTKLTVLGGGGSEVQLVQSGAEMKKPGASLKLSCKASGYTFIDYYVYW | |
| | MRQAPGQGLESMGWINPNSGGTNYAQKFQGRVTMTRDTSISTAYMELSRL | |
| | RSDDTAMYYCARSQRDGYMDYWGQGTLVTVSSGSRGGGGSGGGGSGGGGS | |
| | LEMAQSALTQPASVSASPGQSIAISCTGTSSDVGWYQQHPGKAPKLMIYE | |
| | DSKRPSGVSNRFSGSKSGNTASLTISGLQAEDEADYYCSSNTRSSTLVFG | |
| | GGTKLTVLGGGGSQVQLVESGGGLVHPGGSLRLSCAASGFTFRSHSMNWV | |
| | RQAPGKGLEWVSSISSDSTYTYYADSVKGRFTISRDNAKNSLYLQMNSLR | |
| | AEDTAVYYCARSGGQWKYYDYWGQGTLVTVSSESKYGPPCPPCPAPPVAG | |
| | PSVFLFPPKPKDTLMISRTPEVTCVVVDVSQEDPEVQFNWYVDGVEVHNA | |
| | KTKPREEQFQSTYRVVSVLTVLHQDWLNGKEYKCKVSNKGLPSSIEKTIS | |
| | KAKGQPREPQVYTLPPSQEEMTKNQVSLTCLVKGFYPSDIAVEWESNGQP | |
| | ENNYKTTPPVLDSDGSFFLYSRLTVDKSRWQEGNVFSCSVMHEALHNHYT | |
| | QKSLSLSLGKMFWVLVVVGGVLACYSLLVTVAFIIFWVKRGRKKLLYIFK | |
| | QPFMRPVQTTQEEDGCSCRFPEEEEGGCELRVKFSRSADAPAYQQGQNQL | |
| | YNELNLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| 39 | QSALTQPASVSASPGQSIAISCTGTSSDVGWYQQHPGKAPKLMIYEDSKR | CAR 10 (aa) |
| | PSGVSNRFSGSKSGNTASLTISGLQAEDEADYYCSSNTRSSTLVFGGGTK | |
| | LTVLGGGGSQVQLVESGGGLVHPGGSLRLSCAASGFTFRSHSMNWVRQAP | |
| | GKGLEWVSSISSDSTYTYYADSVKGRFTISRDNAKNSLYLQMNSLRAEDT | |
| | AVYYCARSGGQWKYYDYWGQGTLVTVSSGSRGGGGSGGGGSGGGGSLEMA | |
| | SSELTQDPAVSVALGQTVRITCQGDSLRSYYASWYQQKPGQAPVLVIYGK | |
| | NNRPSGIPDRFSGSSSGNTASLTITGAQAEDEADYYCNSRDSSGNPPVVF | |
| | GGGTKLTVLGGGGSEVQLVQSGAEMKKPGASLKLSCKASGYTFIDYYVYW | |
| | MRQAPGQGLESMGWINPNSGGTNYAQKFQGRVTMTRDTSISTAYMELSRL | |
| | RSDDTAMYYCARSQRDGYMDYWGQGTLVTVSSESKYGPPCPPCPAPPVAG | |
| | PSVFLFPPKPKDTLMISRTPEVTCVVVDVSQEDPEVQFNWYVDGVEVHNA | |
| | KTKPREEQFQSTYRVVSVLTVLHQDWLNGKEYKCKVSNKGLPSSIEKTIS | |
| | KAKGQPREPQVYTLPPSQEEMTKNQVSLTCLVKGFYPSDIAVEWESNGQP | |
| | ENNYKTTPPVLDSDGSFFLYSRLTVDKSRWQEGNVFSCSVMHEALHNHYT | |
| | QKSLSLSLGKMFWVLVVVGGVLACYSLLVTVAFIIFWVKRGRKKLLYIFK | |
| | QPFMRPVQTTQEEDGCSCRFPEEEEGGCELRVKFSRSADAPAYQQGQNQL | |
| | YNELNLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| 40 | QSALTQPASVSASPGQSIAISCTGTSSDVGWYQQHPGKAPKLMIYEDSKR | CAR 11 (aa) |
| | PSGVSNRFSGSKSGNTASLTISGLQAEDEADYYCSSNTRSSTLVFGGGTK | |
| | LTVLGGGGSSSELTQDPAVSVALGQTVRITCQGDSLRSYYASWYQQKPGQ | |
| | APVLVIYGKNNRPSGIPDRFSGSSSGNTASLTITGAQAEDEADYYCNSRD | |
| | SSGNPPVVFGGGTKLTVLGSRGGGGSGGGGSGGGGSLEMAQVQLVESGGG | |
| | LVHPGGSLRLSCAASGFTFRSHSMNWVRQAPGKGLEWVSSISSDSTYTYY | |
| | ADSVKGRFTISRDNAKNSLYLQMNSLRAEDTAVYYCARSGGQWKYYDYWG | |
| | QGTLVTVSSGGGGSEVQLVQSGAEMKKPGASLKLSCKASGYTFIDYYVYW | |
| | MRQAPGQGLESMGWINPNSGGTNYAQKFQGRVTMTRDTSISTAYMELSRL | |
| | RSDDTAMYYCARSQRDGYMDYWGQGTLVTVSSESKYGPPCPPCPAPPVAG | |
| | PSVFLFPPKPKDTLMISRTPEVTCVVVDVSQEDPEVQFNWYVDGVEVHNA | |
| | KTKPREEQFQSTYRVVSVLTVLHQDWLNGKEYKCKVSNKGLPSSIEKTIS | |
| | KAKGQPREPQVYTLPPSQEEMTKNQVSLTCLVKGFYPSDIAVEWESNGQP | |
| | ENNYKTTPPVLDSDGSFFLYSRLTVDKSRWQEGNVFSCSVMHEALHNHYT | |
| | QKSLSLSLGKMFWVLVVVGGVLACYSLLVTVAFIIFWVKRGRKKLLYIFK | |
| | QPFMRPVQTTQEEDGCSCRFPEEEEGGCELRVKESRSADAPAYQQGQNQL | |
| | YNELNLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| 41 | QVQLVESGGGLVHPGGSLRLSCAASGFTFRSHSMNWVRQAPGKGLEWVSS | CAR 7 (aa) |
| | ISSDSTYTYYADSVKGRFTISRDNAKNSLYLQMNSLRAEDTAVYYCARSG | |
| | GQWKYYDYWGQGTLVTVSSGGGGSGGGGSQSALTQPASVSASPGQSIAIS | |
| | CTGTSSDVGWYQQHPGKAPKLMIYEDSKRPSGVSNRFSGSKSGNTASLTI | |
| | SGLQAEDEADYYCSSNTRSSTLVFGGGTKLTVLGSRGGGGSGGGGSGGGG | |
| | SLEMAEVQLVQSGAEMKKPGASLKLSCKASGYTFIDYYVYWMRQAPGQGL | |
| | ESMGWINPNSGGTNYAQKFQGRVTMTRDTSISTAYMELSRLRSDDTAMYY | |
| | CARSQRDGYMDYWGQGTLVTVSSGGGGSGGGGSSSELTQDPAVSVALGQT | |
| | VRITCQGDSLRSYYASWYQQKPGQAPVLVIYGKNNRPSGIPDRFSGSSSG | |
| | NTASLTITGAQAEDEADYYCNSRDSSGNPPVVFGGGTKLTVLESKYGPPC | |
| | PPCPAPPVAGPSVFLFPPKPKDTLMISRTPEVTCVVVDVSQEDPEVQFNW | |
| | YVDGVEVHNAKTKPREEQFQSTYRVVSVLTVLHQDWLNGKEYKCKVSNKG | |
| | LPSSIEKTISKAKGQPREPQVYTLPPSQEEMTKNQVSLTCLVKGFYPSDI | |
| | AVEWESNGQPENNYKTTPPVLDSDGSFFLYSRLTVDKSRWQEGNVFSCSV | |
| | MHEALHNHYTQKSLSLSLGKMFWVLVVVGGVLACYSLLVTVAFIIFWVKR | |
| | GRKKLLYIFKQPFMRPVQTTQEEDGCSCRFPEEEEGGCELRVKFSRSADA | |
| | PAYQQGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHMQALP | |
| | PR | |
| 42 | EVQLVQSGAEMKKPGASLKLSCKASGYTFIDYYVYWMRQAPGQGLESMGW | CAR 12 (aa) |
| | INPNSGGTNYAQKFQGRVTMTRDTSISTAYMELSRLRSDDTAMYYCARSQ | |
| | RDGYMDYWGQGTLVTVSSGGGGSGGGGSQVQLVESGGGLVHPGGSLRLSC | |
| | AASGFTFRSHSMNWVRQAPGKGLEWVSSISSDSTYTYYADSVKGRFTISR | |
| | DNAKNSLYLQMNSLRAEDTAVYYCARSGGQWKYYDYWGQGTLVTVSSGSR | |
| | GGGGSGGGGSGGGGSLEMASSELTQDPAVSVALGQTVRITCQGDSLRSYY | |
| | ASWYQQKPGQAPVLVIYGKNNRPSGIPDRFSGSSSGNTASLTITGAQAED | |
| | EADYYCNSRDSSGNPPVVFGGGTKLTVLGGGGSGGGGSQSALTQPASVSA | |
| | SPGQSIAISCTGTSSDVGWYQQHPGKAPKLMIYEDSKRPSGVSNRFSGSK | |
| | SGNTASLTISGLQAEDEADYYCSSNTRSSTLVFGGGTKLTVLESKYGPPC | |
| | PPCPAPPVAGPSVFLFPPKPKDTLMISRTPEVTCVVVDVSQEDPEVQFNW | |
| | YVDGVEVHNAKTKPREEQFQSTYRVVSVLTVLHQDWLNGKEYKCKVSNKG | |
| | LPSSIEKTISKAKGQPREPQVYTLPPSQEEMTKNQVSLTCLVKGFYPSDI | |
| | AVEWESNGQPENNYKTTPPVLDSDGSFFLYSRLTVDKSRWQEGNVFSCSV | |
| | MHEALHNHYTQKSLSLSLGKMFWVLVVVGGVLACYSLLVTVAFIIFWVKR | |
| | GRKKLLYIFKQPFMRPVQTTQEEDGCSCRFPEEEEGGCELRVKFSRSADA | |
| | PAYQQGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHMQALP | |
| | PR | |
| 43 | QSALTQPASVSASPGQSIAISCTGTSSDVGWYQQHPGKAPKLMIYEDSKR | CAR 13 (aa) |
| | PSGVSNRFSGSKSGNTASLTISGLQAEDEADYYCSSNTRSSTLVFGGGTK | |
| | LTVLGGGGSGGGGSQVQLVESGGGLVHPGGSLRLSCAASGFTFRSHSMNW | |
| | VRQAPGKGLEWVSSISSDSTYTYYADSVKGRFTISRDNAKNSLYLQMNSL | |
| | RAEDTAVYYCARSGGQWKYYDYWGQGTLVTVSSGSRGGGGSGGGGSGGGG | |
| | SLEMASSELTQDPAVSVALGQTVRITCQGDSLRSYYASWYQQKPGQAPVL | |
| | VIYGKNNRPSGIPDRFSGSSSGNTASLTITGAQAEDEADYYCNSRDSSGN | |
| | PPVVFGGGTKLTVLGGGGSGGGGSEVQLVQSGAEMKKPGASLKLSCKASG | |
| | YTFIDYYVYWMRQAPGQGLESMGWINPNSGGTNYAQKFQGRVTMTRDTSI | |
| | STAYMELSRLRSDDTAMYYCARSQRDGYMDYWGQGTLVTVSSESKYGPPC | |
| | PPCPAPPVAGPSVFLFPPKPKDTLMISRTPEVTCVVVDVSQEDPEVQFNW | |
| | YVDGVEVHNAKTKPREEQFQSTYRVVSVLTVLHQDWLNGKEYKCKVSNKG | |
| | LPSSIEKTISKAKGQPREPQVYTLPPSQEEMTKNQVSLTCLVKGFYPSDI | |
| | AVEWESNGQPENNYKTTPPVLDSDGSFFLYSRLTVDKSRWQEGNVFSCSV | |
| | MHEALHNHYTQKSLSLSLGKMFWVLVVVGGVLACYSLLVTVAFIIFWVKR | |
| | GRKKLLYIFKQPFMRPVQTTQEEDGCSCRFPEEEEGGCELRVKFSRSADA | |
| | PAYQQGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHMQALP | |
| | PR | |
| 44 | QSALTQPASVSASPGQSIAISCTGTSSDVGWYQQHPGKAPKLMIYEDSKR | CAR 14 (aa) |
| | PSGVSNRFSGSKSGNTASLTISGLQAEDEADYYCSSNTRSSTLVFGGGTK | |
| | LTVLGGGGSGGGGSQVQLVESGGGLVHPGGSLRLSCAASGFTFRSHSMNW | |
| | VRQAPGKGLEWVSSISSDSTYTYYADSVKGRFTISRDNAKNSLYLQMNSL | |
| | RAEDTAVYYCARSGGQWKYYDYWGQGTLVTVSSGSTSGSGKPGSGEGSTK | |
| | GSSELTQDPAVSVALGQTVRITCQGDSLRSYYASWYQQKPGQAPVLVIYG | |
| | KNNRPSGIPDRFSGSSSGNTASLTITGAQAEDEADYYCNSRDSSGNPPVV | |
| | FGGGTKLTVLGGGGSGGGGSEVQLVQSGAEMKKPGASLKLSCKASGYTFI | |
| | DYYVYWMRQAPGQGLESMGWINPNSGGTNYAQKFQGRVTMTRDTSISTAY | |
| | MELSRLRSDDTAMYYCARSQRDGYMDYWGQGTLVTVSSESKYGPPCPPCP | |
| | APPVAGPSVFLFPPKPKDTLMISRTPEVTCVVVDVSQEDPEVQFNWYVDG | |
| | VEVHNAKTKPREEQFQSTYRVVSVLTVLHQDWLNGKEYKCKVSNKGLPSS | |
| | IEKTISKAKGQPREPQVYTLPPSQEEMTKNQVSLTCLVKGFYPSDIAVEW | |
| | ESNGQPENNYKTTPPVLDSDGSFFLYSRLTVDKSRWQEGNVFSCSVMHEA | |
| | LHNHYTQKSLSLSLGKMFWVLVVVGGVLACYSLLVTVAFIIFWVKRGRKK | |
| | LLYIFKQPFMRPVQTTQEEDGCSCRFPEEEEGGCELRVKFSRSADAPAYQ | |
| | QGQNQLYNELNLGRREEYDVLDKRRGRDPEMGGKPRRKNPQEGLYNELQK | |
| | DKMAEAYSEIGMKGERRRGKGHDGLYQGLSTATKDTYDALHMQALPPR | |
| 45 | QVQLVESGGGLVHPGGSLRLSCAASGFTFRSHSMNWVRQAPGKGLEWVSS | GPRC5D |
| | ISSDSTYTYYADSVKGRFTISRDNAKNSLYLQMNSLRAEDTAVYYCARSG | scFv |
| | GQWKYYDYWGQGTLVTVSSGSRGGGGSGGGGSGGGGSLEMASSELTQDPA | |
| | VSVALGQTVRITCQGDSLRSYYASWYQQKPGQAPVLVIYGKNNRPSGIPD | |
| | RFSGSSSGNTASLTITGAQAEDEADYYCNSRDSSGNPPVVFGGGTKLTVL | |
| 46 | SSELTQDPAVSVALGQTVRITCQGDSLRSYYASWYQQKPGQAPVLVIYGK | GPRC5D |
| | NNRPSGIPDRFSGSSSGNTASLTITGAQAEDEADYYCNSRDSSGNPPVVF | scFv |
| | GGGTKLTVLGSRGGGGSGGGGSGGGGSLEMAQVQLVESGGGLVHPGGSLR | |
| | LSCAASGFTFRSHSMNWVRQAPGKGLEWVSSISSDSTYTYYADSVKGRFT | |
| | ISRDNAKNSLYLQMNSLRAEDTAVYYCARSGGQWKYYDYWGQGTLVTVSS | |
| 47 | QSALTQPASVSASPGQSIAISCTGTSSDVGWYQQHPGKAPKLMIYEDSKR | BCMAscFv |
| | PSGVSNRFSGSKSGNTASLTISGLQAEDEADYYCSSNTRSSTLVFGGGTK | |
| | LTVLGSRGGGGSGGGGSGGGGSLEMAEVQLVQSGAEMKKPGASLKLSCKA | |
| | SGYTFIDYYVYWMRQAPGQGLESMGWINPNSGGTNYAQKFQGRVTMTRDT | |
| | SISTAYMELSRLRSDDTAMYYCARSQRDGYMDYWGQGTLVTVSS | |
| 48 | EVQLVQSGAEMKKPGASLKLSCKASGYTFIDYYVYWMRQAPGQGLESMGW | BCMA scFv |
| | INPNSGGTNYAQKFQGRVTMTRDTSISTAYMELSRLRSDDTAMYYCARSQ | |
| | RDGYMDYWGQGTLVTVSSGSRGGGGSGGGGSGGGGSLEMAQSALTQPASV | |
| | SASPGQSIAISCTGTSSDVGWYQQHPGKAPKLMIYEDSKRPSGVSNRFSG | |
| | SKSGNTASLTISGLQAEDEADYYCSSNTRSSTLVFGGGTKLTVL | |
| 49 | gagtctaaatacggaccgccttgtcctccttgtccagctcctcctgttgc | IgG4/IgG2 |
| | cggaccttccgtgttcctgtttcctccaaagcctaaggacaccctgatga | hinge- |
| | tcagcaggacccctgaagtgacctgcgtggtggtggatgtgtcccaagag | IgG2/IgG4 |
| | gatcccgaggtgcagttcaattggtacgtggacggcgtggaagtgcacaa | CH2- IgG4 |
| | cgccaagaccaagcctagagaggaacagttccagagcacctacagagtgg | CH3 spacer |
| | tgtccgtgctgacagtgctgcaccaggattggctgaacggcaaagagtac | (nt) |
| | aagtgcaaggtgtccaacaagggcctgcctagcagcatcgagaaaaccat | |
| | ctccaaggccaagggccagccaagagagccccaggtttacacactgcctc | |
| | caagccaagaggaaatgaccaagaatcaggtgtccctgacatgcctggtc | |
| | aagggcttctacccctccgatatcgccgtggaatgggagagcaatggcca | |
| | gcctgagaacaactacaagaccacacctcctgtgctggacagcgacggca | |
| | gtttcttcctgtatagtagactcaccgtggataaatcaagatggcaagag | |
| | ggcaacgtgttcagctgcagcgtgatgcacgaggccctgcacaaccacta | |
| | cacccagaaaagcctgagcctgtctctgggcaaa | |
| 50 | gaatctaagtacggaccgccttgtcctccttgtcccgctcctcctgttgc | IgG4/IgG2 |
| | cggaccttccgtgttcctgtttcctccaaagcctaaggacaccctgatga | hinge- |
| | tcagcaggacccctgaagtgacctgcgtggtggtggatgtgtcccaagag | IgG2/IgG4 |
| | gatcccgaggtgcagttcaactggtatgtggacggcgtggaagtgcacaa | CH2- IgG4 |
| | cgccaagaccaagcctagagaggaacagttccagagcacctacagagtgg | CH3 spacer |
| | tgtccgtgctgacagtgctgcaccaggattggctgaacggcaaagagtac | (nt) |
| | aagtgcaaggtgtccaacaagggcctgcctagcagcatcgagaaaaccat | |
| | ctccaaggccaagggccagccaagagagccccaggtttacacactgcctc | |
| | caagccaagaggaaatgaccaagaatcaggtgtccctgacatgcctggtc | |
| | aagggcttctacccctccgatatcgccgtggaatgggagagcaatggcca | |
| | gcctgagaacaactacaagaccacacctcctgtgctggacagcgacggca | |
| | gtttcttcctgtatagtagactcaccgtggataaatcaagatggcaagag | |
| | ggcaacgtgttcagctgcagcgtgatgcacgaggccctgcacaaccacta | |
| | cacccagaaaagcctgagcctgtctctgggcaag | |
| 51 | gaatctaagtacggaccgccctgccccccttgccct | Spacer |
| | | (IgG4hinge) |
| | | (nt) |
| 52 | ESKYGPPCPPCPAPEFLGGPSVFLFPPKPKDTLMISRTPEVTCVVVDVSQ | Hinge-CH2- |
| | EDPEVQFNWYVDGVEVHNAKTKPREEQFNSTYRVVSVLTVLHQDWLNGKE | CH3 spacer |
| | YKCKVSNKGLPSSIEKTISKAKGQPREPQVYTLPPSQEEMTKNQVSLTCL | (aa) |
| | VKGFYPSDIAVEWESNGQPENNYKTTPPVLDSDGSFFLYSRLTVDKSRWQ | |
| | EGNVFSCSVMHEALHNHYTQKSLSLSLGK | |
| 53 | attgaagttatgtatcctcctccttacctagacaatgagaagagcaatgg | CD28 |
| | aaccattatccatgtgaaagggaaacacctttgtccaagtcccctatttc | ectodomain |
| | ccggaccttctaagccc | spacer (nt) |
| 54 | IEVMYPPPYLDNEKSNGTIIHVKGKHLCPSPLFPGPSKP | CD28 |
| | | ectodomain |
| | | spacer (aa) |
| 55 | agagtcaagttttccaggtccgccgacgctccagcctaccagcaggggca | CD3-zeta |
| | gaaccagctgtacaacgagctgaacctgggcagaagggaagagtacgacg | derived |
| | tcctggataagcggagaggccgggaccctgagatgggcggcaagcctcgg | intracellular |
| | cggaagaacccccaggaaggcctgtataacgaactgcagaaagacaagat | signaling |
| | ggccgaggcctacagcgagatcggcatgaagggcgagcggaggcggggca | domain (nt) |
| | agggccacgacggcctgtatcagggcctgtccaccgccaccaaggatacc | |
| | tacgacgccctgcacatgcaggccctgcccccaagg | |
| 56 | agagtgaagttcagcagatccgccgacgctccagcctatcagcagggcca | CD3-zeta |
| | aaaccagctgtacaacgagctgaacctggggagaagagaagagtacgacg | derived |
| | tgctggataagcggagaggcagagatcctgaaatgggcggcaagcccaga | intracellular |
| | cggaagaatcctcaagagggcctgtataatgagctgcagaaagacaagat | signaling |
| | ggccgaggcctacagcgagatcggaatgaagggcgagcgcagaagaggca | domain (nt) |
| | agggacacgatggactgtaccagggcctgagcaccgccaccaaggatacc | |
| | tatgacgcactgcacatgcaggccctgccacctaga | |
| 57 | aagcgggggagaaagaaactgctgtatattttcaaacagccctttatgag | 4-1BB- |
| | acctgtgcagactacccaggaggaagacggatgcagctgtaggtttcccg | derived |
| | aggaagaggaaggaggctgtgagctg | intracellular |
| | | co-signaling |
| | | sequence (nt) |
| 58 | aagcggggcagaaagaagctgctctacatcttcaagcagcccttcatgcg | 4-1BB- |
| | gcccgtgcagaccacacaagaggaagatggctgctcctgcagattccccg | derived |
| | aggaagaagaaggcggctgcgagctg | intracellular |
| | | co-signaling |
| | | sequence (nt) |
| 59 | MYKDCIESTGDYFLLCDAEGPWGIILESLAILGIVVTILLLLAFLFLMRK | GPRC5D |
| | IQDCSQWNVLPTQLLFLLSVLGLFGLAFAFIIELNQQTAPVRYFLFGVLF | protein |
| | ALCFSCLLAHASNLVKLVRGCVSFSWTTILCIAIGCSLLQIIIATEYVTL | (Uniprot |
| | IMTRGMMFVNMTPCQLNVDFVVLLVYVLFLMALTFFVSKATFCGPCENWK | Q9NZD1) |
| | QHGRLIFITVLFSIIIWVVWISMLLRGNPQFQRQPQWDDPVVCIALVTNA | |
| | WVFLLLYIVPELCILYRSCRQECPLQGNACPVTAYQHSFQVENQELSRAR | |
| | DSDGAEEDVALTSYGTPIQPQTVDPTQECFIPQAKLSPQQDAGGV | |
| 60 | MLQMAGQCSQNEYFDSLLHACIPCQLRCSSNTPPLTCQRYCNASVTNSVK | BCMA |
| | GTNAILWTCLGLSLIISLAVFVLMFLLRKINSEPLKDEFKNTGSGLLGMA | protein |
| | NIDLEKSRTGDEIILPRGLEYTVEECTCEDCIKSKPKVDSDHCFPLPAME | (Uniprot |
| | EGATILVTTKTNDYCKSLPAALSATEIEKSISAR | Q02223) |
| 61 | ggatctgcgatcgctccggtgcccgtcagtgggcagagcgcacatcgccc | EF1alpha |
| | acagtccccgagaagttggggggaggggtcggcaattgaaccggtgccta | promoter with |
| | gagaaggtggcgcggggtaaactgggaaagtgatgtcgtgtactggctcc | HTLV1 |
| | gcctttttcccgagggtgggggagaaccgtatataagtgcagtagtcgcc | ehancer |
| | gtgaacgttctttttcgcaacgggtttgccgccagaacacagctgaagct | |
| | tcgaggggctcgcatctctccttcacgcgcccgccgccctacctgaggcc | |
| | gccatccacgccggttgagtcgcgttctgccgcctcccgcctgtggtgcc | |
| | tcctgaactgcgtccgccgtctaggtaagtttaaagctcaggtcgagacc | |
| | gggcctttgtccggcgctcccttggagcctacctagactcagccggctct | |
| | ccacgctttgcctgaccctgcttgctcaactctacgtctttgtttcgttt | |
| | tctgttctgcgccgttacagatccaagctgtgaccggcgcctac | |
| 62 | aatcaacctctggattacaaaatttgtgaaagattgactggtattcttaa | Woodchuck |
| | ctatgttgctccttttacgctatgtggatacgctgctttaatgcctttgt | Hepatitis |
| | atcatgctattgcttcccgtatggctttcattttctcctccttgtataaa | Virus (WHP) |
| | tcctggttgctgtctctttatgaggagttgtggcccgttgtcaggcaacg | Posttranscript |
| | tggcgtggtgtgcactgtgtttgctgacgcaacccccactggttggggca | ional |
| | ttgccaccacctgtcagctcctttccgggactttcgctttccccctccct | Regulatory |
| | attgccacggcggaactcatcgccgcctgccttgcccgctgctggacagg | Element |
| | ggctcggctgttgggcactgacaattccgtggtgttgtcggggaaatcat | (WPRE) |
| | cgtcctttccttggctgctcgcctgtgttgccacctggattctgcgcggg | |
| | acgtccttctgctacgtcccttcggccctcaatccagcggaccttccttc | |
| | ccgcggcctgctgccggctctgcggcctcttccgcgtcttcgccttcgcc | |
| | ctcagacgagtcggatctccctttgggccgcctccccgc | |
| 63 | VKQTLNFDLLKLAGDVESNPGP | F2A peptide |
| | | (aa) |
| 64 | GSGVKQTLNFDLLKLAGDVESNPGP | F2A peptide |
| | | (aa) |
| 65 | QCTNYALLKLAGDVESNPGP | E2A peptide |
| | | (aa) |
| 66 | GSGQCTNYALLKLAGDVESNPGP | E2A peptide |
| | | (aa) |
| 67 | EGRGSLLTCGDVEENPGP | T2A peptide |
| | | (aa) |
| 68 | GSGEGRGSLLTCGDVEENPGP | T2A peptide |
| | | (aa) |
| 69 | LEGGGEGRGSLLTCGDVEENPGPR | T2A peptide |
| | | (aa) |
| 70 | ATNFSLLKQAGDVEENPGP | P2A peptide |
| | | (aa) |
| 71 | GSGATNFSLLKQAGDVEENPGP | P2A peptide |
| | | (aa) |
| 72 | MPLLLLLPLLWAGALA | CD33 signal |
| | | sequence |
| 73 | gaatctaagtacggaccgccctgccctccctgccctgctcctcctgtggc | IgG4/IgG2 |
| | tggaccaagcgtgttcctgtttccacctaagcctaaagataccctgatga | hinge- |
| | tttcccgcacacctgaagtgacttgcgtggtcgtggacgtgagccaggag | IgG2/IgG4 |
| | gatccagaagtgcagttcaactggtacgtggacggcgtggaagtccacaa | CH2- IgG4 |
| | tgctaagactaaaccccgagaggaacagtttcagtcaacttaccgggtcg | CH3 spacer |
| | tgagcgtgctgaccgtcctgcatcaggattggctgaacgggaaggagtat | (nt) |
| | aagtgcaaagtgtctaataagggactgcctagctccatcgagaaaacaat | |
| | tagtaaggcaaaagggcagcctcgagaaccacaggtgtataccctgcccc | |
| | ctagccaggaggaaatgaccaagaaccaggtgtccctgacatgtctggtc | |
| | aaaggcttctatccaagtacatcgccgtggagtgggaatcaaatgggcag | |
| | cccgagaacaattacaagaccacaccacccgtgctggactctgatggaag | |
| | tttctttctgtattccaggctgaccgtggataaatctcgctggcaggagg | |
| | gcaacgtgttctcttgcagtgtcatgcacgaagccctgcacaatcattat | |
| | acacagaagtcactgagcctgtccctgggcaaa | |
| 74 | gagtctaaatacggaccgccttgtcctccttgtcccgctcctcctgttgc | IgG4/IgG2 |
| | cggaccttccgtgttcctgtttcctccaaagcctaaggacaccctgatga | hinge- |
| | tcagcaggacccctgaagtgacctgcgtggtggtggatgtgtcccaagag | IgG2/IgG4 |
| | gatcccgaggtgcagttcaactggtatgtggacggcgtggaagtgcacaa | CH2- IgG4 |
| | cgccaagaccaagcctagagaggaacagttccagagcacctacagagtgg | CH3 spacer |
| | tgtccgtgctgacagtgctgcaccaggattggctgaacggcaaagagtac | (nt) |
| | aagtgcaaggtgtccaacaagggcctgcctagcagcatcgagaaaaccat | |
| | ctccaaggccaagggccagccaagagagccccaggtttacacactgcctc | |
| | caagccaagaggaaatgaccaagaatcaggtgtccctgacatgcctggtc | |
| | aagggcttctacccctccgatatcgccgtggaatgggagagcaatggcca | |
| | gcctgagaacaactacaagaccacacctcctgtgctggacagcgacggca | |
| | gtttcttcctgtatagtagactcaccgtggataaatcaagatggcaagag | |
| | ggcaacgtgttcagctgcagcgtgatgcacgaggccctgcacaaccacta | |
| | cacccagaaaagcctgagcctgtctctgggcaag | |
| 75 | ASTKGPSVFPLAPCSRSTSESTAALGCLVKDYFPEPVTVSWNSGALTSGV | Human IgG4 |
| | HTFPAVLQSSGLYSLSSVVTVPSSSLGTKTYTCNVDHKPSNTKVDKRVES | Fc (Uniprot |
| | KYGPPCPSCPAPEFLGGPSVFLFPPKPKDTLMISRTPEVTCVVVDVSQED | P01861) |
| | PEVQFNWYVDGVEVHNAKTKPREEQFNSTYRVVSVLTVLHQDWLNGKEYK | |
| | CKVSNKGLPSSIEKTISKAKGQPREPQVYTLPPSQEEMTKNQVSLTCLVK | |
| | GFYPSDIAVEWESNGQPENNYKTTPPVLDSDGSFFLYSRLTVDKSRWQEG | |
| | NVFSCSVMHEALHNHYTQKSLSLSLGK | |
| 76 | ASTKGPSVFPLAPCSRSTSESTAALGCLVKDYFPEPVTVSWNSGALTSGV | Human IgG2 |
| | HTFPAVLQSSGLYSLSSVVTVPSSNFGTQTYTCNVDHKPSNTKVDKTVER | Fc (Uniprot |
| | KCCVECPPCPAPPVAGPSVFLFPPKPKDTLMISRTPEVTCVVVDVSHEDP | P01859) |
| | EVQFNWYVDGVEVHNAKTKPREEQFNSTFRVVSVLTVVHQDWLNGKEYKC | |
| | KVSNKGLPAPIEKTISKTKGQPREPQVYTLPPSREEMTKNQVSLTCLVKG | |
| | FYPSDISVEWESNGQPENNYKTTPPMLDSDGSFFLYSKLTVDKSRWQQGN | |
| | VFSCSVMHEALHNHYTQKSLSLSPGK | |
| 77 | SSELTQDPAVSVALGQTVRITCQGDSLRSYYASWYQQKPGQAPVLVIYGK | extracellular |
| | NNRPSGIPDRFSGSSSGNTASLTITGAQAEDEADYYCNSRDSSGNPPVVF | binding |
| | GGGTKLTVLGSRGGGGSGGGGSGGGGSLEMAQVQLVESGGGLVHPGGSLR | domain |
| | LSCAASGFTFRSHSMNWVRQAPGKGLEWVSSISSDSTYTYYADSVKGRFT | |
| | ISRDNAKNSLYLQMNSLRAEDTAVYYCARSGGQWKYYDYWGQGTLVTVSS | |
| | EAAAKEVQLVQSGAEMKKPGASLKLSCKASGYTFIDYYVYWMRQAPGQGL | |
| | ESMGWINPNSGGTNYAQKFQGRVTMTRDTSISTAYMELSRLRSDDTAMYY | |
| | CARSQRDGYMDYWGQGTLVTVSSGSRGGGGSGGGGSGGGGSLEMAQSALT | |
| | QPASVSASPGQSIAISCTGTSSDVGWYQQHPGKAPKLMIYEDSKRPSGVS | |
| | NRFSGSKSGNTASLTISGLQAEDEADYYCSSNTRSSTLVFGGGTKLTVL | |
| 78 | QVQLVESGGGLVHPGGSLRLSCAASGFTFRSHSMNWVRQAPGKGLEWVSS | extracellular |
| | ISSDSTYTYYADSVKGRFTISRDNAKNSLYLQMNSLRAEDTAVYYCARSG | binding |
| | GQWKYYDYWGQGTLVTVSSGSRGGGGSGGGGSGGGGSLEMASSELTQDPA | domain |
| | VSVALGQTVRITCQGDSLRSYYASWYQQKPGQAPVLVIYGKNNRPSGIPD | |
| | RFSGSSSGNTASLTITGAQAEDEADYYCNSRDSSGNPPVVFGGGTKLTVL | |
| | GGGGSEVQLVQSGAEMKKPGASLKLSCKASGYTFIDYYVYWMRQAPGQGL | |
| | ESMGWINPNSGGTNYAQKFQGRVTMTRDTSISTAYMELSRLRSDDTAMYY | |
| | CARSQRDGYMDYWGQGTLVTVSSGSRGGGGSGGGGSGGGGSLEMAQSALT | |
| | QPASVSASPGQSIAISCTGTSSDVGWYQQHPGKAPKLMIYEDSKRPSGVS | |
| | NRFSGSKSGNTASLTISGLQAEDEADYYCSSNTRSSTLVFGGGTKLTVL | |
| 79 | SSELTQDPAVSVALGQTVRITCQGDSLRSYYASWYQQKPGQAPVLVIYGK | extracellular |
| | NNRPSGIPDRFSGSSSGNTASLTITGAQAEDEADYYCNSRDSSGNPPVVF | binding |
| | GGGTKLTVLGSRGGGGSGGGGSGGGGSLEMAQVQLVESGGGLVHPGGSLR | domain |
| | LSCAASGFTFRSHSMNWVRQAPGKGLEWVSSISSDSTYTYYADSVKGRFT | |
| | ISRDNAKNSLYLQMNSLRAEDTAVYYCARSGGQWKYYDYWGQGTLVTVSS | |
| | GGGGSGGGGSGGGGSGGGGSEVQLVQSGAEMKKPGASLKLSCKASGYTFI | |
| | DYYVYWMRQAPGQGLESMGWINPNSGGTNYAQKFQGRVTMTRDTSISTAY | |
| | MELSRLRSDDTAMYYCARSQRDGYMDYWGQGTLVTVSSGSRGGGGSGGGG | |
| | SGGGGSLEMAQSALTQPASVSASPGQSIAISCTGTSSDVGWYQQHPGKAP | |
| | KLMIYEDSKRPSGVSNRFSGSKSGNTASLTISGLQAEDEADYYCSSNTRS | |
| | STLVFGGGTKLTVL | |
| 80 | QVQLVESGGGLVHPGGSLRLSCAASGFTFRSHSMNWVRQAPGKGLEWVSS | extracellular |
| | ISSDSTYTYYADSVKGRFTISRDNAKNSLYLQMNSLRAEDTAVYYCARSG | binding |
| | GQWKYYDYWGQGTLVTVSSGSRGGGGSGGGGSGGGGSLEMASSELTQDPA | domain |
| | VSVALGQTVRITCQGDSLRSYYASWYQQKPGQAPVLVIYGKNNRPSGIPD | |
| | RFSGSSSGNTASLTITGAQAEDEADYYCNSRDSSGNPPVVFGGGTKLTVL | |
| | GGGGSGGGGSGGGGSGGGGSEVQLVQSGAEMKKPGASLKLSCKASGYTFI | |
| | DYYVYWMRQAPGQGLESMGWINPNSGGTNYAQKFQGRVTMTRDTSISTAY | |
| | MELSRLRSDDTAMYYCARSQRDGYMDYWGQGTLVTVSSGSRGGGGSGGGG | |
| | SGGGGSLEMAQSALTQPASVSASPGQSIAISCTGTSSDVGWYQQHPGKAP | |
| | KLMIYEDSKRPSGVSNRFSGSKSGNTASLTISGLQAEDEADYYCSSNTRS | |
| | STLVFGGGTKLTVL | |
| 81 | EVQLVQSGAEMKKPGASLKLSCKASGYTFIDYYVYWMRQAPGQGLESMGW | extracellular |
| | INPNSGGTNYAQKFQGRVTMTRDTSISTAYMELSRLRSDDTAMYYCARSQ | binding |
| | RDGYMDYWGQGTLVTVSSEAAAKSSELTQDPAVSVALGQTVRITCQGDSL | domain |
| | RSYYASWYQQKPGQAPVLVIYGKNNRPSGIPDRFSGSSSGNTASLTITGA | |
| | QAEDEADYYCNSRDSSGNPPVVFGGGTKLTVLGSRGGGGSGGGGSGGGGS | |
| | LEMAQVQLVESGGGLVHPGGSLRLSCAASGFTFRSHSMNWVRQAPGKGLE | |
| | WVSSISSDSTYTYYADSVKGRFTISRDNAKNSLYLQMNSLRAEDTAVYYC | |
| | ARSGGQWKYYDYWGQGTLVTVSSEAAAKQSALTQPASVSASPGQSIAISC | |
| | TGTSSDVGWYQQHPGKAPKLMIYEDSKRPSGVSNRFSGSKSGNTASLTIS | |
| | GLQAEDEADYYCSSNTRSSTLVFGGGTKLTVL | |
| 82 | QSALTQPASVSASPGQSIAISCTGTSSDVGWYQQHPGKAPKLMIYEDSKR | extracellular |
| | PSGVSNRFSGSKSGNTASLTISGLQAEDEADYYCSSNTRSSTLVFGGGTK | binding |
| | LTVLEAAAKQVQLVESGGGLVHPGGSLRLSCAASGFTFRSHSMNWVRQAP | domain |
| | GKGLEWVSSISSDSTYTYYADSVKGRFTISRDNAKNSLYLQMNSLRAEDT | |
| | AVYYCARSGGQWKYYDYWGQGTLVTVSSGSTSGSGKPGSGEGSTKGSSEL | |
| | TQDPAVSVALGQTVRITCQGDSLRSYYASWYQQKPGQAPVLVIYGKNNRP | |
| | SGIPDRFSGSSSGNTASLTITGAQAEDEADYYCNSRDSSGNPPVVFGGGT | |
| | KLTVLEAAAKEVQLVQSGAEMKKPGASLKLSCKASGYTFIDYYVYWMRQA | |
| | PGQGLESMGWINPNSGGTNYAQKFQGRVTMTRDTSISTAYMELSRLRSDD | |
| | TAMYYCARSQRDGYMDYWGQGTLVTVSS | |
| 83 | QVQLVESGGGLVHPGGSLRLSCAASGFTFRSHSMNWVRQAPGKGLEWVSS | extracellular |
| | ISSDSTYTYYADSVKGRFTISRDNAKNSLYLQMNSLRAEDTAVYYCARSG | binding |
| | GQWKYYDYWGQGTLVTVSSGGGGSQSALTQPASVSASPGQSIAISCTGTS | domain |
| | SDVGWYQQHPGKAPKLMIYEDSKRPSGVSNRFSGSKSGNTASLTISGLQA | |
| | EDEADYYCSSNTRSSTLVFGGGTKLTVLGSRGGGGSGGGGSGGGGSLEMA | |
| | EVQLVQSGAEMKKPGASLKLSCKASGYTFIDYYVYWMRQAPGQGLESMGW | |
| | INPNSGGTNYAQKFQGRVTMTRDTSISTAYMELSRLRSDDTAMYYCARSQ | |
| | RDGYMDYWGQGTLVTVSSGGGGSSSELTQDPAVSVALGQTVRITCQGDSL | |
| | RSYYASWYQQKPGQAPVLVIYGKNNRPSGIPDRFSGSSSGNTASLTITGA | |
| | QAEDEADYYCNSRDSSGNPPVVFGGGTKLTVL | |
| 84 | SSELTQDPAVSVALGQTVRITCQGDSLRSYYASWYQQKPGQAPVLVIYGK | extracellular |
| | NNRPSGIPDRFSGSSSGNTASLTITGAQAEDEADYYCNSRDSSGNPPVVF | binding |
| | GGGTKLTVLGGGGSEVQLVQSGAEMKKPGASLKLSCKASGYTFIDYYVYW | domain |
| | MRQAPGQGLESMGWINPNSGGTNYAQKFQGRVTMTRDTSISTAYMELSRL | |
| | RSDDTAMYYCARSQRDGYMDYWGQGTLVTVSSGSRGGGGSGGGGSGGGGS | |
| | LEMAQSALTQPASVSASPGQSIAISCTGTSSDVGWYQQHPGKAPKLMIYE | |
| | DSKRPSGVSNRFSGSKSGNTASLTISGLQAEDEADYYCSSNTRSSTLVFG | |
| | GGTKLTVLGGGGSQVQLVESGGGLVHPGGSLRLSCAASGFTFRSHSMNWV | |
| | RQAPGKGLEWVSSISSDSTYTYYADSVKGRFTISRDNAKNSLYLQMNSLR | |
| | AEDTAVYYCARSGGQWKYYDYWGQGTLVTVSS | |
| 85 | QSALTQPASVSASPGQSIAISCTGTSSDVGWYQQHPGKAPKLMIYEDSKR | extracellular |
| | PSGVSNRFSGSKSGNTASLTISGLQAEDEADYYCSSNTRSSTLVFGGGTK | binding |
| | LTVLGGGGSQVQLVESGGGLVHPGGSLRLSCAASGFTFRSHSMNWVRQAP | domain |
| | GKGLEWVSSISSDSTYTYYADSVKGRFTISRDNAKNSLYLQMNSLRAEDT | |
| | AVYYCARSGGQWKYYDYWGQGTLVTVSSGSRGGGGSGGGGSGGGGSLEMA | |
| | SSELTQDPAVSVALGQTVRITCQGDSLRSYYASWYQQKPGQAPVLVIYGK | |
| | NNRPSGIPDRFSGSSSGNTASLTITGAQAEDEADYYCNSRDSSGNPPVVF | |
| | GGGTKLTVLGGGGSEVQLVQSGAEMKKPGASLKLSCKASGYTFIDYYVYW | |
| | MRQAPGQGLESMGWINPNSGGTNYAQKFQGRVTMTRDTSISTAYMELSRL | |
| | RSDDTAMYYCARSQRDGYMDYWGQGTLVTVSS | |
| 86 | QSALTQPASVSASPGQSIAISCTGTSSDVGWYQQHPGKAPKLMIYEDSKR | extracellular |
| | PSGVSNRFSGSKSGNTASLTISGLQAEDEADYYCSSNTRSSTLVFGGGTK | binding |
| | LTVLGGGGSSSELTQDPAVSVALGQTVRITCQGDSLRSYYASWYQQKPGQ | domain |
| | APVLVIYGKNNRPSGIPDRFSGSSSGNTASLTITGAQAEDEADYYCNSRD | |
| | SSGNPPVVFGGGTKLTVLGSRGGGGSGGGGSGGGGSLEMAQVQLVESGGG | |
| | LVHPGGSLRLSCAASGFTFRSHSMNWVRQAPGKGLEWVSSISSDSTYTYY | |
| | ADSVKGRFTISRDNAKNSLYLQMNSLRAEDTAVYYCARSGGQWKYYDYWG | |
| | QGTLVTVSSGGGGSEVQLVQSGAEMKKPGASLKLSCKASGYTFIDYYVYW | |
| | MRQAPGQGLESMGWINPNSGGTNYAQKFQGRVTMTRDTSISTAYMELSRL | |
| | RSDDTAMYYCARSQRDGYMDYWGQGTLVTVSS | |
| 87 | QVQLVESGGGLVHPGGSLRLSCAASGFTFRSHSMNWVRQAPGKGLEWVSS | extracellular |
| | ISSDSTYTYYADSVKGRFTISRDNAKNSLYLQMNSLRAEDTAVYYCARSG | binding |
| | GQWKYYDYWGQGTLVTVSSGGGGSGGGGSQSALTQPASVSASPGQSIAIS | domain |
| | CTGTSSDVGWYQQHPGKAPKLMIYEDSKRPSGVSNRFSGSKSGNTASLTI | |
| | SGLQAEDEADYYCSSNTRSSTLVFGGGTKLTVLGSRGGGGSGGGGSGGGG | |
| | SLEMAEVQLVQSGAEMKKPGASLKLSCKASGYTFIDYYVYWMRQAPGQGL | |
| | ESMGWINPNSGGTNYAQKFQGRVTMTRDTSISTAYMELSRLRSDDTAMYY | |
| | CARSQRDGYMDYWGQGTLVTVSSGGGGSGGGGSSSELTQDPAVSVALGQT | |
| | VRITCQGDSLRSYYASWYQQKPGQAPVLVIYGKNNRPSGIPDRFSGSSSG | |
| | NTASLTITGAQAEDEADYYCNSRDSSGNPPVVFGGGTKLTVL | |
| 88 | EVQLVQSGAEMKKPGASLKLSCKASGYTFIDYYVYWMRQAPGQGLESMGW | extracellular |
| | INPNSGGTNYAQKFQGRVTMTRDTSISTAYMELSRLRSDDTAMYYCARSQ | binding |
| | RDGYMDYWGQGTLVTVSSGGGGSGGGGSQVQLVESGGGLVHPGGSLRLSC | domain |
| | AASGFTFRSHSMNWVRQAPGKGLEWVSSISSDSTYTYYADSVKGRFTISR | |
| | DNAKNSLYLQMNSLRAEDTAVYYCARSGGQWKYYDYWGQGTLVTVSSGSR | |
| | GGGGSGGGGSGGGGSLEMASSELTQDPAVSVALGQTVRITCQGDSLRSYY | |
| | ASWYQQKPGQAPVLVIYGKNNRPSGIPDRFSGSSSGNTASLTITGAQAED | |
| | EADYYCNSRDSSGNPPVVFGGGTKLTVLGGGGSGGGGSQSALTQPASVSA | |
| | SPGQSIAISCTGTSSDVGWYQQHPGKAPKLMIYEDSKRPSGVSNRFSGSK | |
| | SGNTASLTISGLQAEDEADYYCSSNTRSSTLVFGGGTKLTVL | |
| 89 | QSALTQPASVSASPGQSIAISCTGTSSDVGWYQQHPGKAPKLMIYEDSKR | extracellular |
| | PSGVSNRFSGSKSGNTASLTISGLQAEDEADYYCSSNTRSSTLVFGGGTK | binding |
| | LTVLGGGGSGGGGSQVQLVESGGGLVHPGGSLRLSCAASGFTFRSHSMNW | domain |
| | VRQAPGKGLEWVSSISSDSTYTYYADSVKGRFTISRDNAKNSLYLQMNSL | |
| | RAEDTAVYYCARSGGQWKYYDYWGQGTLVTVSSGSRGGGGSGGGGSGGGG | |
| | SLEMASSELTQDPAVSVALGQTVRITCQGDSLRSYYASWYQQKPGQAPVL | |
| | VIYGKNNRPSGIPDRFSGSSSGNTASLTITGAQAEDEADYYCNSRDSSGN | |
| | PPVVFGGGTKLTVLGGGGSGGGGSEVQLVQSGAEMKKPGASLKLSCKASG | |
| | YTFIDYYVYWMRQAPGQGLESMGWINPNSGGTNYAQKFQGRVTMTRDTSI | |
| | STAYMELSRLRSDDTAMYYCARSQRDGYMDYWGQGTLVTVSS | |
| 90 | QSALTQPASVSASPGQSIAISCTGTSSDVGWYQQHPGKAPKLMIYEDSKR | extracellular |
| | PSGVSNRFSGSKSGNTASLTISGLQAEDEADYYCSSNTRSSTLVFGGGTK | binding |
| | LTVLGGGGSGGGGSQVQLVESGGGLVHPGGSLRLSCAASGFTFRSHSMNW | domain |
| | VRQAPGKGLEWVSSISSDSTYTYYADSVKGRFTISRDNAKNSLYLQMNSL | |
| | RAEDTAVYYCARSGGQWKYYDYWGQGTLVTVSSGSTSGSGKPGSGEGSTK | |
| | GSSELTQDPAVSVALGQTVRITCQGDSLRSYYASWYQQKPGQAPVLVIYG | |
| | KNNRPSGIPDRFSGSSSGNTASLTITGAQAEDEADYYCNSRDSSGNPPVV | |
| | FGGGTKLTVLGGGGSGGGGSEVQLVQSGAEMKKPGASLKLSCKASGYTFI | |
| | DYYVYWMRQAPGQGLESMGWINPNSGGTNYAQKFQGRVTMTRDTSISTAY | |
| | MELSRLRSDDTAMYYCARSQRDGYMDYWGQGTLVTVSS | |
| 91 | atggtgctgcagacccaggtgttcatcagcctgctgctgtggatctccgg | human IgG - |
| | agcatacgga | kappa signal |
| | | sequence (nt) |
| 92 | MVLQTQVFISLLLWISGAYG | human IgG - |
| | | kappa signal |
| | | peptide(aa) |
| 93 | atggtgctgcagacccaggtgttcatcagcctgctgctgtggatctctgg | human IgG - |
| | cgcctacggc | kappa signal |
| | | sequence (nt) |
| 94 | atggtgctgcagacccaggtgttcatcagcctgctgctgtggatctctgg | human IgG - |
| | cgcctatgga | kappa signal |
| | | sequence (nt) |
| 95 | atggtgctgcagacacaggtgttcatctccctgctgctgtggatctctgg | human IgG - |
| | agcatacgga | kappa signal |
| | | sequence (nt) |
| 96 | atggtgctgcagacacaggtgttcatcagcctgctgctgtggatctccgg | human IgG - |
| | agcatacgga | kappa signal |
| | | sequence (nt) |
| 97 | atgcttctcctggtgacaagccttctgctctgtgagttaccacacccagc | GMCSFR |
| | attcctcctgatccca | alpha chain |
| | | signal |
| | | sequence |
| 98 | MLLLVTSLLLCELPHPAFLLIP | GMCSFR |
| | | alpha chain |
| | | signal peptide |
| 99 | MALPVTALLLPLALLLHA | CD8 alpha |
| | | signal peptide |
| 100 | RSKRSRLLHSDYMNMTPRRPGPTRKHYQPYAPPRDFAAYRS | CD28 co- |
| | | stimulatory |
| | | domain |
| 101 | QSALTQPASVSASPGQSIAISCTGTSSDVGWYQQHPGKAPKLMIYEDSKR | Anti-BCMA |
| | PSGVSNRFSGSKSGNTASLTISGLQAEDEADYYCSSNTRSSTLVFGGGTK | CAR |
| | LTVLGSRGGGGSGGGGSGGGGSLEMAEVQLVQSGAEMKKPGASLKLSCKA | |
| | SGYTFIDYYVYWMRQAPGQGLESMGWINPNSGGTNYAQKFQGRVTMTRDT | |
| | SISTAYMELSRLRSDDTAMYYCARSQRDGYMDYWGQGTLVTVSSESKYGP | |
| | PCPPCPAPPVAGPSVFLFPPKPKDTLMISRTPEVTCVVVDVSQEDPEVQF | |
| | NWYVDGVEVHNAKTKPREEQFQSTYRVVSVLTVLHQDWLNGKEYKCKVSN | |
| | KGLPSSIEKTISKAKGQPREPQVYTLPPSQEEMTKNQVSLTCLVKGFYPS | |
| | DIAVEWESNGQPENNYKTTPPVLDSDGSFFLYSRLTVDKSRWQEGNVESC | |
| | SVMHEALHNHYTQKSLSLSLGKMFWVLVVVGGVLACYSLLVTVAFIIFWV | |
| | KRGRKKLLYIFKQPFMRPVQTTQEEDGCSCRFPEEEEGGCELRVKESRSA | |
| | DAPAYQQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQA | |
| | LPPR | |
| 102 | SSELTQDPAVSVALGQTVRITCQGDSLRSYYASWYQQKPGQAPVLVIYGK | Anti- |
| | NNRPSGIPDRFSGSSSGNTASLTITGAQAEDEADYYCNSRDSSGNPPVVF | GPRC5D |
| | GGGTKLTVLGSRGGGGSGGGGSGGGGSLEMAQVQLVESGGGLVHPGGSLR | CAR |
| | LSCAASGFTFRSHSMNWVRQAPGKGLEWVSSISSDSTYTYYADSVKGRFT | |
| | ISRDNAKNSLYLQMNSLRAEDTAVYYCARSGGQWKYYDYWGQGTLVTVSS | |
| | ESKYGPPCPPCPAPPVAGPSVFLFPPKPKDTLMISRTPEVTCVVVDVSQE | |
| | DPEVQFNWYVDGVEVHNAKTKPREEQFQSTYRVVSVLTVLHQDWLNGKEY | |
| | KCKVSNKGLPSSIEKTISKAKGQPREPQVYTLPPSQEEMTKNQVSLTCLV | |
| | KGFYPSDIAVEWESNGQPENNYKTTPPVLDSDGSFFLYSRLTVDKSRWQE | |
| | GNVFSCSVMHEALHNHYTQKSLSLSLGKMFWVLVVVGGVLACYSLLVTVA | |
| | FIIFWVKRGRKKLLYIFKQPFMRPVQTTQEEDGCSCRFPEEEEGGCELRV | |
| | KFSRSADAPAYQQGQNQLYNELNLGRREEYDVLDKRRGRDPEMGGKPRRK | |
| | NPQEGLYNELQKDKMAEAYSEIGMKGERRRGKGHDGLYQGLSTATKDTYD | |
| | ALHMQALPPR | |
| 103 | ctcgagggcggcggagagggcagaggaagtcttctaacatgcggtgacgt | T2A peptide |
| | ggaggagaatcccggccctagg | (nt) |
| 104 | cttgaaggtggtggcgaaggcagaggcagcctgcttacatgcggagatgt | T2A peptide |
| | ggaagagaaccccggacctaga | (nt) |
| 105 | atgccgctgctgctactgctgcccctgctgtgggcaggggctctagcttc | CAR 1 (nt) |
| | ttctgagctcacccaagatcctgccgtgtctgtggctctgggccaaacag | |
| | tgcggattacctgtcagggcgatagcctgagaagctactacgccagctgg | |
| | taccaacagaagccaggacaggctcccgtgctcgtcatttatggcaagaa | |
| | caacagaccatccggcatccccgatcggttttccggaagcagctctggca | |
| | atactgcctccctcaccatcactggcgcccaagcagaagatgaagcagac | |
| | tactattgtaactccagagacagctccggcaatcctcctgtggtgttcgg | |
| | aggcggaacaaaactcaccgtcctcggcagccggggtggaggtggaagcg | |
| | gcggtggtggctccggaggagggggtagcctcgagatggcacaggtccaa | |
| | ctcgtggaatcaggaggtggacttgttcaccccggcggaagcctgagact | |
| | gtcttgtgccgcttccggattcacattccggtcccactccatgaattggg | |
| | tccgacaagctcccggcaaaggccttgaatgggtgtccagcatcagcagc | |
| | gacagcacctacacctactatgccgacagcgtgaagggaaggttcacaat | |
| | ctctcgggacaacgccaagaacagcctgtacctgcagatgaactccctca | |
| | gagccgaggatacagctgtgtattactgtgctagaagtggcggccagtgg | |
| | aagtactacgactactggggacaaggcacactcgtgacagttagctctga | |
| | ggccgcagccaaagaagtgcagctggtgcagtctggcgccgagatgaaga | |
| | aacctggcgcctctctgaagctgagctgcaaggccagcggctacaccttc | |
| | atcgactactacgtgtactggatgcggcaggcccctggacagggactcga | |
| | atctatgggctggatcaaccccaatagcggcggcaccaattacgcccaga | |
| | aattccagggcagagtgaccatgaccagagacaccagcatcagcaccgcc | |
| | tacatggaactgagccggctgagatccgacgacaccgccatgtactactg | |
| | cgccagatctcagcgcgacggctacatggattattggggccagggaaccc | |
| | tggtcaccgtgtccagcggatctagaggtggcggaggatctggcggcgga | |
| | ggaagcggaggcggcggatctcttgaaatggctcagtctgccctgacaca | |
| | gcctgccagcgttagtgctagtcccggacagtctatcgccatcagctgta | |
| | ccggcaccagctctgacgttggctggtatcagcagcaccctggcaaggcc | |
| | cctaagctgatgatctacgaggacagcaagaggcccagcggcgtgtccaa | |
| | tagattcagcggcagcaagagcggcaacaccgccagcctgacaattagcg | |
| | gactgcaggccgaggacgaggccgattactactgcagcagcaacacccgg | |
| | tccagcacactggtttttggcggaggcaccaagctgacagtgctggagtc | |
| | taaatacggaccgccttgtcctccatgtcctgctcctccagttgccggac | |
| | cttccgtgttcctgtttcctccaaagcctaaggacaccctgatgatcagc | |
| | agaacccctgaagtgacctgcgtggtggtggacgtgtcccaagaggatcc | |
| | tgaggtgcagttcaactggtatgtggacggcgtggaagtgcacaacgcca | |
| | agaccaagcctagagaggaacagttccagagcacctacagagtggtgtcc | |
| | gtgctgacagtgctgcaccaggattggctgaacggcaaagagtacaagtg | |
| | caaggtgtccaacaagggcctgcctagcagcatcgagaaaaccatcagca | |
| | aggccaagggccagccaagagaaccccaggtgtacacactgcctccaagc | |
| | caagaggaaatgaccaagaaccaggtgtccctgacctgcctggtcaaggg | |
| | cttctacccttccgatatcgccgtggaatgggagagcaatggccagcctg | |
| | agaacaactacaagaccacacctcctgtgctggacagcgacggctcattc | |
| | ttcctgtacagccggctgaccgtggacaagagcagatggcaagagggcaa | |
| | cgtgttcagctgcagcgtgatgcacgaggccctgcacaaccactacaccc | |
| | agaagtctctgagcctgagcctgggcaagatgttctgggtgctcgttgtt | |
| | gttggcggcgtgctggcctgttactccctgctggttaccgtggccttcat | |
| | catcttttgggtcaagcggggcagaaagaagctgctctacatcttcaagc | |
| | agcccttcatgcggcccgtgcagaccacacaagaggaagatggctgctcc | |
| | tgcagattccccgaggaagaagaaggcggctgcgagctgagagtgaagtt | |
| | cagcagatccgccgacgctccagcctatcagcagggacagaaccagctgt | |
| | acaacgagctgaacctggggagaagagaagagtacgacgtgctggataag | |
| | cggagaggcagagatcctgagatgggcggcaagcccagacggaagaatcc | |
| | tcaagagggcctgtataatgagctgcagaaagacaagatggccgaggcct | |
| | acagcgagatcggaatgaagggcgagcgcagaagaggcaagggacacgat | |
| | ggactgtaccagggactgagcaccgccaccaaggatacctatgacgcact | |
| | gcacatgcaggccctgccacctaga | |
| 106 | atgccgctgctgctactgctgcccctgctgtgggcaggggctctagctca | CAR 2 (nt) |
| | ggtccaactcgtggaatcaggaggtggacttgttcaccccggcggaagcc | |
| | tgagactgtcttgtgccgcttccggattcacattccggtcccactccatg | |
| | aattgggtccgacaagctcccggcaaaggccttgaatgggtgtccagcat | |
| | cagcagcgacagcacctacacctactatgccgacagcgtgaagggaaggt | |
| | tcacaatctctcgggacaacgccaagaacagcctgtacctgcagatgaac | |
| | tccctcagagccgaggatacagctgtgtattactgtgctagaagtggcgg | |
| | ccagtggaagtactacgactactggggacaaggcacactcgtgacagtta | |
| | gctctggcagccggggtggaggtggaagcggcggtggtggctccggagga | |
| | gggggtagcctcgagatggcatcttctgagctcacccaagatcctgccgt | |
| | gtctgtggctctgggccaaacagtgcggattacctgtcagggcgatagcc | |
| | tgagaagctactacgccagctggtaccaacagaagccaggacaggctccc | |
| | gtgctcgtcatttatggcaagaacaacagaccatccggcatccccgatcg | |
| | gttttccggaagcagctctggcaatactgcctccctcaccatcactggcg | |
| | cccaagcagaagatgaagcagactactattgtaactccagagacagctcc | |
| | ggcaatcctcctgtggtgttcggaggcggaacaaaactcaccgtcctcgg | |
| | tggcggaggatctgaagtgcagctggtgcagtctggcgccgagatgaaga | |
| | aacctggcgcctctctgaagctgagctgcaaggccagcggctacaccttc | |
| | atcgactactacgtgtactggatgcggcaggcccctggacagggactcga | |
| | atctatgggctggatcaaccccaatagcggcggcaccaattacgcccaga | |
| | aattccagggcagagtgaccatgaccagagacaccagcatcagcaccgcc | |
| | tacatggaactgagccggctgagatccgacgacaccgccatgtactactg | |
| | cgccagatctcagcgcgacggctacatggattattggggccagggaaccc | |
| | tggtcaccgtgtccagcggatctagaggtggcggaggatctggcggcgga | |
| | ggaagcggaggcggcggatctcttgaaatggctcagtctgccctgacaca | |
| | gcctgccagcgttagtgctagtcccggacagtctatcgccatcagctgta | |
| | ccggcaccagctctgacgttggctggtatcagcagcaccctggcaaggcc | |
| | cctaagctgatgatctacgaggacagcaagaggcccagcggcgtgtccaa | |
| | tagattcagcggcagcaagagcggcaacaccgccagcctgacaattagcg | |
| | gactgcaggccgaggacgaggccgattactactgcagcagcaacacccgg | |
| | tccagcacactggtttttggcggaggcaccaagctgacagtgctggagtc | |
| | taaatacggaccgccttgtcctccatgtcctgctcctccagttgccggac | |
| | cttccgtgttcctgtttcctccaaagcctaaggacaccctgatgatcagc | |
| | agaacccctgaagtgacctgcgtggtggtggacgtgtcccaagaggatcc | |
| | tgaggtgcagttcaactggtatgtggacggcgtggaagtgcacaacgcca | |
| | agaccaagcctagagaggaacagttccagagcacctacagagtggtgtcc | |
| | gtgctgacagtgctgcaccaggattggctgaacggcaaagagtacaagtg | |
| | caaggtgtccaacaagggcctgcctagcagcatcgagaaaaccatcagca | |
| | aggccaagggccagccaagagaaccccaggtgtacacactgcctccaagc | |
| | caagaggaaatgaccaagaaccaggtgtccctgacctgcctggtcaaggg | |
| | cttctacccttccgatatcgccgtggaatgggagagcaatggccagcctg | |
| | agaacaactacaagaccacacctcctgtgctggacagcgacggctcattc | |
| | ttcctgtacagccggctgaccgtggacaagagcagatggcaagagggcaa | |
| | cgtgttcagctgcagcgtgatgcacgaggccctgcacaaccactacaccc | |
| | agaagtctctgagcctgagcctgggcaagatgttctgggtgctcgttgtt | |
| | gttggcggcgtgctggcctgttactccctgctggttaccgtggccttcat | |
| | catcttttgggtcaagcggggcagaaagaagctgctctacatcttcaagc | |
| | agcccttcatgcggcccgtgcagaccacacaagaggaagatggctgctcc | |
| | tgcagattccccgaggaagaagaaggcggctgcgagctgagagtgaagtt | |
| | cagcagatccgccgacgctccagcctatcagcagggacagaaccagctgt | |
| | acaacgagctgaacctggggagaagagaagagtacgacgtgctggataag | |
| | cggagaggcagagatcctgagatgggcggcaagcccagacggaagaatcc | |
| | tcaagagggcctgtataatgagctgcagaaagacaagatggccgaggcct | |
| | acagcgagatcggaatgaagggcgagcgcagaagaggcaagggacacgat | |
| | ggactgtaccagggactgagcaccgccaccaaggatacctatgacgcact | |
| | gcacatgcaggccctgccacctaga | |
| 107 | atgccgctgctgctactgctgcccctgctgtgggcaggggctctagcttc | CAR 3 (nt) |
| | ttctgagctcacccaagatcctgccgtgtctgtggctctgggccaaacag | |
| | tgcggattacctgtcagggcgatagcctgagaagctactacgccagctgg | |
| | taccaacagaagccaggacaggctcccgtgctcgtcatttatggcaagaa | |
| | caacagaccatccggcatccccgatcggttttccggaagcagctctggca | |
| | atactgcctccctcaccatcactggcgcccaagcagaagatgaagcagac | |
| | tactattgtaactccagagacagctccggcaatcctcctgtggtgttcgg | |
| | aggcggaacaaaactcaccgtcctcggcagccggggtggaggtggaagcg | |
| | gcggtggtggctccggaggagggggtagcctcgagatggcacaggtccaa | |
| | ctcgtggaatcaggaggtggacttgttcaccccggcggaagcctgagact | |
| | gtcttgtgccgcttccggattcacattccggtcccactccatgaattggg | |
| | tccgacaagctcccggcaaaggccttgaatgggtgtccagcatcagcagc | |
| | gacagcacctacacctactatgccgacagcgtgaagggaaggttcacaat | |
| | ctctcgggacaacgccaagaacagcctgtacctgcagatgaactccctca | |
| | gagccgaggatacagctgtgtattactgtgctagaagtggcggccagtgg | |
| | aagtactacgactactggggacaaggcacactcgtgacagttagctctgg | |
| | tggaggaggtagtggtggcggtggatcaggtggcggcggcagtggtggcg | |
| | gaggatctgaagtgcagctggtgcagtctggcgccgagatgaagaaacct | |
| | ggcgcctctctgaagctgagctgcaaggccagcggctacaccttcatcga | |
| | ctactacgtgtactggatgcggcaggcccctggacagggactcgaatcta | |
| | tgggctggatcaaccccaatagcggcggcaccaattacgcccagaaattc | |
| | cagggcagagtgaccatgaccagagacaccagcatcagcaccgcctacat | |
| | ggaactgagccggctgagatccgacgacaccgccatgtactactgcgcca | |
| | gatctcagcgcgacggctacatggattattggggccagggaaccctggtc | |
| | accgtgtccagcggatctagaggtggcggaggatctggcggcggaggaag | |
| | cggaggcggcggatctcttgaaatggctcagtctgccctgacacagcctg | |
| | ccagcgttagtgctagtcccggacagtctatcgccatcagctgtaccggc | |
| | accagctctgacgttggctggtatcagcagcaccctggcaaggcccctaa | |
| | gctgatgatctacgaggacagcaagaggcccagcggcgtgtccaatagat | |
| | tcagcggcagcaagagcggcaacaccgccagcctgacaattagcggactg | |
| | caggccgaggacgaggccgattactactgcagcagcaacacccggtccag | |
| | cacactggtttttggcggaggcaccaagctgacagtgctggagtctaaat | |
| | acggaccgccttgtcctccatgtcctgctcctccagttgccggaccttcc | |
| | gtgttcctgtttcctccaaagcctaaggacaccctgatgatcagcagaac | |
| | ccctgaagtgacctgcgtggtggtggacgtgtcccaagaggatcctgagg | |
| | tgcagttcaactggtatgtggacggcgtggaagtgcacaacgccaagacc | |
| | aagcctagagaggaacagttccagagcacctacagagtggtgtccgtgct | |
| | gacagtgctgcaccaggattggctgaacggcaaagagtacaagtgcaagg | |
| | tgtccaacaagggcctgcctagcagcatcgagaaaaccatcagcaaggcc | |
| | aagggccagccaagagaaccccaggtgtacacactgcctccaagccaaga | |
| | ggaaatgaccaagaaccaggtgtccctgacctgcctggtcaagggcttct | |
| | acccttccgatatcgccgtggaatgggagagcaatggccagcctgagaac | |
| | aactacaagaccacacctcctgtgctggacagcgacggctcattcttcct | |
| | gtacagccggctgaccgtggacaagagcagatggcaagagggcaacgtgt | |
| | tcagctgcagcgtgatgcacgaggccctgcacaaccactacacccagaag | |
| | tctctgagcctgagcctgggcaagatgttctgggtgctcgttgttgttgg | |
| | cggcgtgctggcctgttactccctgctggttaccgtggccttcatcatct | |
| | tttgggtcaagcggggcagaaagaagctgctctacatcttcaagcagccc | |
| | ttcatgcggcccgtgcagaccacacaagaggaagatggctgctcctgcag | |
| | attccccgaggaagaagaaggcggctgcgagctgagagtgaagttcagca | |
| | gatccgccgacgctccagcctatcagcagggacagaaccagctgtacaac | |
| | gagctgaacctggggagaagagaagagtacgacgtgctggataagcggag | |
| | aggcagagatcctgagatgggcggcaagcccagacggaagaatcctcaag | |
| | agggcctgtataatgagctgcagaaagacaagatggccgaggcctacagc | |
| | gagatcggaatgaagggcgagcgcagaagaggcaagggacacgatggact | |
| | gtaccagggactgagcaccgccaccaaggatacctatgacgcactgcaca | |
| | tgcaggccctgccacctaga | |
| 108 | atgccgctgctgctactgctgcccctgctgtgggcaggggctctagctca | CAR 4 (nt) |
| | ggtccaactcgtggaatcaggaggtggacttgttcaccccggcggaagcc | |
| | tgagactgtcttgtgccgcttccggattcacattccggtcccactccatg | |
| | aattgggtccgacaagctcccggcaaaggccttgaatgggtgtccagcat | |
| | cagcagcgacagcacctacacctactatgccgacagcgtgaagggaaggt | |
| | tcacaatctctcgggacaacgccaagaacagcctgtacctgcagatgaac | |
| | tccctcagagccgaggatacagctgtgtattactgtgctagaagtggcgg | |
| | ccagtggaagtactacgactactggggacaaggcacactcgtgacagtta | |
| | gctctggcagccggggtggaggtggaagcggcggtggtggctccggagga | |
| | gggggtagcctcgagatggcatcttctgagctcacccaagatcctgccgt | |
| | gtctgtggctctgggccaaacagtgcggattacctgtcagggcgatagcc | |
| | tgagaagctactacgccagctggtaccaacagaagccaggacaggctccc | |
| | gtgctcgtcatttatggcaagaacaacagaccatccggcatccccgatcg | |
| | gttttccggaagcagctctggcaatactgcctccctcaccatcactggcg | |
| | cccaagcagaagatgaagcagactactattgtaactccagagacagctcc | |
| | ggcaatcctcctgtggtgttcggaggcggaacaaaactcaccgtcctcgg | |
| | cggtggcggatctggtggaggaggtagcggtggtggtggatctggtggcg | |
| | gaggatctgaagtgcagctggtgcagtctggcgccgagatgaagaaacct | |
| | ggcgcctctctgaagctgagctgcaaggccagcggctacaccttcatcga | |
| | ctactacgtgtactggatgcggcaggcccctggacagggactcgaatcta | |
| | tgggctggatcaaccccaatagcggcggcaccaattacgcccagaaattc | |
| | cagggcagagtgaccatgaccagagacaccagcatcagcaccgcctacat | |
| | ggaactgagccggctgagatccgacgacaccgccatgtactactgcgcca | |
| | gatctcagcgcgacggctacatggattattggggccagggaaccctggtc | |
| | accgtgtccagcggatctagaggtggcggaggatctggcggcggaggaag | |
| | cggaggcggcggatctcttgaaatggctcagtctgccctgacacagcctg | |
| | ccagcgttagtgctagtcccggacagtctatcgccatcagctgtaccggc | |
| | accagctctgacgttggctggtatcagcagcaccctggcaaggcccctaa | |
| | gctgatgatctacgaggacagcaagaggcccagcggcgtgtccaatagat | |
| | tcagcggcagcaagagcggcaacaccgccagcctgacaattagcggactg | |
| | caggccgaggacgaggccgattactactgcagcagcaacacccggtccag | |
| | cacactggtttttggcggaggcaccaagctgacagtgctggagtctaaat | |
| | acggaccgccttgtcctccatgtcctgctcctccagttgccggaccttcc | |
| | gtgttcctgtttcctccaaagcctaaggacaccctgatgatcagcagaac | |
| | ccctgaagtgacctgcgtggtggtggacgtgtcccaagaggatcctgagg | |
| | tgcagttcaactggtatgtggacggcgtggaagtgcacaacgccaagacc | |
| | aagcctagagaggaacagttccagagcacctacagagtggtgtccgtgct | |
| | gacagtgctgcaccaggattggctgaacggcaaagagtacaagtgcaagg | |
| | tgtccaacaagggcctgcctagcagcatcgagaaaaccatcagcaaggcc | |
| | aagggccagccaagagaaccccaggtgtacacactgcctccaagccaaga | |
| | ggaaatgaccaagaaccaggtgtccctgacctgcctggtcaagggcttct | |
| | acccttccgatatcgccgtggaatgggagagcaatggccagcctgagaac | |
| | aactacaagaccacacctcctgtgctggacagcgacggctcattcttcct | |
| | gtacagccggctgaccgtggacaagagcagatggcaagagggcaacgtgt | |
| | tcagctgcagcgtgatgcacgaggccctgcacaaccactacacccagaag | |
| | tctctgagcctgagcctgggcaagatgttctgggtgctcgttgttgttgg | |
| | cggcgtgctggcctgttactccctgctggttaccgtggccttcatcatct | |
| | tttgggtcaagcggggcagaaagaagctgctctacatcttcaagcagccc | |
| | ttcatgcggcccgtgcagaccacacaagaggaagatggctgctcctgcag | |
| | attccccgaggaagaagaaggcggctgcgagctgagagtgaagttcagca | |
| | gatccgccgacgctccagcctatcagcagggacagaaccagctgtacaac | |
| | gagctgaacctggggagaagagaagagtacgacgtgctggataagcggag | |
| | aggcagagatcctgagatgggcggcaagcccagacggaagaatcctcaag | |
| | agggcctgtataatgagctgcagaaagacaagatggccgaggcctacagc | |
| | gagatcggaatgaagggcgagcgcagaagaggcaagggacacgatggact | |
| | gtaccagggactgagcaccgccaccaaggatacctatgacgcactgcaca | |
| | tgcaggccctgccacctaga | |
| 109 | atgccgctgctgctactgctgcccctgctgtgggcaggggctctagctga | CAR 8 (nt) |
| | agtgcagctggtgcagtctggcgccgagatgaagaaacctggcgcctctc | |
| | tgaagctgagctgcaaggccagcggctacaccttcatcgactactacgtg | |
| | tactggatgcggcaggcccctggacagggactcgaatctatgggctggat | |
| | caaccccaatagcggcggcaccaattacgcccagaaattccagggcagag | |
| | tgaccatgaccagagacaccagcatcagcaccgcctacatggaactgagc | |
| | cggctgagatccgacgacaccgccatgtactactgcgccagatctcagcg | |
| | cgacggctacatggattattggggccagggaaccctggtcaccgtgtcca | |
| | gcgaggccgcagccaaatcttctgagctcacccaagatcctgccgtgtct | |
| | gtggctctgggccaaacagtgcggattacctgtcagggcgatagcctgag | |
| | aagctactacgccagctggtaccaacagaagccaggacaggctcccgtgc | |
| | tcgtcatttatggcaagaacaacagaccatccggcatccccgatcggttt | |
| | tccggaagcagctctggcaatactgcctccctcaccatcactggcgccca | |
| | agcagaagatgaagcagactactattgtaactccagagacagctccggca | |
| | atcctcctgtggtgttcggaggcggaacaaaactcaccgtcctcggcagc | |
| | cggggtggaggtggaagcggcggtggtggctccggaggagggggtagcct | |
| | cgagatggcacaggtccaactcgtggaatcaggaggtggacttgttcacc | |
| | ccggcggaagcctgagactgtcttgtgccgcttccggattcacattccgg | |
| | tcccactccatgaattgggtccgacaagctcccggcaaaggccttgaatg | |
| | ggtgtccagcatcagcagcgacagcacctacacctactatgccgacagcg | |
| | tgaagggaaggttcacaatctctcgggacaacgccaagaacagcctgtac | |
| | ctgcagatgaactccctcagagccgaggatacagctgtgtattactgtgc | |
| | tagaagtggcggccagtggaagtactacgactactggggacaaggcacac | |
| | tcgtgacagttagctctgaggccgcagccaaacagtctgccctgacacag | |
| | cctgccagcgttagtgctagtcccggacagtctatcgccatcagctgtac | |
| | cggcaccagctctgacgttggctggtatcagcagcaccctggcaaggccc | |
| | ctaagctgatgatctacgaggacagcaagaggcccagcggcgtgtccaat | |
| | agattcagcggcagcaagagcggcaacaccgccagcctgacaattagcgg | |
| | actgcaggccgaggacgaggccgattactactgcagcagcaacacccggt | |
| | ccagcacactggtttttggcggaggcaccaagctgacagtgctggagtct | |
| | aaatacggaccgccttgtcctccatgtcctgctcctccagttgccggacc | |
| | ttccgtgttcctgtttcctccaaagcctaaggacaccctgatgatcagca | |
| | gaacccctgaagtgacctgcgtggtggtggacgtgtcccaagaggatcct | |
| | gaggtgcagttcaactggtatgtggacggcgtggaagtgcacaacgccaa | |
| | gaccaagcctagagaggaacagttccagagcacctacagagtggtgtccg | |
| | tgctgacagtgctgcaccaggattggctgaacggcaaagagtacaagtgc | |
| | aaggtgtccaacaagggcctgcctagcagcatcgagaaaaccatcagcaa | |
| | ggccaagggccagccaagagaaccccaggtgtacacactgcctccaagcc | |
| | aagaggaaatgaccaagaaccaggtgtccctgacctgcctggtcaagggc | |
| | ttctacccttccgatatcgccgtggaatgggagagcaatggccagcctga | |
| | gaacaactacaagaccacacctcctgtgctggacagcgacggctcattct | |
| | tcctgtacagccggctgaccgtggacaagagcagatggcaagagggcaac | |
| | gtgttcagctgcagcgtgatgcacgaggccctgcacaaccactacaccca | |
| | gaagtctctgagcctgagcctgggcaagatgttctgggtgctcgttgttg | |
| | ttggcggcgtgctggcctgttactccctgctggttaccgtggccttcatc | |
| | atcttttgggtcaagcggggcagaaagaagctgctctacatcttcaagca | |
| | gcccttcatgcggcccgtgcagaccacacaagaggaagatggctgctcct | |
| | gcagattccccgaggaagaagaaggcggctgcgagctgagagtgaagttc | |
| | agcagatccgccgacgctccagcctatcagcagggacagaaccagctgta | |
| | caacgagctgaacctggggagaagagaagagtacgacgtgctggataagc | |
| | ggagaggcagagatcctgagatgggcggcaagcccagacggaagaatcct | |
| | caagagggcctgtataatgagctgcagaaagacaagatggccgaggccta | |
| | cagcgagatcggaatgaagggcgagcgcagaagaggcaagggacacgatg | |
| | gactgtaccagggactgagcaccgccaccaaggatacctatgacgcactg | |
| | cacatgcaggccctgccacctaga | |
| 110 | atgccgctgctgctactgctgcccctgctgtgggcaggggctctagctca | CAR 9 (nt) |
| | gtctgccctgacacagcctgccagcgttagtgctagtcccggacagtcta | |
| | tcgccatcagctgtaccggcaccagctctgacgttggctggtatcagcag | |
| | caccctggcaaggcccctaagctgatgatctacgaggacagcaagaggcc | |
| | cagcggcgtgtccaatagattcagcggcagcaagagcggcaacaccgcca | |
| | gcctgacaattagcggactgcaggccgaggacgaggccgattactactgc | |
| | agcagcaacacccggtccagcacactggtttttggcggaggcaccaagct | |
| | gacagtgctggaggccgcagccaaacaggtccaactcgtggaatcaggag | |
| | gtggacttgttcaccccggcggaagcctgagactgtcttgtgccgcttcc | |
| | ggattcacattccggtcccactccatgaattgggtccgacaagctcccgg | |
| | caaaggccttgaatgggtgtccagcatcagcagcgacagcacctacacct | |
| | actatgccgacagcgtgaagggaaggttcacaatctctcgggacaacgcc | |
| | aagaacagcctgtacctgcagatgaactccctcagagccgaggatacagc | |
| | tgtgtattactgtgctagaagtggcggccagtggaagtactacgactact | |
| | ggggacaaggcacactcgtgacagttagctctggcagcacctccggcagc | |
| | ggcaagcctggcagcggcgagggcagcaccaagggctcttctgagctcac | |
| | ccaagatcctgccgtgtctgtggctctgggccaaacagtgcggattacct | |
| | gtcagggcgatagcctgagaagctactacgccagctggtaccaacagaag | |
| | ccaggacaggctcccgtgctcgtcatttatggcaagaacaacagaccatc | |
| | cggcatccccgatcggttttccggaagcagctctggcaatactgcctccc | |
| | tcaccatcactggcgcccaagcagaagatgaagcagactactattgtaac | |
| | tccagagacagctccggcaatcctcctgtggtgttcggaggcggaacaaa | |
| | actcaccgtcctcgaggccgcagccaaagaagtgcagctggtgcagtctg | |
| | gcgccgagatgaagaaacctggcgcctctctgaagctgagctgcaaggcc | |
| | agcggctacaccttcatcgactactacgtgtactggatgcggcaggcccc | |
| | tggacagggactcgaatctatgggctggatcaaccccaatagcggcggca | |
| | ccaattacgcccagaaattccagggcagagtgaccatgaccagagacacc | |
| | agcatcagcaccgcctacatggaactgagccggctgagatccgacgacac | |
| | cgccatgtactactgcgccagatctcagcgcgacggctacatggattatt | |
| | ggggccagggaaccctggtcaccgtgtccagcgagtctaaatacggaccg | |
| | ccttgtcctccatgtcctgctcctccagttgccggaccttccgtgttcct | |
| | gtttcctccaaagcctaaggacaccctgatgatcagcagaacccctgaag | |
| | tgacctgcgtggtggtggacgtgtcccaagaggatcctgaggtgcagttc | |
| | aactggtatgtggacggcgtggaagtgcacaacgccaagaccaagcctag | |
| | agaggaacagttccagagcacctacagagtggtgtccgtgctgacagtgc | |
| | tgcaccaggattggctgaacggcaaagagtacaagtgcaaggtgtccaac | |
| | aagggcctgcctagcagcatcgagaaaaccatcagcaaggccaagggcca | |
| | gccaagagaaccccaggtgtacacactgcctccaagccaagaggaaatga | |
| | ccaagaaccaggtgtccctgacctgcctggtcaagggcttctacccttcc | |
| | gatatcgccgtggaatgggagagcaatggccagcctgagaacaactacaa | |
| | gaccacacctcctgtgctggacagcgacggctcattcttcctgtacagcc | |
| | ggctgaccgtggacaagagcagatggcaagagggcaacgtgttcagctgc | |
| | agcgtgatgcacgaggccctgcacaaccactacacccagaagtctctgag | |
| | cctgagcctgggcaagatgttctgggtgctcgttgttgttggcggcgtgc | |
| | tggcctgttactccctgctggttaccgtggccttcatcatcttttgggtc | |
| | aagcggggcagaaagaagctgctctacatcttcaagcagcccttcatgcg | |
| | gcccgtgcagaccacacaagaggaagatggctgctcctgcagattccccg | |
| | aggaagaagaaggcggctgcgagctgagagtgaagttcagcagatccgcc | |
| | gacgctccagcctatcagcagggacagaaccagctgtacaacgagctgaa | |
| | cctggggagaagagaagagtacgacgtgctggataagcggagaggcagag | |
| | atcctgagatgggcggcaagcccagacggaagaatcctcaagagggcctg | |
| | tataatgagctgcagaaagacaagatggccgaggcctacagcgagatcgg | |
| | aatgaagggcgagcgcagaagaggcaagggacacgatggactgtaccagg | |
| | gactgagcaccgccaccaaggatacctatgacgcactgcacatgcaggcc | |
| | ctgccacctaga | |
| 111 | atgccgctgctgctactgctgcccctgctgtgggcaggggctctagctca | CAR 5 (nt) |
| | ggtccaactcgtggaatcaggaggtggacttgttcaccccggcggaagcc | |
| | tgagactgtcttgtgccgcttccggattcacattccggtcccactccatg | |
| | aattgggtccgacaagctcccggcaaaggccttgaatgggtgtccagcat | |
| | cagcagcgacagcacctacacctactatgccgacagcgtgaagggaaggt | |
| | tcacaatctctcgggacaacgccaagaacagcctgtacctgcagatgaac | |
| | tccctcagagccgaggatacagctgtgtattactgtgctagaagtggcgg | |
| | ccagtggaagtactacgactactggggacaaggcacactcgtgacagtta | |
| | gctctggtggcggaggatctcagtctgccctgacacagcctgccagcgtt | |
| | agtgctagtcccggacagtctatcgccatcagctgtaccggcaccagctc | |
| | tgacgttggctggtatcagcagcaccctggcaaggcccctaagctgatga | |
| | tctacgaggacagcaagaggcccagcggcgtgtccaatagattcagcggc | |
| | agcaagagcggcaacaccgccagcctgacaattagcggactgcaggccga | |
| | ggacgaggccgattactactgcagcagcaacacccggtccagcacactgg | |
| | tttttggcggaggcaccaagctgacagtgctgggatctagaggtggcgga | |
| | ggatctggcggcggaggaagcggaggcggcggatctcttgaaatggctga | |
| | agtgcagctggtgcagtctggcgccgagatgaagaaacctggcgcctctc | |
| | tgaagctgagctgcaaggccagcggctacaccttcatcgactactacgtg | |
| | tactggatgcggcaggcccctggacagggactcgaatctatgggctggat | |
| | caaccccaatagcggcggcaccaattacgcccagaaattccagggcagag | |
| | tgaccatgaccagagacaccagcatcagcaccgcctacatggaactgagc | |
| | cggctgagatccgacgacaccgccatgtactactgcgccagatctcagcg | |
| | cgacggctacatggattattggggccagggaaccctggtcaccgtgtcca | |
| | gcggtggcggaggatcttcttctgagctcacccaagatcctgccgtgtct | |
| | gtggctctgggccaaacagtgcggattacctgtcagggcgatagcctgag | |
| | aagctactacgccagctggtaccaacagaagccaggacaggctcccgtgc | |
| | tcgtcatttatggcaagaacaacagaccatccggcatccccgatcggttt | |
| | tccggaagcagctctggcaatactgcctccctcaccatcactggcgccca | |
| | agcagaagatgaagcagactactattgtaactccagagacagctccggca | |
| | atcctcctgtggtgttcggaggcggaacaaaactcaccgtcctcgagtct | |
| | aaatacggaccgccttgtcctccatgtcctgctcctccagttgccggacc | |
| | ttccgtgttcctgtttcctccaaagcctaaggacaccctgatgatcagca | |
| | gaacccctgaagtgacctgcgtggtggtggacgtgtcccaagaggatcct | |
| | gaggtgcagttcaactggtatgtggacggcgtggaagtgcacaacgccaa | |
| | gaccaagcctagagaggaacagttccagagcacctacagagtggtgtccg | |
| | tgctgacagtgctgcaccaggattggctgaacggcaaagagtacaagtgc | |
| | aaggtgtccaacaagggcctgcctagcagcatcgagaaaaccatcagcaa | |
| | ggccaagggccagccaagagaaccccaggtgtacacactgcctccaagcc | |
| | aagaggaaatgaccaagaaccaggtgtccctgacctgcctggtcaagggc | |
| | ttctacccttccgatatcgccgtggaatgggagagcaatggccagcctga | |
| | gaacaactacaagaccacacctcctgtgctggacagcgacggctcattct | |
| | tcctgtacagccggctgaccgtggacaagagcagatggcaagagggcaac | |
| | gtgttcagctgcagcgtgatgcacgaggccctgcacaaccactacaccca | |
| | gaagtctctgagcctgagcctgggcaagatgttctgggtgctcgttgttg | |
| | ttggcggcgtgctggcctgttactccctgctggttaccgtggccttcatc | |
| | atcttttgggtcaagcggggcagaaagaagctgctctacatcttcaagca | |
| | gcccttcatgcggcccgtgcagaccacacaagaggaagatggctgctcct | |
| | gcagattccccgaggaagaagaaggcggctgcgagctgagagtgaagttc | |
| | agcagatccgccgacgctccagcctatcagcagggacagaaccagctgta | |
| | caacgagctgaacctggggagaagagaagagtacgacgtgctggataagc | |
| | ggagaggcagagatcctgagatgggcggcaagcccagacggaagaatcct | |
| | caagagggcctgtataatgagctgcagaaagacaagatggccgaggccta | |
| | cagcgagatcggaatgaagggcgagcgcagaagaggcaagggacacgatg | |
| | gactgtaccagggactgagcaccgccaccaaggatacctatgacgcactg | |
| | cacatgcaggccctgccacctaga | |
| 112 | atgccgctgctgctactgctgcccctgctgtgggcaggggctctagcttc | CAR 6 (nt) |
| | ttctgagctcacccaagatcctgccgtgtctgtggctctgggccaaacag | |
| | tgcggattacctgtcagggcgatagcctgagaagctactacgccagctgg | |
| | taccaacagaagccaggacaggctcccgtgctcgtcatttatggcaagaa | |
| | caacagaccatccggcatccccgatcggttttccggaagcagctctggca | |
| | atactgcctccctcaccatcactggcgcccaagcagaagatgaagcagac | |
| | tactattgtaactccagagacagctccggcaatcctcctgtggtgttcgg | |
| | aggcggaacaaaactcaccgtcctcggtggcggaggatctgaagtgcagc | |
| | tggtgcagtctggcgccgagatgaagaaacctggcgcctctctgaagctg | |
| | agctgcaaggccagcggctacaccttcatcgactactacgtgtactggat | |
| | gcggcaggcccctggacagggactcgaatctatgggctggatcaacccca | |
| | atagcggcggcaccaattacgcccagaaattccagggcagagtgaccatg | |
| | accagagacaccagcatcagcaccgcctacatggaactgagccggctgag | |
| | atccgacgacaccgccatgtactactgcgccagatctcagcgcgacggct | |
| | acatggattattggggccagggaaccctggtcaccgtgtccagcggatct | |
| | agaggtggcggaggatctggcggcggaggaagcggaggcggcggatctct | |
| | tgaaatggctcagtctgccctgacacagcctgccagcgttagtgctagtc | |
| | ccggacagtctatcgccatcagctgtaccggcaccagctctgacgttggc | |
| | tggtatcagcagcaccctggcaaggcccctaagctgatgatctacgagga | |
| | cagcaagaggcccagcggcgtgtccaatagattcagcggcagcaagagcg | |
| | gcaacaccgccagcctgacaattagcggactgcaggccgaggacgaggcc | |
| | gattactactgcagcagcaacacccggtccagcacactggtttttggcgg | |
| | aggcaccaagctgacagtgctgggtggcggaggatctcaggtccaactcg | |
| | tggaatcaggaggtggacttgttcaccccggcggaagcctgagactgtct | |
| | tgtgccgcttccggattcacattccggtcccactccatgaattgggtccg | |
| | acaagctcccggcaaaggccttgaatgggtgtccagcatcagcagcgaca | |
| | gcacctacacctactatgccgacagcgtgaagggaaggttcacaatctct | |
| | cgggacaacgccaagaacagcctgtacctgcagatgaactccctcagagc | |
| | cgaggatacagctgtgtattactgtgctagaagtggcggccagtggaagt | |
| | actacgactactggggacaaggcacactcgtgacagttagctctgagtct | |
| | aaatacggaccgccttgtcctccatgtcctgctcctccagttgccggacc | |
| | ttccgtgttcctgtttcctccaaagcctaaggacaccctgatgatcagca | |
| | gaacccctgaagtgacctgcgtggtggtggacgtgtcccaagaggatcct | |
| | gaggtgcagttcaactggtatgtggacggcgtggaagtgcacaacgccaa | |
| | gaccaagcctagagaggaacagttccagagcacctacagagtggtgtccg | |
| | tgctgacagtgctgcaccaggattggctgaacggcaaagagtacaagtgc | |
| | aaggtgtccaacaagggcctgcctagcagcatcgagaaaaccatcagcaa | |
| | ggccaagggccagccaagagaaccccaggtgtacacactgcctccaagcc | |
| | aagaggaaatgaccaagaaccaggtgtccctgacctgcctggtcaagggc | |
| | ttctacccttccgatatcgccgtggaatgggagagcaatggccagcctga | |
| | gaacaactacaagaccacacctcctgtgctggacagcgacggctcattct | |
| | tcctgtacagccggctgaccgtggacaagagcagatggcaagagggcaac | |
| | gtgttcagctgcagcgtgatgcacgaggccctgcacaaccactacaccca | |
| | gaagtctctgagcctgagcctgggcaagatgttctgggtgctcgttgttg | |
| | ttggcggcgtgctggcctgttactccctgctggttaccgtggccttcatc | |
| | atcttttgggtcaagcggggcagaaagaagctgctctacatcttcaagca | |
| | gcccttcatgcggcccgtgcagaccacacaagaggaagatggctgctcct | |
| | gcagattccccgaggaagaagaaggcggctgcgagctgagagtgaagttc | |
| | agcagatccgccgacgctccagcctatcagcagggacagaaccagctgta | |
| | caacgagctgaacctggggagaagagaagagtacgacgtgctggataagc | |
| | ggagaggcagagatcctgagatgggcggcaagcccagacggaagaatcct | |
| | caagagggcctgtataatgagctgcagaaagacaagatggccgaggccta | |
| | cagcgagatcggaatgaagggcgagcgcagaagaggcaagggacacgatg | |
| | gactgtaccagggactgagcaccgccaccaaggatacctatgacgcactg | |
| | cacatgcaggccctgccacctaga | |
| 113 | atgccgctgctgctactgctgcccctgctgtgggcaggggctctagctca | CAR 10 (nt) |
| | gtctgccctgacacagcctgccagcgttagtgctagtcccggacagtcta | |
| | tcgccatcagctgtaccggcaccagctctgacgttggctggtatcagcag | |
| | caccctggcaaggcccctaagctgatgatctacgaggacagcaagaggcc | |
| | cagcggcgtgtccaatagattcagcggcagcaagagcggcaacaccgcca | |
| | gcctgacaattagcggactgcaggccgaggacgaggccgattactactgc | |
| | agcagcaacacccggtccagcacactggtttttggcggaggcaccaagct | |
| | gacagtgctgggtggcggaggatctcaggtccaactcgtggaatcaggag | |
| | gtggacttgttcaccccggcggaagcctgagactgtcttgtgccgcttcc | |
| | ggattcacattccggtcccactccatgaattgggtccgacaagctcccgg | |
| | caaaggccttgaatgggtgtccagcatcagcagcgacagcacctacacct | |
| | actatgccgacagcgtgaagggaaggttcacaatctctcgggacaacgcc | |
| | aagaacagcctgtacctgcagatgaactccctcagagccgaggatacagc | |
| | tgtgtattactgtgctagaagtggcggccagtggaagtactacgactact | |
| | ggggacaaggcacactcgtgacagttagctctggcagccggggtggaggt | |
| | ggaagcggcggtggtggctccggaggagggggtagcctcgagatggcatc | |
| | ttctgagctcacccaagatcctgccgtgtctgtggctctgggccaaacag | |
| | tgcggattacctgtcagggcgatagcctgagaagctactacgccagctgg | |
| | taccaacagaagccaggacaggctcccgtgctcgtcatttatggcaagaa | |
| | caacagaccatccggcatccccgatcggttttccggaagcagctctggca | |
| | atactgcctccctcaccatcactggcgcccaagcagaagatgaagcagac | |
| | tactattgtaactccagagacagctccggcaatcctcctgtggtgttcgg | |
| | aggcggaacaaaactcaccgtcctcggtggcggaggatctgaagtgcagc | |
| | tggtgcagtctggcgccgagatgaagaaacctggcgcctctctgaagctg | |
| | agctgcaaggccagcggctacaccttcatcgactactacgtgtactggat | |
| | gcggcaggcccctggacagggactcgaatctatgggctggatcaacccca | |
| | atagcggcggcaccaattacgcccagaaattccagggcagagtgaccatg | |
| | accagagacaccagcatcagcaccgcctacatggaactgagccggctgag | |
| | atccgacgacaccgccatgtactactgcgccagatctcagcgcgacggct | |
| | acatggattattggggccagggaaccctggtcaccgtgtccagcgagtct | |
| | aaatacggaccgccttgtcctccatgtcctgctcctccagttgccggacc | |
| | ttccgtgttcctgtttcctccaaagcctaaggacaccctgatgatcagca | |
| | gaacccctgaagtgacctgcgtggtggtggacgtgtcccaagaggatcct | |
| | gaggtgcagttcaactggtatgtggacggcgtggaagtgcacaacgccaa | |
| | gaccaagcctagagaggaacagttccagagcacctacagagtggtgtccg | |
| | tgctgacagtgctgcaccaggattggctgaacggcaaagagtacaagtgc | |
| | aaggtgtccaacaagggcctgcctagcagcatcgagaaaaccatcagcaa | |
| | ggccaagggccagccaagagaaccccaggtgtacacactgcctccaagcc | |
| | aagaggaaatgaccaagaaccaggtgtccctgacctgcctggtcaagggc | |
| | ttctacccttccgatatcgccgtggaatgggagagcaatggccagcctga | |
| | gaacaactacaagaccacacctcctgtgctggacagcgacggctcattct | |
| | tcctgtacagccggctgaccgtggacaagagcagatggcaagagggcaac | |
| | gtgttcagctgcagcgtgatgcacgaggccctgcacaaccactacaccca | |
| | gaagtctctgagcctgagcctgggcaagatgttctgggtgctcgttgttg | |
| | ttggcggcgtgctggcctgttactccctgctggttaccgtggccttcatc | |
| | atcttttgggtcaagcggggcagaaagaagctgctctacatcttcaagca | |
| | gcccttcatgcggcccgtgcagaccacacaagaggaagatggctgctcct | |
| | gcagattccccgaggaagaagaaggcggctgcgagctgagagtgaagttc | |
| | agcagatccgccgacgctccagcctatcagcagggacagaaccagctgta | |
| | caacgagctgaacctggggagaagagaagagtacgacgtgctggataagc | |
| | ggagaggcagagatcctgagatgggcggcaagcccagacggaagaatcct | |
| | caagagggcctgtataatgagctgcagaaagacaagatggccgaggccta | |
| | cagcgagatcggaatgaagggcgagcgcagaagaggcaagggacacgatg | |
| | gactgtaccagggactgagcaccgccaccaaggatacctatgacgcactg | |
| | cacatgcaggccctgccacctaga | |
| 114 | atgccgctgctgctactgctgcccctgctgtgggcaggggctctagctca | CAR 11 (nt) |
| | gtctgccctgacacagcctgccagcgttagtgctagtcccggacagtcta | |
| | tcgccatcagctgtaccggcaccagctctgacgttggctggtatcagcag | |
| | caccctggcaaggcccctaagctgatgatctacgaggacagcaagaggcc | |
| | cagcggcgtgtccaatagattcagcggcagcaagagcggcaacaccgcca | |
| | gcctgacaattagcggactgcaggccgaggacgaggccgattactactgc | |
| | agcagcaacacccggtccagcacactggtttttggcggaggcaccaagct | |
| | gacagtgctgggtggcggaggatcttcttctgagctcacccaagatcctg | |
| | ccgtgtctgtggctctgggccaaacagtgcggattacctgtcagggcgat | |
| | agcctgagaagctactacgccagctggtaccaacagaagccaggacaggc | |
| | tcccgtgctcgtcatttatggcaagaacaacagaccatccggcatccccg | |
| | atcggttttccggaagcagctctggcaatactgcctccctcaccatcact | |
| | ggcgcccaagcagaagatgaagcagactactattgtaactccagagacag | |
| | ctccggcaatcctcctgtggtgttcggaggcggaacaaaactcaccgtcc | |
| | tcggcagccggggtggaggtggaagcggcggtggtggctccggaggaggg | |
| | ggtagcctcgagatggcacaggtccaactcgtggaatcaggaggtggact | |
| | tgttcaccccggcggaagcctgagactgtcttgtgccgcttccggattca | |
| | cattccggtcccactccatgaattgggtccgacaagctcccggcaaaggc | |
| | cttgaatgggtgtccagcatcagcagcgacagcacctacacctactatgc | |
| | cgacagcgtgaagggaaggttcacaatctctcgggacaacgccaagaaca | |
| | gcctgtacctgcagatgaactccctcagagccgaggatacagctgtgtat | |
| | tactgtgctagaagtggcggccagtggaagtactacgactactggggaca | |
| | aggcacactcgtgacagttagctctggtggcggaggatctgaagtgcagc | |
| | tggtgcagtctggcgccgagatgaagaaacctggcgcctctctgaagctg | |
| | agctgcaaggccagcggctacaccttcatcgactactacgtgtactggat | |
| | gcggcaggcccctggacagggactcgaatctatgggctggatcaacccca | |
| | atagcggcggcaccaattacgcccagaaattccagggcagagtgaccatg | |
| | accagagacaccagcatcagcaccgcctacatggaactgagccggctgag | |
| | atccgacgacaccgccatgtactactgcgccagatctcagcgcgacggct | |
| | acatggattattggggccagggaaccctggtcaccgtgtccagcgagtct | |
| | aaatacggaccgccttgtcctccatgtcctgctcctccagttgccggacc | |
| | ttccgtgttcctgtttcctccaaagcctaaggacaccctgatgatcagca | |
| | gaacccctgaagtgacctgcgtggtggtggacgtgtcccaagaggatcct | |
| | gaggtgcagttcaactggtatgtggacggcgtggaagtgcacaacgccaa | |
| | gaccaagcctagagaggaacagttccagagcacctacagagtggtgtccg | |
| | tgctgacagtgctgcaccaggattggctgaacggcaaagagtacaagtgc | |
| | aaggtgtccaacaagggcctgcctagcagcatcgagaaaaccatcagcaa | |
| | ggccaagggccagccaagagaaccccaggtgtacacactgcctccaagcc | |
| | aagaggaaatgaccaagaaccaggtgtccctgacctgcctggtcaagggc | |
| | ttctacccttccgatatcgccgtggaatgggagagcaatggccagcctga | |
| | gaacaactacaagaccacacctcctgtgctggacagcgacggctcattct | |
| | tcctgtacagccggctgaccgtggacaagagcagatggcaagagggcaac | |
| | gtgttcagctgcagcgtgatgcacgaggccctgcacaaccactacaccca | |
| | gaagtctctgagcctgagcctgggcaagatgttctgggtgctcgttgttg | |
| | ttggcggcgtgctggcctgttactccctgctggttaccgtggccttcatc | |
| | atcttttgggtcaagcggggcagaaagaagctgctctacatcttcaagca | |
| | gcccttcatgcggcccgtgcagaccacacaagaggaagatggctgctcct | |
| | gcagattccccgaggaagaagaaggcggctgcgagctgagagtgaagttc | |
| | agcagatccgccgacgctccagcctatcagcagggacagaaccagctgta | |
| | caacgagctgaacctggggagaagagaagagtacgacgtgctggataagc | |
| | ggagaggcagagatcctgagatgggcggcaagcccagacggaagaatcct | |
| | caagagggcctgtataatgagctgcagaaagacaagatggccgaggccta | |
| | cagcgagatcggaatgaagggcgagcgcagaagaggcaagggacacgatg | |
| | gactgtaccagggactgagcaccgccaccaaggatacctatgacgcactg | |
| | cacatgcaggccctgccacctaga | |
| 115 | atgccgctgctgctactgctgcccctgctgtgggcaggggctctagctca | CAR 7 (nt) |
| | ggtccaactcgtggaatcaggaggtggacttgttcaccccggcggaagcc | |
| | tgagactgtcttgtgccgcttccggattcacattccggtcccactccatg | |
| | aattgggtccgacaagctcccggcaaaggccttgaatgggtgtccagcat | |
| | cagcagcgacagcacctacacctactatgccgacagcgtgaagggaaggt | |
| | tcacaatctctcgggacaacgccaagaacagcctgtacctgcagatgaac | |
| | tccctcagagccgaggatacagctgtgtattactgtgctagaagtggcgg | |
| | ccagtggaagtactacgactactggggacaaggcacactcgtgacagtta | |
| | gctctggtggaggtggaagcggcggtggtggctcccagtctgccctgaca | |
| | cagcctgccagcgttagtgctagtcccggacagtctatcgccatcagctg | |
| | taccggcaccagctctgacgttggctggtatcagcagcaccctggcaagg | |
| | cccctaagctgatgatctacgaggacagcaagaggcccagcggcgtgtcc | |
| | aatagattcagcggcagcaagagcggcaacaccgccagcctgacaattag | |
| | cggactgcaggccgaggacgaggccgattactactgcagcagcaacaccc | |
| | ggtccagcacactggtttttggcggaggcaccaagctgacagtgctggga | |
| | tctagaggtggcggaggatctggcggcggaggaagcggaggcggcggatc | |
| | tcttgaaatggctgaagtgcagctggtgcagtctggcgccgagatgaaga | |
| | aacctggcgcctctctgaagctgagctgcaaggccagcggctacaccttc | |
| | atcgactactacgtgtactggatgcggcaggcccctggacagggactcga | |
| | atctatgggctggatcaaccccaatagcggcggcaccaattacgcccaga | |
| | aattccagggcagagtgaccatgaccagagacaccagcatcagcaccgcc | |
| | tacatggaactgagccggctgagatccgacgacaccgccatgtactactg | |
| | cgccagatctcagcgcgacggctacatggattattggggccagggaaccc | |
| | tggtcaccgtgtccagcggtggaggtggaagcggcggtggtggctcctct | |
| | tctgagctcacccaagatcctgccgtgtctgtggctctgggccaaacagt | |
| | gcggattacctgtcagggcgatagcctgagaagctactacgccagctggt | |
| | accaacagaagccaggacaggctcccgtgctcgtcatttatggcaagaac | |
| | aacagaccatccggcatccccgatcggttttccggaagcagctctggcaa | |
| | tactgcctccctcaccatcactggcgcccaagcagaagatgaagcagact | |
| | actattgtaactccagagacagctccggcaatcctcctgtggtgttcgga | |
| | ggcggaacaaaactcaccgtcctcgagtctaaatacggaccgccttgtcc | |
| | tccatgtcctgctcctccagttgccggaccttccgtgttcctgtttcctc | |
| | caaagcctaaggacaccctgatgatcagcagaacccctgaagtgacctgc | |
| | gtggtggtggacgtgtcccaagaggatcctgaggtgcagttcaactggta | |
| | tgtggacggcgtggaagtgcacaacgccaagaccaagcctagagaggaac | |
| | agttccagagcacctacagagtggtgtccgtgctgacagtgctgcaccag | |
| | gattggctgaacggcaaagagtacaagtgcaaggtgtccaacaagggcct | |
| | gcctagcagcatcgagaaaaccatcagcaaggccaagggccagccaagag | |
| | aaccccaggtgtacacactgcctccaagccaagaggaaatgaccaagaac | |
| | caggtgtccctgacctgcctggtcaagggcttctacccttccgatatcgc | |
| | cgtggaatgggagagcaatggccagcctgagaacaactacaagaccacac | |
| | ctcctgtgctggacagcgacggctcattcttcctgtacagccggctgacc | |
| | gtggacaagagcagatggcaagagggcaacgtgttcagctgcagcgtgat | |
| | gcacgaggccctgcacaaccactacacccagaagtctctgagcctgagcc | |
| | tgggcaagatgttctgggtgctcgttgttgttggcggcgtgctggcctgt | |
| | tactccctgctggttaccgtggccttcatcatcttttgggtcaagcgggg | |
| | cagaaagaagctgctctacatcttcaagcagcccttcatgcggcccgtgc | |
| | agaccacacaagaggaagatggctgctcctgcagattccccgaggaagaa | |
| | gaaggcggctgcgagctgagagtgaagttcagcagatccgccgacgctcc | |
| | agcctatcagcagggacagaaccagctgtacaacgagctgaacctgggga | |
| | gaagagaagagtacgacgtgctggataagcggagaggcagagatcctgag | |
| | atgggcggcaagcccagacggaagaatcctcaagagggcctgtataatga | |
| | gctgcagaaagacaagatggccgaggcctacagcgagatcggaatgaagg | |
| | gcgagcgcagaagaggcaagggacacgatggactgtaccagggactgagc | |
| | accgccaccaaggatacctatgacgcactgcacatgcaggccctgccacc | |
| | taga | |
| 116 | atgccgctgctgctactgctgcccctgctgtgggcaggggctctagctga | CAR 12 (nt) |
| | agtgcagctggtgcagtctggcgccgagatgaagaaacctggcgcctctc | |
| | tgaagctgagctgcaaggccagcggctacaccttcatcgactactacgtg | |
| | tactggatgcggcaggcccctggacagggactcgaatctatgggctggat | |
| | caaccccaatagcggcggcaccaattacgcccagaaattccagggcagag | |
| | tgaccatgaccagagacaccagcatcagcaccgcctacatggaactgagc | |
| | cggctgagatccgacgacaccgccatgtactactgcgccagatctcagcg | |
| | cgacggctacatggattattggggccagggaaccctggtcaccgtgtcca | |
| | gcggtggaggtggaagcggcggtggtggctcccaggtccaactcgtggaa | |
| | tcaggaggtggacttgttcaccccggcggaagcctgagactgtcttgtgc | |
| | cgcttccggattcacattccggtcccactccatgaattgggtccgacaag | |
| | ctcccggcaaaggccttgaatgggtgtccagcatcagcagcgacagcacc | |
| | tacacctactatgccgacagcgtgaagggaaggttcacaatctctcggga | |
| | caacgccaagaacagcctgtacctgcagatgaactccctcagagccgagg | |
| | atacagctgtgtattactgtgctagaagtggcggccagtggaagtactac | |
| | gactactggggacaaggcacactcgtgacagttagctctggcagccgggg | |
| | tggaggtggaagcggcggtggtggctccggaggagggggtagcctcgaga | |
| | tggcatcttctgagctcacccaagatcctgccgtgtctgtggctctgggc | |
| | caaacagtgcggattacctgtcagggcgatagcctgagaagctactacgc | |
| | cagctggtaccaacagaagccaggacaggctcccgtgctcgtcatttatg | |
| | gcaagaacaacagaccatccggcatccccgatcggttttccggaagcagc | |
| | tctggcaatactgcctccctcaccatcactggcgcccaagcagaagatga | |
| | agcagactactattgtaactccagagacagctccggcaatcctcctgtgg | |
| | tgttcggaggcggaacaaaactcaccgtcctcggtggaggtggaagcggc | |
| | ggtggtggctcccagtctgccctgacacagcctgccagcgttagtgctag | |
| | tcccggacagtctatcgccatcagctgtaccggcaccagctctgacgttg | |
| | gctggtatcagcagcaccctggcaaggcccctaagctgatgatctacgag | |
| | gacagcaagaggcccagcggcgtgtccaatagattcagcggcagcaagag | |
| | cggcaacaccgccagcctgacaattagcggactgcaggccgaggacgagg | |
| | ccgattactactgcagcagcaacacccggtccagcacactggtttttggc | |
| | ggaggcaccaagctgacagtgctggagtctaaatacggaccgccttgtcc | |
| | tccatgtcctgctcctccagttgccggaccttccgtgttcctgtttcctc | |
| | caaagcctaaggacaccctgatgatcagcagaacccctgaagtgacctgc | |
| | gtggtggtggacgtgtcccaagaggatcctgaggtgcagttcaactggta | |
| | tgtggacggcgtggaagtgcacaacgccaagaccaagcctagagaggaac | |
| | agttccagagcacctacagagtggtgtccgtgctgacagtgctgcaccag | |
| | gattggctgaacggcaaagagtacaagtgcaaggtgtccaacaagggcct | |
| | gcctagcagcatcgagaaaaccatcagcaaggccaagggccagccaagag | |
| | aaccccaggtgtacacactgcctccaagccaagaggaaatgaccaagaac | |
| | caggtgtccctgacctgcctggtcaagggcttctacccttccgatatcgc | |
| | cgtggaatgggagagcaatggccagcctgagaacaactacaagaccacac | |
| | ctcctgtgctggacagcgacggctcattcttcctgtacagccggctgacc | |
| | gtggacaagagcagatggcaagagggcaacgtgttcagctgcagcgtgat | |
| | gcacgaggccctgcacaaccactacacccagaagtctctgagcctgagcc | |
| | tgggcaagatgttctgggtgctcgttgttgttggcggcgtgctggcctgt | |
| | tactccctgctggttaccgtggccttcatcatcttttgggtcaagcgggg | |
| | cagaaagaagctgctctacatcttcaagcagcccttcatgcggcccgtgc | |
| | agaccacacaagaggaagatggctgctcctgcagattccccgaggaagaa | |
| | gaaggcggctgcgagctgagagtgaagttcagcagatccgccgacgctcc | |
| | agcctatcagcagggacagaaccagctgtacaacgagctgaacctgggga | |
| | gaagagaagagtacgacgtgctggataagcggagaggcagagatcctgag | |
| | atgggcggcaagcccagacggaagaatcctcaagagggcctgtataatga | |
| | gctgcagaaagacaagatggccgaggcctacagcgagatcggaatgaagg | |
| | gcgagcgcagaagaggcaagggacacgatggactgtaccagggactgagc | |
| | accgccaccaaggatacctatgacgcactgcacatgcaggccctgccacc | |
| | taga | |
| 117 | atgccgctgctgctactgctgcccctgctgtgggcaggggctctagctca | CAR 13 (nt) |
| | gtctgccctgacacagcctgccagcgttagtgctagtcccggacagtcta | |
| | tcgccatcagctgtaccggcaccagctctgacgttggctggtatcagcag | |
| | caccctggcaaggcccctaagctgatgatctacgaggacagcaagaggcc | |
| | cagcggcgtgtccaatagattcagcggcagcaagagcggcaacaccgcca | |
| | gcctgacaattagcggactgcaggccgaggacgaggccgattactactgc | |
| | agcagcaacacccggtccagcacactggtttttggcggaggcaccaagct | |
| | gacagtgctgggtggaggtggaagcggcggtggtggctcccaggtccaac | |
| | tcgtggaatcaggaggtggacttgttcaccccggcggaagcctgagactg | |
| | tcttgtgccgcttccggattcacattccggtcccactccatgaattgggt | |
| | ccgacaagctcccggcaaaggccttgaatgggtgtccagcatcagcagcg | |
| | acagcacctacacctactatgccgacagcgtgaagggaaggttcacaatc | |
| | tctcgggacaacgccaagaacagcctgtacctgcagatgaactccctcag | |
| | agccgaggatacagctgtgtattactgtgctagaagtggcggccagtgga | |
| | agtactacgactactggggacaaggcacactcgtgacagttagctctggc | |
| | agccggggtggaggtggaagcggcggtggtggctccggaggagggggtag | |
| | cctcgagatggcatcttctgagctcacccaagatcctgccgtgtctgtgg | |
| | ctctgggccaaacagtgcggattacctgtcagggcgatagcctgagaagc | |
| | tactacgccagctggtaccaacagaagccaggacaggctcccgtgctcgt | |
| | catttatggcaagaacaacagaccatccggcatccccgatcggttttccg | |
| | gaagcagctctggcaatactgcctccctcaccatcactggcgcccaagca | |
| | gaagatgaagcagactactattgtaactccagagacagctccggcaatcc | |
| | tcctgtggtgttcggaggcggaacaaaactcaccgtcctcggtggaggtg | |
| | gaagcggcggtggtggctccgaagtgcagctggtgcagtctggcgccgag | |
| | atgaagaaacctggcgcctctctgaagctgagctgcaaggccagcggcta | |
| | caccttcatcgactactacgtgtactggatgcggcaggcccctggacagg | |
| | gactcgaatctatgggctggatcaaccccaatagcggcggcaccaattac | |
| | gcccagaaattccagggcagagtgaccatgaccagagacaccagcatcag | |
| | caccgcctacatggaactgagccggctgagatccgacgacaccgccatgt | |
| | actactgcgccagatctcagcgcgacggctacatggattattggggccag | |
| | ggaaccctggtcaccgtgtccagcgagtctaaatacggaccgccttgtcc | |
| | tccatgtcctgctcctccagttgccggaccttccgtgttcctgtttcctc | |
| | caaagcctaaggacaccctgatgatcagcagaacccctgaagtgacctgc | |
| | gtggtggtggacgtgtcccaagaggatcctgaggtgcagttcaactggta | |
| | tgtggacggcgtggaagtgcacaacgccaagaccaagcctagagaggaac | |
| | agttccagagcacctacagagtggtgtccgtgctgacagtgctgcaccag | |
| | gattggctgaacggcaaagagtacaagtgcaaggtgtccaacaagggcct | |
| | gcctagcagcatcgagaaaaccatcagcaaggccaagggccagccaagag | |
| | aaccccaggtgtacacactgcctccaagccaagaggaaatgaccaagaac | |
| | caggtgtccctgacctgcctggtcaagggcttctacccttccgatatcgc | |
| | cgtggaatgggagagcaatggccagcctgagaacaactacaagaccacac | |
| | ctcctgtgctggacagcgacggctcattcttcctgtacagccggctgacc | |
| | gtggacaagagcagatggcaagagggcaacgtgttcagctgcagcgtgat | |
| | gcacgaggccctgcacaaccactacacccagaagtctctgagcctgagcc | |
| | tgggcaagatgttctgggtgctcgttgttgttggcggcgtgctggcctgt | |
| | tactccctgctggttaccgtggccttcatcatcttttgggtcaagcgggg | |
| | cagaaagaagctgctctacatcttcaagcagcccttcatgcggcccgtgc | |
| | agaccacacaagaggaagatggctgctcctgcagattccccgaggaagaa | |
| | gaaggcggctgcgagctgagagtgaagttcagcagatccgccgacgctcc | |
| | agcctatcagcagggacagaaccagctgtacaacgagctgaacctgggga | |
| | gaagagaagagtacgacgtgctggataagcggagaggcagagatcctgag | |
| | atgggcggcaagcccagacggaagaatcctcaagagggcctgtataatga | |
| | gctgcagaaagacaagatggccgaggcctacagcgagatcggaatgaagg | |
| | gcgagcgcagaagaggcaagggacacgatggactgtaccagggactgagc | |
| | accgccaccaaggatacctatgacgcactgcacatgcaggccctgccacc | |
| | taga | |
| 118 | atgccgctgctgctactgctgcccctgctgtgggcaggggctctagctca | CAR 14 (nt) |
| | gtctgccctgacacagcctgccagcgttagtgctagtcccggacagtcta | |
| | tcgccatcagctgtaccggcaccagctctgacgttggctggtatcagcag | |
| | caccctggcaaggcccctaagctgatgatctacgaggacagcaagaggcc | |
| | cagcggcgtgtccaatagattcagcggcagcaagagcggcaacaccgcca | |
| | gcctgacaattagcggactgcaggccgaggacgaggccgattactactgc | |
| | agcagcaacacccggtccagcacactggtttttggcggaggcaccaagct | |
| | gacagtgctgggtggaggtggaagcggcggtggtggctcccaggtccaac | |
| | tcgtggaatcaggaggtggacttgttcaccccggcggaagcctgagactg | |
| | tcttgtgccgcttccggattcacattccggtcccactccatgaattgggt | |
| | ccgacaagctcccggcaaaggccttgaatgggtgtccagcatcagcagcg | |
| | acagcacctacacctactatgccgacagcgtgaagggaaggttcacaatc | |
| | tctcgggacaacgccaagaacagcctgtacctgcagatgaactccctcag | |
| | agccgaggatacagctgtgtattactgtgctagaagtggcggccagtgga | |
| | agtactacgactactggggacaaggcacactcgtgacagttagctctggc | |
| | agcacctccggcagcggcaagcctggcagcggcgagggcagcaccaaggg | |
| | ctcttctgagctcacccaagatcctgccgtgtctgtggctctgggccaaa | |
| | cagtgcggattacctgtcagggcgatagcctgagaagctactacgccagc | |
| | tggtaccaacagaagccaggacaggctcccgtgctcgtcatttatggcaa | |
| | gaacaacagaccatccggcatccccgatcggttttccggaagcagctctg | |
| | gcaatactgcctccctcaccatcactggcgcccaagcagaagatgaagca | |
| | gactactattgtaactccagagacagctccggcaatcctcctgtggtgtt | |
| | cggaggcggaacaaaactcaccgtcctcggtggaggtggaagcggcggtg | |
| | gtggctccgaagtgcagctggtgcagtctggcgccgagatgaagaaacct | |
| | ggcgcctctctgaagctgagctgcaaggccagcggctacaccttcatcga | |
| | ctactacgtgtactggatgcggcaggcccctggacagggactcgaatcta | |
| | tgggctggatcaaccccaatagcggcggcaccaattacgcccagaaattc | |
| | cagggcagagtgaccatgaccagagacaccagcatcagcaccgcctacat | |
| | ggaactgagccggctgagatccgacgacaccgccatgtactactgcgcca | |
| | gatctcagcgcgacggctacatggattattggggccagggaaccctggtc | |
| | accgtgtccagcgagtctaaatacggaccgccttgtcctccatgtcctgc | |
| | tcctccagttgccggaccttccgtgttcctgtttcctccaaagcctaagg | |
| | acaccctgatgatcagcagaacccctgaagtgacctgcgtggtggtggac | |
| | gtgtcccaagaggatcctgaggtgcagttcaactggtatgtggacggcgt | |
| | ggaagtgcacaacgccaagaccaagcctagagaggaacagttccagagca | |
| | cctacagagtggtgtccgtgctgacagtgctgcaccaggattggctgaac | |
| | ggcaaagagtacaagtgcaaggtgtccaacaagggcctgcctagcagcat | |
| | cgagaaaaccatcagcaaggccaagggccagccaagagaaccccaggtgt | |
| | acacactgcctccaagccaagaggaaatgaccaagaaccaggtgtccctg | |
| | acctgcctggtcaagggcttctacccttccgatatcgccgtggaatggga | |
| | gagcaatggccagcctgagaacaactacaagaccacacctcctgtgctgg | |
| | acagcgacggctcattcttcctgtacagccggctgaccgtggacaagagc | |
| | agatggcaagagggcaacgtgttcagctgcagcgtgatgcacgaggccct | |
| | gcacaaccactacacccagaagtctctgagcctgagcctgggcaagatgt | |
| | tctgggtgctcgttgttgttggcggcgtgctggcctgttactccctgctg | |
| | gttaccgtggccttcatcatcttttgggtcaagcggggcagaaagaagct | |
| | gctctacatcttcaagcagcccttcatgcggcccgtgcagaccacacaag | |
| | aggaagatggctgctcctgcagattccccgaggaagaagaaggcggctgc | |
| | gagctgagagtgaagttcagcagatccgccgacgctccagcctatcagca | |
| | gggacagaaccagctgtacaacgagctgaacctggggagaagagaagagt | |
| | acgacgtgctggataagcggagaggcagagatcctgagatgggcggcaag | |
| | cccagacggaagaatcctcaagagggcctgtataatgagctgcagaaaga | |
| | caagatggccgaggcctacagcgagatcggaatgaagggcgagcgcagaa | |
| | gaggcaagggacacgatggactgtaccagggactgagcaccgccaccaag | |
| | gatacctatgacgcactgcacatgcaggccctgccacctaga | |
| 119 | atgcctctgctgctgcttctgcctcttctttgggctggtgctctggctca | CAR 5 (nt) |
| | ggtgcagctggttgaatctggcggaggactggttcaccctggcggatctc | CO/SSE |
| | tgagactgtcttgtgccgccagcggcttcaccttccgatcccactccatg | |
| | aactgggtccgacaggcccctggcaaaggccttgaatgggtgtccagcat | |
| | cagcagcgacagcacctacacctactacgccgacagcgtgaagggcagat | |
| | tcaccatctccagagacaacgccaagaacagcctgtacctgcagatgaac | |
| | tccctgagagccgaggacaccgccgtgtactactgtgctagaagcggcgg | |
| | acagtggaagtactacgactattggggccagggcaccctggtcacagttt | |
| | ctagcggaggcggaggctctcagtctgctctgacacagcctgcctccgtg | |
| | tctgcttctcctggacagtctatcgccatctcctgtaccggcaccagctc | |
| | tgacgttggctggtatcagcagcacccaggcaaggcccctaagctgatga | |
| | tctacgaggacagcaagaggcccagcggcgtgtccaatcgattcagcggc | |
| | agcaagagcggcaacaccgccagcctgacaattagcggactgcaggccga | |
| | ggacgaggccgattactactgcagcagcaacacccggtccagcacactgg | |
| | tttttggcggaggcaccaagctgacagtgctgggaagtagaggcggcgga | |
| | ggtagtggtggtggcggtagtggtggcggaggatctcttgaaatggccga | |
| | ggttcagctggtgcagtctggcgccgagatgaagaaacctggcgcctctc | |
| | tgaagctgagctgcaaggcctctggctacaccttcatcgactactacgtg | |
| | tactggatgcggcaggccccaggacagggactcgaatctatgggctggat | |
| | caaccccaatagcggcggcaccaattacgcccagaaattccagggcagag | |
| | tgaccatgaccagggacaccagcatctccaccgcctacatggaactgagc | |
| | cggctgagatccgacgatacagccatgtactattgcgccagaagccagcg | |
| | cgacggctacatggattattggggacaaggcacactcgtgaccgtgtcaa | |
| | gtggcggtggtggatctagcagcgagctgacacaggatccagccgtgtct | |
| | gttgccctgggccagacagtgcggattacctgtcaaggcgacagcctgcg | |
| | gagctactatgcctcctggtatcaacagaagcccggccaggctcctgtgc | |
| | tggtcatctacggcaagaacaacagacccagcggcatccccgatagattc | |
| | tccggctctagctctggcaacacagcttccctgacaatcactggcgccca | |
| | ggccgaagatgaagccgactattactgtaacagccgggacagcagcggca | |
| | accctcctgttgtttttggaggcggaacaaaactgaccgtgctggagtct | |
| | aaatacggaccgccttgtcctccatgtcctgctcctcccgtggctggccc | |
| | ttccgtgtttctgttccctccaaagcctaaggacaccctgatgatcagca | |
| | gaacccctgaagtgacctgcgtggtggtcgacgtgtcccaagaggatcct | |
| | gaggtgcagttcaactggtatgtggacggcgtggaagtgcacaatgccaa | |
| | gaccaagcctagagaggaacagttccagagcacctatagagtggtgtccg | |
| | tgctgaccgtgctgcaccaggattggctgaacggcaaagagtacaagtgc | |
| | aaggtgtccaacaagggactgcccagcagcatcgagaaaaccatctccaa | |
| | ggccaagggccagcctagggaaccccaggtttacacactgcctccaagcc | |
| | aagaggaaatgaccaagaaccaggtgtccctgacctgcctggtcaagggc | |
| | ttctacccttccgatatcgccgtggaatgggagagcaatggccagccaga | |
| | gaacaactacaagaccacacctcctgtgctggatagcgacggctcattct | |
| | tcctgtactcccgactgaccgtggacaagagcagatggcaagagggcaat | |
| | gtgttcagctgcagcgtgatgcacgaggccctgcacaaccactacaccca | |
| | gaagtctctgagcctgagcctgggcaagatgttctgggtgctcgttgttg | |
| | ttggcggcgtgctggcctgttactccctgctggttaccgtggccttcatc | |
| | atcttttgggtcaagcggggcagaaagaagctgctctacatcttcaagca | |
| | gcccttcatgcggcccgtgcagaccacacaagaggaagatggctgctcct | |
| | gccgattccccgaggaagaagaaggcggctgcgagctgagagtgaagttc | |
| | agcagatccgctgacgcccctgcttaccagcagggccagaaccagctgta | |
| | taacgagctgaatctggggcgcagagaagagtacgacgtgctggacaagc | |
| | ggagaggcagagatcctgagatgggcggcaagcccagacggaagaatcct | |
| | caagagggcctctacaacgagctgcagaaagacaagatggccgaggccta | |
| | cagcgagatcggaatgaagggcgaacgcagaagaggaaagggccacgacg | |
| | gactgtatcagggactgagcaccgccaccaaggatacctatgacgcactg | |
| | cacatgcaggccctgccacctaga | |
| 120 | atgcctttgttgctgctgctgcctcttctgtgggctggtgctctggctca | CAR 11 (nt) |
| | gtctgctctgacacagcctgcctccgtgtctgcttctcctggacagtcta | CO/SSE |
| | tcgccatcagctgtaccggcaccagctctgacgttggctggtatcagcag | |
| | caccctggcaaggcccctaagctgatgatctacgaggacagcaagaggcc | |
| | cagcggcgtgtccaatcgcttcagcggcagcaagagcggcaacaccgcca | |
| | gcctgacaattagcggactgcaggccgaggacgaggccgattactactgc | |
| | agctccaacaccagatccagcacactggtgtttggcggaggcaccaagct | |
| | gacagttcttggcggcggaggaagcagctccgagctgacacaagatcctg | |
| | ccgtgtctgtggccctgggccagacagttagaatcacctgtcagggcgac | |
| | agcctgcggagctactacgcctcttggtatcaacagaagcccggacaggc | |
| | ccctgtgctggtcatctacggcaagaacaacagacccagcggcatccccg | |
| | atagattctccggctctagctctggcaataccgcctctctgacaatcact | |
| | ggcgcccaggccgaagatgaagccgactattactgtaacagccgggacag | |
| | cagcggcaaccctcctgttgtttttggaggcggaacaaagctgaccgtgc | |
| | tgggctctagaggtggcggaggtagcggaggcggaggatctggcggtggt | |
| | ggatctctggaaatggctcaggtgcagctggtggaatctggtggcggact | |
| | tgttcaccctggcggaagcctgagactgtcttgtgccgccagcggcttca | |
| | ccttccggtcccactccatgaactgggtccgacaggctcctggcaaaggc | |
| | ctggaatgggtgtccagcatcagcagcgatagcacctacacctactatgc | |
| | cgacagcgtgaagggcagattcaccatcagcagagacaacgccaagaata | |
| | gcctgtacctgcagatgaacagcctgagagccgaggacaccgccgtgtac | |
| | tactgtgctagaagcggcggacagtggaagtactacgactattggggcca | |
| | gggcaccctggtcacagtttctagtggtggtggcggcagcgaagtccagc | |
| | tggttcaatctggcgccgagatgaagaagcctggcgcttctctgaagctg | |
| | agctgcaaggcctccggctacacctttatcgactactacgtgtactggat | |
| | gcggcaggccccaggacagggactcgaatctatgggctggatcaacccca | |
| | atagcggcggcaccaattacgcccagaaattccagggcagagtgaccatg | |
| | accagggacaccagcatctccaccgcctacatggaactgagccggctgag | |
| | atccgacgatacagccatgtactattgcgccagaagccagcgcgacggct | |
| | acatggattattggggacaaggcacactcgtgaccgtgtcctctgagtct | |
| | aaatacggaccgccttgtcctccatgtcctgctcctcccgtggctggccc | |
| | ttccgtgtttctgttccctccaaagcctaaggacaccctgatgatcagca | |
| | gaacccctgaagtgacctgcgtggtggtcgacgtgtcccaagaggatcct | |
| | gaggtgcagttcaactggtatgtggacggcgtggaagtgcacaatgccaa | |
| | gaccaagcctagagaggaacagttccagagcacctatagagtggtgtccg | |
| | tgctgaccgtgctgcaccaggattggctgaacggcaaagagtacaagtgc | |
| | aaggtgtccaacaagggactgcccagcagcatcgagaaaaccatctccaa | |
| | ggccaagggccagcctagggaaccccaggtttacacactgcctccaagcc | |
| | aagaggaaatgaccaagaaccaggtgtccctgacctgcctggtcaagggc | |
| | ttctacccttccgatatcgccgtggaatgggagagcaatggccagccaga | |
| | gaacaactacaagaccacacctcctgtgctggatagcgacggctcattct | |
| | tcctgtactcccgactgaccgtggacaagagcagatggcaagagggcaat | |
| | gtgttcagctgcagcgtgatgcacgaggccctgcacaaccactacaccca | |
| | gaagtctctgagcctgagcctgggcaagatgttctgggtgctcgttgttg | |
| | ttggcggcgtgctggcctgttactccctgctggttaccgtggccttcatc | |
| | atcttttgggtcaagcggggcagaaagaagctgctctacatcttcaagca | |
| | gcccttcatgcggcccgtgcagaccacacaagaggaagatggctgctcct | |
| | gccgattccccgaggaagaagaaggcggctgcgagctgagagtgaagttc | |
| | agcagatccgctgacgcccctgcttaccagcagggccagaaccagctgta | |
| | taacgagctgaatctggggcgcagagaagagtacgacgtgctggacaagc | |
| | ggagaggcagagatcctgagatgggcggcaagcccagacggaagaatcct | |
| | caagagggcctctacaacgagctgcagaaagacaagatggccgaggccta | |
| | cagcgagatcggaatgaagggcgaacgcagaagaggaaagggccacgacg | |
| | gactgtatcagggactgagcaccgccaccaaggatacctatgacgcactg | |
| | cacatgcaggccctgccacctaga | |

What is claimed:

1. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus:
(i) one of the VH region and the VL region of the GPRC5D-binding domain, one of the VH region and the VL region of the BCMA-binding domain, the other of the VH region and the VL region of the BCMA-binding domain, and the other of the VH region and the VL region of the GPRC5D-binding domain; or
(ii) one of the VH region and the VL region of the BCMA-binding domain, one of the VH region and the VL region of the GPRC5D-binding domain, the other of the VH region and the VL region of the GPRC5D-binding domain, and the other of the VH region and the VL region of the BCMA-binding domain;
(b) a spacer;
(c) a transmembrane domain; and
(d) an intracellular signaling domain.

2. The bispecific CAR of claim 1, wherein the extracellular domain comprises, in order from amino to carboxy terminus one of the VH region and the VL region of the GPRC5D-binding domain, one of the VH region and the VL region of the BCMA-binding domain, the other of the VH region and the VL region of the BCMA-binding domain, and the other of the VH region and the VL region of the GPRC5D-binding domain.

3. The bispecific CAR of claim 1 or claim 2, wherein the extracellular domain comprises, in order from amino to carboxy terminus: the VH region of the GPRC5D-binding domain, the VH region of the BCMA-binding domain, the VL region of the BCMA-binding domain, and the VL region of the GPRC5D-binding domain.

4. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus: the VH region of the GPRC5D-binding domain, the VH region of the BCMA-binding domain, the VL region of the BCMA-binding domain, and the VL region of the GPRC5D-binding domain;
(b) a spacer;
(c) a transmembrane domain; and
(d) an intracellular signaling domain.

5. The bispecific CAR of claim 1 or claim 2, wherein the extracellular domain comprises, in order from amino to carboxy terminus: the VH region of the GPRC5D-binding domain, the VL region of the BCMA-binding domain, the VH region of the BCMA-binding domain, and the VL region of the GPRC5D-binding domain.

6. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus: the VH region of the GPRC5D-binding domain, the VL region of the BCMA-binding domain, the VH region of the BCMA-binding domain, and the VL region of the GPRC5D-binding domain;
(b) a spacer;
(c) a transmembrane domain; and
(d) an intracellular signaling domain.

7. The bispecific CAR of claim 1 or claim 2, wherein the extracellular domain comprises, in order from amino to carboxy terminus: the VL region of the GPRC5D-binding domain, the VH region of the BCMA-binding domain, the VL region of the BCMA-binding domain, and the VH region of the GPRC5D-binding domain.

8. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VL region of the GPRC5D-binding domain, the VH region of the BCMA-binding domain, the VL region of the BCMA-binding domain, and the VH region of the GPRC5D-binding domain;
(b) a spacer;
(c) a transmembrane domain; and
(d) an intracellular signaling domain.

9. The bispecific CAR of claim 1 or claim 2, wherein the extracellular domain comprises in order from amino to carboxy terminus: the VL region of the GPRC5D-binding domain, the VL region of the BCMA-binding domain, the VH region of the BCMA-binding domain, and the VH region of the GPRC5D-binding domain.

10. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VL region of the GPRC5D-binding domain, the VL region of the BCMA-binding domain, the VH region of the BCMA-binding domain, and the VH region of the GPRC5D-binding domain;
(b) a spacer;
(c) a transmembrane domain; and
(d) an intracellular signaling domain.

11. The bispecific CAR of claim 1, wherein the extracellular domain comprises, in order from amino to carboxy terminus one of the VH region and the VL region of the BCMA-binding domain, one of the VH region and the VL region of the GPRC5D-binding domain, the other of the VH region and the VL region of the GPRC5D-binding domain, and the other of the VH region and the VL region of the BCMA-binding domain.

12. The bispecific CAR of claim 1 or claim 11, wherein the extracellular domain comprises in order from amino to carboxy terminus: the VH region of the BCMA-binding domain, the VH region of the GPRC5D-binding domain, the VL region of the GPRC5D-binding domain, and the VL region of the BCMA-binding domain.

13. A bispecific chimeric antigen receptor (CAR), comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VH region of the BCMA-binding domain, the VH region of the GPRC5D-binding domain, the VL region of the GPRC5D-binding domain, and the VL region of the BCMA-binding domain;
(b) a spacer;
(c) a transmembrane domain; and
(d) an intracellular signaling domain.

14. The bispecific CAR of claim 1 or claim 11, wherein the extracellular domain comprises in order from amino to carboxy terminus: the VH region of the BCMA-binding domain, the VL region of the GPRC5D-binding domain, the VH region of the GPRC5D-binding domain, and the VL region of the BCMA-binding domain.

15. A bispecific chimeric antigen receptor (CAR), comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VH region of the BCMA-binding domain, the VL region of the GPRC5D-binding domain, the VH region of the GPRC5D-binding domain, and the VL region of the BCMA-binding domain;
(b) a spacer;
(c) a transmembrane domain; and
(d) an intracellular signaling domain.

16. The bispecific CAR of claim 1 or claim 11, wherein the extracellular domain comprises in order from amino to carboxy terminus: the VL region of the BCMA-binding domain, the VH region of the GPRC5D-binding domain, the VL region of the GPRC5D-binding domain, and the VH region of the BCMA-binding domain.

17. A bispecific chimeric antigen receptor comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VL region of the BCMA-binding domain, the VH region of the GPRC5D-binding domain, the VL region of the GPRC5D-binding domain, and the VH region of the BCMA-binding domain;
(b) a spacer;
(c) a transmembrane domain; and
(d) an intracellular signaling domain.

18. The bispecific CAR of claim 1 or claim 11, wherein the extracellular domain comprises in order from amino to carboxy terminus: the VL region of the BCMA-binding domain, the VL region of the GPRC5D-binding domain, the VH region of the GPRC5D-binding domain, and the VH region of the BCMA-binding domain.

19. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises in order from amino to carboxy terminus: the VL region of the BCMA-binding domain, the VL region of the GPRC5D-binding domain, the VH region of the GPRC5D-binding domain, and the VH region of the BCMA-binding domain;
(b) a spacer;
(c) a transmembrane domain; and
(d) an intracellular signaling domain.

20. The bispecific CAR of any one of claims 1-19, wherein (a) the VH region or the VL region of the GPRC5D-binding domain; and (b) the VH region or the VL region of the BCMA-binding domain are joined by a linker.

21. The bispecific CAR of claim 20, wherein the linker is a flexible peptide linker.

22. The bispecific CAR of claim 20 or claim 21, wherein the linker is 4 to 12 amino acids in length.

23. The bispecific CAR of any of claims 20-22, wherein the linker is or comprises the amino acid sequence set forth in SEQ ID NO:19, SEQ ID NO:21, or SEQ ID NO:22.

24. The bispecific CAR of any one of claims 1-23, wherein:
(a) the VH region and the VL region of the GPRC5D-binding domain are joined by a linker; or
(b) the VH region and the VL region of the BCMA-binding domain are joined by a linker.

25. The bispecific CAR of claim 24, wherein the linker comprises the amino acid sequence set forth in SEQ ID NO:17 or SEQ ID NO:18.

26. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus:
(i) the VH region of the GPRC5D-binding domain;
(ii) the linker set forth in SEQ ID NO:21;
(iii) the VL region of the BCMA-binding domain;
(iv) the linker set forth in SEQ ID NO:17;
(v) the VH region of the BCMA-binding domain;
(vi) the linker set forth in SEQ ID NO:21; and
(vii) the VL region of the GPRC5D-binding domain;
(b) a spacer;
(c) a transmembrane domain; and
(d) an intracellular signaling domain.

27. A bispecific chimeric antigen receptor (CAR) comprising:
(a) an extracellular domain comprising (i) a GPRC5D-binding domain that binds to GPRC5D comprising a heavy chain variable (VH) region and a light chain variable (VL) region; and (ii) a BCMA-binding domain that binds to BCMA comprising a VH region and a VL region, wherein the extracellular domain comprises, in order from amino to carboxy terminus: one of the VH region and the VL region of the BCMA-binding domain; the other of the VH region and the VL region of the BCMA-binding domain; one of the VH region and the VL region of the GPRC5D-binding domain; and the other of the VH region and the VL region of the GPRC5D-binding domain;
(b) a spacer;
(c) a transmembrane domain; and
(d) an intracellular signaling domain.

28. A bispecific chimeric antigen receptor (CAR) comprising:
(a) an extracellular domain comprising (i) a GPRC5D-binding domain that binds to GPRC5D comprising a heavy chain variable (VH) region and a light chain variable (VL) region; and (ii) a BCMA-binding domain that binds to BCMA comprising a VH region and a VL region, wherein the extracellular domain comprises, in order from amino to carboxy terminus: the VL region of the GPRC5D-binding domain; the VH region of the GPRC5D-binding domain; one of the VH region and the VL region of the BCMA-binding domain; and the other of the VH and the VL region of the BCMA-binding domain;
(b) a spacer;
(c) a transmembrane domain; and
(d) an intracellular signaling domain.

29. The bispecific CAR of claim 27 or claim 28, wherein the GPRC5D-binding region and the BCMA-binding region are joined by a linker.

30. The bispecific CAR of claim 29, wherein the linker is a flexible peptide linker.

31. The bispecific CAR of claim 29 or claim 30, wherein the linker is 4 to 12 amino acids in length.

32. The bispecific CAR of any one of claims 29-31, wherein the linker comprises the amino acid sequence set forth in SEQ ID NO:19, SEQ ID NO:21, or SEQ ID NO:24.

33. The bispecific CAR of any one of claims 27-32, wherein the VH region and the VL region of the BCMA-binding domain are joined by a linker comprising the amino acid sequence set forth in SEQ ID NO:17.

34. A bispecific chimeric antigen receptor (CAR) comprising:
(a) an extracellular domain comprising (i) a GPRC5D-binding domain that binds to GPRC5D comprising a heavy chain variable (VH) region and a light chain variable (VL) region; and (ii) a BCMA-binding domain that binds to BCMA comprising a VH region and a VL region, wherein the extracellular domain comprises in order from amino to carboxy terminus: the VH region of the GPRC5D-binding domain; the VL region of the GPRC5D-binding domain; one of the VH region and the VL region of the BCMA-binding domain; and the other of the VH and the VL region of the BCMA-binding domain;
(b) a spacer;
(c) a transmembrane domain; and
(d) an intracellular signaling domain,
wherein the GPRC5D-binding domain and the BCMA-binding domain are joined by a linker comprising the sequence set forth in SEQ ID NO:19 or SEQ ID NO:21.

35. The bispecific CAR of any one of claims 1-34, wherein the VH region of the GPRC5D− binding domain comprises a CDR-1, a CDR-2, and a CDR-3 comprising the amino acid sequences set forth in SEQ ID NO:1, SEQ ID NO:2, and SEQ ID NO:3, respectively.

36. The bispecific CAR of any one of claims 1-35, wherein the VL region of the GPRC5D− binding domain comprises a CDR-1, a CDR-2, and a CDR-3 comprising the amino acid sequences set forth in SEQ ID NO:4, SEQ ID NO:5, and SEQ ID NO:6, respectively.

37. The bispecific CAR of any one of claims 1-36, wherein the VH region of the GPRC5D− binding domain comprises a CDR-1, a CDR-2, and a CDR-3 comprising the amino acid sequences set forth in SEQ ID NO:1, SEQ ID NO:2, and SEQ ID NO:3, respectively; and the VL region of the GPRC5D-binding domain comprises a CDR-1, a CDR-2, and a CDR-3 comprising the amino acid sequences set forth in SEQ ID NO:4, SEQ ID NO:5, and SEQ ID NO:6, respectively.

38. The bispecific CAR of any one of claims 1-37, wherein the VH region of the GPRC5D− binding domain an amino acid sequence having at least about 90% sequence identity to the amino acid sequence set forth in SEQ ID NO:7.

39. The bispecific CAR of any one of claims 1-38, wherein the VL region of the GPRC5D− binding domain comprises an amino acid sequence having at least about 90% sequence identity to the amino acid sequence set forth in SEQ ID NO:8.

40. The bispecific CAR of any one of claims 1-39, wherein the VH region of the GPRC5D− binding domain comprises an amino acid sequence having at least about 90% sequence identity to the amino acid sequence set forth in SEQ ID NO:7; and the VL region of the GPRC5D-binding domain comprises an amino acid sequence having at least about 90% sequence identity to the amino acid sequence set forth in SEQ ID NO:8.

41. The bispecific CAR of any one of claims 1-40, wherein the VH region of the GPRC5D− binding domain comprises the amino acid sequence set forth in SEQ ID NO:7.

42. The bispecific CAR of any one of claims 1-41, wherein the VL region of the GPRC5D− binding domain comprises the amino acid sequence set forth in SEQ ID NO:8.

43. The bispecific CAR of any one of claims 1-42, wherein the VH region of the GPRC5D− binding domain comprises the amino acid sequence set forth in SEQ ID NO:7; and the VL region of the GPRC5D-binding domain comprises the amino acid sequence set forth in SEQ ID NO:8.

44. The bispecific CAR of any one of claims 1-43, wherein the VH region of the BCMA-binding domain comprises a CDR-1, a CDR-2, and a CDR-3 comprising the amino acid sequences set forth in SEQ ID NO:9, SEQ ID NO:10, and SEQ ID NO: 11, respectively.

45. The bispecific CAR of any one of claims 1-44, wherein the VL region of the BCMA-binding domain comprises a CDR-1, a CDR-2, and a CDR-3 comprising the amino acid sequences set forth in SEQ ID NO:12, SEQ ID NO:13, and SEQ ID NO:14, respectively.

46. The bispecific CAR of any one of claims 1-45, wherein the VH region of the BCMA-binding domain comprises an amino acid sequence having at least about 90% sequence identity to the amino acid sequence set forth in SEQ ID NO:15.

47. The bispecific CAR of any one of claims 1-46, wherein the VL region of the BCMA-binding domain comprises an amino acid sequence having at least about 90% sequence identity to the amino acid sequence set forth in SEQ ID NO:16.

48. The bispecific CAR of any one of claims 1-47, wherein the VH region of the BCMA-binding domain comprises an amino acid sequence having at least about 90% sequence identity to the amino acid sequence set forth in SEQ ID NO:15; and the VL region of the BCMA-binding domain comprises an amino acid sequence having at least about 90% sequence identity to the amino acid sequence set forth in SEQ ID NO:16.

49. The bispecific CAR of any one of claims 1-48, wherein the VH region of the BCMA-binding domain comprises the amino acid sequence set forth in SEQ ID NO:15.

50. The bispecific CAR of any one of claims 1-49, wherein the VL region of the BCMA-binding domain comprises the amino acid sequence set forth in SEQ ID NO:16.

51. The bispecific CAR of any one of claims 1-50, wherein the VH region of the BCMA-binding domain comprises the amino acid sequence set forth in SEQ ID NO:15; and the VL region of the BCMA-binding domain comprises the amino acid sequence set forth in SEQ ID NO:16.

52. The bispecific CAR of any one of claims 1, 20-25, and 35-51, wherein the extracellular binding domain comprises the amino acid sequence set forth in any one of SEQ ID NO:77, 78, 79, and 80.

53. The bispecific CAR of any one of claims 1, 20-25, and 35-51, wherein the extracellular binding domain comprises the amino acid sequence set forth in SEQ ID NO:81, 82, 83, 84, 85, 86, 87, 88, 89, and 90.

54. The bispecific CAR of any one of claims 1, 2, 5, 6, 20-26, 35-51 and 53, wherein the extracellular binding domain comprises the amino acid sequence set forth in SEQ ID NO: 83.

55. The bispecific CAR of any one of claims 1, 2, 7, 8, 20-25, 35-51 and 53, wherein the extracellular binding domain comprises the amino acid sequence set forth in SEQ ID NO: 84.

56. The bispecific CAR of any one of claims 1, 2, 5, 6, 20-25, 35-51 and 53, wherein the extracellular binding domain comprises the amino acid sequence set forth in SEQ ID NO: 87.

57. The bispecific CAR of any one of claims 1, 11, 14, 15, 20-25, 35-51 and 53, wherein the extracellular binding domain comprises the amino acid sequence set forth in SEQ ID NO: 81.

58. The bispecific CAR of any one of claims 1, 11, 16, 17, 20-25, 35-51 and 53, wherein the extracellular binding domain comprises the amino acid sequence set forth in SEQ ID NO: 85.

59. The bispecific CAR of any one of claims 1, 11, 18-25, 35-51 and 53, wherein the extracellular binding domain comprises the amino acid sequence set forth in SEQ ID NO: 86.

60. The bispecific CAR of any one of claims 1, 11, 16, 17, 20-25, 35-51 and 53, wherein the extracellular binding domain comprises the amino acid sequence set forth in SEQ ID NO: 90.

61. The bispecific CAR of any one of claims 1-60, wherein the spacer comprises at least a portion of an immunoglobulin or a variant thereof.

62. The bispecific CAR of any one of claims 1-61, wherein the spacer comprises a hinge region of an immunoglobulin or a variant thereof.

63. The bispecific CAR of claim 62, wherein the hinge region of an immunoglobulin is an IgG4 hinge region, optionally a human IgG4 hinge region, or a variant thereof.

64. The bispecific CAR of any one of claims 1-63, wherein the spacer is less than at or about 15 amino acids in length.

65. The bispecific CAR of any one of claims 1-64, wherein the spacer is between 12 and 15 amino acids in length.

66. The bispecific CAR of any one of claims 1-65, wherein the spacer comprises the amino acid sequence set forth in SEQ ID NO:25, or an amino acid sequence having at least about 90% sequence identity to the amino acid sequence set forth in SEQ ID NO:25.

67. The bispecific CAR of any one of claims 1-64, wherein the spacer is between 200 and 250 amino acids in length, optionally between 220 and 240 amino acids in length.

68. The bispecific CAR of any one of claim 1-64 and 67, wherein the spacer comprises a hinge region of an immunoglobulin, a CH2 region of an immunoglobulin or a chimeric CH2 region of two different immunoglobulins, and a CH3 region of an immunoglobulin.

69. The bispecific CAR of any one of claims 1-64, 67, and 68, wherein the spacer comprises IgG4 hinge region or a variant thereof, a chimeric CH2 region comprising a portion of an IgG4 CH2 and a portion of an IgG2 CH2 (IgG2/4 CH2 region), and an IgG4 CH3 region.

70. The bispecific CAR of any one of claims 1-64 and 67-69, wherein the spacer comprises the amino acid sequence set forth in SEQ ID NO:27, or an amino acid sequence having at least about 90% sequence identity to the amino acid sequence set forth in SEQ ID NO:27.

71. The bispecific CAR of any one of claims 1-70, wherein the transmembrane domain is or comprises a transmembrane domain from CD4, CD28, or CD8, optionally from human CD4, human CD28 or human CD8.

72. The bispecific CAR of any one of claims 1-71, wherein the transmembrane domain is or comprises a transmembrane domain from human CD28.

73. The bispecific CAR of any one of claims 1-72, wherein the transmembrane domain comprises the amino acid sequence set forth in SEQ ID NO:28, or an amino acid sequence having at least about 90% sequence identity to the amino acid sequence set forth in SEQ ID NO:28.

74. The bispecific CAR of any one of claims 1-73, wherein the intracellular signaling domain is a domain from a T cell receptor (TCR) component or comprises an immunoreceptor tyrosine-based activation motif (ITAM).

75. The bispecific CAR of any one of claims 1-74, wherein the intracellular signaling domain comprises a cytoplasmic signaling domain of a CD3-zeta chain, optionally a human CD3-zeta chain.

76. The bispecific CAR of any one of claims 1-75, wherein the intracellular signaling domain comprises the amino acid sequence set forth in SEQ ID NO:30, or an amino acid sequence having at least about 90% sequence identity to the amino acid sequence set forth in SEQ ID NO:30.

77. The bispecific CAR of any one of claims 1-76, wherein the intracellular signaling domain further comprises a costimulatory signaling region.

78. The bispecific CAR of claim 77, wherein the costimulatory signaling region is located between the transmembrane region and the intracellular signaling domain.

79. The bispecific CAR of claim 77 or claim 78, wherein the costimulatory signaling region comprises an intracellular signaling domain of a T cell costimulatory molecule or a signaling portion thereof.

80. The bispecific CAR of any one of claims 77-79, wherein the costimulatory signaling region comprises an intracellular signaling domain of CD28, 4-1BB, or ICOS, or a signaling portion thereof, optionally human CD28, human 4-1BB, or human ICOS.

81. The bispecific CAR of any one of claims 77-80, wherein the costimulatory signaling region comprises an intracellular signaling domain of 4-1BB or a signaling portion thereof, optionally human 4-1BB.

82. The bispecific CAR of any one of claims 68-72, wherein the costimulatory signaling region comprises the amino acid sequence set forth in SEQ ID NO:29, or an amino acid sequence having at least about 90% sequence identity to the amino acid sequence set forth in SEQ ID NO:29.

83. The bispecific CAR of any one of claims 1-82, wherein the CAR comprises the amino acid sequence that has at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 98% sequence identity to any one of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, or SEQ ID NO:44.

84. The bispecific CAR of any one of claims 1-83, wherein the CAR comprises the amino acid sequence set forth in SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, or SEQ ID NO:44.

85. The bispecific CAR of claim 84, wherein the CAR comprises the amino acid sequence set forth in SEQ ID NO:37.

86. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus:
(i) the VH region of the GPRC5D-binding domain comprising a CDR-1, a CDR-2, and a CDR-3 comprising the amino acid sequences set forth in SEQ ID NO:1, SEQ ID NO:2, and SEQ ID NO:3, respectively;
(ii) the linker set forth in SEQ ID NO:21;
(iii) the VL region of the BCMA-binding domain comprising a CDR-1, a CDR-2, and a CDR-3 comprising the amino acid sequences set forth in SEQ ID NO:12, SEQ ID NO:13, and SEQ ID NO:14, respectively;
(iv) the linker set forth in SEQ ID NO:17;
(v) the VH region of the BCMA-binding domain comprising a CDR-1, a CDR-2, and a CDR-3 comprising the amino acid sequences set forth in SEQ ID NO:9, SEQ ID NO:10, and SEQ ID NO:11, respectively;
(vi) the linker set forth in SEQ ID NO:21; and
(vii) the VL region of the GPRC5D-binding domain comprising a CDR-1, a CDR-2, and a CDR-3 comprising the amino acid sequences set forth in SEQ ID NO:4, SEQ ID NO:5, and SEQ ID NO:6, respectively;
(b) a spacer comprising the amino acid sequence set forth in SEQ ID NO:27;
(c) a transmembrane domain comprising the amino acid sequence set forth in SEQ ID NO:28; and
(d) an intracellular signaling domain comprising the amino acid sequences set forth in SEQ ID NOS:29 and 30.

87. The bispecific CAR of claim 86, wherein the extracellular binding domain comprises the amino acid sequence set forth in SEQ ID NO: 83.

88. The bispecific CAR of claim 86 or claim 87, wherein the CAR comprises the amino acid sequence set forth in SEQ ID NO:37.

89. The bispecific CAR of any of claims 76-88, which is encoded by the nucleotide sequence set forth in SEQ ID NO:119.

90. A bispecific chimeric antigen receptor (CAR) comprising:
(a) an extracellular domain comprising a GPRC5D-binding domain that binds to GPRC5D comprising a heavy chain variable (VH) region and a light chain variable (VL) region; and a BCMA-binding domain that binds to BCMA comprising a VH region and a VL region, wherein the extracellular domain comprises, in order from amino to carboxy terminus:
(i) the VL region of the BCMA-binding domain comprising a CDR-1, a CDR-2, and a CDR-3 comprising the amino acid sequences set forth in SEQ ID NO:12, SEQ ID NO:13, and SEQ ID NO:14, respectively;
(ii) the linker set forth in SEQ ID NO:21;
(vii) the VL region of the GPRC5D-binding domain comprising a CDR-1, a CDR-2, and a CDR-3 comprising the amino acid sequences set forth in SEQ ID NO:4, SEQ ID NO:5, and SEQ ID NO:6, respectively;
(iv) the linker set forth in SEQ ID NO:17;
(i) the VH region of the GPRC5D-binding domain comprising a CDR-1, a CDR-2, and a CDR-3 comprising the amino acid sequences set forth in SEQ ID NO:1, SEQ ID NO:2, and SEQ ID NO:3, respectively;
(vi) the linker set forth in SEQ ID NO:21; and
(v) the VH region of the BCMA-binding domain comprising a CDR-1, a CDR-2, and a CDR-3 comprising the amino acid sequences set forth in SEQ ID NO:9, SEQ ID NO:10, and SEQ ID NO:11, respectively;
(b) a spacer comprising the amino acid sequence set forth in SEQ ID NO:27;
(c) a transmembrane domain comprising the amino acid sequence set forth in SEQ ID NO:28; and
(d) an intracellular signaling domain comprising the amino acid sequences set forth in SEQ ID NOS:29 and 30.

91. The bispecific CAR of claim 90, wherein the extracellular binding domain comprises the amino acid sequence set forth in SEQ ID NO: 86.

92. The bispecific CAR of claim 90 or claim 91, wherein the CAR comprises the amino acid sequence set forth in SEQ ID NO:40.

93. The bispecific CAR of any one of claims 90-92, which is encoded by the polynucleotide sequence set forth in SEQ ID NO:120.

94. A polynucleotide encoding the CAR of any one of claims 1-88 and 90.

95. The polynucleotide of claim 94, comprising the nucleotide sequence set forth in any one of SEQ ID NOS:105-120.

96. A polynucleotide comprising the nucleotide sequence set forth in any one of SEQ ID NOS:105-120.

97. The polynucleotide of any of claims 94-96, wherein the polynucleotide is optimized by splice site elimination.

98. The polynucleotide of any of claims 94-97, wherein the polynucleotide is codon-optimized for expression in a human cell.

99. The polynucleotide of any of claims 94-98, comprising the nucleotide sequence set forth in SEQ ID NO:119 or SEQ ID NO:120.

100. The polynucleotide of any of claims 94-99, comprising the nucleotide sequence set forth in SEQ ID NO:119.

101. The polynucleotide of any of claims 94-100, comprising the nucleotide sequence set forth in SEQ ID NO:120.

102. A vector comprising the polynucleotide of any one of claims 94-101.

103. The vector of claim 102, which is a viral vector.

104. The vector of claim 102 or claim 103, which is a retroviral vector.

105. The vector of any one of claims 102-104, which is a lentiviral vector or an adeno-associated viral (AAV) vector.

106. A cell comprising the CAR of any one of claims 1-93.

107. A cell comprising the polynucleotide of any one of claims 90-101 or the vector or any one of claims 102-105.

108. The cell of claim 106 or claim 107, wherein the cell is an immune cell.

109. The cell of any one of claims 106-108, wherein the cell is a lymphocyte.

110. The cell of any one of claims 106-109, wherein the cell is a NK cell or a T cell.

111. The cell of any one of claims 106-110, wherein the cell is a T cell.

112. The cell of claim 111, wherein the T cell is a CD4+ T cell or a CD8+ T cell.

113. The cell of claim 111 or claim 112, wherein the T cell is a primary T cell.

114. The cell of claim 106 or claim 107, wherein the cell is a stem cell.

115. The cell of any claim 114, wherein the stem cell is a multipotent and pluripotent stem cell.

116. The cell of claim 114 or claim 115, wherein the stem cell is an induced pluripotent stem cell (iPSC).

117. The cell of any one of claims 106-112, wherein the cell has been differentiated from an induced pluripotent stem cell.

118. The cell of any one of claims 106-117, wherein the cell is an allogeneic cell.

119. The cell of any one of claims 106-118, wherein the cell is engineered to be hypoimmune.

120. The cell of any one of claims 98-119, wherein the cell exhibits cytotoxic activity against GPRC5D+ cells, BCMA+ cells, or GPRC5D+/BCMA+ cells.

121. A composition comprising a plurality of the cell of any one of claims 106-120.

122. The composition of claim 121, further comprising a pharmaceutically acceptable excipient.

123. A pharmaceutical composition comprising a plurality of the cell of any one of claims 106-120, and a pharmaceutically acceptable excipient.

124. The composition of any one of claims 121-123, wherein the composition comprises CD4+ T cells and CD8+ T cells.

125. The composition of claim 124, wherein the composition comprises a ratio of CD4+ T cells to CD8+ T cells that is between about 1:3 and about 3:1, optionally between about 1:2 and about 2:1, further optionally about 1:1.

126. The composition of claim 124 or claim 125, wherein the composition comprises a ratio of CD4+ T cells to CD8+ T cells that is between about 1:3 and about 3:1.

127. The composition of any one of claims 124-126, wherein the composition comprises a ratio of CD4+ T cells to CD8+ T cells that is about 1:1.

128. The composition of any one of claims 121-127, wherein greater than about 90%, greater than about 95% or greater than about 99% of cells in the composition are CD3+ T cells.

129. The composition of any one of claims 121-128, wherein at least about 40%, at least about 50%, at least about 60%, at least about 70%, at least about 80%, or at least about 90% of cells in the composition express the CAR.

130. The composition of any one of claims 121-129, wherein, among a plurality of the cells in the composition expressing the CAR, less than about 10%, about 9%, about 8%, about 7%, about 5%, about 4%, about 3%, about 2%, or about 1% of the cells exhibit tonic signaling.

131. The composition of any one of claims 121-130, wherein the composition comprises between about 1.0×10⁷ CAR-expressing T cells and 1.2×10⁹ CAR-expressing T cells, between about 1.0×10⁷ CAR-expressing T cells and 6.5×10⁸ CAR-expressing T cells, between about 1.5×10⁷ CAR-expressing T cells and 6.5×10⁸ CAR-expressing T cells, between about 1.5×10⁷ CAR-expressing T cells and 6.0×10⁸ CAR-expressing T cells, between about 2.5×10⁷ CAR-expressing T cells and 6.0×10⁸ CAR-expressing T cells, between about 5.0×10⁷ CAR-expressing T cells and 6.0×10⁸ CAR-expressing T cells, between about 1.25×10⁷ CAR-expressing T cells and 1.2×10⁹ CAR-expressing T cells, between about 1.5×10⁷ CAR-expressing T cells and 1.2×10⁹ CAR-expressing T cells, between about 5.0×10⁷ CAR-expressing T cells and 4.5×10⁸ CAR-expressing T cells, or between about 1.5×10⁸ CAR-expressing T cells and 3.0×10⁸ CAR-expressing T cells, each inclusive.

132. The composition of any one of claims 121-131, wherein the composition comprises at or about 1.5×10⁷, at or about 2.5×10⁷, at or about 5.0×10⁷, at or about 7.5×10⁷, at or about 1.0×10⁸, at or about 1.25×10⁸, at or about 1.5×10⁸, at or about 1.75×10⁸, at or about 2×10⁸, at or about 2.25×10⁸, at or about 2.5×10⁸, at or about 3.0×10⁸, at or about 3.5×10⁸, at or about 4×10⁸, at or about 4.5×10⁸, at or about 6.0×10⁸, at or about 8.0×10⁸, or at or about 1.2×10⁹ CAR-expressing T cells.

133. A method of treating a disease or condition comprising administering the cell of any one of claims 106-120 or the composition of any one of claims 121-132 to a subject.

134. The method of claim 133, wherein the cell is administered to the subject at a dose of from at or about 1×10⁷ CAR-expressing T cells and 1×10⁸ CAR-expressing T cells

135. The method of claim 133, wherein the cell is administered to the subject at a dose of from or from about 2.5×10⁷ CAR-expressing T cells to about 4.5×10⁸ CAR-expressing T cells.

136. The method of any one of claims 133-135, wherein the cell is administered to the subject at a dose of or about 2.5×10⁷ CAR-expressing T cells.

137. The method of any one of claims 133-135, wherein the cell is administered to the subject at a dose of or about 7.5×10⁷ CAR-expressing T cells.

138. The method of any one of claims 133-135, wherein the cell is administered to the subject at a dose of or about 1.5×10⁸ CAR-expressing T cells.

139. The method of any one of claims 133-135, wherein the cell is administered to the subject at a dose of or about 3.0×10⁸ CAR-expressing T cells.

140. The method of any one of claims 133-135, wherein the cell is administered to the subject at a dose of or about 4.5×10⁸ CAR-expressing T cells.

141. The method of any one of claims 133-140, further comprising administering a lymphodepleting therapy to the subject prior to administration of the dose of the CAR-expressing T cells.

142. The method of any one of claims 133-141, wherein the lymphodepleting therapy is completed within about 7 days prior to initiation of the administration of the dose of the CAR-expressing T cells.

143. The method of any one of claims 133-142, wherein the administration of the lymphodepleting therapy is completed within about 2 to 7 days prior to initiation of the administration of the dose of engineered T cells.

144. The method of any one of claims 133-143, wherein the lymphodepleting therapy comprises the administration of fludarabine and/or cyclophosphamide.

145. The method of any one of claims 133-144, wherein the lymphodepleting therapy comprises the administration of fludarabine and cyclophosphamide.

146. The method of any one of claims 133-145, wherein the lymphodepleting therapy comprises administration of cyclophosphamide at or about 200-400 mg/m² inclusive daily.

147. The method of any one of claims 133-146, wherein the lyphodepleting therapy comprises administration of cyclophosphamide at or about 300 mg/m² daily.

148. The method of any one of claims 133-145, wherein the lyphodepleting therapy comprises administration of fludarabine at or about 20-40 mg/m² inclusive daily.

149. The method of any one of claims 133-146 and 148, wherein the lyphodepleting therapy comprises administration of fludarabine at or about 30 mg/m² daily.

150. The method of any one of claims 133-149, wherein the lyphodepleting therapy comprises administration of fludarabine and cyclophosphamide for 2-4 days.

151. The method of any one of claims 133-150, wherein the lyphodepleting therapy comprises administration of fludarabine and cyclophosphamide for 3 days.

152. The method of any one of claims 133-143 wherein the lymphodepleting therapy comprises the administration of bendamustine.

153. The method of any one of claims 133-143 and 152, wherein the lymphodepleting therapy comprises administration of bendamustine at or about 50-130 mg/m² inclusive daily.

154. The method of any one of claims 133-143, 152, and 153, wherein the lyphodepleting therapy comprises administration of bendamustine at or about 90 mg/m² daily.

155. The method of any one of claims 133-143 and 152-154, wherein the lyphodepleting therapy comprises administration of bendamustine for 1-3 days.

156. The method of any one of claims 133-143 and 152-155, wherein the lyphodepleting therapy comprises administration of bendamustine for 2 days.

157. The method of any one of claims 133-156, wherein the disease or condition is a cancer, optionally a plasma cell malignancy.

158. The method of any one of claims 133-157, wherein the disease or condition is a BCMA-expressing cancer and/or a GPRC5D-expressing cancer.

159. The method of any one of claims 133-158, wherein the disease or condition is a multiple myeloma.

160. The method of any one of claims 133-159, wherein the disease or condition is a relapsed/refractory multiple myeloma (RRMM).

161. The method of any one of claims 133-160, wherein the subject has received one or more prior therapies.

162. The method of any one of claims 133-161, wherein, the subject has received at least 1, but no more than 3, prior therapies.

163. The method of claim 161 or claim 162, wherein the prior therapies are an proteasome inhibitor, an immumodulatory agent, an anti-CD38 antibody, a prior therapy that included autologous hematopoietic stem cell transplantation (HSCT), or a combination of any of the foregoing.

164. Use of the cell of any one of claims 106-120 or the composition of any one of claims 121-132 for manufacture of a medicament for treating a disease or condition in a subject.

165. Use of the cell of any one of claims 106-120 or the composition of any one of claims 121-132 for treatment of a disease or condition in a subject.

166. The use of claim 164 or claim 165, wherein the disease or condition is a cancer, optionally a plasma cell malignancy.

167. The use of any one of claims 164-166, wherein the disease or condition is a BCMA-expressing cancer and/or a GPRC5D-expressing cancer.

168. The use of any one of claims 164-167, wherein the disease or condition is a multiple myeloma.

169. The use of any one of claims 164-168, wherein the disease or condition is a relapsed/refractory multiple myeloma (RRMM).

170. The use of any one of claims 164-169, wherein the subject has received one or more prior therapies.

171. The use of any one of claims 164-169, wherein, the subject has received at least 1, but no more than 3, prior therapies.

172. The use of claim 170 or claim 171, wherein the prior therapies are an proteasome inhibitor, an immumodulatory agent, an anti-CD38 antibody, a prior therapy that included autologous hematopoietic stem cell transplantation (HSCT), or a combination of any of the foregoing.

173. The cell of any one of claims 106-120 or the composition of any one of claims 121-132 for treatment of a disease or condition in a subject.

174. The cell or composition for use of claim 173, wherein the disease or condition is a cancer, optionally a plasma cell malignancy.

175. The cell or composition for use of claim 173 or claim 174, wherein the disease or condition is a BCMA-expressing cancer and/or a GPRC5D-expressing cancer.

176. The cell or composition for use of any one of claims 173-175, wherein the disease or condition is a multiple myeloma.

177. The cell or composition for use of any one of claims 173-176, wherein the disease or condition is a relapsed/refractory multiple myeloma (RRMM).

178. The cell or composition of any one of claims 173-177, wherein the subject has received one or more prior therapies.

179. The cell or composition of any one of claims 173-178, wherein, the subject has received at least 1, but no more than 3, prior therapies.

180. The cell or composition of claim 178 or claim 179, wherein the prior therapies are an proteasome inhibitor, an immumodulatory agent, an anti-CD38 antibody, a prior therapy that included autologous hematopoietic stem cell transplantation (HSCT), or a combination of any of the foregoing.

181. A kit comprising the CAR of any one of claims 1-93, the polynucleotide of any one of claims 94-101, the vector of any one of claims 102-105, the cell of any one of claims 106-120, or the composition of any one of claims 121-132, and instructions for use, optionally wherein the instructions are for administering the CAR, the cell, or the composition.

182. The kit of claim 181, wherein the instructions specify administering the CAR, the cell, or the composition to a subject having a disease or disorder.

183. An article of manufacture comprising the CAR of any one of claims 1-93, the polynucleotide of any one of claims 94-101, the vector of any one of claims 102-105, the cell of any one of claims 106-120, the composition of any one of claims 121-132, or the kit of claim 181 or claim 182.


SEQUENCE LISTING (120 sequences)

SEQ ID NO: 1 - protein, 5 aa, Synthetic construct
  REGION 1..5: note = CDRH1
SHSMN

SEQ ID NO: 2 - protein, 17 aa, Synthetic construct
  REGION 1..17: note = CDRH2
SISSDSTYTYYADSVKG

SEQ ID NO: 3 - protein, 10 aa, Synthetic construct
  REGION 1..10: note = CDRH3
SGGQWKYYDY

SEQ ID NO: 4 - protein, 11 aa, Synthetic construct
  REGION 1..11: note = CRDL1
QGDSLRSYYAS

SEQ ID NO: 5 - protein, 7 aa, Synthetic construct
  REGION 1..7: note = CDRL2
GKNNRPS

SEQ ID NO: 6 - protein, 12 aa, Synthetic construct
  REGION 1..12: note = CDRL3
NSRDSSGNPPVV

SEQ ID NO: 7 - protein, 119 aa, Synthetic construct
  REGION 1..119: note = GPRC5D VH
QVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSS

SEQ ID NO: 8 - protein, 109 aa, Synthetic construct
  REGION 1..109: note = GPRC5D VL
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVL

SEQ ID NO: 9 - protein, 5 aa, Synthetic construct
  REGION 1..5: note = CDRH1
DYYVY

SEQ ID NO: 10 - protein, 17 aa, Synthetic construct
  REGION 1..17: note = CDRH2
WINPNSGGTNYAQKFQG

SEQ ID NO: 11 - protein, 9 aa, Synthetic construct
  REGION 1..9: note = CDRH3
SQRDGYMDY

SEQ ID NO: 12 - protein, 8 aa, Synthetic construct
  REGION 1..8: note = CRDL1
TGTSSDVG

SEQ ID NO: 13 - protein, 7 aa, Synthetic construct
  REGION 1..7: note = CDRL2
EDSKRPS

SEQ ID NO: 14 - protein, 10 aa, Synthetic construct
  REGION 1..10: note = CDRL3
SSNTRSSTLV

SEQ ID NO: 15 - protein, 118 aa, Synthetic construct
  REGION 1..118: note = BCMA VH
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 16 - protein, 104 aa, Synthetic construct
  REGION 1..104: note = BCMA VL
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVL

SEQ ID NO: 17 - protein, 22 aa, Synthetic construct
  REGION 1..22: note = linker
GSRGGGGSGGGGSGGGGSLEMA

SEQ ID NO: 18 - protein, 18 aa, Synthetic construct
  REGION 1..18: note = linker
GSTSGSGKPGSGEGSTKG

SEQ ID NO: 19 - protein, 5 aa, Synthetic construct
  REGION 1..5: note = linker
EAAAK

SEQ ID NO: 20 - protein, 4 aa, Synthetic construct
  REGION 1..4: note = 3GSlinker
GGGS

SEQ ID NO: 21 - protein, 5 aa, Synthetic construct
  REGION 1..5: note = 4GS linker
GGGGS

SEQ ID NO: 22 - protein, 10 aa, Synthetic construct
  REGION 1..10: note = linker
GGGGSGGGGS

SEQ ID NO: 23 - protein, 15 aa, Synthetic construct
  REGION 1..15: note = linker
GGGGSGGGGSGGGGS

SEQ ID NO: 24 - protein, 20 aa, Synthetic construct
  REGION 1..20: note = linker
GGGGSGGGGSGGGGSGGGGS

SEQ ID NO: 25 - protein, 12 aa, Synthetic construct
  REGION 1..12: note = short spacer
ESKYGPPCPPCP

SEQ ID NO: 26 - protein, 119 aa, Synthetic construct
  REGION 1..119: note = medium spacer (hinge-CH3 119 aa)
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 228 aa, Synthetic construct
  REGION 1..228: note = long spacer (IgG4/IgG2 hinge- IgG2/IgG4 CH2- IgG4 CH3 spacer; 228 aa)
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 28 aa, Synthetic construct
  REGION 1..28: note = CD28 transmembrane domain
MFWVLVVVGGVLACYSLLVTVAFIIFWV

SEQ ID NO: 29 - protein, 42 aa, Synthetic construct
  REGION 1..42: note = 4-1BB-derived intracellular co-signaling sequence (aa)
KRGRKKLLYIFKQPFMRPVQTTQEEDGCSCRFPEEEEGGCEL

SEQ ID NO: 30 - protein, 112 aa, Synthetic construct
  REGION 1..112: note = CD3-zeta derived intracellular signaling domain (aa)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 909 aa, Synthetic construct
  REGION 1..909: note = CAR 1 (aa)
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSEAAAKEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGSRGGGGSGGGGSGGGGSLEMAQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 909 aa, Synthetic construct
  REGION 1..909: note = CAR 2 (aa)
QVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSRGGGGSGGGGSGGGGSLEMASSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGSRGGGGSGGGGSGGGGSLEMAQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 924 aa, Synthetic construct
  REGION 1..924: note = CAR 3 (aa)
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGGGGSGGGGSGGGGSGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGSRGGGGSGGGGSGGGGSLEMAQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 924 aa, Synthetic construct
  REGION 1..924: note = CAR 4 (aa)
QVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSRGGGGSGGGGSGGGGSLEMASSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGGGGSGGGGSGGGGSGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGSRGGGGSGGGGSGGGGSLEMAQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 892 aa, Synthetic construct
  REGION 1..892: note = CAR 8 (aa)
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSEAAAKSSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSEAAAKQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 888 aa, Synthetic construct
  REGION 1..888: note = CAR 9 (aa)
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLEAAAKQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSTSGSGKPGSGEGSTKGSSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLEAAAKEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 892 aa, Synthetic construct
  REGION 1..892: note = CAR 5 (aa)
QVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGGGGSQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 892 aa, Synthetic construct
  REGION 1..892: note = CAR 6 (aa)
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGSRGGGGSGGGGSGGGGSLEMAQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGGGGSQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 892 aa, Synthetic construct
  REGION 1..892: note = CAR 10 (aa)
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGGGGSQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSRGGGGSGGGGSGGGGSLEMASSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 892 aa, Synthetic construct
  REGION 1..892: note = CAR 11 (aa)
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 902 aa, Synthetic construct
  REGION 1..902: note = CAR 7 (aa)
QVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGGGGSGGGGSQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 902 aa, Synthetic construct
  REGION 1..902: note = CAR 12 (aa)
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGGGGSGGGGSQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSRGGGGSGGGGSGGGGSLEMASSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGGGGSGGGGSQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 902 aa, Synthetic construct
  REGION 1..902: note = CAR 13 (aa)
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGGGGSGGGGSQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSRGGGGSGGGGSGGGGSLEMASSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGGGGSGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 898 aa, Synthetic construct
  REGION 1..898: note = CAR 14 (aa)
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGGGGSGGGGSQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSTSGSGKPGSGEGSTKGSSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGGGGSGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 250 aa, Synthetic construct
  REGION 1..250: note = GPRC5D scFv
QVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSRGGGGSGGGGSGGGGSLEMASSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVL

SEQ ID NO: 46 - protein, 250 aa, Synthetic construct
  REGION 1..250: note = GPRC5D scFv
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSS

SEQ ID NO: 47 - protein, 244 aa, Synthetic construct
  REGION 1..244: note = BCMAscFv
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 48 - protein, 244 aa, Synthetic construct
  REGION 1..244: note = BCMA scFv
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGSRGGGGSGGGGSGGGGSLEMAQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVL

SEQ ID NO: 49 - DNA, 684 bp, Synthetic construct
  misc_feature 1..684: note = IgG4/IgG2 hinge- IgG2/IgG4 CH2- IgG4 CH3 spacer (nt)
  source 1..684: mol_type = other DNA
gagtctaaatacggaccgccttgtcctccttgtccagctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaattggtacgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaaa

SEQ ID NO: 50 - DNA, 684 bp, Synthetic construct
  misc_feature 1..684: note = IgG4/IgG2 hinge- IgG2/IgG4 CH2- IgG4 CH3 spacer (nt)
  source 1..684: mol_type = other DNA
gaatctaagtacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaag

SEQ ID NO: 51 - DNA, 36 bp, Synthetic construct
  misc_feature 1..36: note = Spacer (IgG4hinge) (nt)
  source 1..36: mol_type = other DNA
gaatctaagtacggaccgccctgccccccttgccct

SEQ ID NO: 52 - protein, 229 aa, Synthetic construct
  REGION 1..229: note = Hinge-CH2-CH3 spacer (aa)
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - DNA, 117 bp, Synthetic construct
  misc_feature 1..117: note = CD28 ectodomain spacer (nt)
  source 1..117: mol_type = other DNA
attgaagttatgtatcctcctccttacctagacaatgagaagagcaatggaaccattatccatgtgaaagggaaacacctttgtccaagtcccctatttcccggaccttctaagccc

SEQ ID NO: 54 - protein, 39 aa, Synthetic construct
  REGION 1..39: note = CD28 ectodomain spacer (aa)
IEVMYPPPYLDNEKSNGTIIHVKGKHLCPSPLFPGPSKP

SEQ ID NO: 55 - DNA, 336 bp, Synthetic construct
  misc_feature 1..336: note = CD3-zeta derived intracellular signaling domain (nt)
  source 1..336: mol_type = other DNA
agagtcaagttttccaggtccgccgacgctccagcctaccagcaggggcagaaccagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 56 - DNA, 336 bp, Synthetic construct
  misc_feature 1..336: note = CD3-zeta derived intracellular signaling domain (nt)
  source 1..336: mol_type = other DNA
agagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 57 - DNA, 126 bp, Synthetic construct
  misc_feature 1..126: note = 4-1BB-derived intracellular co-signaling sequence (nt)
  source 1..126: mol_type = other DNA
aagcgggggagaaagaaactgctgtatattttcaaacagccctttatgagacctgtgcagactacccaggaggaagacggatgcagctgtaggtttcccgaggaagaggaaggaggctgtgagctg

SEQ ID NO: 58 - DNA, 126 bp, Synthetic construct
  misc_feature 1..126: note = 4-1BB-derived intracellular co-signaling sequence (nt)
  source 1..126: mol_type = other DNA
aagcggggcagaaagaagctgctctacatcttcaagcagcccttcatgcggcccgtgcagaccacacaagaggaagatggctgctcctgcagattccccgaggaagaagaaggcggctgcgagctg

SEQ ID NO: 59 - protein, 345 aa, Synthetic construct
  REGION 1..345: note = GPRC5D protein (Uniprot Q9NZD1)
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGV

SEQ ID NO: 60 - protein, 184 aa, Synthetic construct
  REGION 1..184: note = BCMA protein (Uniprot Q02223)
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 61 - DNA, 544 bp, Synthetic construct
  misc_feature 1..544: note = EF1alpha promoter with HTLV1 ehancer
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 62 - DNA, 589 bp, Synthetic construct
  misc_feature 1..589: note = Woodchuck Hepatitis Virus (WHP) Posttranscriptional Regulatory Element (WPRE)
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 63 - protein, 22 aa, Synthetic construct
  REGION 1..22: note = F2A peptide (aa)
VKQTLNFDLLKLAGDVESNPGP

SEQ ID NO: 64 - protein, 25 aa, Synthetic construct
  REGION 1..25: note = F2A peptide (aa)
GSGVKQTLNFDLLKLAGDVESNPGP

SEQ ID NO: 65 - protein, 20 aa, Synthetic construct
  REGION 1..20: note = E2A peptide (aa)
QCTNYALLKLAGDVESNPGP

SEQ ID NO: 66 - protein, 23 aa, Synthetic construct
  REGION 1..23: note = E2A peptide (aa)
GSGQCTNYALLKLAGDVESNPGP

SEQ ID NO: 67 - protein, 18 aa, Synthetic construct
  REGION 1..18: note = T2A peptide (aa)
EGRGSLLTCGDVEENPGP

SEQ ID NO: 68 - protein, 21 aa, Synthetic construct
  REGION 1..21: note = T2A peptide (aa)
GSGEGRGSLLTCGDVEENPGP

SEQ ID NO: 69 - protein, 24 aa, Synthetic construct
  REGION 1..24: note = T2A peptide (aa)
LEGGGEGRGSLLTCGDVEENPGPR

SEQ ID NO: 70 - protein, 19 aa, Synthetic construct
  REGION 1..19: note = P2A peptide (aa)
ATNFSLLKQAGDVEENPGP

SEQ ID NO: 71 - protein, 22 aa, Synthetic construct
  REGION 1..22: note = P2A peptide (aa)
GSGATNFSLLKQAGDVEENPGP

SEQ ID NO: 72 - protein, 16 aa, Synthetic construct
  REGION 1..16: note = CD33 signal sequence
MPLLLLLPLLWAGALA

SEQ ID NO: 73 - DNA, 683 bp, Synthetic construct
  misc_feature 1..683: note = IgG4/IgG2 hinge- IgG2/IgG4 CH2- IgG4 CH3 spacer (nt)
  source 1..683: mol_type = other DNA
gaatctaagtacggaccgccctgccctccctgccctgctcctcctgtggctggaccaagcgtgttcctgtttccacctaagcctaaagataccctgatgatttcccgcacacctgaagtgacttgcgtggtcgtggacgtgagccaggaggatccagaagtgcagttcaactggtacgtggacggcgtggaagtccacaatgctaagactaaaccccgagaggaacagtttcagtcaacttaccgggtcgtgagcgtgctgaccgtcctgcatcaggattggctgaacgggaaggagtataagtgcaaagtgtctaataagggactgcctagctccatcgagaaaacaattagtaaggcaaaagggcagcctcgagaaccacaggtgtataccctgccccctagccaggaggaaatgaccaagaaccaggtgtccctgacatgtctggtcaaaggcttctatccaagtacatcgccgtggagtgggaatcaaatgggcagcccgagaacaattacaagaccacaccacccgtgctggactctgatggaagtttctttctgtattccaggctgaccgtggataaatctcgctggcaggagggcaacgtgttctcttgcagtgtcatgcacgaagccctgcacaatcattatacacagaagtcactgagcctgtccctgggcaaa

SEQ ID NO: 74 - DNA, 684 bp, Synthetic construct
  misc_feature 1..684: note = IgG4/IgG2 hinge- IgG2/IgG4 CH2- IgG4 CH3 spacer (nt)
  source 1..684: mol_type = other DNA
gagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaag

SEQ ID NO: 75 - protein, 327 aa, Homo sapiens
  REGION 1..327: note = Human IgG4 Fc (Uniprot P01861)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 326 aa, Homo sapiens
  REGION 1..326: note = Human IgG2 Fc (Uniprot P01859)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 77 - protein, 499 aa, Synthetic construct
  REGION 1..499: note = extracellular binding domain
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSEAAAKEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGSRGGGGSGGGGSGGGGSLEMAQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVL

SEQ ID NO: 78 - protein, 499 aa, Synthetic construct
  REGION 1..499: note = extracellular binding domain
QVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSRGGGGSGGGGSGGGGSLEMASSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGSRGGGGSGGGGSGGGGSLEMAQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVL

SEQ ID NO: 79 - protein, 514 aa, Synthetic construct
  REGION 1..514: note = extracellular binding domain
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGGGGSGGGGSGGGGSGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGSRGGGGSGGGGSGGGGSLEMAQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVL

SEQ ID NO: 80 - protein, 514 aa, Synthetic construct
  REGION 1..514: note = extracellular binding domain
QVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSRGGGGSGGGGSGGGGSLEMASSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGGGGSGGGGSGGGGSGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGSRGGGGSGGGGSGGGGSLEMAQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVL

SEQ ID NO: 81 - protein, 482 aa, Synthetic construct
  REGION 1..482: note = extracellular binding domain
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSEAAAKSSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSEAAAKQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVL

SEQ ID NO: 82 - protein, 478 aa, Synthetic construct
  REGION 1..478: note = extracellular binding domain
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLEAAAKQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSTSGSGKPGSGEGSTKGSSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLEAAAKEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 83 - protein, 482 aa, Synthetic construct
  REGION 1..482: note = extracellular binding domain
QVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGGGGSQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVL

SEQ ID NO: 84 - protein, 482 aa, Synthetic construct
  REGION 1..482: note = extracellular binding domain
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGSRGGGGSGGGGSGGGGSLEMAQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGGGGSQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSS

SEQ ID NO: 85 - protein, 482 aa, Synthetic construct
  REGION 1..482: note = extracellular binding domain
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGGGGSQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSRGGGGSGGGGSGGGGSLEMASSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 86 - protein, 482 aa, Synthetic construct
  REGION 1..482: note = extracellular binding domain
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 87 - protein, 492 aa, Synthetic construct
  REGION 1..492: note = extracellular binding domain
QVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGGGGSGGGGSQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVL

SEQ ID NO: 88 - protein, 492 aa, Synthetic construct
  REGION 1..492: note = extracellular binding domain
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSGGGGSGGGGSQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSRGGGGSGGGGSGGGGSLEMASSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGGGGSGGGGSQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVL

SEQ ID NO: 89 - protein, 492 aa, Synthetic construct
  REGION 1..492: note = extracellular binding domain
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGGGGSGGGGSQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSRGGGGSGGGGSGGGGSLEMASSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGGGGSGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 90 - protein, 488 aa, Synthetic construct
  REGION 1..488: note = extracellular binding domain
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGGGGSGGGGSQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSTSGSGKPGSGEGSTKGSSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGGGGSGGGGSEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 91 - DNA, 60 bp, Homo sapiens
  misc_feature 1..60: note = human IgG -kappa signal sequence (nt)
  source 1..60: mol_type = unassigned DNA
atggtgctgcagacccaggtgttcatcagcctgctgctgtggatctccggagcatacgga

SEQ ID NO: 92 - protein, 20 aa, Homo sapiens
  REGION 1..20: note = human IgG -kappa signal peptide(aa)
MVLQTQVFISLLLWISGAYG

SEQ ID NO: 93 - DNA, 60 bp, Homo sapiens
  misc_feature 1..60: note = human IgG -kappa signal sequence (nt)
  source 1..60: mol_type = unassigned DNA
atggtgctgcagacccaggtgttcatcagcctgctgctgtggatctctggcgcctacggc

SEQ ID NO: 94 - DNA, 60 bp, Homo sapiens
  misc_feature 1..60: note = human IgG -kappa signal sequence (nt)
  source 1..60: mol_type = unassigned DNA
atggtgctgcagacccaggtgttcatcagcctgctgctgtggatctctggcgcctatgga

SEQ ID NO: 95 - DNA, 60 bp, Homo sapiens
  misc_feature 1..60: note = human IgG -kappa signal sequence (nt)
  source 1..60: mol_type = unassigned DNA
atggtgctgcagacacaggtgttcatctccctgctgctgtggatctctggagcatacgga

SEQ ID NO: 96 - DNA, 60 bp, Homo sapiens
  misc_feature 1..60: note = human IgG -kappa signal sequence (nt)
  source 1..60: mol_type = unassigned DNA
atggtgctgcagacacaggtgttcatcagcctgctgctgtggatctccggagcatacgga

SEQ ID NO: 97 - DNA, 66 bp, Synthetic construct
  misc_feature 1..66: note = GMCSFR alpha chain signal sequence
  source 1..66: mol_type = other DNA
atgcttctcctggtgacaagccttctgctctgtgagttaccacacccagcattcctcctgatccca

SEQ ID NO: 98 - protein, 22 aa, Synthetic construct
  REGION 1..22: note = GMCSFR alpha chain signal peptide
MLLLVTSLLLCELPHPAFLLIP

SEQ ID NO: 99 - protein, 18 aa, Synthetic construct
  REGION 1..18: note = CD8 alpha signal peptide
MALPVTALLLPLALLLHA

SEQ ID NO: 100 - protein, 41 aa, Synthetic construct
  REGION 1..41: note = CD28 co-stimulatory domain
RSKRSRLLHSDYMNMTPRRPGPTRKHYQPYAPPRDFAAYRS

SEQ ID NO: 101 - protein, 654 aa, Synthetic construct
  REGION 1..654: note = Anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 660 aa, Synthetic construct
  REGION 1..660: note = Anti-GPRC5D CAR
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - DNA, 72 bp, Synthetic construct
  misc_feature 1..72: note = T2A peptide (nt)
  source 1..72: mol_type = other DNA
ctcgagggcggcggagagggcagaggaagtcttctaacatgcggtgacgtggaggagaatcccggccctagg

SEQ ID NO: 104 - DNA, 72 bp, Synthetic construct
  misc_feature 1..72: note = T2A peptide (nt)
  source 1..72: mol_type = other DNA
cttgaaggtggtggcgaaggcagaggcagcctgcttacatgcggagatgtggaagagaaccccggacctaga

SEQ ID NO: 105 - DNA, 2775 bp, Synthetic construct
  misc_feature 1..2775: note = CAR 1 (nt)
  source 1..2775: mol_type = other DNA
atgccgctgctgctactgctgcccctgctgtgggcaggggctctagcttcttctgagctcacccaagatcctgccgtgtctgtggctctgggccaaacagtgcggattacctgtcagggcgatagcctgagaagctactacgccagctggtaccaacagaagccaggacaggctcccgtgctcgtcatttatggcaagaacaacagaccatccggcatccccgatcggttttccggaagcagctctggcaatactgcctccctcaccatcactggcgcccaagcagaagatgaagcagactactattgtaactccagagacagctccggcaatcctcctgtggtgttcggaggcggaacaaaactcaccgtcctcggcagccggggtggaggtggaagcggcggtggtggctccggaggagggggtagcctcgagatggcacaggtccaactcgtggaatcaggaggtggacttgttcaccccggcggaagcctgagactgtcttgtgccgcttccggattcacattccggtcccactccatgaattgggtccgacaagctcccggcaaaggccttgaatgggtgtccagcatcagcagcgacagcacctacacctactatgccgacagcgtgaagggaaggttcacaatctctcgggacaacgccaagaacagcctgtacctgcagatgaactccctcagagccgaggatacagctgtgtattactgtgctagaagtggcggccagtggaagtactacgactactggggacaaggcacactcgtgacagttagctctgaggccgcagccaaagaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcggatctagaggtggcggaggatctggcggcggaggaagcggaggcggcggatctcttgaaatggctcagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctggagtctaaatacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctaga

SEQ ID NO: 106 - DNA, 2775 bp, Synthetic construct
  misc_feature 1..2775: note = CAR 2 (nt)
  source 1..2775: mol_type = other DNA
atgccgctgctgctactgctgcccctgctgtgggcaggggctctagctcaggtccaactcgtggaatcaggaggtggacttgttcaccccggcggaagcctgagactgtcttgtgccgcttccggattcacattccggtcccactccatgaattgggtccgacaagctcccggcaaaggccttgaatgggtgtccagcatcagcagcgacagcacctacacctactatgccgacagcgtgaagggaaggttcacaatctctcgggacaacgccaagaacagcctgtacctgcagatgaactccctcagagccgaggatacagctgtgtattactgtgctagaagtggcggccagtggaagtactacgactactggggacaaggcacactcgtgacagttagctctggcagccggggtggaggtggaagcggcggtggtggctccggaggagggggtagcctcgagatggcatcttctgagctcacccaagatcctgccgtgtctgtggctctgggccaaacagtgcggattacctgtcagggcgatagcctgagaagctactacgccagctggtaccaacagaagccaggacaggctcccgtgctcgtcatttatggcaagaacaacagaccatccggcatccccgatcggttttccggaagcagctctggcaatactgcctccctcaccatcactggcgcccaagcagaagatgaagcagactactattgtaactccagagacagctccggcaatcctcctgtggtgttcggaggcggaacaaaactcaccgtcctcggtggcggaggatctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcggatctagaggtggcggaggatctggcggcggaggaagcggaggcggcggatctcttgaaatggctcagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctggagtctaaatacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctaga

SEQ ID NO: 107 - DNA, 2820 bp, Synthetic construct
  misc_feature 1..2820: note = CAR 3 (nt)
  source 1..2820: mol_type = other DNA
atgccgctgctgctactgctgcccctgctgtgggcaggggctctagcttcttctgagctcacccaagatcctgccgtgtctgtggctctgggccaaacagtgcggattacctgtcagggcgatagcctgagaagctactacgccagctggtaccaacagaagccaggacaggctcccgtgctcgtcatttatggcaagaacaacagaccatccggcatccccgatcggttttccggaagcagctctggcaatactgcctccctcaccatcactggcgcccaagcagaagatgaagcagactactattgtaactccagagacagctccggcaatcctcctgtggtgttcggaggcggaacaaaactcaccgtcctcggcagccggggtggaggtggaagcggcggtggtggctccggaggagggggtagcctcgagatggcacaggtccaactcgtggaatcaggaggtggacttgttcaccccggcggaagcctgagactgtcttgtgccgcttccggattcacattccggtcccactccatgaattgggtccgacaagctcccggcaaaggccttgaatgggtgtccagcatcagcagcgacagcacctacacctactatgccgacagcgtgaagggaaggttcacaatctctcgggacaacgccaagaacagcctgtacctgcagatgaactccctcagagccgaggatacagctgtgtattactgtgctagaagtggcggccagtggaagtactacgactactggggacaaggcacactcgtgacagttagctctggtggaggaggtagtggtggcggtggatcaggtggcggcggcagtggtggcggaggatctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcggatctagaggtggcggaggatctggcggcggaggaagcggaggcggcggatctcttgaaatggctcagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctggagtctaaatacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctaga

SEQ ID NO: 108 - DNA, 2820 bp, Synthetic construct
  misc_feature 1..2820: note = CAR 4 (nt)
  source 1..2820: mol_type = other DNA
atgccgctgctgctactgctgcccctgctgtgggcaggggctctagctcaggtccaactcgtggaatcaggaggtggacttgttcaccccggcggaagcctgagactgtcttgtgccgcttccggattcacattccggtcccactccatgaattgggtccgacaagctcccggcaaaggccttgaatgggtgtccagcatcagcagcgacagcacctacacctactatgccgacagcgtgaagggaaggttcacaatctctcgggacaacgccaagaacagcctgtacctgcagatgaactccctcagagccgaggatacagctgtgtattactgtgctagaagtggcggccagtggaagtactacgactactggggacaaggcacactcgtgacagttagctctggcagccggggtggaggtggaagcggcggtggtggctccggaggagggggtagcctcgagatggcatcttctgagctcacccaagatcctgccgtgtctgtggctctgggccaaacagtgcggattacctgtcagggcgatagcctgagaagctactacgccagctggtaccaacagaagccaggacaggctcccgtgctcgtcatttatggcaagaacaacagaccatccggcatccccgatcggttttccggaagcagctctggcaatactgcctccctcaccatcactggcgcccaagcagaagatgaagcagactactattgtaactccagagacagctccggcaatcctcctgtggtgttcggaggcggaacaaaactcaccgtcctcggcggtggcggatctggtggaggaggtagcggtggtggtggatctggtggcggaggatctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcggatctagaggtggcggaggatctggcggcggaggaagcggaggcggcggatctcttgaaatggctcagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctggagtctaaatacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctaga

SEQ ID NO: 109 - DNA, 2724 bp, Synthetic construct
  misc_feature 1..2724: note = CAR 8 (nt)
  source 1..2724: mol_type = other DNA
atgccgctgctgctactgctgcccctgctgtgggcaggggctctagctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgaggccgcagccaaatcttctgagctcacccaagatcctgccgtgtctgtggctctgggccaaacagtgcggattacctgtcagggcgatagcctgagaagctactacgccagctggtaccaacagaagccaggacaggctcccgtgctcgtcatttatggcaagaacaacagaccatccggcatccccgatcggttttccggaagcagctctggcaatactgcctccctcaccatcactggcgcccaagcagaagatgaagcagactactattgtaactccagagacagctccggcaatcctcctgtggtgttcggaggcggaacaaaactcaccgtcctcggcagccggggtggaggtggaagcggcggtggtggctccggaggagggggtagcctcgagatggcacaggtccaactcgtggaatcaggaggtggacttgttcaccccggcggaagcctgagactgtcttgtgccgcttccggattcacattccggtcccactccatgaattgggtccgacaagctcccggcaaaggccttgaatgggtgtccagcatcagcagcgacagcacctacacctactatgccgacagcgtgaagggaaggttcacaatctctcgggacaacgccaagaacagcctgtacctgcagatgaactccctcagagccgaggatacagctgtgtattactgtgctagaagtggcggccagtggaagtactacgactactggggacaaggcacactcgtgacagttagctctgaggccgcagccaaacagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctggagtctaaatacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctaga

SEQ ID NO: 110 - DNA, 2712 bp, Synthetic construct
  misc_feature 1..2712: note = CAR 9 (nt)
  source 1..2712: mol_type = other DNA
atgccgctgctgctactgctgcccctgctgtgggcaggggctctagctcagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctggaggccgcagccaaacaggtccaactcgtggaatcaggaggtggacttgttcaccccggcggaagcctgagactgtcttgtgccgcttccggattcacattccggtcccactccatgaattgggtccgacaagctcccggcaaaggccttgaatgggtgtccagcatcagcagcgacagcacctacacctactatgccgacagcgtgaagggaaggttcacaatctctcgggacaacgccaagaacagcctgtacctgcagatgaactccctcagagccgaggatacagctgtgtattactgtgctagaagtggcggccagtggaagtactacgactactggggacaaggcacactcgtgacagttagctctggcagcacctccggcagcggcaagcctggcagcggcgagggcagcaccaagggctcttctgagctcacccaagatcctgccgtgtctgtggctctgggccaaacagtgcggattacctgtcagggcgatagcctgagaagctactacgccagctggtaccaacagaagccaggacaggctcccgtgctcgtcatttatggcaagaacaacagaccatccggcatccccgatcggttttccggaagcagctctggcaatactgcctccctcaccatcactggcgcccaagcagaagatgaagcagactactattgtaactccagagacagctccggcaatcctcctgtggtgttcggaggcggaacaaaactcaccgtcctcgaggccgcagccaaagaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctaga

SEQ ID NO: 111 - DNA, 2724 bp, Synthetic construct
  misc_feature 1..2724: note = CAR 5 (nt)
  source 1..2724: mol_type = other DNA
atgccgctgctgctactgctgcccctgctgtgggcaggggctctagctcaggtccaactcgtggaatcaggaggtggacttgttcaccccggcggaagcctgagactgtcttgtgccgcttccggattcacattccggtcccactccatgaattgggtccgacaagctcccggcaaaggccttgaatgggtgtccagcatcagcagcgacagcacctacacctactatgccgacagcgtgaagggaaggttcacaatctctcgggacaacgccaagaacagcctgtacctgcagatgaactccctcagagccgaggatacagctgtgtattactgtgctagaagtggcggccagtggaagtactacgactactggggacaaggcacactcgtgacagttagctctggtggcggaggatctcagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcggtggcggaggatcttcttctgagctcacccaagatcctgccgtgtctgtggctctgggccaaacagtgcggattacctgtcagggcgatagcctgagaagctactacgccagctggtaccaacagaagccaggacaggctcccgtgctcgtcatttatggcaagaacaacagaccatccggcatccccgatcggttttccggaagcagctctggcaatactgcctccctcaccatcactggcgcccaagcagaagatgaagcagactactattgtaactccagagacagctccggcaatcctcctgtggtgttcggaggcggaacaaaactcaccgtcctcgagtctaaatacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctaga

SEQ ID NO: 112 - DNA, 2724 bp, Synthetic construct
  misc_feature 1..2724: note = CAR 6 (nt)
  source 1..2724: mol_type = other DNA
atgccgctgctgctactgctgcccctgctgtgggcaggggctctagcttcttctgagctcacccaagatcctgccgtgtctgtggctctgggccaaacagtgcggattacctgtcagggcgatagcctgagaagctactacgccagctggtaccaacagaagccaggacaggctcccgtgctcgtcatttatggcaagaacaacagaccatccggcatccccgatcggttttccggaagcagctctggcaatactgcctccctcaccatcactggcgcccaagcagaagatgaagcagactactattgtaactccagagacagctccggcaatcctcctgtggtgttcggaggcggaacaaaactcaccgtcctcggtggcggaggatctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcggatctagaggtggcggaggatctggcggcggaggaagcggaggcggcggatctcttgaaatggctcagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggtggcggaggatctcaggtccaactcgtggaatcaggaggtggacttgttcaccccggcggaagcctgagactgtcttgtgccgcttccggattcacattccggtcccactccatgaattgggtccgacaagctcccggcaaaggccttgaatgggtgtccagcatcagcagcgacagcacctacacctactatgccgacagcgtgaagggaaggttcacaatctctcgggacaacgccaagaacagcctgtacctgcagatgaactccctcagagccgaggatacagctgtgtattactgtgctagaagtggcggccagtggaagtactacgactactggggacaaggcacactcgtgacagttagctctgagtctaaatacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctaga

SEQ ID NO: 113 - DNA, 2724 bp, Synthetic construct
  misc_feature 1..2724: note = CAR 10 (nt)
  source 1..2724: mol_type = other DNA
atgccgctgctgctactgctgcccctgctgtgggcaggggctctagctcagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggtggcggaggatctcaggtccaactcgtggaatcaggaggtggacttgttcaccccggcggaagcctgagactgtcttgtgccgcttccggattcacattccggtcccactccatgaattgggtccgacaagctcccggcaaaggccttgaatgggtgtccagcatcagcagcgacagcacctacacctactatgccgacagcgtgaagggaaggttcacaatctctcgggacaacgccaagaacagcctgtacctgcagatgaactccctcagagccgaggatacagctgtgtattactgtgctagaagtggcggccagtggaagtactacgactactggggacaaggcacactcgtgacagttagctctggcagccggggtggaggtggaagcggcggtggtggctccggaggagggggtagcctcgagatggcatcttctgagctcacccaagatcctgccgtgtctgtggctctgggccaaacagtgcggattacctgtcagggcgatagcctgagaagctactacgccagctggtaccaacagaagccaggacaggctcccgtgctcgtcatttatggcaagaacaacagaccatccggcatccccgatcggttttccggaagcagctctggcaatactgcctccctcaccatcactggcgcccaagcagaagatgaagcagactactattgtaactccagagacagctccggcaatcctcctgtggtgttcggaggcggaacaaaactcaccgtcctcggtggcggaggatctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctaga

SEQ ID NO: 114 - DNA, 2724 bp, Synthetic construct
  misc_feature 1..2724: note = CAR 11 (nt)
  source 1..2724: mol_type = other DNA
atgccgctgctgctactgctgcccctgctgtgggcaggggctctagctcagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggtggcggaggatcttcttctgagctcacccaagatcctgccgtgtctgtggctctgggccaaacagtgcggattacctgtcagggcgatagcctgagaagctactacgccagctggtaccaacagaagccaggacaggctcccgtgctcgtcatttatggcaagaacaacagaccatccggcatccccgatcggttttccggaagcagctctggcaatactgcctccctcaccatcactggcgcccaagcagaagatgaagcagactactattgtaactccagagacagctccggcaatcctcctgtggtgttcggaggcggaacaaaactcaccgtcctcggcagccggggtggaggtggaagcggcggtggtggctccggaggagggggtagcctcgagatggcacaggtccaactcgtggaatcaggaggtggacttgttcaccccggcggaagcctgagactgtcttgtgccgcttccggattcacattccggtcccactccatgaattgggtccgacaagctcccggcaaaggccttgaatgggtgtccagcatcagcagcgacagcacctacacctactatgccgacagcgtgaagggaaggttcacaatctctcgggacaacgccaagaacagcctgtacctgcagatgaactccctcagagccgaggatacagctgtgtattactgtgctagaagtggcggccagtggaagtactacgactactggggacaaggcacactcgtgacagttagctctggtggcggaggatctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctaga

SEQ ID NO: 115 - DNA, 2754 bp, Synthetic construct
  misc_feature 1..2754: note = CAR 7 (nt)
  source 1..2754: mol_type = other DNA
atgccgctgctgctactgctgcccctgctgtgggcaggggctctagctcaggtccaactcgtggaatcaggaggtggacttgttcaccccggcggaagcctgagactgtcttgtgccgcttccggattcacattccggtcccactccatgaattgggtccgacaagctcccggcaaaggccttgaatgggtgtccagcatcagcagcgacagcacctacacctactatgccgacagcgtgaagggaaggttcacaatctctcgggacaacgccaagaacagcctgtacctgcagatgaactccctcagagccgaggatacagctgtgtattactgtgctagaagtggcggccagtggaagtactacgactactggggacaaggcacactcgtgacagttagctctggtggaggtggaagcggcggtggtggctcccagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcggtggaggtggaagcggcggtggtggctcctcttctgagctcacccaagatcctgccgtgtctgtggctctgggccaaacagtgcggattacctgtcagggcgatagcctgagaagctactacgccagctggtaccaacagaagccaggacaggctcccgtgctcgtcatttatggcaagaacaacagaccatccggcatccccgatcggttttccggaagcagctctggcaatactgcctccctcaccatcactggcgcccaagcagaagatgaagcagactactattgtaactccagagacagctccggcaatcctcctgtggtgttcggaggcggaacaaaactcaccgtcctcgagtctaaatacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctaga

SEQ ID NO: 116 - DNA, 2754 bp, Synthetic construct
  misc_feature 1..2754: note = CAR 12 (nt)
  source 1..2754: mol_type = other DNA
atgccgctgctgctactgctgcccctgctgtgggcaggggctctagctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcggtggaggtggaagcggcggtggtggctcccaggtccaactcgtggaatcaggaggtggacttgttcaccccggcggaagcctgagactgtcttgtgccgcttccggattcacattccggtcccactccatgaattgggtccgacaagctcccggcaaaggccttgaatgggtgtccagcatcagcagcgacagcacctacacctactatgccgacagcgtgaagggaaggttcacaatctctcgggacaacgccaagaacagcctgtacctgcagatgaactccctcagagccgaggatacagctgtgtattactgtgctagaagtggcggccagtggaagtactacgactactggggacaaggcacactcgtgacagttagctctggcagccggggtggaggtggaagcggcggtggtggctccggaggagggggtagcctcgagatggcatcttctgagctcacccaagatcctgccgtgtctgtggctctgggccaaacagtgcggattacctgtcagggcgatagcctgagaagctactacgccagctggtaccaacagaagccaggacaggctcccgtgctcgtcatttatggcaagaacaacagaccatccggcatccccgatcggttttccggaagcagctctggcaatactgcctccctcaccatcactggcgcccaagcagaagatgaagcagactactattgtaactccagagacagctccggcaatcctcctgtggtgttcggaggcggaacaaaactcaccgtcctcggtggaggtggaagcggcggtggtggctcccagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctggagtctaaatacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctaga

SEQ ID NO: 117 - DNA, 2754 bp, Synthetic construct
  misc_feature 1..2754: note = CAR 13 (nt)
  source 1..2754: mol_type = other DNA
atgccgctgctgctactgctgcccctgctgtgggcaggggctctagctcagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggtggaggtggaagcggcggtggtggctcccaggtccaactcgtggaatcaggaggtggacttgttcaccccggcggaagcctgagactgtcttgtgccgcttccggattcacattccggtcccactccatgaattgggtccgacaagctcccggcaaaggccttgaatgggtgtccagcatcagcagcgacagcacctacacctactatgccgacagcgtgaagggaaggttcacaatctctcgggacaacgccaagaacagcctgtacctgcagatgaactccctcagagccgaggatacagctgtgtattactgtgctagaagtggcggccagtggaagtactacgactactggggacaaggcacactcgtgacagttagctctggcagccggggtggaggtggaagcggcggtggtggctccggaggagggggtagcctcgagatggcatcttctgagctcacccaagatcctgccgtgtctgtggctctgggccaaacagtgcggattacctgtcagggcgatagcctgagaagctactacgccagctggtaccaacagaagccaggacaggctcccgtgctcgtcatttatggcaagaacaacagaccatccggcatccccgatcggttttccggaagcagctctggcaatactgcctccctcaccatcactggcgcccaagcagaagatgaagcagactactattgtaactccagagacagctccggcaatcctcctgtggtgttcggaggcggaacaaaactcaccgtcctcggtggaggtggaagcggcggtggtggctccgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctaga

SEQ ID NO: 118 - DNA, 2742 bp, Synthetic construct
  misc_feature 1..2742: note = CAR 14 (nt)
  source 1..2742: mol_type = other DNA
atgccgctgctgctactgctgcccctgctgtgggcaggggctctagctcagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggtggaggtggaagcggcggtggtggctcccaggtccaactcgtggaatcaggaggtggacttgttcaccccggcggaagcctgagactgtcttgtgccgcttccggattcacattccggtcccactccatgaattgggtccgacaagctcccggcaaaggccttgaatgggtgtccagcatcagcagcgacagcacctacacctactatgccgacagcgtgaagggaaggttcacaatctctcgggacaacgccaagaacagcctgtacctgcagatgaactccctcagagccgaggatacagctgtgtattactgtgctagaagtggcggccagtggaagtactacgactactggggacaaggcacactcgtgacagttagctctggcagcacctccggcagcggcaagcctggcagcggcgagggcagcaccaagggctcttctgagctcacccaagatcctgccgtgtctgtggctctgggccaaacagtgcggattacctgtcagggcgatagcctgagaagctactacgccagctggtaccaacagaagccaggacaggctcccgtgctcgtcatttatggcaagaacaacagaccatccggcatccccgatcggttttccggaagcagctctggcaatactgcctccctcaccatcactggcgcccaagcagaagatgaagcagactactattgtaactccagagacagctccggcaatcctcctgtggtgttcggaggcggaacaaaactcaccgtcctcggtggaggtggaagcggcggtggtggctccgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctaga

SEQ ID NO: 119 - DNA, 2724 bp, Synthetic construct
  misc_feature 1..2724: note = CAR 5 (nt) CO/SSE
  source 1..2724: mol_type = other DNA
atgcctctgctgctgcttctgcctcttctttgggctggtgctctggctcaggtgcagctggttgaatctggcggaggactggttcaccctggcggatctctgagactgtcttgtgccgccagcggcttcaccttccgatcccactccatgaactgggtccgacaggcccctggcaaaggccttgaatgggtgtccagcatcagcagcgacagcacctacacctactacgccgacagcgtgaagggcagattcaccatctccagagacaacgccaagaacagcctgtacctgcagatgaactccctgagagccgaggacaccgccgtgtactactgtgctagaagcggcggacagtggaagtactacgactattggggccagggcaccctggtcacagtttctagcggaggcggaggctctcagtctgctctgacacagcctgcctccgtgtctgcttctcctggacagtctatcgccatctcctgtaccggcaccagctctgacgttggctggtatcagcagcacccaggcaaggcccctaagctgatgatctacgaggacagcaagaggcccagcggcgtgtccaatcgattcagcggcagcaagagcggcaacaccgccagcctgacaattagcggactgcaggccgaggacgaggccgattactactgcagcagcaacacccggtccagcacactggtttttggcggaggcaccaagctgacagtgctgggaagtagaggcggcggaggtagtggtggtggcggtagtggtggcggaggatctcttgaaatggccgaggttcagctggtgcagtctggcgccgagatgaagaaacctggcgcctctctgaagctgagctgcaaggcctctggctacaccttcatcgactactacgtgtactggatgcggcaggccccaggacagggactcgaatctatgggctggatcaaccccaatagcggcggcaccaattacgcccagaaattccagggcagagtgaccatgaccagggacaccagcatctccaccgcctacatggaactgagccggctgagatccgacgatacagccatgtactattgcgccagaagccagcgcgacggctacatggattattggggacaaggcacactcgtgaccgtgtcaagtggcggtggtggatctagcagcgagctgacacaggatccagccgtgtctgttgccctgggccagacagtgcggattacctgtcaaggcgacagcctgcggagctactatgcctcctggtatcaacagaagcccggccaggctcctgtgctggtcatctacggcaagaacaacagacccagcggcatccccgatagattctccggctctagctctggcaacacagcttccctgacaatcactggcgcccaggccgaagatgaagccgactattactgtaacagccgggacagcagcggcaaccctcctgttgtttttggaggcggaacaaaactgaccgtgctggagtctaaatacggaccgccttgtcctccatgtcctgctcctcccgtggctggcccttccgtgtttctgttccctccaaagcctaaggacaccctgatgatcagcagaacccctgaagtgacctgcgtggtggtcgacgtgtcccaagaggatcctgaggtgcagttcaactggtatgtggacggcgtggaagtgcacaatgccaagaccaagcctagagaggaacagttccagagcacctatagagtggtgtccgtgctgaccgtgctgcaccaggattggctgaacggcaaagagtacaagtgcaaggtgtccaacaagggactgcccagcagcatcgagaaaaccatctccaaggccaagggccagcctagggaaccccaggtttacacactgcctccaagccaagaggaaatgaccaagaaccaggtgtccctgacctgcctggtcaagggcttctacccttccgatatcgccgtggaatgggagagcaatggccagccagagaacaactacaagaccacacctcctgtgctggatagcgacggctcattcttcctgtactcccgactgaccgtggacaagagcagatggcaagagggcaatgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgccgattccccgaggaagaagaaggcggctgcgagctgagagtgaagttcagcagatccgctgacgcccctgcttaccagcagggccagaaccagctgtataacgagctgaatctggggcgcagagaagagtacgacgtgctggacaagcggagaggcagagatcctgagatgggcggcaagcccagacggaagaatcctcaagagggcctctacaacgagctgcagaaagacaagatggccgaggcctacagcgagatcggaatgaagggcgaacgcagaagaggaaagggccacgacggactgtatcagggactgagcaccgccaccaaggatacctatgacgcactgcacatgcaggccctgccacctaga

SEQ ID NO: 120 - DNA, 2724 bp, Synthetic construct
  misc_feature 1..2724: note = CAR 11 (nt) CO/SSE
  source 1..2724: mol_type = other DNA
atgcctttgttgctgctgctgcctcttctgtgggctggtgctctggctcagtctgctctgacacagcctgcctccgtgtctgcttctcctggacagtctatcgccatcagctgtaccggcaccagctctgacgttggctggtatcagcagcaccctggcaaggcccctaagctgatgatctacgaggacagcaagaggcccagcggcgtgtccaatcgcttcagcggcagcaagagcggcaacaccgccagcctgacaattagcggactgcaggccgaggacgaggccgattactactgcagctccaacaccagatccagcacactggtgtttggcggaggcaccaagctgacagttcttggcggcggaggaagcagctccgagctgacacaagatcctgccgtgtctgtggccctgggccagacagttagaatcacctgtcagggcgacagcctgcggagctactacgcctcttggtatcaacagaagcccggacaggcccctgtgctggtcatctacggcaagaacaacagacccagcggcatccccgatagattctccggctctagctctggcaataccgcctctctgacaatcactggcgcccaggccgaagatgaagccgactattactgtaacagccgggacagcagcggcaaccctcctgttgtttttggaggcggaacaaagctgaccgtgctgggctctagaggtggcggaggtagcggaggcggaggatctggcggtggtggatctctggaaatggctcaggtgcagctggtggaatctggtggcggacttgttcaccctggcggaagcctgagactgtcttgtgccgccagcggcttcaccttccggtcccactccatgaactgggtccgacaggctcctggcaaaggcctggaatgggtgtccagcatcagcagcgatagcacctacacctactatgccgacagcgtgaagggcagattcaccatcagcagagacaacgccaagaatagcctgtacctgcagatgaacagcctgagagccgaggacaccgccgtgtactactgtgctagaagcggcggacagtggaagtactacgactattggggccagggcaccctggtcacagtttctagtggtggtggcggcagcgaagtccagctggttcaatctggcgccgagatgaagaagcctggcgcttctctgaagctgagctgcaaggcctccggctacacctttatcgactactacgtgtactggatgcggcaggccccaggacagggactcgaatctatgggctggatcaaccccaatagcggcggcaccaattacgcccagaaattccagggcagagtgaccatgaccagggacaccagcatctccaccgcctacatggaactgagccggctgagatccgacgatacagccatgtactattgcgccagaagccagcgcgacggctacatggattattggggacaaggcacactcgtgaccgtgtcctctgagtctaaatacggaccgccttgtcctccatgtcctgctcctcccgtggctggcccttccgtgtttctgttccctccaaagcctaaggacaccctgatgatcagcagaacccctgaagtgacctgcgtggtggtcgacgtgtcccaagaggatcctgaggtgcagttcaactggtatgtggacggcgtggaagtgcacaatgccaagaccaagcctagagaggaacagttccagagcacctatagagtggtgtccgtgctgaccgtgctgcaccaggattggctgaacggcaaagagtacaagtgcaaggtgtccaacaagggactgcccagcagcatcgagaaaaccatctccaaggccaagggccagcctagggaaccccaggtttacacactgcctccaagccaagaggaaatgaccaagaaccaggtgtccctgacctgcctggtcaagggcttctacccttccgatatcgccgtggaatgggagagcaatggccagccagagaacaactacaagaccacacctcctgtgctggatagcgacggctcattcttcctgtactcccgactgaccgtggacaagagcagatggcaagagggcaatgtgttcagctgcagcgtgatgcacgaggccctgcacaaccactacacccagaagtctctgagcctgagcctgggcaagatgttctgggtgctcgttgttgttggcggcgtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgccgattccccgaggaagaagaaggcggctgcgagctgagagtgaagttcagcagatccgctgacgcccctgcttaccagcagggccagaaccagctgtataacgagctgaatctggggcgcagagaagagtacgacgtgctggacaagcggagaggcagagatcctgagatgggcggcaagcccagacggaagaatcctcaagagggcctctacaacgagctgcagaaagacaagatggccgaggcctacagcgagatcggaatgaagggcgaacgcagaagaggaaagggccacgacggactgtatcagggactgagcaccgccaccaaggatacctatgacgcactgcacatgcaggccctgccacctaga